(19) United States
(12) Patent Application Publication
(10) Pub. No.: US 2024/0051985 A1
(43) Pub. Date: Feb. 15, 2024

(54) METTL3 MODULATORS

(71) Applicant: ACCENT THERAPEUTICS, INC., Lexington, MA (US)

(72) Inventors: Matthew H. Daniels, Somerville, MA (US); Thomas Andrew Wynn, Lexington, MA (US); Brian Andrew Sparling, Melrose, MA (US); Ernest Allen Sickmier, Needham, MA (US); Andrew Stewart Tasker, Simi Valley, CA (US); James Edward John Mills, Sheffield (GB)

(21) Appl. No.: 18/031,920

(22) PCT Filed: Oct. 13, 2021
(86) PCT No.: PCT/US2021/054822

§ 371 Date: Apr. 14, 2023

Related U.S. Application Data
(60) Provisional application No. 63/091,529, filed on Oct. 14, 2020.

(51) Int. Cl. C07H 19/23; A61P 35/00

(52) U.S. Cl. CPC C07H 19/23; A61P 35/00

(57) ABSTRACT

Provided are compounds of Formula (I′) or (I) below, or pharmaceutically acceptable salts thereof, and methods for their use and production.

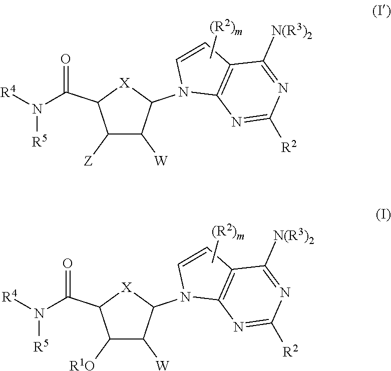

### RELATED APPLICATION

This application claims the benefit of the filing date, under 35 U.S.C. § 119(e), of U.S. Provisional Application No. 63/091,529, filed on Oct. 14, 2020, the entire contents of which are incorporated herein by reference.

### TECHNICAL FIELD

This invention relates to compounds that are METTL3 modulating agents, and methods of making and using such compounds.

### BACKGROUND

Among all RNA modifications, N⁶-methyladenosine (m⁶A) is the most abundant mRNA internal modification. It plays important roles in the biogenesis and functions of RNA. m6A deposition on mRNA is regulated by the dynamic interplay between RNA specific methylase (“writers”), binding proteins (“readers”), and demethylases (“erasers”) (Ying Yang, Cell Research volume 28, pages 616-624, 2018). m⁶A methylation is controlled by a large RNA methyltransferase complex (MTase), composed of the methyltransferase-like 3 and 14 (METTL3 and METTL14) proteins and their cofactor, Wilms' tumor 1-associated protein (WTAP). METTL3 is the catalytic component that forms a heterodimer with METTL14, which facilitates the interactions with its target mRNA.

METTL3 has been demonstrated to modulate embryonic development, cell reprogramming, spermatogenesis, regulation of T cell homeostasis and endothelial-to-hematopoietic transition via methylation of specific target transcripts. Aberrant METTL3 expression has been associated with various pathophysiology, such as cancer, obesity, infection, inflammation and immune response (Sibbritt et al., 2013). AML is one of the cancers with the highest expression of both METTL3 and METTL14. Both genes were found upregulated in all subtypes of AML compared to normal hematopoietic cells.

Despite recent advances in METTL3 research, there is still a great need for small molecule METTL3 inhibitors as potential therapeutic agent for treating diseases that are responsive to modulation of METTL3 activities.

### SUMMARY

In accordance with the purpose(s) of the invention, as embodied and broadly described herein, the invention, in an aspect, relates to compounds useful as METTL3 modulators, pharmaceutical compositions, methods of making and methods of treating disorders using the same. In some embodiments, the compounds of the invention are METTL3 inhibitors.

In a first aspect, the present invention provides a compound of formula (I′):

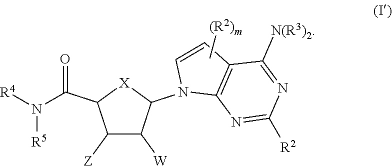

or a pharmaceutically acceptable salt thereof, wherein:
X is selected from O and CH₂;
R¹ is selected from H, C₁₋₆alkyl and —C(═O)—C₁₋₆alkyl;
Z is H and W is —OR¹, or Z is —OR¹ and W is selected from H, halo, —OR¹, C₁₋₆alkyl and —NH₂,
R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl, 5 to 6-membered heteroaryl, halo, —CN, —OR^{2a}, N(R^{2a})₂, and —C(═O)N(R^{2a})₂, wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, —C₁₋₆alkyl-C₁₋₃alkoxy, C₁₋₆haloalkyl, C₃₋₈cycloalkyl, 4 to 7-membered heterocycloalkyl, phenyl, 5- to 6-membered heteroaryl, halo, —CN, —OR^{2a}, —C(═O)N(R^{2a})₂, and —N(R^{2a})₂;
R^{2a}, for each occurrence, is independently selected from H, C₁₋₆alkyl, C₃₋₈cycloalkyl, and 4 to 6-membered heterocycloalkyl;
R³, for each occurrence, is H or C₁₋₆alkyl optionally substituted with 1 to 3 substituents independently selected from C₃₋₆cycloalkyl, phenyl and halo;
R⁴ is H, C₁₋₆alkyl, C₃₋₈cycloalkyl, 4 to 6-membered heterocycloalkyl or 5 to 6-membered heteroaryl, wherein the C₁₋₆alkyl, C₃₋₈cycloalkyl, 4 to 6-membered heterocycloalkyl and 5 to 6-membered heteroaryl represented by R⁴ are each optionally substituted with 1 to 4 substituents independently selected from C₁₋₆alkyl, —CN, —N(R^{4a})₂, —OR^{4a}, and —C(O)OR^{4a};
R^{4a} is H or C₁₋₄alkyl optionally substituted with —OH or C₁₋₆alkoxy,
R⁵ is H, C₁₋₆alkyl, ring A, or —C₁₋₆alkylene-ring A, each of which is optionally substituted with 1 to 4 R⁶;
or R⁴ and R⁵ together with the N atom from which they are attached form a 4 to 10-membered heterocycloalkyl optionally containing an additional heteroatom selected from 0, N and S, wherein the heterocycloalkyl is optionally substituted with 1 to 3 R⁶; or the heterocycloalkyl is optionally fused with a phenyl or a 5 to 6-membered heteroaryl;
ring A is C₃₋₈cycloalkyl, phenyl, 4 to 6-membered heterocycloalkyl, 7 to 10-membered spiro or bridged bicyclic heterocycloalkyl, 5 to 6-membered heteroaryl, or 8 to 10-membered bicyclic heteroaryl, each of which is optionally substituted with 1 to 4 R⁶;
R⁶, for each occurrence, is independently C₁₋₆alkyl, C₃₋₈cycloalkyl, phenyl, 4 to 7-membered heterocycloalkyl, 5 to 6-membered heteroaryl, halo, oxo, —CN, —N(R^{6a})₂, —OR^{6a}, —C(═O)R^{6a}, —C(═O)N(R^{6a})₂, —S(═O)₂R^{6a}, —S(═O)₂N(R^{6a})₂, —NR^{6a}C(═O)R^{6a}, —NR^{6a}C(═O)OR^{6a}, —NR^{6a}C(═O)N(R^{6a})₂, —NR^{6a}S(═O)₂R^{6a}, —C(═O)OR^{6a}, wherein the C₁₋₆alkyl, C₃₋₈cycloalkyl, 4 to 7-membered heterocycloalkyl, 5 to 6-membered heteroaryl and phenyl represented by R⁶ are each optionally substituted with 1 to 3 substituents independently selected from C₁₋₆alkyl, C₃₋₅cycloalkyl, 5 to 6-membered heterocycloalkyl optionally substituted with 1 to 2 oxo, phenyl, halo, —OR^{6a}, —N(R^{6a})₂, and —C(═O)N(R^{6a})₂, wherein the C₁₋₆alkyl is optionally substituted with 1 to 3 substitutents independently selected from halo and OH;
or two R⁶ together with the intervening atoms on ring A form a phenyl, 5 to 6-membered heteroaryl, or 4 to 7-membered heterocycloalkyl fused with ring A, each of which is optionally substituted with 1 to 3 substituents independently selected from C₁₋₆alkyl, halo, oxo, —OR^{6a}, —N(R^{6a})₂, and —C(═O)N(R^{6a})₂;
R^{6a}, for each occurrence, is independently selected from H, C₁₋₆alkyl, C₃₋₈cycloalkyl, 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆haloalkyl, C₁₋₆alkoxy, C₃₋₆cycloalkyl, 5 to 6-membered heterocycloalkyl, and phenyl; and
m is 1 or 2.

In a second aspect, the compound is represented by formula (I′), or a pharmaceutically acceptable salt thereof, wherein:
X is selected from O and CH₂;
R¹ is selected from H, C₁₋₆alkyl and —C(═O)—C₁₋₆alkyl;
Z is H and W is —OR¹; or Z is —OR¹, and W is selected from H, halo, —OR¹, C₁₋₆alkyl and —NH₂;
R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl, 5 to 6-membered heteroaryl, halo, —CN, —OR^{2a}, —N(R^{2a})₂, and —C(═O)N(R^{2a})₂, wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, —C₁₋₆alkyl-C₁₋₃alkoxy, C₁₋₆haloalkyl, C₃₋₈cycloalkyl, 4 to 7-membered heterocycloalkyl, phenyl, 5- to 6-membered heteroaryl, halo, —CN, —OR^{2a}, —C(═O)N(R^{2a})₂, and —N(R^{2a})₂;
R^{2a}, for each occurrence, is independently selected from H, C₁₋₆alkyl, C₃₋₈cycloalkyl, and 4 to 6-membered heterocycloalkyl;
R³, for each occurrence, is H or C₁₋₆alkyl optionally substituted with 1 to 3 substituents independently selected from C₃₋₆cycloalkyl, phenyl and halo;
R⁴ is H, C₁₋₆alkyl, C₃₋₈cycloalkyl, 4 to 6-membered heterocycloalkyl or 5 to 6-membered heteroaryl, wherein the C₁₋₆alkyl, C₃₋₈cycloalkyl, 4 to 6-membered heterocycloalkyl and 5 to 6-membered heteroaryl represented by R⁴ are each optionally substituted with 1 to 4 substituents independently selected from C₁₋₆alkyl, —CN, —N(R^{4a})₂, —OR^{4a}, and —C(O)OR^{4a};
R^{4a} is H or C₁₋₄alkyl optionally substituted with —OH or C₁₋₆alkoxy,
R⁵ is C₁₋₆alkyl, ring A, or —C₁₋₆alkylene-ring A, each of which is optionally substituted with 1 to 4 R⁶;
or R⁴ and R⁵ together with the N atom from which they are attached form a 4 to 10-membered heterocycloalkyl optionally containing an additional heteroatom selected from O, N and S, wherein the heterocycloalkyl is optionally substituted with 1 to 3 R⁶;
ring A is C₃₋₈cycloalkyl, phenyl, 4 to 6-membered heterocycloalkyl, 5 to 6-membered heteroaryl, or 8- to 10-membered bicyclic heteroaryl, each of which is optionally substituted with 1 to 4 R⁶;
R⁶, for each occurrence, is independently C₁₋₆alkyl, C₃₋₈cycloalkyl, phenyl, 4 to 7-membered heterocycloalkyl, 5 to 6-membered heteroaryl, halo, oxo, —CN, —N(R^{6a})₂, —OR^{6a}, —C(═O)R^{6a}, —C(═O)N(R^{6a})₂, —S(═O)₂R^{6a}, —S(═O)₂N(R^{6a})₂, —NR^{6a}C(═O)R^{6a}, —NR^{6a}C(═O)NR^{6a}, —NR^{6a}S(═O)₂R^{6a}, —C(═O)OR^{6a}, wherein the C₁₋₆alkyl, C₃₋₈cycloalkyl, 4 to 7-membered heterocycloalkyl, 5 to 6-membered heteroaryl and phenyl represented by R⁶ are each optionally substituted with 1 to 3 substituents independently selected from C₁₋₆alkyl, halo, —OR^{6a}, —N(R^{6a})₂, and —C(═O)N(R^{6a})₂, wherein the C₁₋₆alkyl is optionally substituted with 1 to 3 substitutents independently selected from halo and OH;
or two R⁶ together with the intervening atoms on ring A form a phenyl, 5 to 6-membered heteroaryl, or 4 to 6-membered heterocycloalkyl fused with ring A, each of which is optionally substituted with 1 to 3 substituents independently C₁₋₆alkyl, halo, —OR^{6a}, —N(R^{6a})₂, and —C(═O)N(R^{6a})₂;
R^{6a}, for each occurrence, is independently selected from H, C₁₋₆alkyl, C₃₋₈cycloalkyl, 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆haloalkyl, C₁₋₆alkoxy, C₃₋₆cycloalkyl; and
m is 1 or 2.

The present invention also provides a pharmaceutical composition comprising at least one compound described herein, or a pharmaceutically acceptable salt thereof, and at least one pharmaceutically acceptable excipient.

In one embodiment, the invention is a method of treating a disorder responsive to inhibition of METTL3 activity in a subject comprising administering to said subject an effective amount of at least one compound described herein, or a pharmaceutically acceptable salt thereof.

The present invention also includes the use of at least one compound described herein, or a pharmaceutically acceptable salt thereof, for the manufacture of a medicament for the treatment of a disorder responsive to inhibition of METTL3 activity. Also provided is a compound described herein, or a pharmaceutically acceptable salt thereof for use in treating a disorder responsive to inhibition of METTL3 activity.

Other features or advantages will be apparent from the following detailed description of several embodiments, and also from the appended claims.

### DETAILED DESCRIPTION

It has been found that the compounds of the present invention are useful as METTL3 inhibitors. The compounds according to the invention and compositions thereof, may be useful for the treatment of autoimmune diseases, cancer, inflammatory diseases, and infectious diseases, such as viral infections.

In a first embodiment of the present invention, the compound is represented by formula (I′), or a pharmaceutically acceptable salt thereof, wherein the definitions for the variables are as defined in the first aspect described above.

In a second embodiment of the present invention, the compound is represented by formula (I′), or a pharmaceutically acceptable salt thereof, wherein the definitions for the variables are as defined in the second aspect described above.

In a third embodiment of the present invention, the compound is represented by formula (I):

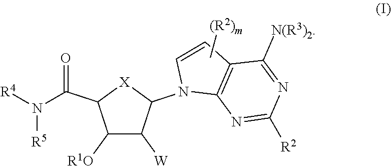

or a pharmaceutically acceptable salt thereof, wherein the definitions for the variables are as defined in the first or second embodiment.

In a fourth embodiment of the present invention, the compound is represented by formula (II):

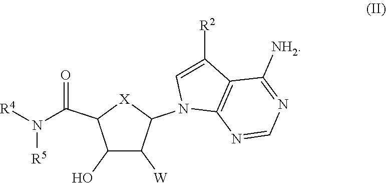

or a pharmaceutically acceptable salt thereof, wherein W is H, F or OH; and the definitions for the other variables are as defined in the third embodiment.

In a fifth embodiment of the present invention, the compound is represented by the following formula:

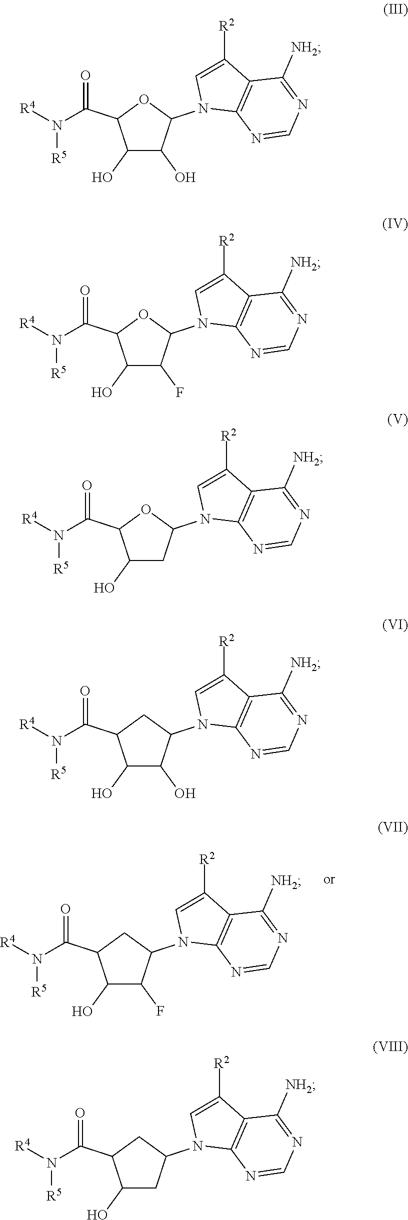

or a pharmaceutically acceptable salt thereof; and the definitions for the variables are as defined in the fourth embodiment.

In a sixth embodiment of the present invention, the compound is represented by the following formula:

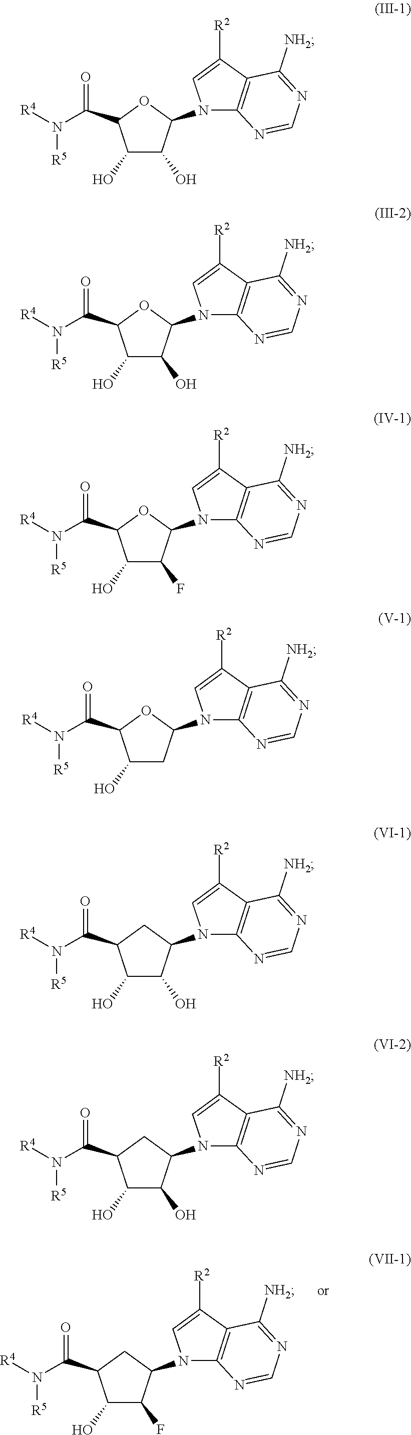

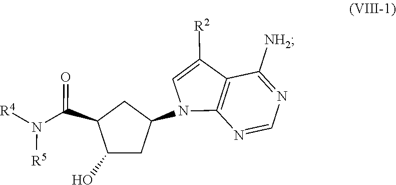

or a pharmaceutically acceptable salt thereof; and the definitions for the variables are as defined in the fifth embodiment.

In a seventh embodiment, the compound is represented by the following formula:

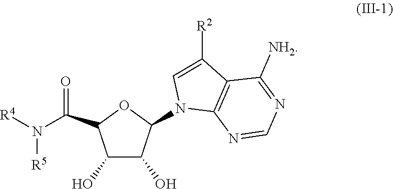

or a pharmaceutically acceptable salt thereof; and the definitions for the variables are as defined in the fifth embodiment.

In an eighth embodiment of the present invention, the compound is represented by formula (I′), (I), (II), (III), (IV), (V), (VI), (VII), (VIII), (III-1), (III-2), (IV-1), (V-1), (VI-1), (VI-2), (VII-1), or (VIII-1), or a pharmaceutically acceptable salt thereof, wherein R² is H, halo, —CN, C₁₋₆alkyl, C₃₋₈cycloalkyl, 4 to 6-membered heterocycloalkyl, or 5 to 6-membered heteroaryl, wherein the C₁₋₆alkyl, C₃₋₈cycloalkyl, 4 to 6-membered heterocycloalkyl, and 5 to 6-membered heteroaryl are each optionally substituted with 1 to 3 substituents independently selected from halo, C₁₋₄alkyl, C₁₋₄haloalkyl and C₃₋₆cycloalkyl; and the definitions for the other variables are as defined in the first, second, third, fourth, fifth, sixth, or seventh embodiment.

In a specific embodiment of the eighth embodiment, R² is halo, —CN, C₁₋₆alkyl, C₃₋₈cycloalkyl, 4 to 6-membered heterocycloalkyl, or 5 to 6-membered heteroaryl, wherein the C₁₋₆alkyl, C₃₋₈cycloalkyl, 4 to 6-membered heterocycloalkyl, and 5 to 6-membered heteroaryl are each optionally substituted with 1 to 3 substituents independently selected from halo, C₁₋₄alkyl, C₁₋₄haloalkyl and C₃₋₆cycloalkyl.

In another specific embodiment, R² is halo, —CN, cyclopentyl, 5-membered heterocycloalkyl or 5-membered heteroaryl, wherein the cyclopentyl, 5-membered heterocycloalkyl and 5-membered heteroaryl are each optionally substituted with 1 to 3 substituents independently selected from halo, C₁₋₄alkyl and C₁₋₄haloalkyl, and the definitions for the other variables are as defined in the eighth embodiment.

In another specific embodiment, R² is halo, —CN, cyclopentyl, pyrazolyl, or tetrahydrofuranyl; and the the definitions for the other variables are as defined in the eighth embodiment.

In yet another specific embodiment, R² is —CN, cyclopentyl,

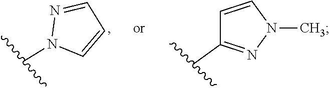 [SMILES: *n1cccn1 ; *c1ccn(C)n1]

and the definitions for the other variables are as defined in the eighth embodiment.

In another specific embodiment of the eighth embodiment, R² is H, —CN,

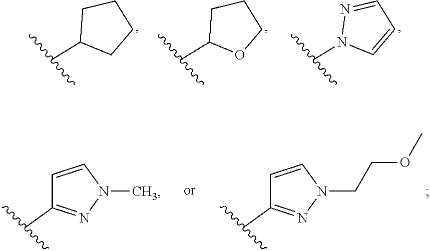

and the definitions for the other variables are as defined in the eighth embodiment.

In a more specific embodiment, R² is

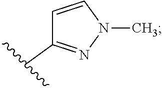 [SMILES: *c1ccn(C)n1]

and the definitions for the other variables are as defined in the eighth embodiment.

In another more specific embodiment, R² is H; and the definitions for the other variables are as defined in the eighth embodiment.

In a ninth embodiment of the present invention, the compound is represented by formula (I′), (I), (II), (III), (IV), (V), (VI), (VII), (VIII), (III-1), (III-2), (IV-1), (V-1), (VI-1), (VI-2), (VII-1), or (VIII-1), or a pharmaceutically acceptable salt thereof, wherein R⁴ is H, C₁₋₄alkyl, or 5 to 6-membered heterocycloalkyl, wherein the C₁₋₄alkyl and 5 to 6-membered heterocycloalkyl are each optionally substituted with 1 or 2 substituents independently selected from C₁₋₃alkyl, —CN, N(R^{4a})₂, OR^{4a}, and C(O)OR^{4a}; and R^{4a} is H or C₁₋₃alkyl optionally substituted with —OH or C₁₋₃alkoxy; and the definitions for the other variables are as defined in the first, second, third, fourth, fifth, sixth, seventh, or eighth embodiment, or any one of the specific embodiments of the eighth embodiment.

In a specific embodiment of the ninth embodiment, R⁴ is H or C₁₋₄alkyl optionally substituted with 1 or 2 substituents independently selected from —CN, N(R^{4a})₂, OR^{4a}, and C(O)OR^{4a}; and R^{4a} is H or C₁₋₃alkyl optionally substituted with —OH or C₁₋₃alkoxy; and the definitions for the other variables are as defined in the ninth embodiment.

In another specific embodiment of the ninth embodiment, R⁴ is H, CH₂CH₃,

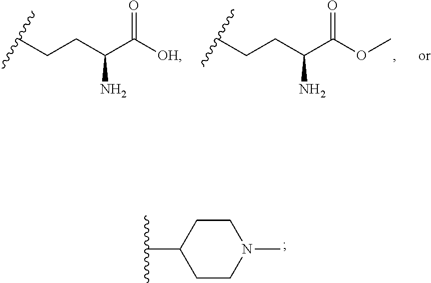

and the definitions for the other variables are as defined in the ninth embodiment.

In a more specific embodiment, R⁴ is

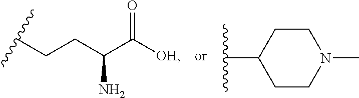

and the definitions for the other variables are as defined in the ninth embodiment.

In another more specific embodiment, R⁴ is H; and the definitions for the other variables are as defined in the ninth embodiment.

In a tenth embodiment of the present invention, the compound is represented by formula (I′), (I), (II), (III), (IV), (V), (VI), (VII), (VIII), (III-1), (III-2), (IV-1), (V-1), (VI-1), (VI-2), (VII-1), or (VIII-1), or a pharmaceutically acceptable salt thereof, wherein Ring A is cyclopropyl, cyclobutyl, cyclopentyl, cyclohexanyl, azetidinyl, pyrrolidinyl, imidazolidinyl, tetrahydrofuranyl, tetrahydrothiophenyl, piperidinyl, piperazinyl, morpholinyl, tetrahydropyranyl, azaspiro[3.3]heptanyl, 2-azaspiro[3.3]heptanyl, 2-azaspiro[3.5]nonanyl, octahydroindolizinyl, (1R,5S)-8-methyl-8-azabicyclo[3.2.1]octanyl, phenyl, pyrazolyl, imidazolyl, tetrazolyl, isoxazolyl, thiazolyl, 1,3,4-thiadiazolyl, 1,2,4-thiadiazolyl, pyridinyl, pyrimidinyl, pyrazinyl, quinolinyl, isoquinolinyl, 1H-benzo[d]imidazolyl, benzo[d]oxazolyl, benzo[d]thiazolyl, or benzo[c][1,2,5]thiadiazolyl, each of which is optionally substituted with 1 to 3 R⁶; and the definitions for the other variables are as defined in the first, second, third, fourth, fifth, sixth, seventh, eighth, or ninth embodiment, or any one of the specific embodiments of the eighth or ninth embodiment.

In an eleventh embodiment of the present invention, the compound is represented by formula (I′), (I), (II), (III), (IV), (V), (VI), (VII), (VIII), (III-1), (III-2), (IV-1), (V-1), (VI-1), (VI-2), (VII-1), or (VIII-1), or a pharmaceutically acceptable salt thereof, wherein R⁵ is H, C₁₋₄alkyl, —C₁₋₃alkylene-C₃₋₆cycloalkyl, —C₁₋₃alkylene-(4 to 6-membered heterocycloalkyl), —C₁₋₄alkylene-phenyl, —C₁₋₃alkylene-(5 to 6-membered heteroaryl), C₃₋₆cycloalkyl, 4 to 6-membered heterocycloalkyl, 7 to 10-membered spiro or bridged bicyclic heterocycloalkyl, phenyl, 5 to 6-membered heteroaryl, or 8- to 10-membered bicyclic heteroaryl, each of which is optionally substituted with 1 to 3 R⁶; or R⁴ and R⁵ together with the N atom from which they are attached form a 5 to 7-membered heterocycloalkyl optionally containing an additional heteroatom selected from O and N, wherein the heterocycloalkyl is optionally substituted with 1 to 3 R⁶; or the heterocycloalkyl is optionally fused with a phenyl or a 5 to 6-membered heteroaryl; and the definitions for the other variables are as defined in the first, second, third, fourth, fifth, sixth, seventh, eighth, ninth, or tenth embodiment, or any one of the specific embodiments of the eighth or ninth embodiment.

In a twelfth embodiment of the present invention, the compound is represented by formula (I′), (I), (II), (III), (IV), (V), (VI), (VII), (VIII), (III-1), (III-2), (IV-1), (V-1), (VI-1), (VI-2), (VII-1), or (VIII-1), or a pharmaceutically acceptable salt thereof, wherein R⁵ is C₁₋₄alkyl, phenyl, 4 to 6-membered heterocycloalkyl, 5 to 6-membered heteroaryl, 8- to 10-membered bicyclic heteroaryl, C₃₋₆cycloalkyl, C₃₋₆cycloalkyl fused with 5 to 6-membered heteroaryl, —C₁₋₃alkylene-phenyl, —C₁₋₃alkylene-(5 to 6-membered heteroaryl), —C₁₋₃alkylene-C₃₋₆cycloalkyl or —C₁₋₃alkylene-(4 to 6-membered heterocycloalkyl), each of which is optionally substituted with 1 to 3 R⁶; or R⁴ and R⁵ together with the N atom from which they are attached form a 6 or 7-membered heterocycloalkyl optionally containing an additional heteroatom selected from O and N, wherein the heterocycloalkyl is optionally substituted with 1 to 3 R⁶; and the definitions for the other variables are as defined in the first, second, third, fourth, fifth, sixth, seventh, eighth, ninth, tenth or eleventh embodiment, or any one of the specific embodiments of the eighth or ninth embodiment.

In a specific embodiment of the eleventh embodiment, R⁵ is H, —CH₃, —CH₂CH₃, —CH₂CH₂CH₃, —CH(CH₃)CH₃, —(CH₂)₃CH₃, —CH₂-cyclohexane, —CH₂CH₂-cyclohexane, —CH₂-azetidine, —CH₂-pyrrolidine, —(CH₂)₃-pyrrolidine, —CH₂CH₂-imidazolidine, —CH₂-tetrahydrofuran, —CH₂-piperidine, —CH(CH₃)-piperidine, —CH₂-(tetrahydropyran), —CH₂CH₂-(tetrahydropyran), —CH₂CH₂-morpholine, —CH₂-phenyl, —CH₂CH₂-phenyl, —CH(CH₃)-phenyl, —CH(CH₂CH₃)-phenyl, —CH₂CH(CH₃)-phenyl, —CH(CH₃)CH₂CH₂-phenyl, —CH₂—CH₂-imidazole, —(CH₂)₃-imidazole, —(CH₂)₃-tetrazole, —CH₂-isoxazole, —(CH₂)₃-isoxazole, —CH₂-pyridine, —CH₂—CH₂-pyridine, —CH₂-pyrazine, —CH₂—CH₂-pyrimidine, cyclopropyl, cyclobutyl, cyclopentyl, cyclohexanyl, azetidinyl, pyrrolidinyl, tetrahydrothiophenyl, piperidinyl, piperazinyl, tetrahydropyranyl, azaspiro[3.3]heptanyl, 2-azaspiro[3.3]heptanyl, 2-azaspiro[3.5]nonanyl, octahydroindolizinyl, (1R,5S)-8-methyl-8-azabicyclo[3.2.1]octanyl, phenyl, pyrazolyl, isoxazolyl, thiazolyl, 1,3,4-thiadiazolyl, 1,2,4-thiadiazolyl, pyridinyl, pyrimidinyl, pyrazinyl, quinolinyl, isoquinolinyl, 1H-benzo[d]imidazolyl, benzo[d]oxazolyl, benzo[d]thiazolyl, or benzo[c][1,2,5]thiadiazolyl, each of which is optionally substituted with 1 to 3 R⁶; or
R⁵ is —CH₂-naphthalene, —CH₂CH₂-naphthalene, naphthalenyl, 2,3-dihydro-1H-indenyl, 4,5,6,7-tetrahydro-1H-benzo[d]imidazolyl, 5,6,7,8-tetrahydroimidazo[1,2-a]pyridinyl, 4,5,6,7-tetrahydrobenzo[d]thiazolyl, 5,6,7,8-tetrahydroquinazolinyl, 1,2,3,4-tetrahydroquinolinyl, or 3,4-dihydro-2H-benzo[b][1,4]dioxepinyl, each of which is optionally substituted with 1 to 3 substituents independently selected from C₁₋₆alkyl, halo, oxo, —OR^{6a}, —N(R^{6a})₂, and —C(═O)N(R^{6a})₂; and the definitions for the other variables are as defined in the eleventh embodiment.

In a specific embodiment of the twelfth embodiment, R⁵ is —CH₃, —CH₂CH₃, —CH₂CH₂CH₃, phenyl, benzyl, —CH₂CH₂phenyl, pyrazole, isoxazole, pyridine, pyrimidine, quinoline, 1H-benzo[d]imidazole, 4,5,6,7-tetrahydro-1H-benzo[d]imidazole, 4,5,6,7-tetrahydrobenzo[d]thiazole, pyrrolidine, piperidine, piperazine, tetrahydro-2H-pyran, azaspiro[3.3]heptane, cyclohexane, —CH₂-cyclohexane, —CH₂-azetidine, —CH₂-pyridine, —CH₂-pyrazine, —CH₂-piperidine, CH₂-(tetrahydro-2H-pyran), —CH₂—CH₂-imidazole, —CH₂—CH₂-pyridine, —CH₂—CH₂-pyrimidine, —CH(CH₃)-piperidine, —(CH₂)₃-pyrrolidine, each of which is optionally substituted with 1 to 3 R⁶; or R⁴ and R⁵ together with the N atom from which they are attached form piperidine or piperazine ring, each or which is optionally substituted with 1 to 3 R⁶; and the definitions for the other variables are as defined in the twelfth embodiment.

In a more specific embodiment, R⁵ is

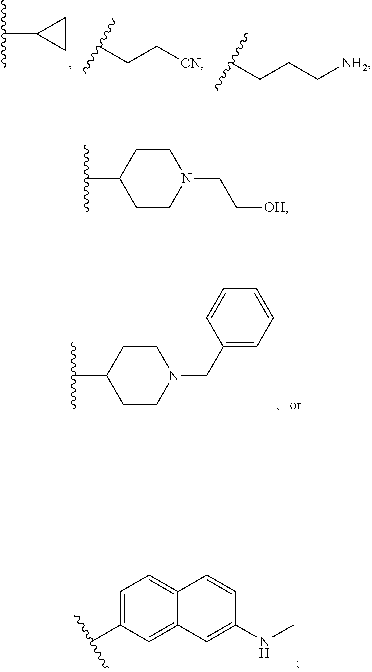

and the definitions for the other variables are as defined in the twelfth embodiment.

In another more specific embodiment, R⁵ is represented by the following formula:

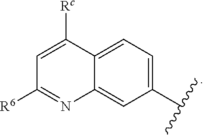

wherein R^{c} is selected from H, halo, C₁₋₄alkyl, —OR^{c1} and —N(R^{c1})₂, and R^{c1}, for each occurrence, is independently H or C₁₋₄alkyl optionally substituted with C₃₋₆cycloalkyl or phenyl; and the definitions for the other variables are as defined in the twelfth embodiment.

In a thirteenth embodiment of the present invention, the compound is represented by formula (I′), (I), (II), (III), (IV), (V), (VI), (VII), (VIII), (III-1), (III-2), (IV-1), (V-1), (VI-1), (VI-2), (VII-1), or (VIII-1), or a pharmaceutically acceptable salt thereof, wherein R⁴ and R⁵ together with the N atom from which they are attached form pyrrolidine, piperidine or piperazine ring, each or which is optionally substituted with 1 to 3 R⁶, or each of which is optionally fused with a phenyl or a 5 to 6-membered heteroaryl; and the definitions for the other variables are as defined in the first, second, third, fourth, fifth, sixth, seventh, eighth, ninth, tenth, eleventh, or twelfth embodiment, or any one of the specific embodiments of the eighth, ninth, eleventh, or twelfth embodiment.

In a specific embodiment of the thirteenth embodiment, R⁴ and R⁵ together with the N atom from which they are attached form one of the following cyclic rings:

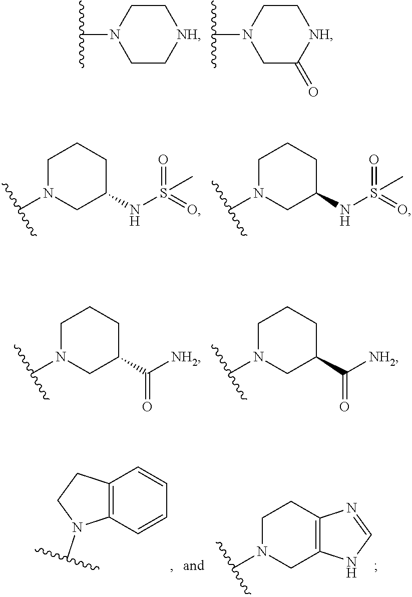

and the definitions for the other variables are as defined in the thirteenth embodiment.

In a fourteenth embodiment of the present invention, the compound is represented by formula (I′), (I), (II), (III), (IV), (V), (VI), (VII), (VIII), (III-1), (III-2), (IV-1), (V-1), (VI-1), (VI-2), (VII-1), or (VIII-1), or a pharmaceutically acceptable salt thereof, wherein:
R⁶ is halo, oxo, C₁₋₄alkyl, —CN, —C(═O)R^{6a}, —C(═O)OR^{6a}, —C(═O)N(R^{6a})₂, —N(R^{6a})₂, —NR^{6a}C(═O)R^{6a}, —NR^{6a}C(═O)OR^{6a}, —NR^{6a}C(═O)NR^{6a}, —NR^{6a}S(═O)₂R^{6a}, —OR^{6a}, —S(═O)₂R^{6a}, —S(═O)₂N(R^{6a})₂, C₃₋₄cycloalkyl, 4 to 6-membered heterocycloalkyl, phenyl, or 5 to 6-membered heteroaryl, wherein the C₁₋₄alkyl, 4 to 6-membered heterocycloalkyl, and 5 to 6-membered heteroaryl, are each optionally substituted with 1 to 3 substituents independently selected from halo, C₁₋₃alkyl, C₃₋₄cycloalkyl, 5 to 6-membered heterocycloalkyl substituted with 2 oxo, phenyl, —OR^{6a}, and N(R^{6a})₂,
R^{6a} is H, C₁₋₃alkyl, C₃₋₆cycloalkyl, 4 to 6-membered heterocycloalkyl, phenyl, or 5 to 6-membered heteroaryl, wherein the C₁₋₃alkyl, C₃₋₆cycloalkyl, and 4 to 6-membered heterocycloalkyl are each optionally substituted with 1 to 3 substituents independently selected from halo, —OH, —CN, C₁₋₄alkyl, C₁₋₄haloalkyl, C₁₋₄alkoxy, C₃₋₆cycloalkyl, 5 to 6-membered heterocycloalkyl, and phenyl; and the definitions for the other variables are as defined in the first, second, third, fourth, fifth, sixth, seventh, eighth, ninth, tenth, eleventh, twelfth or thirteenth embodiment, or any one of the specific embodiments of the eighth, ninth, eleventh, twelfth, or thirteenth embodiment.

In a specific embodiment of the fourteenth embodiment,
R⁶ is C₁₋₄alkyl, halo, oxo, —OR^{6a}, —N(R^{6a})₂, —C(═O)R^{6a}, —C(═O)N(R^{6a})₂, —NR^{6a}C(═O)NR^{6a}, —NR^{6a}S(═O)₂R^{6a}, or 4 to 6-membered heterocycloalkyl, wherein the C₁₋₄alkyl and 4 to 6-membered heterocycloalkyl are each optionally substituted with 1 to 3 substituents independently selected from halo, —OR^{6a}, and N(R^{6a})₂,
R^{6a} is H, C₁₋₃alkyl, C₃₋₆cycloalkyl, or 4 to 6-membered heterocycloalkyl, wherein the C₁₋₃alkyl, C₃₋₆cycloalkyl, and 4 to 6-membered heterocycloalkyl are each optionally substituted with 1 to 3 substituents independently selected from halo, —OH, —CN, C₁₋₄alkyl, C₁₋₄haloalkyl, C₁₋₄alkoxy, C₃₋₆cycloalkyl; and the definitions for the other variables are as defined in the fourteenth embodiment.

In another specific embodiment of the fourteenth embodiment, R⁶ is Cl, F, Br, oxo, —CH₃, —CH₂CH₃, isopropyl, butyl, cyclobutyl, —CH₂(cyclobutane), —CF₃, —CH₂CHF₂, —CH₂CH₂OH, —CH₂CH₂OCH₃, —CN, —CH₂CH₂NH₂, —CH₂CH₂N(CH₃)₂, —(CH₂)₃N(CH₃)₂,

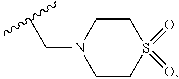 [SMILES: *CN1CCS(=O)(=O)CC1]

—C(═O)CH₃, —C(═O)OH, —C(═O)OCH₃,

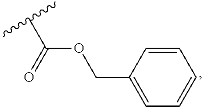 [SMILES: *C(=O)OCc1ccccc1]

—C(═O)NH₂, —C(═O)NHCH₃,

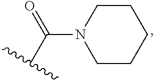 [SMILES: *C(=O)N1CCCCC1]

—NH₂, —NHCH₃, —N(CH₃)₂, —NHCH₂CH₃, —NH(CH₂-cyclopropane), —NH(cyclobutane), —NHCH₂CHF₂, —NHCH₂CH₂OCH₃,

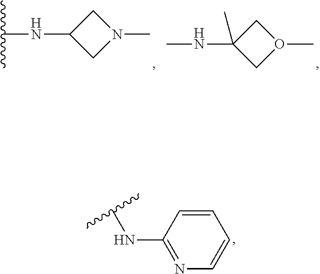

—NHC(═O)CH₃, —NHC(═O)NHCH₃,

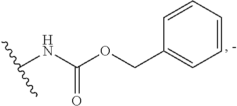 [SMILES: *NC(=O)OCc1ccccc1]

NHS(═O)₂CH₃, azetidinyl,

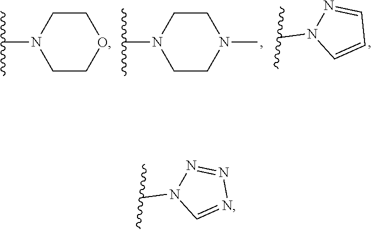

—OH, —OCH₃, —OCH(CH₃)₂, —OCHF₂, —OCF₃, —OCH₂CH₂OH,

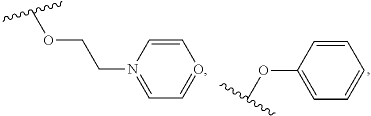

—S(═O)₂CH₃, —S(═O)₂N(CH₃)₂,

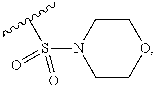 [SMILES: *S(=O)(=O)N1CCOCC1]

phenyl, benzyl, or pyridinyl; and the definitions for the other variables are as defined in the fourteenth embodiment.

In another more specific embodiment, R⁶ is F, oxo, —CH₃, —CH₂CH₃, —CH₂CHF₂, —CH₂CH₂OH, —CH₂CH₂OCH₃, —CH₂CH₂N(CH₃)₂, —NH₂, —NHCH₃, —N(CH₃)₂, —NHCH₂CH₃, —NHCH₂CHF₂, —NHCH₂CH₂OCH₃, —NH(cyclobutyl), —NH(CH₂cyclopropyl),

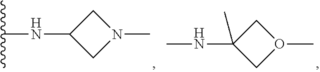

azetidine, —NHC(═O)NHCH₃, —NHS(═O)₂CH₃, —OH, —OCH₃, —C(═O)NH₂, or —C(═O)CH₃; and the definitions for the other variables are as defined in the fourteenth embodiment.

In a fifteenth embodiment of the present invention, the compound is represented by the following formula:

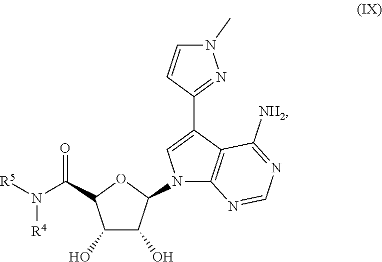

or a pharmaceutically acceptable salt thereof, wherein:
R⁴ is H, C₁₋₄alkyl, or 5 to 6-membered heterocycloalkyl, wherein the C₁₋₄alkyl and 5 to 6-membered heterocycloalkyl are each optionally substituted with 1 or 2 substituents independently selected from C₁₋₃alkyl, —CN, N(R^{4a})₂, OR^{4a}, and C(O)OR^{4a};
R^{4a} is H or C₁₋₃alkyl optionally substituted with —OH or C₁₋₃alkoxy;
R⁵ is C₁₋₄alkyl, 4 to 6-membered heterocycloalkyl, 8- to 10-membered bicyclic heteroaryl, or C₃₋₆cycloalkyl, each of which is optionally substituted with 1 or 2 R⁶;
R⁶ is —CN, —N(R^{6a})₂, or C₁₋₄alkyl optionally substituted with phenyl or —OR^{6a}; and
R^{6a} is H or C₁₋₃alkyl; and the definitions for the other variables are as defined in the first embodiment.

In a specific embodiment of the fifteenth embodiment,
R⁴ is H,

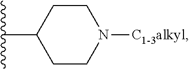

or C₁₋₄alkyl optionally substituted with 1 or 2 substituents independently selected from —N(R^{4a})₂ and C(O)OR^{4a};
R^{4a} is H or C₁₋₃alkyl;
R⁵ is cyclopropyl,

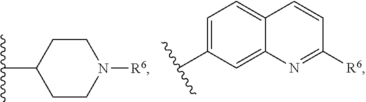

or C₁₋₄alkyl optionally substituted with —CN or —N(R^{6a})₂;
R⁶ is —N(R^{6a})₂ or C₁₋₄alkyl optionally substituted with phenyl or —OR^{6a}; and
R^{6a} is H or C₁₋₃alkyl; and the definitions for the other variables are as defined in the fifteenth embodiment.

In a more specific embodiment of the fifteenth embodiment,
(i) R⁴ is H or C₁₋₄alkyl optionally substituted with 1 or 2 substituents independently selected from —N(R^{4a})₂ and C(O)OR^{4a}; and
   R⁵

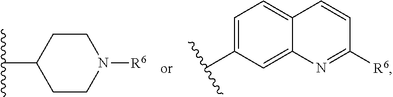

or
(ii) R⁴ is

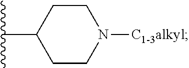

and
   R⁵ is cyclopropyl, or C₁₋₄alkyl optionally substituted with —CN or —N(R^{6a})₂; and the definitions for the other variables are as defined in the fifteenth embodiment.

In another more specific embodiment, R^{4a} is H; and R^{6a} is H or —CH₃; and the definitions for the other variables are as defined in the fifteenth embodiment.

In another specific embodiment of the fifteenth embodiment,
R⁴ is H,

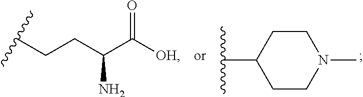

R⁵ is

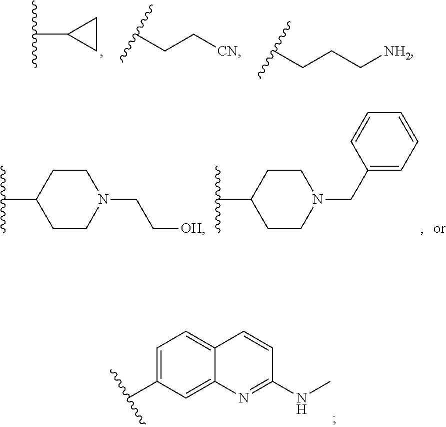

and the definitions for the other variables are as defined in the fifteenth embodiment.

In a sixteenth embodiment of the present invention, the compound of the present invention is selected from the compounds described in the Exemplifications section, e.g., compounds I-1 to I-235, or a pharmaceutically acceptable salt thereof.

### Definitions

As used herein, the term “alkyl” refers to a fully saturated branched or unbranched hydrocarbon moiety. Preferably the alkyl comprises 1 to 20 carbon atoms, more preferably 1 to 16 carbon atoms, 1 to 10 carbon atoms, 1 to 6 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, or n-decyl.

The number of carbon atoms in a group is specified herein by the prefix “Cₓ₋ₓₓ”, wherein x and xx are integers. For example, “C₁₋₄alkyl” is an alkyl group which has from 1 to 4 carbon atoms; and C₁₋₄haloalkyl is a haloalkyl group which has from 1 to 4 carbon atoms.

As used herein, the term “alkenyl” refers to an olefinically unsaturated branched or linear group having at least one double bond. Preferably the alkenyl comprises 2 to 20 carbon atoms, more preferably 2 to 16 carbon atoms, 2 to 10 carbon atoms, 2 to 6 carbon atoms, or 2 to 4 carbon atoms. Alkenyl groups include, but are not limited to, propenyl, 1,3-butadienyl, 1-butenyl, hexenyl, pentenyl, heptenyl, octenyl and the like.

As used herein, the term “alkynyl” refers to an unsaturated branched or linear group having at least one triple bond. Preferably the alkynyl comprises 2 to 20 carbon atoms, more preferably 2 to 16 carbon atoms, 2 to 10 carbon atoms, 2 to 6 carbon atoms, or 2 to 4 carbon atoms. Alkynyl groups include, but are not limited to, propynyl, 1-butynyl, hexynyl, pentynyl, hexynyl, heptynyl, octynyl and the like.

As used herein, the term “carbocyclyl” refers to saturated or partially unsaturated (but not aromatic) monocyclic, bicyclic or tricyclic hydrocarbon groups of 3-14 carbon atoms, preferably 3-9, or more preferably 3-8 carbon atoms. Carbocyclyls include fused, bridged, or spiro ring systems. The term “carbocyclyl” encompasses cycloalkyl groups. The term “cycloalkyl” refers to completely saturated monocyclic, bicyclic or tricyclic hydrocarbon groups of 3-12 carbon atoms, preferably 3-9, or more preferably 3-8 carbon atoms. Exemplary monocyclic carbocyclyl groups include, but are not limited to, cyclopropyl, cyclobutyl, cyclopentyl, cyclopentenyl, cyclohexyl or cyclohexenyl. Exemplary bicyclic carbocyclyl groups include bornyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, or bicyclo[2.2.2]octyl. Exemplary tricyclic carbocyclyl groups include adamantyl.

As used herein, the term “halocycloalkyl” refers to a cycloalkyl, as defined herein, that is substituted by one or more halo groups as defined herein. Preferably the halocycloalkyl can be monohalocycloalkyl, dihalocycloalkyl or polyhalocycloalkyl including perhalocycloalkyl. A monohalocycloalkyl can have one iodo, bromo, chloro or fluoro substituent. Dihalocycloalkyl and polyhalocycloalkyl groups can be substituted with two or more of the same halo groups or a combination of different halo groups.

As used herein, the term “cycloalkenyl” refers to a partially unsaturated monocyclic, bicyclic or tricyclic hydrocarbon groups having 3-12 ring carbon atoms, preferably 3-9, or more preferably 3-8 carbon atoms, and having one or more double bonds. Exemplary monocyclic cycloalkenyl groups include, but are not limited to, cyclopentenyl, cyclopentadienyl, cyclohexenyl, and the like. Exemplary bicyclic cycloalkenyl groups include, but are not limited to, bicyclo[2.2.1]hept-5-enyl and bicycle[2.2.2]oct-2-enyl.

As used herein, the term “haloalkyl” refers to an alkyl, as defined herein, that is substituted by one or more halo groups as defined herein. Preferably, the haloalkyl can be monohaloalkyl, dihaloalkyl or polyhaloalkyl including perhaloalkyl. A monohaloalkyl can have one iodo, bromo, chloro or fluoro substituent. Dihaloalkyl and polyhaloalkyl groups can be substituted with two or more of the same halo groups or a combination of different halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms. Preferred haloalkyl groups are trifluoromethyl and difluoromethyl.

“Halogen” or “halo” may be fluoro, chloro, bromo or iodo.

The term “aryl” refers to monocyclic, bicyclic or tricyclic aromatic hydrocarbon groups having from 6 to 14 ring carbon atoms. In one embodiment, the term aryl refers to monocyclic or bicyclic aromatic hydrocarbon groups having from 6 to 10 carbon atoms. Representative examples of aryl groups include phenyl (Ph), naphthyl, fluorenyl, and anthracenyl.

The term “aryl” also refers to a bicyclic or tricyclic group in which at least one ring is aromatic and is fused to one or two non-aromatic hydrocarbon ring(s). Nonlimiting examples include tetrahydronaphthalene, dihydronaphthalenyl and indanyl.

As used herein, the term “heterocyclyl” refers to a saturated or unsaturated, non-aromatic monocyclic, bicyclic or tricyclic ring system which has from 3- to 15-ring members at least one of which is a heteroatom, and up to 10 of which may be heteroatoms, wherein the heteroatoms are independently selected from O, S and N, and wherein N and S can be optionally oxidized to various oxidation states. In one embodiment, a heterocyclyl is a 3-8-membered monocyclic. In another embodiment, a heterocyclyl is a 6-12-membered bicyclic. In yet another embodiment, a heterocyclyl is a 10-15-membered tricyclic ring system. The heterocyclyl group can be attached at a heteroatom or a carbon atom. Heterocyclyls include fused or bridged ring systems. The term “heterocyclyl” encompasses heterocycloalkyl and heterocycloalkenyl groups. The term “heterocycloalkyl” refers to completely saturated monocyclic, bicyclic or tricyclic heterocyclyl comprising 3-15 ring members, at least one of which is a heteroatom, and up to 10 of which may be heteroatoms, wherein the heteroatoms are independently selected from O, S and N, and wherein N and S can be optionally oxidized to various oxidation states. In one embodiment, a heterocyclyl is a 4 to 9-membered heterocycloalkyl. Examples of heterocyclyls include dihydrofuranyl, [1,3]dioxolane, 1,4-dioxane, 1,4-dithiane, piperazinyl, 1,3-dioxolane, imidazolidinyl, imidazolinyl, pyrrolidine, dihydropyran, oxathiolane, dithiolane, 1,3-dioxane, 1,3-dithianyl, oxathianyl, thiomorpholinyl, oxiranyl, aziridinyl, oxetanyl, azetidinyl, tetrahydrofuranyl, pyrrolidinyl, tetrahydropyranyl, piperidinyl, morpholinyl, piperazinyl, azepinyl, oxapinyl, oxazepinyl and diazepinyl. The term “heterocycloalkenyl” refers to partially unsaturated monocyclic, bicyclic or tricyclic heterocyclyl comprising 3-15 ring members, with at least one double bond and at least one of the ring members is a heteroatom, and up to 10 of which may be heteroatoms, wherein the heteroatoms are independently selected from O, S and N, and wherein N and S can be optionally oxidized to various oxidation states. In one embodiment, a heterocyclyl is a 4 to 7-membered heterocycloalkenyl. Examples of heterocycloalkenyl include 1,2,3,4-tetrahydropyridinyl, 1,2-dihydropyridinyl, 1,4-dihydropyridinyl, 1,2, 3, 6-tetrahydro-pyridinyl, 1,4,5,6-tetrahydro-pyrimidinyl, 2-pyrrolinyl, 3-pyrrolinyl, 2-imidazolinyl, 2-pyrazolinyl, 3,4-dihydro-2H-pyran, dihydrofuranyl, fluoro-dihydro-furyl group, dihydro-thienyl and dihydro-thiopyran-yl.

As used herein, the term “heteroaryl” refers to a 5-14 membered monocyclic-, bicyclic-, or tricyclic-ring system, having 1 to 10 heteroatoms independently selected from N, O or S, wherein N and S can be optionally oxidized to various oxidation states, and wherein at least one ring in the ring system is aromatic. In one embodiment, the heteroaryl is a 5 to 6-membered monocyclic heteroaromatic ring (also refers to as “5 to 6-membered heteroaryl”). Examples of monocyclic heteroaryl groups include pyridyl, thienyl, furanyl, pyrrolyl, pyrazolyl, imidazoyl, oxazolyl, isoxazolyl, thiazolyl, isothiazolyl, triazolyl, oxadiazolyl, thiadiazolyl and tetrazolyl. In another embodiment, the heteroaryl is an 8 to 10-membered bicyclic heteroaromatic ring (also refers to as “8 to 10-membered bycyclic heteroaryl”). Examples of bicyclic heteroaryl groups include quinolinyl, quinozalinyl, phthalazinyl, quinoxalinyl, cinnolinyl, naphthyridinyl, pyridoprimidinyl, pyridopyrazinyl, pteridinyl, indolyl, isoindolyl, indolizinyl, indazolyl, benzoimidazolyl, benzotriazolyl, benzooxazolyl, benzoisoxazolyl, benzothiazolyl, benzofuranyl, isobenzofuranyl, benzothiophenyl, benzothiadiazolyl, azaindolyl, purine, imidazopyridinyl, pyrrolopyrimidinyl, imidazopyridazinyl, imidazopyrazinyl, pyrazolopyrimidinyl, pyrazolopyridinyl, pyrazolotriazinyl, oxazolopyridinyl, isoxazolopyridinyl, thiazolopyridinyl, isothiazolopyridinyl, indolyl, benzofuranyl, quinolyl, isoquinolyl indazolyl, indolinyl, isoindolyl, indolizinyl, benzamidazolyl and quinolinyl.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy, cyclohexyloxy and the like. Preferably, alkoxy groups have about 1-6 carbon atoms, more preferably about 1-4 carbon atoms.

The term “bicyclic” or “bicyclic ring system,” as used herein, can include a fused ring system, a bridged ring system, or a spiro ring system.

The term “fused ring system,” as used herein, is a ring system that has two or three rings (preferably two rings) independently selected from carbocyclyl, heterocyclyl, aryl or heteroaryl rings that share one side A fused ring system may have from 4-15 ring members, preferably form 5-10 ring members. Examples of fused ring systems include octahydroisoquinolin-2(1H)-yl, 2,3-dihydro-1H-indenyl, octahydro-1H-pyrido[1,2-a]pyrazinyl, and decahydroisoquinolinyl).

The term “bridged ring system,” as used herein, is a ring system that has a carbocyclyl or heterocyclyl ring wherein two non-adjacent atoms of the ring are connected (bridged) by one or more (preferably from one to three) atoms. A bridged ring system can have more than one bridge within the ring system (e.g., adamantyl). A bridged ring system may have from 6-10 ring members, preferably from 7-10 ring members. Examples of bridged ring systems include adamantly, 9-azabicyclo[3.3.1]nonan-9-yl, 8-azabicyclo[3.2.1]octanyl, bicyclo[2.2.2]octanyl, 3-azabicyclo[3.1.1]heptanyl, bicyclo[2.2.1]heptanyl, (1R,5S)-bicyclo[3.2.1]octanyl, 3-azabicyclo[3.3.1]nonanyl, and bicyclo[2.2.1]heptanyl. More preferably, the bridged ring system is selected from the group consisting of 9-azabicyclo[3.3.1]nonan-9-yl, 8-azabicyclo[3.2.1]octanyl, and bicyclo[2.2.2]octanyl.

The term “spiro ring system,” as used herein, is a ring system that has two rings each of which are independently selected from a carbocyclyl or a heterocyclyl, wherein the two ring structures having one atom in common. Spiro ring systems have from 5 to 14 ring members. Example of spiro ring systems include 2-azaspiro[3.3]heptanyl, spiropentanyl, 2-oxa-6-azaspiro[3.3]heptanyl, 2,7-diazaspiro[3.5]nonanyl, 2-oxa-7-azaspiro[3.5]nonanyl, 6-oxa-9-azaspiro[4.5]decanyl, 6-oxa-2-azaspiro[3.4]octanyl, 5-azaspiro[2.3]hexanyl and 2,8-diazaspiro[4.5]decanyl.

The term “spiroheterocycloalkyl” as used herein, is a heterocycloalkyl that has one ring atom in common with the group to which it is attached. Spiroheterocycloalkyl groups may have from 3 to 15 ring members. In a preferred embodiment, the spiroheterocycloalkyl has from 3 to 8 ring atoms selected from carbon, nitrogen, sulfur and oxygen and is monocyclic.

In cases where a compound provided herein is sufficiently basic or acidic to form stable nontoxic acid or base salts, preparation and administration of the compounds as pharmaceutically acceptable salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, or α-glycerophosphate. Inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Pharmaceutically acceptable base addition salts can be prepared from inorganic and organic bases. Salts from inorganic bases, can include but are not limited to, sodium, potassium, lithium, ammonium, calcium or magnesium salts. Salts derived from organic bases can include, but are not limited to, salts of primary, secondary or tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocycloalkyl amines, diheterocycloalkyl amines, triheterocycloalkyl amines, or mixed di- and tri-amines where at least two of the substituents on the amine can be different and can be alkyl, substituted alkyl, alkenyl, substituted alkenyl, cycloalkyl, substituted cycloalkyl, cycloalkenyl, substituted cycloalkenyl, aryl, heteroaryl, or heterocycloalkyl and the like. Also included are amines where the two or three substituents, together with the amino nitrogen, form a heterocycloalkyl or heteroaryl group. Non-limiting examples of amines can include, isopropylamine, trimethyl amine, diethyl amine, tri(iso-propyl) amine, tri(n-propyl) amine, ethanolamine, 2-dimethylaminoethanol, trimethamine, lysine, arginine, histidine, caffeine, procaine, hydrabamine, choline, betaine, ethylenediamine, glucosamine, N-alkylglucamines, theobromine, purines, piperazine, piperidine, morpholine, or N-ethylpiperidine, and the like. Other carboxylic acid derivatives can be useful, for example, carboxylic acid amides, including carboxamides, lower alkyl carboxamides, or dialkyl carboxamides, and the like.

The compounds or pharmaceutically acceptable salts thereof as described herein, can contain one or more asymmetric centers in the molecule. In accordance with the present disclosure any structure that does not designate the stereochemistry is to be understood as embracing all the various stereoisomers (e.g., diastereomers and enantiomers) in pure or substantially pure form, as well as mixtures thereof (such as a racemic mixture, or an enantiomerically enriched mixture). It is well known in the art how to prepare such optically active forms (for example, resolution of the racemic form by recrystallization techniques, synthesis from optically-active starting materials, by chiral synthesis, or chromatographic separation using a chiral stationary phase).

When a particular stereoisomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired stereoisomer relative to the combined weight of all stereoisomers.

When a particular enantiomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired enantiomer relative to the combined weight of all stereoisomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomers at least 20%, 30%, 40%, 50%, 60%, 70%, 80%, 90%, 95%, 97%, 99%, 99.5% or 99.9%. The stereoisomeric purity the weight percent of the desired stereoisomers encompassed by the name or structure relative to the combined weight of all of the stereoisomers.

When a disclosed compound is named or depicted by structure without indicating the stereochemistry, and the compound has one chiral center, it is to be understood that the name or structure encompasses one enantiomer of compound in pure or substantially pure form, as well as mixtures thereof (such as a racemic mixture of the compound and mixtures enriched in one enantiomer relative to its corresponding optical isomer).

When a disclosed compound is named or depicted by structure without indicating the stereochemistry and, e.g., the compound has at least two chiral centers, it is to be understood that the name or structure encompasses one stereoisomer in pure or substantially pure form, as well as mixtures thereof (such as mixtures of stereoisomers, and mixtures of stereoisomers in which one or more stereoisomers is enriched relative to the other stereoisomer(s)).

The disclosed compounds may exist in tautomeric forms and mixtures and separate individual tautomers are contemplated. In addition, some compounds may exhibit polymorphism.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

In one embodiment, the present invention provides deuterated compounds described herein or a pharmaceutically acceptable salt thereof.

Another embodiment is a pharmaceutical composition comprising at least one compound described herein, or a pharmaceutically acceptable salt thereof, and at least one pharmaceutically acceptable carrier.

The compounds described herein have METTL3 modulating activity. In one embodiment, the compounds described herein have METTL3 inhibitory activity. In one embodiment, the compounds described herein are selective METTL3 inhibitors. In one embodiment, the compounds described herein have inhibitory activities against METTL3 that are higher than inhibitory activities against other protein targets, such as protein arginine N-methyltransferase 5 (PRMT5). In one embodiment, the compounds described herein have METTL3 inhibitory activities that are at least 2, 3, 5, 10, 15, 20, 30, 40, 50, 75, 100, 200, 400 or 1000 times greater than their inhibitory activities towards PRMT5.

In some embodiments, the METTL3 inhibitors described herein have an IC₅₀ value of less than 1 μM, less than 750 nM, less than 500 nM, less than 250 nM or less than 100 nM.

As used herein, “METTL3 modulating activity” refers to the ability of a compound or composition to induce a detectable change in METTL3 activity in vivo or in vitro (e.g., at least 10% increase or decrease in METTL3 activity as measured by a given assay such as the bioassay described in the examples and known in the art). A decrease in METLL3 activity is METTL3 inhibitory activity.

### Methods of Use

In one aspect, the present invention discloses a method of treating a disease or disorder responsive to inhibition of METTL3 activity in a subject comprising administering to the subject an effective amount of the compound described herein, or a pharmaceutically acceptable salt thereof.

In one embodiment, the disease or disorder is an infection, such as, a viral infection. In a specific embodiment, the viral infection is caused by RNA virus or retrovirus. Examples of viral infections include, but are not limited to, Dengue, Yellow Fever, Japanese encephalitis, Zika virus, Ebola virus, severe acute respiratory syndrome (SARS), rabies, HIV, influenza, hepatitis C, hepatitis E, West Nile fever, polio, measles, COVID-19, and Middle East respiratory syndrome (MERS-CoV).

In one embodiment, the disease or disorder is a cancer.

The term “cancer” includes diseases or disorders involving abnormal cell growth and/or proliferation.

In some embodiments, the cancer is selected from glioblastoma, leukemia, stomach cancer, prostate cancer, colorectal cancer, endometrial cancer, breast cancer, pancreatic cancer, kidney cancer, lung cancer, bladder cancer, ovarian cancer, esophageal/upper aerodigestive cancer, NHL, multiple myeloma, mesothelioma and sarcoma.

In a specific embodiment, the cancer is acute myeloid leukemia.

As used herein, the term “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or ‘treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

The effective dose of a compound provided herein, or a pharmaceutically acceptable salt thereof, administered to a subject can be 10 μg-500 mg.

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the mammal. Administering a compound described herein, or a pharmaceutically acceptable salt thereof, to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound that metabolizes within or on a surface of the body of the mammal to a compound described herein, or a pharmaceutically acceptable salt thereof.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

The compounds of the invention may also be administered intravenously or intraperitoneally by infusion or injection. Solutions of the active compound or its salts can be prepared in water, optionally mixed with a nontoxic surfactant.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds or pharmaceutically acceptable salts thereof as described herein can be dissolved or dispersed at effective levels, optionally with the aid of non-toxic surfactants.

Useful dosages of a compound or pharmaceutically acceptable salt thereof as described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound or pharmaceutically acceptable salt thereof as described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

Compounds or pharmaceutically acceptable salt thereof as described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

The disclosed method can include a kit comprising a compound or pharmaceutically acceptable salt thereof as described herein and instructional material which can describe administering a compound or pharmaceutically acceptable salt thereof as described herein or a composition comprising a compound or pharmaceutically acceptable salt thereof as described herein to a cell or a subject. This should be construed to include other embodiments of kits that are known to those skilled in the art, such as a kit comprising a (such as sterile) solvent for dissolving or suspending a compound or pharmaceutically acceptable salt thereof as described herein or composition prior to administering a compound or pharmaceutically acceptable salt thereof as described herein or composition to a cell or a subject. In some embodiments, the subject can be a human.

### EXEMPLIFICATIONS

### Instrument Details (and Conditions)

1. ¹H NMR or ¹⁹F NMR, NOESY spectra were recorded on Bruker AV□ 400.
2. LCMS measurement was run on Agilent 1200 HPLC/6100 SQ System using the follow conditions:
Method A: Mobile Phase: A: Water (0.01% TFA) B: Acetonitrile (0.01% TFA); Gradient Phase: 5% B to 95% B within 1.4 min, 95% B with 1.6 min (total runtime: 3 min); Flow Rate: 2.0 mL/min; Column: SunFire C18, 4.6*50 mm, 3.5 μm; Column Temperature: 40° C. Detectors: ADC ELSD, DAD (214 nm and 254 nm), ES-API.
Method B: Mobile Phase: A: Water (10 mM NH₄HCO₃) B: Acetonitrile; Gradient Phase: 5% to 95% B within 1.4 min, 95% B with 1.6 min (total runtime: 3 min); Flow Rate: 2.0 mL/min; Column: XBridge C18, 4.6*50 mm, 3.5 um; Column Temperature: 40° C. Detectors: ADC ELSD, DAD (214 nm and 254 nm), MSD (ES-API).
3. HPLC was taken on Agilent LC 1200 series.
Method A: Mobile Phase: A: Water (0.01% TFA) B: Acetonitrile (0.01% TFA); Gradient Phase: 5% B to 95% B within 9.5 min, 95% B with 5 min (total runtime: 14.5 min); Flow Rate: 1.0 mL/min; Column: SunFire C18, 4.6*100 mm, 3.5 μm; Column Temperature: 40° C. Detectors: ADC ELSD, DAD (214 nm and 254 nm), ES-API.
4. Prep-HPLC:
Instrument: Gilson 281 (PHG-009)
Column: Xtimate Prep C18 10 μm 21.2×250 mm
Method A: Mobile Phase: A: water (0.01% FA) B: acetonitrile
Method B: Mobile Phase: A: Water (10 mmol NH4HCO3); B: acetonitrile
Flow Rate (ml/min): 30.00
Detective Wavelength (nm): 214/254.

### Synthesis of Intermediates

### 1. Synthetic Scheme for Intermediate A

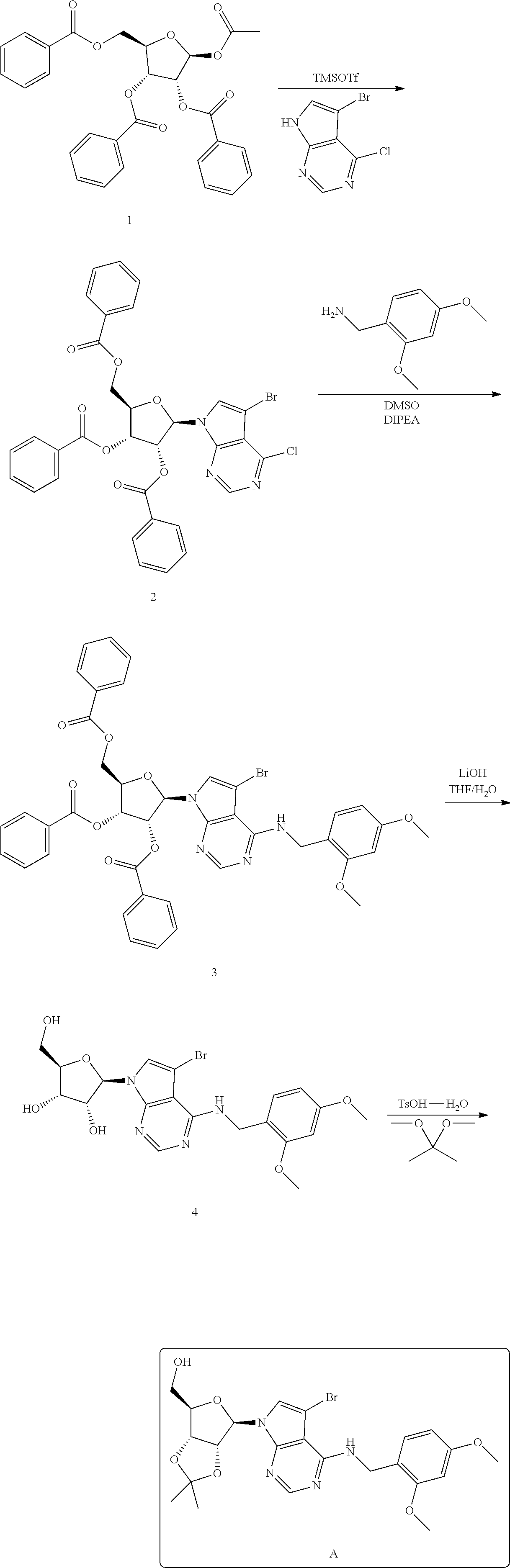

### 1.1 Synthesis of Compound 2

To a solution of 5-bromo-4-chloro-7H-pyrrolo[2,3-d]pyrimidine (189 g, 0.79 mol, 0.9 eq) in anhydrous MeCN (2 L, total 5V) under N₂ was added BSA (194 ml, 0.79 mol, 1 eq) at room temperature. After stirring for 15 minutes, (2S,3R,4R,5R)-2-acetoxy-5-((benzoyloxy)methyl)tetrahydrofuran-3,4-diyl dibenzoate 1 (400 g, 0.79 mol, 1 eq) and TMSOTf (144 ml, 0.79 mol, 1 eq) were added. The mixture was heated to 80° C. and stirred for 3 hours. The reaction mixture was then cooled to room temperature and EtOAc (2 L) and sat. NaHCO₃ solution (1 L) were added. The layers were separated; the organic layer was washed with brine, dried over Na₂SO₄ and concentrated in vacuo to give the crude product, which was purified by flash column chromatography (silica gel; eluting with 20-25% EtOAc in hexanes) to give (2R,3R,4R,5R)-2-((benzoyloxy)methyl)-5-(5-bromo-4-chloro-7H-pyrrolo[2,3-d]pyrimidin-7-yl)tetrahydrofuran-3,4-diyl dibenzoate as an off-white solid (300 g, 56%). ESI LCMS m/z=677 (M+2), ¹H NMR (400 MHz, DMSO-d6): 8.62 (d, J=2.4 Hz, 1H), 8.32 (s, 1H), 7.96 (dd, J=16.9, 7.8 Hz, 4H), 7.85 (d, J=7.8 Hz, 2H), 7.71-7.56 (m, 3H), 7.47 (dq, J=25.2, 7.7 Hz, 6H), 6.73 (d, J=4.8 Hz, 1H), 6.31 (t, J=5.7 Hz, 1H), 6.15 (d, J=5.9 Hz, 1H), 4.84 (ddd, J=27.1, 10.6, 3.9 Hz, 2H), 4.69 (dd, J=12.4, 4.9 Hz, 1H).

### 1.2 Synthesis of Compound 3

To a solution of 2R,3R,4R,5R)-2-((benzoyloxy)methyl)-5-(5-bromo-4-chloro-7H-pyrrolo[2,3-d]pyrimidin-7-yl)tetrahydrofuran-3,4-diyl dibenzoate 2 (300 g, 0.44 mol, 1 eq) in DMSO (2 L, 6.66 V) was added DIPEA (80 g, 0.61 mol, 1.4 eq) and 2,4-dimethoxybenzylamine (89 g, 0.52 mol, 1.2 eq). Upon completion of the addition, the reaction mixture was heated slowly to 110° C. and stirred for 3 hours. The mixture was then cooled to room temperature and poured into water (3500 mL). The solid which precipitated was collected by filtration and the filter cake was washed with water (1 L). The wet filter cake was then dissolved in EtOAc (1 L) and the solution was washed with water (1 L), brine (500 mL), dried over Na₂SO₄ and concentrated in vacuo to afford (2R,3R,4R,5R)-2-((benzoyloxy)methyl)-5-(5-bromo-4-((2,4-dimethoxybenzyl)amino)-7H-pyrrolo[2,3-d]pyrimidin-7-yl)tetrahydrofuran-3,4-diyl dibenzoate as an off-white solid (350 g, 98%). The product was used in the next step without further purification. ESI LCMS m/z=808 (M+2). ¹H NMR (400 MHz, DMSO-d6): 8.17-8.11 (m, 1H), 8.04-7.97 (m, 2H), 7.93 (d, J=7.4 Hz, 2H), 7.85 (d, J=7.8 Hz, 2H), 7.74 (s, 1H), 7.64 (dt, J=13.5, 7.6 Hz, 3H), 7.56-7.39 (m, 6H), 7.12 (d, J=8.2 Hz, 1H), 6.92 (t, J=6.2 Hz, 1H), 6.62-6.53 (m, 2H), 6.48-6.41 (m, 1H), 6.30 (t, J=5.4 Hz, 1H), 6.11 (t, J=5.1 Hz, 1H), 4.84-4.73 (m, 2H), 4.64 (d, J=6.5 Hz, 3H), 3.91-3.82 (m, 3H), 3.76-3.68 (m, 3H).

### 1.3 Synthesis of Compound 4

To a solution of (2R,3R,4R,5R)-2-((benzoyloxy)methyl)-5-(5-bromo-4-((2,4-dimethoxybenzyl)amino)-7H-pyrrolo[2,3-d]pyrimidin-7-yl)tetrahydrofuran-3,4-diyl dibenzoate 3 (350 g, 0.44 mol, 1 eq) in THE (3 L) and water (500 mL) was added LiOH (225 g, 5.35 mol, 12 eq). Upon completion of the addition, the reaction mixture was stirred at 45° C. for 3 hours. The reaction mixture was then diluted with water and the mixture was extracted with EtOAc (2×1 L). The organic extracts were washed with sat. NaHCO₃ (1 L), brine (1 L), dried over Na₂SO₄ and concentrated in vacuo to give the crude product, which was then triturated with hexane, isolated by filtration and dried in an oven at 45° C. to give (2R,3R,4S,5R)-2-(5-bromo-4-((2,4-dimethoxybenzyl)amino)-7H-pyrrolo[2,3-d]pyrimidin-7-yl)-5-(hydroxymethyl)tetrahydrofuran-3,4-diol as an off-white solid (210 g, 98%). The product was used in the next step without further purification. ESI LCMS m/z=495 (M+1). ¹H NMR (400 MHz, DMSO-d6): 8.17 (s, 1H), 7.67 (s, 1H), 7.13 (d, J=8.3 Hz, 1H), 6.86 (s, 1H), 6.59 (d, J=2.4 Hz, 1H), 6.45 (dd, J=8.3, 2.4 Hz, 1H), 6.04 (d, J=6.1 Hz, 1H), 5.32 (d, J=6.3 Hz, 1H), 5.21-5.06 (m, 2H), 4.65 (d, J=6.0 Hz, 2H), 4.34 (d, J=5.5 Hz, 1H), 4.07 (d, J=3.4 Hz, 1H), 3.86 (s, 3H), 3.73 (d, J=0.6 Hz, 3H), 3.65-3.46 (m, 2H).

### 1.4 Synthesis of Intermediate A

To a solution of (2R,3R,4S,5R)-2-(5-bromo-4-((2,4-dimethoxybenzyl)amino)-7H-pyrrolo[2,3-d]pyrimidin-7-yl)-5-(hydroxymethyl)tetrahydrofuran-3,4-diol 4 (215 g, 0.44 mol, 1 eq) in THF (2150 mL, 10V) at room temperature was added 2,2-dimethoxypropane (267 mL, 2.17 mol, 5 eq) and p-toluenesulfonic acid monohydrate (8.3 g, 0.044 mol, 0.1 eq). Upon completion of the addition, the reaction mixture was stirred at 60° C. for 16 hours. The mixture was then diluted with a sat. NaHCO₃ solution (3 L) and extracted with EtOAc (2×1.5 L). The organic extracts were washed with brine (1000 mL), dried over Na₂SO₄ and concentrated in vacuo to obtain the crude product, which was purified by flash column chromatography (silica gel; eluting with 10-20% EtOAc in hexane: DCM (7:3)) to give ((3aR,4R,6R,6aR)-6-(5-bromo-4-((2,4-dimethoxybenzyl)amino)-7H-pyrrolo[2,3-d]pyrimidin-7-yl)-2,2-dimethyltetrahydrofuro[3,4-d][1,3]dioxol-4-yl)methanol (Intermediate A, 130 g, 56%) ESI LCMS m/z=537 (M+2). ¹H NMR (DMSO): 8.20 (d, J=1.3 Hz, 1H), 7.77-7.56 (m, 1H), 7.13 (d, J=8.2 Hz, 1H), 6.88 (t, J=6.0 Hz, 1H), 6.67-6.35 (m, 2H), 6.20 (d, J=3.2 Hz, 1H), 5.28-4.98 (m, 2H), 4.90 (dd, J=6.4, 2.7 Hz, 1H), 4.65 (d, J=5.8 Hz, 2H), 4.13 (dd, J=4.9, 3.0 Hz, 1H), 3.79 (dd, J=51.2, 1.4 Hz, 6H), 3.55 (d, J=4.4 Hz, 2H), 1.53 (s, 3H), 1.30 (s, 4H).

### 2. Synthetic Scheme for Intermediate B

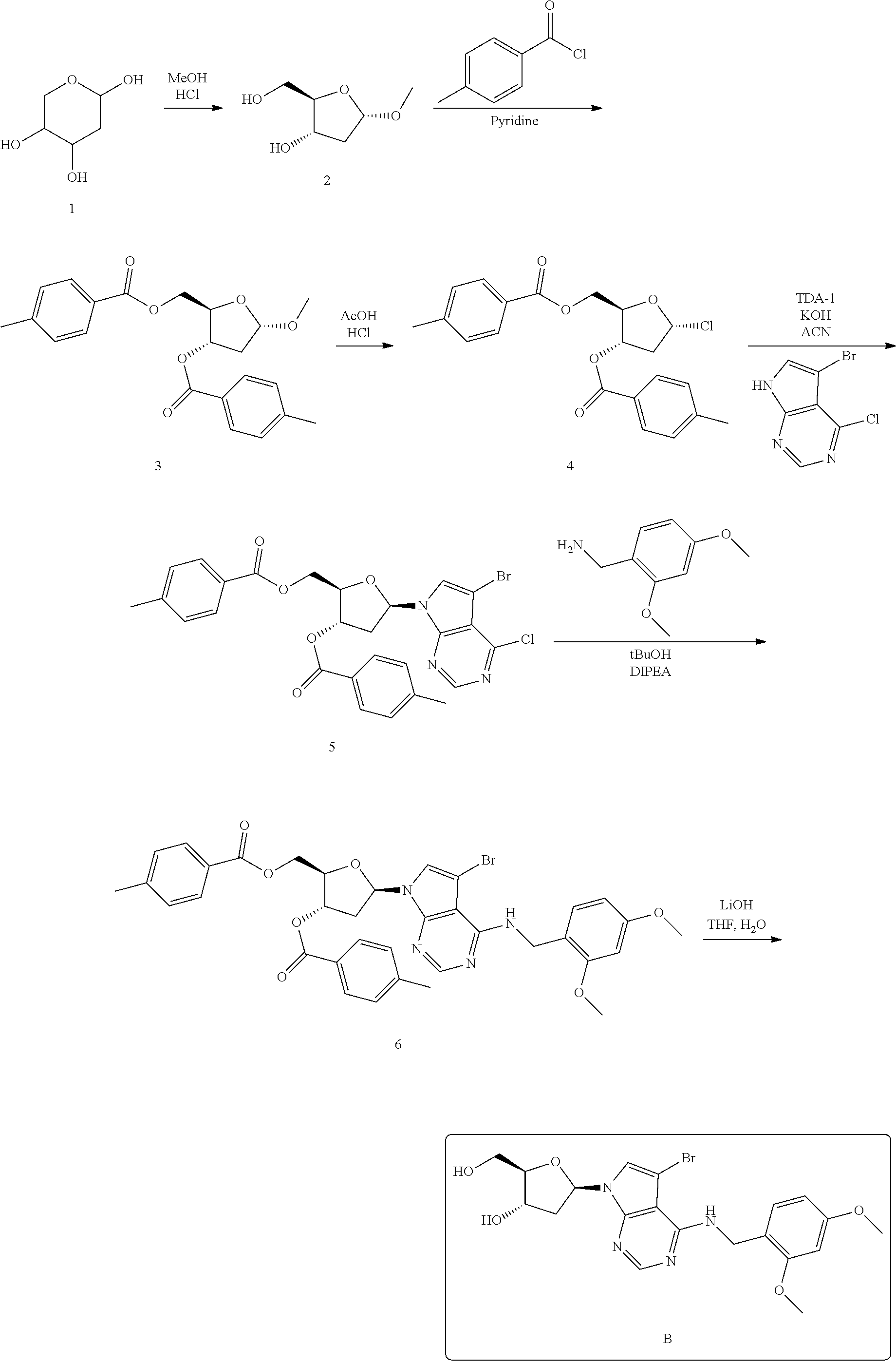

### 2.1 Synthesis of Compound 2

To a solution of deoxyribose (100 g, 0.746 mol, 1 eq) in methanol (1200 mL) was added 1% HCl in methanol (200 mL) at room temperature. The reaction mixture was stirred at room temperature for 30 min. NaHCO₃ (40 g) was then added and the resulting suspension was stirred for 30 min. The reaction mixture was filtered and the filtrate was concentrated in vacuo to obtain (2R,3S,5S)-2-(hydroxymethyl)-5-methoxytetrahydrofuran-3-ol as an orange oil (110 g, 100%).

### 2.2 Synthesis of Compound 3

To a solution of (2R,3S,5S)-2-(hydroxymethyl)-5-methoxytetrahydrofuran-3-ol 1 (110 g, 0.746 mol, 1 eq) in pyridine (660 mL) was added p-toluoyl chloride (247.4 g, 1.6 mol, 2.15 eq) at 0° C. The reaction mixture was allowed to warm to room temperature and stirred for a further 16 hours. Water (1 L) was added and the mixture extracted with DCM (3×300 mL). The combined organic extracts were then washed with a saturated NaHCO₃ solution (2×500 mL), 2N HCl (2×500 mL), brine (500 mL), dried over Na₂SO₄ and then concentrated in vacuo to give (2R,3S,5S)-5-methoxy-2-(((4-methylbenzoyl)oxy)methyl)tetrahydrofuran-3-yl 4-methylbenzoate as an brown oil (253 g, 89%).

### 2.3 Synthesis of Compound 4

To a solution of (2R,3S,5S)-5-methoxy-2-(((4-methylbenzoyl)oxy)methyl)tetrahydrofuran-3-yl 4-methylbenzoate (250 g, 0.65 mol, 1 eq) in acetic acid (393 mL) was added a freshly prepared solution of saturated HCl in acetic acid (using acetyl chloride and acetic acid) (618 mL) slowly at room temperature. After completion of the addition, further acetyl chloride (50 mL) was added slowly at room temperature. The reaction mixture was stirred for a further 30 minutes, upon which time a colourless precipitate formed that was isolated by filtration to give (2R,3S,5R)-5-chloro-2-(((4-methylbenzoyl)oxy)methyl)tetrahydrofuran-3-yl 4-methylbenzoate as a colourless solid (145 g, 58%). LCMS: 407 (M+18). ¹H NMR (400 MHz, CDCl₃): 8.10-7.84 (m, 5H), 7.34-7.20 (m, 5H), 6.52 (d, J=5.1 Hz, 1H), 5.61 (ddd, J=7.4, 2.8, 1.1 Hz, 1H), 4.91 (q, J=3.5 Hz, 1H), 4.75-4.61 (m, 2H), 2.92 (ddd, J=15.0, 7.5, 5.2 Hz, 1H), 2.79 (dd, J=15.2, 1.1 Hz, 1H), 2.47 (d, J=4.8 Hz, 6H).

### 2.4 Synthesis of Compound 5

A suspension of KOH powder (52 g, 0.926 mol, 2 eq) and tris[2-(2-methoxyethoxy)ethyl]amine (7.5 g, 0.023 mol, 0.05 eq) in anhydrous MeCN (1.8 L) was degassed by bubbling N₂ through it for 15 minutes. 5-Bromo-4-chloro-7H-pyrrolo[2,3-d]pyrimidine (107.6 g, 0.463 mol, 1 eq) was added and the reaction mixture was stirred at room temperature for 30 minutes. A solution of (2R,3S,5R)-5-chloro-2-(((4-methylbenzoyl)oxy)methyl)tetrahydrofuran-3-yl 4-methylbenzoate 3 (180 g, 0.463 mol, 1 eq) in anhydrous MeCN (1800 mL) was then added slowly at room temperature. The reaction mixture was stirred for 16 hours at room temperature. The reaction mixture was diluted with water (5 L) and the compound was extracted with EtOAc (3×1 L). The combined organic extracts were washed with brine (1.5 L), dried over Na₂SO₄ and concentrated in vacuo to give the crude product, which was purified by flash chromatography (silica gel; gradient elution 0-25% EtOAc:hexanes+DCM (7:3)) to obtain (2R,3S,5R)-5-(5-bromo-4-chloro-7H-pyrrolo[2,3-d]pyrimidin-7-yl)-2-(((4-methylbenzoyl)oxy)methyl)tetrahydrofuran-3-yl 4-methylbenzoate as an off-white solid (170 g, 62.8%). LC-MS: 608 (M+23). ¹H NMR (400 MHz, CDCl₃): 8.62 (s, 1H), 8.07-7.86 (m, 4H), 7.48 (s, 1H), 7.33-7.25 (m, 5H), 6.81 (t, J=7.0 Hz, 1H), 5.75 (td, J=4.3, 2.4 Hz, 1H), 4.83-4.59 (m, 3H), 2.79 (dd, J=7.1, 4.3 Hz, 2H), 2.44 (d, J=6.5 Hz, 6H).

### 2.5 Synthesis of Compound 6

To a solution of (2R,3S,5R)-5-(5-bromo-4-chloro-7H-pyrrolo[2,3-d]pyrimidin-7-yl)-2-(((4-methylbenzoyl)oxy)methyl)tetrahydrofuran-3-yl 4-methylbenzoate (140 g, 0.24 mol, 1 eq) in DMSO (1400 mL) was added DIPEA (40.2 g, 0.31 mol, 1.3 eq) and 2,4-dimethoxybenzylamine (52 g, 0.31 mol, 1.3 eq). Upon completion of the addition, the reaction mixture was slowly brought to 80° C. and stirred for 16 hours. The mixture was then diluted with water (3000 mL) and the compound was extracted with DCM (3×500 mL). The organic layer was washed with brine (1 L), dried over Na₂SO₄ and concentrated under reduced pressure to obtain (2R,3S,5R)-5-(5-bromo-4-((2,4-dimethoxybenzyl)amino)-7H-pyrrolo[2,3-d]pyrimidin-7-yl)-2-(((4-methylbenzoyl)oxy)methyl)tetrahydrofuran-3-yl 4-methylbenzoate as a yellow oil (155 g, 90%). LC-MS: 715 (M+1). ¹H NMR (400 MHz, CDCl₃): 8.36 (s, 1H), 8.04-7.86 (m, 4H), 7.34-7.28 (m, 5H), 7.06 (s, 1H), 6.79 (t, J=7.1 Hz, 1H), 6.64 (t, J=5.9 Hz, 1H), 6.52 (d, J=2.4 Hz, 1H), 6.46 (dd, J=8.2, 2.4 Hz, 1H), 5.79-5.61 (m, 1H), 4.77 (d, J=5.8 Hz, 2H), 4.74-4.61 (m, 2H), 4.57 (td, J=3.8, 2.3 Hz, 1H), 3.90 (s, 3H), 3.82 (s, 3H), 2.71 (dd, J=7.2, 4.2 Hz, 2H), 2.45 (d, J=6.3 Hz, 6H).

### 2.6 Synthesis of Intermediate B

To a solution of (2R,3S,5R)-5-(5-bromo-4-((2,4-dimethoxybenzyl)amino)-7H-pyrrolo[2,3-d]pyrimidin-7-yl)-2-(((4-methylbenzoyl)oxy)methyl)tetrahydrofuran-3-yl 4-methylbenzoate-(155 g, 0.216 mol, 1 eq) in THF (1240 mL) and water (310 mL) was added LiOH (45.5 g, 1.08 mol, 5 eq). Upon completion of the addition, the reaction mixture was stirred at room temperature for 16 hours. The mixture was diluted with water (1 L) and extracted with DCM (3×300 mL). The combined organic extracts were washed with a saturated NaHCO₃ solution (500 mL), brine (500 mL), dried over Na₂SO₄ and concentrated in vacuo to give the crude product which was purified by flash column chromatography (silica gel; eluting with 0-8% MeOH in DCM) to afford (2R,3S,5R)-5-(5-bromo-4-((2,4-dimethoxybenzyl)amino)-7H-pyrrolo[2,3-d]pyrimidin-7-yl)-2-(hydroxymethyl)tetrahydrofuran-3-ol (Intermediate B) as a light yellow solid (100 g, 96%). LCMS: 479 (M+1). ¹H NMR (400 MHz, DMSO-d6): 8.17 (s, 1H), 7.65 (s, 1H), 7.13 (d, J=8.2 Hz, 1H), 6.85 (t, J=6.0 Hz, 1H), 6.59 (d, J=2.3 Hz, 1H), 6.51 (dd, J=8.0, 6.0 Hz, 1H), 6.45 (dd, J=8.3, 2.4 Hz, 1H), 5.33-5.20 (m, 1H), 5.02 (t, J=5.5 Hz, 1H), 4.65 (d, J=5.9 Hz, 2H), 4.33 (q, J=3.1 Hz, 1H), 3.86 (s, 3H), 3.84, 3.78 (m, 1H), 3.73 (d, J=1.0 Hz, 3H), 3.62-3.44 (m, 2H), 2.45 (td, J=7.6, 3.9 Hz, 1H), 2.17 (ddd, J=13.0, 6.0, 2.8 Hz, 1H).

### Synthesis of Compounds

### A. General Method A

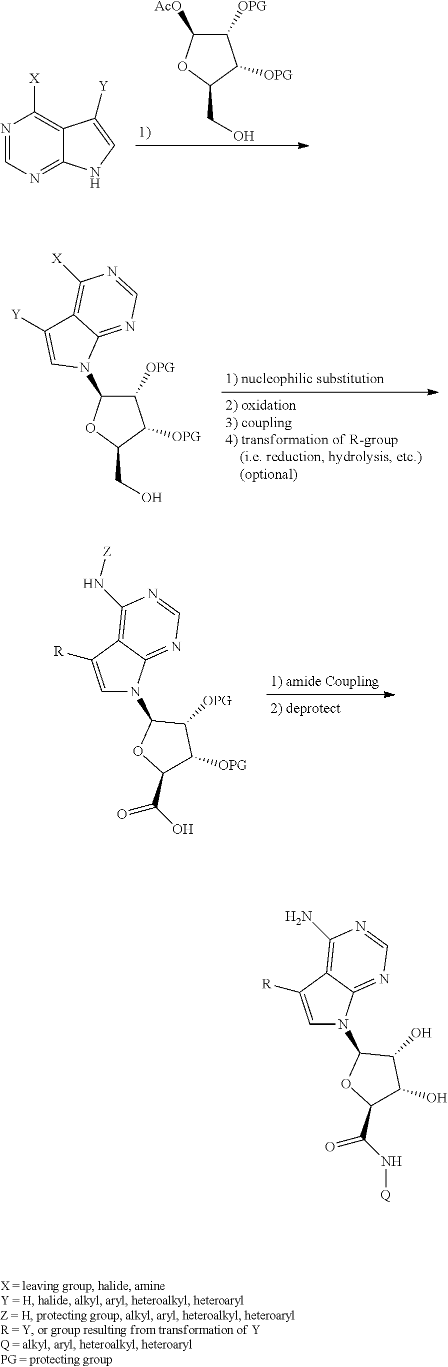

### Example 1: Synthetic Scheme for Compound I-1

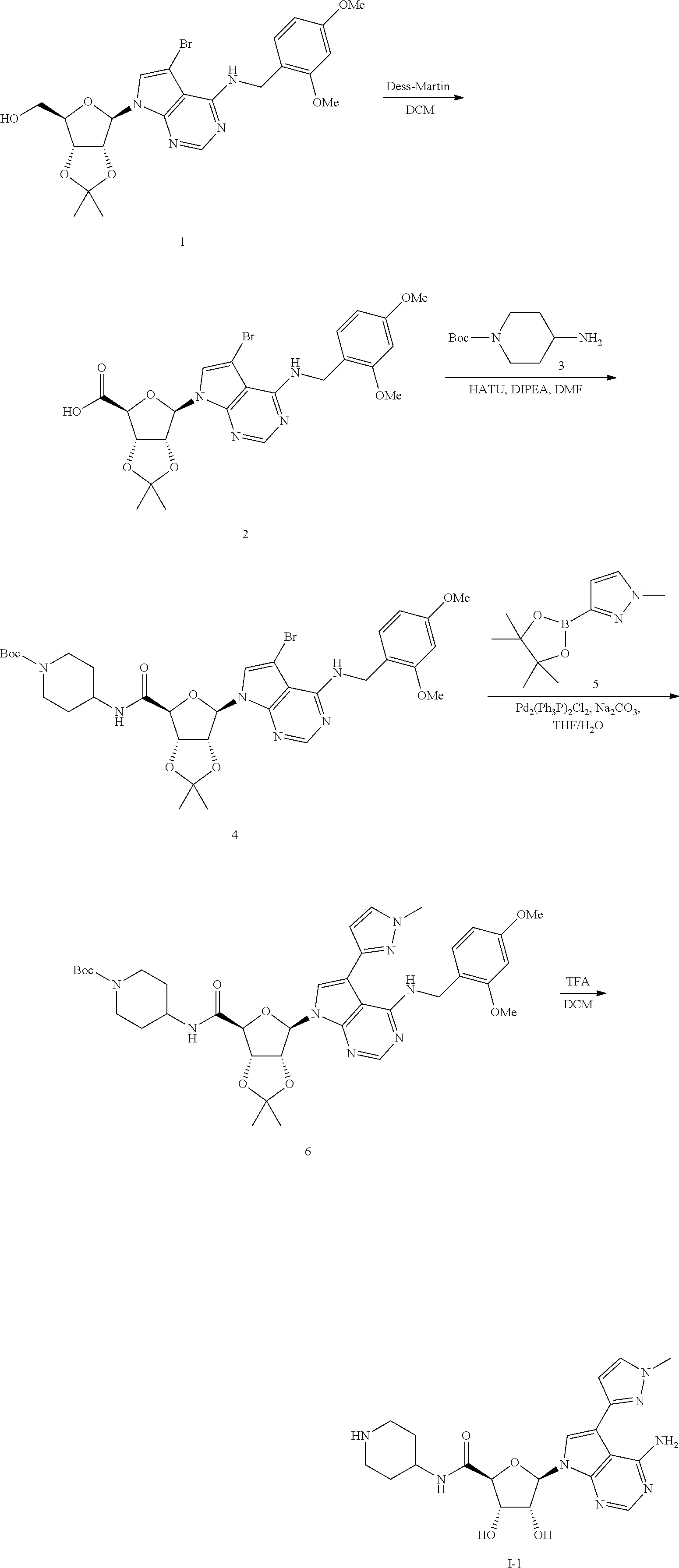

### 1.1 Synthesis of Compound 2

Dess Martin (7.33 g, 17.3 mmol) was added to the solution of [(3aR,4R,6R,6aR)-6-(5-bromo-4-{[(2,4-dimethoxyphenyl)methyl]amino}-7H-pyrrolo[2,3-d]pyrimidin-7-yl)-2,2-dimethyl-tetrahydro-2H-furo[3,4-d][1,3]dioxol-4-yl]methanol (1, Intermediate A, 3.1 g, 5.79 mmol) in DCM (50 mL), the reaction mixture was stirred at room temperature for 36 hours. The mixture was diluted with water (300 mL) and extracted with DCM (120 mL×2). Combined organic layers were washed with brine (60 mL), and dried with anhydrous Na₂SO₄. The solvent was removed under reduced pressure to get crude. The residue was purified by silica gel column chromatography (silica, 60 g, DCM/MeOH: 0˜30%) to get the target compound (3aS,4S,6R,6aR)-6-(5-bromo-4-{[(2,4-dimethoxyphenyl)methyl]amino}-7H-pyrrolo[2,3-d]pyrimidin-7-yl)-2,2-dimethyl-tetrahydro-2H-furo[3,4-d][1,3]dioxole-4-carboxylic acid (2.80 g, 5.09 mmol) as a yellow solid. ESI LCMS m/z=549.2 [M+1]⁺.

1.2 Synthesis of compound 4

(3aS,4S,6R,6aR)-6-(5-Bromo-4-{1[(2,4-dimethoxyphenyl)methyl]amino}-7H-pyrrolo[2,3-d]pyrimidin-7-yl)-2,2-dimethyl-tetrahydro-2H-furo[3,4-d][1,3]dioxole-4-carboxylic acid (200 mg, 364 μmol), tert-butyl 4-aminopiperidine-1-carboxylate (182 mg, 909 μmol), N,N-diisopropylethylamine (140 mg, 1.09 mmol) were mixed in DMF (3 mL). Then HATU (278 mg, 728 μmol) was added to the solution. The reaction mixture was stirred at room temperature for 12 hours. The mixture was diluted with water (60 mL) and extracted with EA (60 mL×2). Combined organic layers were washed with brine (60 mL), and dried with anhydrous Na₂SO₄. The solvent was removed under reduced pressure to get crude. The residue was purified by silica gel column chromatography (silica, 12 g, EA/PE: 0˜100%) to get the target compound tert-butyl 4-[(3aS,4S,6R,6aR)-6-(5-bromo-4-{[(2,4-dimethoxyphenyl)methyl]amino}-7H-pyrrolo[2,3-d]pyrimidin-7-yl)-2,2-dimethyl-tetrahydro-2H-furo[3,4-d][1,3]dioxole-4-amido]piperidine-1-carboxylate (170 mg, 232 μmol) as a yellow oil. ESI LCMS m/z=375.0 [M/2+1]⁺.

1.3 Synthesis of compound 6

tert-Butyl 4-[(3aS,4S,6R,6aR)-6-(5-bromo-4-{1[(2,4-dimethoxyphenyl)methyl]amino}-7H-pyrrolo[2,3-d]pyrimidin-7-yl)-2,2-dimethyl-tetrahydro-2H-furo[3,4-d][1,3]dioxole-4-amido]piperidine-1-carboxylate (170 mg, 232 μmol), 1-methyl-3-(4,4,5,5-tetramethyl-1,3,2-dioxaborolan-2-yl)-1H-pyrazole (90.7 mg, 436 μmol), palladium(2+) bis(triphenylphosphine) dichloride (15.3 mg, 21.8 μmol) and Na₂CO₃ (46.2 mg, 436 μmol) were mixed in THF/H₂O (4 mL), the reaction mixture was stirred at 75° C. for 12 hours under nitrogen atmosphere. The reaction was removed under reduced pressure to get crude. The residue was purified by silica gel column chromatography (silica, 12 g, EA/PE: 0˜50%) to get the target compound tert-butyl 4-[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-amido]piperidine-1-carboxylate (40.0 mg, 54.5 μmol) as a yellow oil. ESI LCMS m/z=733.3 [M+1]⁺.

1.4 Synthesis of compound I-1

TFA (1 ml) was added to the solution of tert-butyl 4-[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-amido]piperidine-1-carboxylate (40 mg, 54.5 μmol) in DCM (2 mL). The reaction mixture was stirred at room temperature for 2 hours. The mixture was concentrated under reduced pressure, the residue was neutralized by 3 mL 7M NH₃ in MeOH to pH=8, and then filtered. The filtrate was purified by Prep-HPLC to get (2S,3S,4R,5R)-5-[4-amino-5-(1-methyl-1H-pyrazol-3-yl)-7H-pyrrolo[2,3-d]pyrimidin-7-yl]-3,4-dihydroxy-N-(piperidin-4-yl)oxolane-2-carboxamide (5.70 mg, 12.8 μmol) as a light yellow solid. LC-MS m/z=443.0 [M+H]⁺. ¹H NMR (400 MHz, DMSO-d6) δ ppm 9.08 (s, 1H), 8.39 (d, J=8.2 Hz, 1H), 8.13 (s, 1H), 7.91 (s, 1H), 7.76 (d, J=2.2 Hz, 1H), 7.30 (s, 2H), 6.56 (d, J=2.2 Hz, 1H), 6.00 (d, J=7.5 Hz, 1H), 5.63 (d, J=4.2 Hz, 1H), 5.44 (d, J=6.5 Hz, 1H), 4.65 (d, J=5.0 Hz, 1H), 4.27 (d, J=1.4 Hz, 1H), 4.13 (s, 1H), 3.89 (s, 3H), 3.72 (s, 2H), 2.91 (s, 2H), 1.70 (dd, J=42.5, 11.5 Hz, 2H), 1.37-1.21 (m, 2H).

### Example 2: Synthetic Scheme for Compound I-10

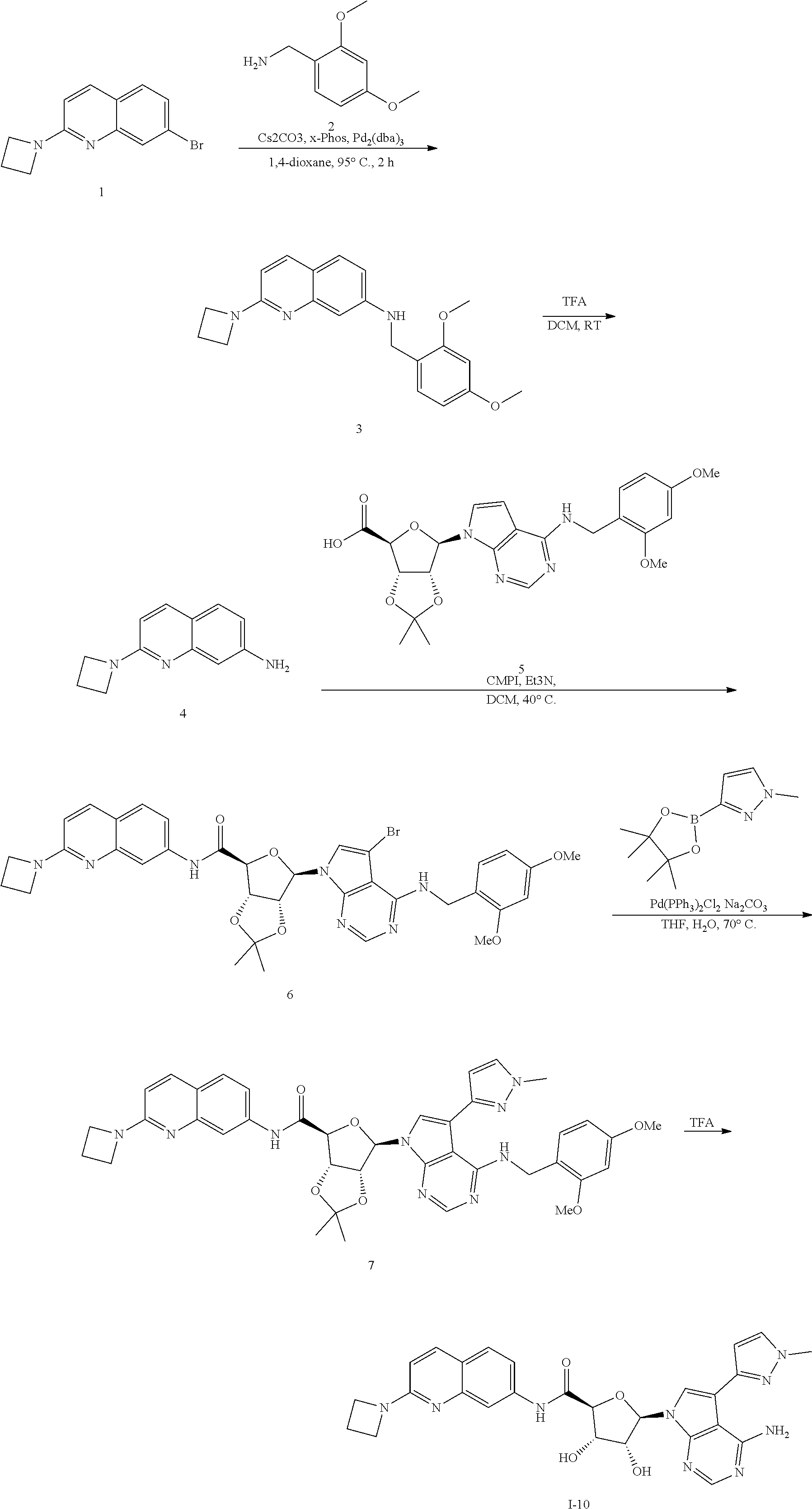

### 2.1 Synthesis of Compound 3

A mixture of 2-(azetidin-1-yl)-7-bromoquinoline (500 mg, 1.9 mmol), (2,4-dimethoxyphenyl)methanamine (476 mg, 2.85 mmol), and Cs₂CO₃ (1.85 g, 5.7 mmol), x-Phos (180 mg, 0.38 mmol), Pd₂(dba)₃ (347 mg, 0.38 mmol) in 20 mL of 1,4-dioxane, was heated to 95° C., under N₂, for 2 h. Then 50 mL of water was added into the above reaction mixture, extracted with EA (100 mL), the organic layer was dried with Na₂SO₄, filtered and concentrated, the residue was purified by flash chromatography, eluent (PE\EA: 2\1), afford 2-(azetidin-1-yl)-N-(2,4-dimethoxybenzyl)quinolin-7-amine (450 mg, 1.29 mmol) as a yellow solid. ESI LCMS m/z=350 [M+1]⁺.

### 2.2 Synthesis of Compound 4

A mixture of 2-(azetidin-1-yl)-N-(2,4-dimethoxybenzyl)quinolin-7-amine (450 mg, 1.29 mmol) in 4 mL of TFA was stirred at room temperature, for 3 h. Then the mixture was concentrated under reduced pressure, the residue was neutralized with 7M NH₃ in MeOH, to PH=8, concentrated to get 2-(azetidin-1-yl)quinolin-7-amine (180 mg, 0.904 mmol) as a yellow solid, yield: 70%, ESI LCMS m/z=200 [M+1]⁺.

### 2.3 Synthesis of Compound 6

To a solution of 2-(azetidin-1-yl)quinolin-7-amine (180 mg, 0.904 mmol) in methylene chloride (2 mL) was added (3aS,4S,6R,6aR)-6-(5-bromo-4-{[(2,4-dimethoxyphenyl)methyl]amino}-7H-pyrrolo[2,3-d]pyrimidin-7-yl)-2,2-dimethyl-tetrahydro-2H-furo[3,4-d][1,3]dioxole-4-carboxylic acid (495 mg, 0.904 mmol), 2-chloro-1-methylpyridin-1-ium iodide (459 mg, 1.8 mmol) and triethylamine (540 mg, 5.4 μmol), the mixture was stirred at 60° C. for 18 h. LC-MS analysis indicated that reaction worked well. Then 30 mL of water was added into the above reaction, extracted with EA (100 mL), the organic layer was dried with Na₂SO₄, filtered and concentrated, the residue was purified by flash chromatography, eluent: (PE\EA:1\2), afford (3aS,4S,6R,6aR)-N-(2-(azetidin-1-yl)quinolin-7-yl)-6-(5-bromo-4-((2,4-dimethoxybenzyl)amino)-7H-pyrrolo[2,3-d]pyrimidin-7-yl)-2,2-dimethyltetrahydrofuro[3,4-d][1,3]dioxole-4-carboxamide (200 mg, 0.27 mmol) as a yellow solid. Yield: 30%, ESI LCMS m/z=730 [M+1]⁺.

### 2.4 Synthesis of Compound 7

To A solution of (3aS,4S,6R,6aR)-N-[2-(azetidin-1-yl)quinolin-7-yl]-6-(5-bromo-4-{[(2,4-dimethoxyphenyl)methyl]amino}-7H-pyrrolo[2,3-d]pyrimidin-7-yl)-2,2-dimethyl-tetrahydro-2H-furo[3,4-d][1,3]dioxole-4-carboxamide (180 mg, 246 μmol) in tetrahydrofuran (5 mL) was added 1-methyl-3-(4,4,5,5-tetramethyl-1,3,2-dioxaborolan-2-yl)-1H-pyrazole (102 mg, 491 μmol), sodium carbonate (129 mg, 1.22 mmol) in water (2 mL) and palladium(2+) bis(ethane) methane bis(triphenylphosphine) dichloride (38.2 mg, 49.1 μmol) at rt, then the mixture was stirred at 60° C. for 18 h, under N₂. LC-MS analysis indicated that reaction worked well. The mixture was diluted with water (40 mL) and extracted with EA (40 mL×3). The organic layer was separated, washed with H₂O (40 mL) and brine (30 mL). The combined organics were dried (Na₂SO₄), evaporated in vacuum and the residue was purified by silica gel column chromatography, eluent: (DCM:MeOH=10:1), afford the product (3aS,4S,6R,6aR)-N-[2-(azetidin-1-yl)quinolin-7-yl]-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxamide (150 mg, 204 umol). Yield: 83%, ESI LCMS m/z=732 [M+1]⁺.

### 2.5 Synthesis of Compound I-10:

A mixture of (3aS,4S,6R,6aR)-N-[2-(azetidin-1-yl)quinolin-7-yl]-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxamide (150 mg, 204 μmol) and trifluoroacetic acid (6 mL) was stirred at room temperature for 8 h. The mixture was concentrated under reduced pressure and neutralized with 7M NH₃ in MeOH to PH:8, Then concentrated, the residue was purified by Pre-HPLC to get the target (2S,3S,4R,5R)-5-[4-amino-5-(1-methyl-1H-pyrazol-3-yl)-7H-pyrrolo[2,3-d]pyrimidin-7-yl]-N-[2-(azetidin-1-yl)quinolin-7-yl]-3,4-dihydroxyoxolane-2-carboxamide (38.4 mg, 70.9 μmol) as a white solid. ESI LCMS m/z=542.22 [M+H]⁺. ¹H NMR (400 MHz, DMSO-d6) δ ppm 10.46 (s, 1H), 9.16 (br, 1H), 8.07 (s, 1H), 8.02 (s, 1H), 8.01 (s, 1H), 7.93 (d, J=8.8 Hz, 1H), 7.75 (d, J=2.4 Hz, 1H), 7.65 (d, J=8.8 Hz, 1H), 7.34-7.31 (m, 2H), 6.61-6.57 (m, 2H), 6.16 (d, J=7.2 Hz, 1H), 5.75 (d, J=4.8 Hz, 1H), 5.56 (d, J=6.4 Hz, 1H), 4.67-4.64 (m, 1H), 4.54 (s, 1H), 4.36-4.35 (m, 1H), 4.10-4.06 (m, 4H), 3.89 (s, 1H), 2.38-2.34 (m, 2H).

### B. General Method B

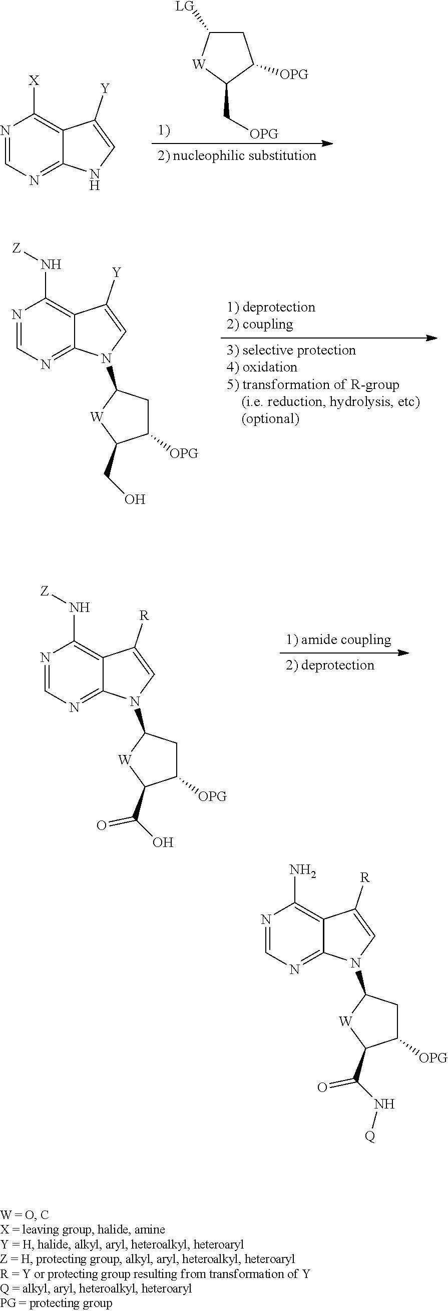

### Example 3: Synthetic Scheme for Compound I-17

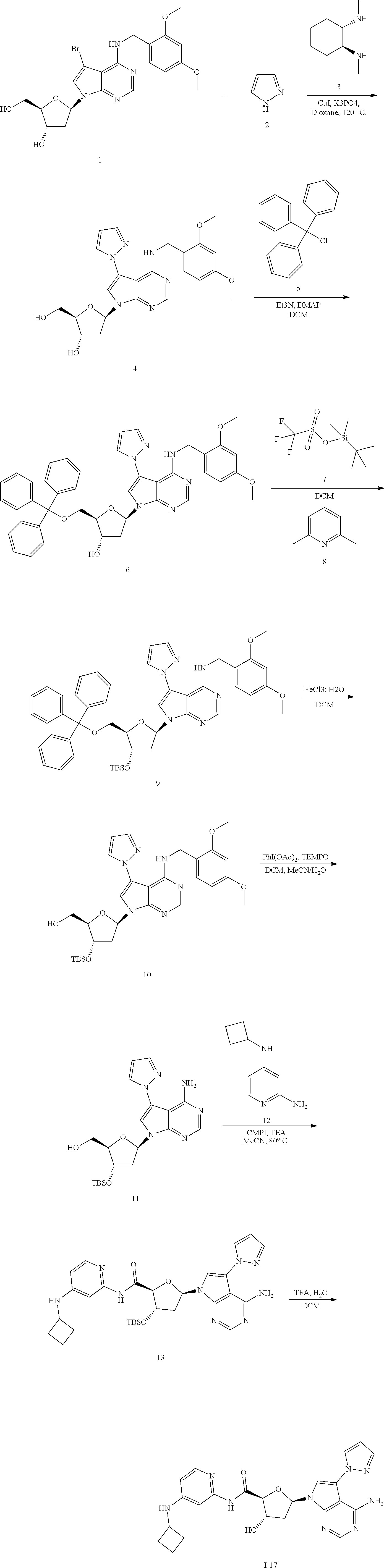

3.1 Synthesis of compound 4 To a solution of (2R,3S,5R)-5-(5-bromo-4-{[(2,4-dimethoxyphenyl)methyl]amino}-7H-pyrrolo[2,3-d]pyrimidin-7-yl)-2-(hydroxymethyl)oxolan-3-ol (1, Intermediate B, 5 g, 10.4 mmol) in Dioxane (40 mL), was added 1H-pyrazole (2, 4.83 g, 70.9 mmol), tripotassium phosphate (8.6 g, 40.5 mmol), (1S,2S)—N,N′-Dimethyl-1,2-cyclohexanediamine (3, 1.6 g, 11.2 mmol) and CuI (2.5 g, 13.1 mmol) at rt and stirred at 120° C. for 40 h. LC-MS analysis indicated that reaction was well. The solvent was removed under reduced pressure and the residue purified by silica gel column chromatography (DCM/MeOH=100/9) to give (2R,3S,5R)-5-(4-{[(2,4-dimethoxyphenyl)methyl]amino}-5-(1H-pyrazol-1-yl)-7H-pyrrolo[2,3-d]pyrimidin-7-yl)-2-(hydroxymethyl)oxolan-3-ol (4, 4.77 g, 10.2 mmol) as a brown solid. ESI LCMS m/z=467.1 [M+1]⁺.

### 3.2 Synthesis of Compound 6

To a solution of (2R,3S,5R)-5-(4-{[(2,4-dimethoxyphenyl)methyl]amino}-5-(1H-pyrazol-1-yl)-7H-pyrrolo[2,3-d]pyrimidin-7-yl)-2-(hydroxymethyl)oxolan-3-ol (4, 3 g, 6.43 mmol) and triethylamine (2.1 g, 20.7 mmol) in DCM (100 mL), was added DMAP (12 mg, 98.2 μmol) and (chlorodiphenylmethyl)benzene (5, 4 g, 14.3 mmol) and stirred at 50° C. for 1 h. TLC (PE/EA=1/1) showed the reaction was completed. The solvent was removed under reduced pressure and the residue purified by silica gel column chromatography (EA 60%) to give (2R,3S,5R)-5-(4-{[(2,4-dimethoxyphenyl)methyl]amino}-5-(1H-pyrazol-1-yl)-7H-pyrrolo[2,3-d]pyrimidin-7-yl)-2-[(triphenylmethoxy)methyl]oxolan-3-ol (6, 3.90 g, 5.50 mmol) as a yellow solid.

### 3.3 Synthesis of Compound 9

To a solution of (2R,3S,5R)-5-(4-{1[(2,4-dimethoxyphenyl)methyl]amino}-5-(1H-pyrazol-1-yl)-7H-pyrrolo[2,3-d]pyrimidin-7-yl)-2-[(triphenylmethoxy)methyl]oxolan-3-ol (6, 3.9 g, 5.50 mmol) in DCM (100 mL), was added lutidine (8, 3.85 g, 35.9 mmol) and then tert-butyldimethylsilyl trifluoromethanesulfonate (7, 6.1 g, 23.0 mmol) at 15° C. and stirred at 15° C. for 10 mins. TLC (PE/EA=3/1) showed the reaction was completed. The solvent was removed under reduced pressure and the residue purified by silica gel column chromatography (PE/EA=4/1) to give 7-[(2R,4S,5R)-4-[(tert-butyldimethylsilyl)oxy]-5-[(triphenylmethoxy)methyl]oxolan-2-yl]-N-[(2,4-dimethoxyphenyl)methyl]-5-(1H-pyrazol-1-yl)-7H-pyrrolo[2,3-d]pyrimidin-4-amine (9, 3.30 g, 4.00 mmol) as a yellow solid.

### 3.4 Synthesis of Compound 10

To a solution of 7-[(2R,4S,5R)-4-[(tert-butyldimethylsilyl)oxy]-5-[(triphenylmethoxy)methyl]oxolan-2-yl]-N-[(2,4-dimethoxyphenyl)methyl]-5-(1H-pyrazol-1-yl)-7H-pyrrolo[2,3-d]pyrimidin-4-amine (9, 2.17 g, 2.63 mmol) in DCM (40 mL), was added iron(III) chloride (1.06 g, 6.53 mmol) and H₂O (0.15 mL) at rt and stirred at rt for 7 mins. TLC (PE/EA=3/1) showed the reaction was completed. The mixture was diluted with H₂O (120 mL) and DCM (80 mL). The organic layer was separated, washed with NaHCO₃ aq (80 mL), H₂O (100 mL) and brine (60 mL), and dried (Na₂SO₄). The solvent was removed under reduced pressure and the residue purified by silica gel column chromatography (PE/EA=3/1) to give [(2R,3S,5R)-3-[(tert-butyldimethylsilyl)oxy]-5-(4-{[(2,4-dimethoxyphenyl)methyl]amino}-5-(1H-pyrazol-1-yl)-7H-pyrrolo[2,3-d]pyrimidin-7-yl)oxolan-2-yl]methanol (10, 1.03 g, 1.77 mmol) as a yellow solid.

### 3.5 Synthesis of Compound 11

BAIB (707 mg, 2.08 mmol) and TEMPO (36.2 mg, 232 μmol) were added to a solution of [(2R,3S,5R)-3-[(tert-butyldimethylsilyl)oxy]-5-(4-{[(2,4-dimethoxyphenyl)methyl]amino}-5-(1H-pyrazol-1-yl)-7H-pyrrolo[2,3-d]pyrimidin-7-yl)oxolan-2-yl]methanol (450 mg, 774 μmol) in DCM (8 mL) at room temperature. The mixture was stirred about 1 h, then CH₃CN/H₂O (1/1.1 mL) was added to above solution. Then reaction mixture was stirred at room temperature for 12 hours. The solvent was removed under reduced pressure to get crude. The crude was washed with PE (20 mL) for three times and triturated with THF/PE (1/10, 10 mL) to get (2S,3S,5R)-5-[4-amino-5-(1H-pyrazol-1-yl)-7H-pyrrolo[2,3-d]pyrimidin-7-yl]-3-[(tert-butyldimethylsilyl)oxy]oxolane-2-carboxylic acid (255 mg, 573 μmol) as a gray solid. ESI LCMS m/z=445.0 [M+1]⁺.

### 3.6 Synthesis of Compound 13

To a solution of (2S,3S,5R)-5-[4-amino-5-(1H-pyrazol-1-yl)-7H-pyrrolo[2,3-d]pyrimidin-7-yl]-3-[(tert-butyldimethylsilyl)oxy]oxolane-2-carboxylic acid (100 mg, 224 μmol) and N4-cyclobutylpyridine-2,4-diamine (43.7 mg, 268 μmol) in MeCN (5 mL) was added 2-chloro-1-methylpyridin-1-ium iodide (114 mg, 448 μmol), triethylamine (90.6 mg, 896 μmol). The reaction mixture was stirred at 80° C. for 3 h. LCMS showed the reaction worked well. The mixture was added with water (50 ml) and extracted with DCM (50 ml×3). The combined organic layers were dried (Na₂SO₄) and concentrated to dryness. The solvent was removed under reduced pressure to get crude. The mixture was purified by silica gel column (MeOH/DCM=8%) to get (2S,3S,5R)-5-[4-amino-5-(1H-pyrazol-1-yl)-7H-pyrrolo[2,3-d]pyrimidin-7-yl]-3-[(tert-butyldimethylsilyl)oxy]-N-[4-(cyclobutylamino)pyridin-2-yl]oxolane-2-carboxamide (100 mg, 169 μmol) as yellow solid. ESI LCMS m/z=590.0 [M+1]⁺.

### 3.7 Synthesis of Compound I-17:

To a solution of (2S,3S,5R)-5-[4-amino-5-(1H-pyrazol-1-yl)-7H-pyrrolo[2,3-d]pyrimidin-7-yl]-3-[(tert-butyldimethylsilyl)oxy]-N-[4-(cyclobutylamino)pyridin-2-yl]oxolane-2-carboxamide (100 mg, 169 μmol) in DCM (1 mL) was added TFA (6 mL) and 2 drops of water. The mixture was stirred at room temperature for 3 h. LCMS showed the reaction was completed. Then the mixture was concentrated to get crude, neutralized by saturated sodium bicarbonate solution. Then filter off to get crude solid. The crude was purified by Prep-HPLC to afford (2S,3S,5R)-5-[4-amino-5-(1H-pyrazol-1-yl)-7H-pyrrolo[2,3-d]pyrimidin-7-yl]-N-[4-(cyclobutylamino)pyridin-2-yl]-3-hydroxyoxolane-2-carboxamide (40.5 mg, 85.1 μmol) as white solid. ESI LCMS m/z=476.0 [M+1]⁺. ¹H NMR (400 MHz, DMSO-d6) δ ppm 10.12 (s, 1H), 8.33 (s, 1H), 8.31 (s, 1H), 7.99 (s, 1H), 7.86 (d, J=1.6 Hz, 1H), 7.79 (s, 1H), 7.36 (s, 1H), 6.97 (d, J=5.6 Hz, 1H), 6.96-6.92 (m, 1H), 6.85-6.82 (m, 1H), 6.68-6.64 (m, 1H), 6.58 (s, 1H), 6.24 (s, 1H), 5.83 (d, J=2.8 Hz, 1H), 4.60 (s, 1H), 4.88 (s, 1H), 3.86-3.82 (m, 1H), 3.30 (s, 1H), 2.77-2.75 (m, 1H), 2.33-2.24 (m, 4H), 1.89-1.70 (m, 4H).

### Example 4: Synthetic Scheme for Compound I-31

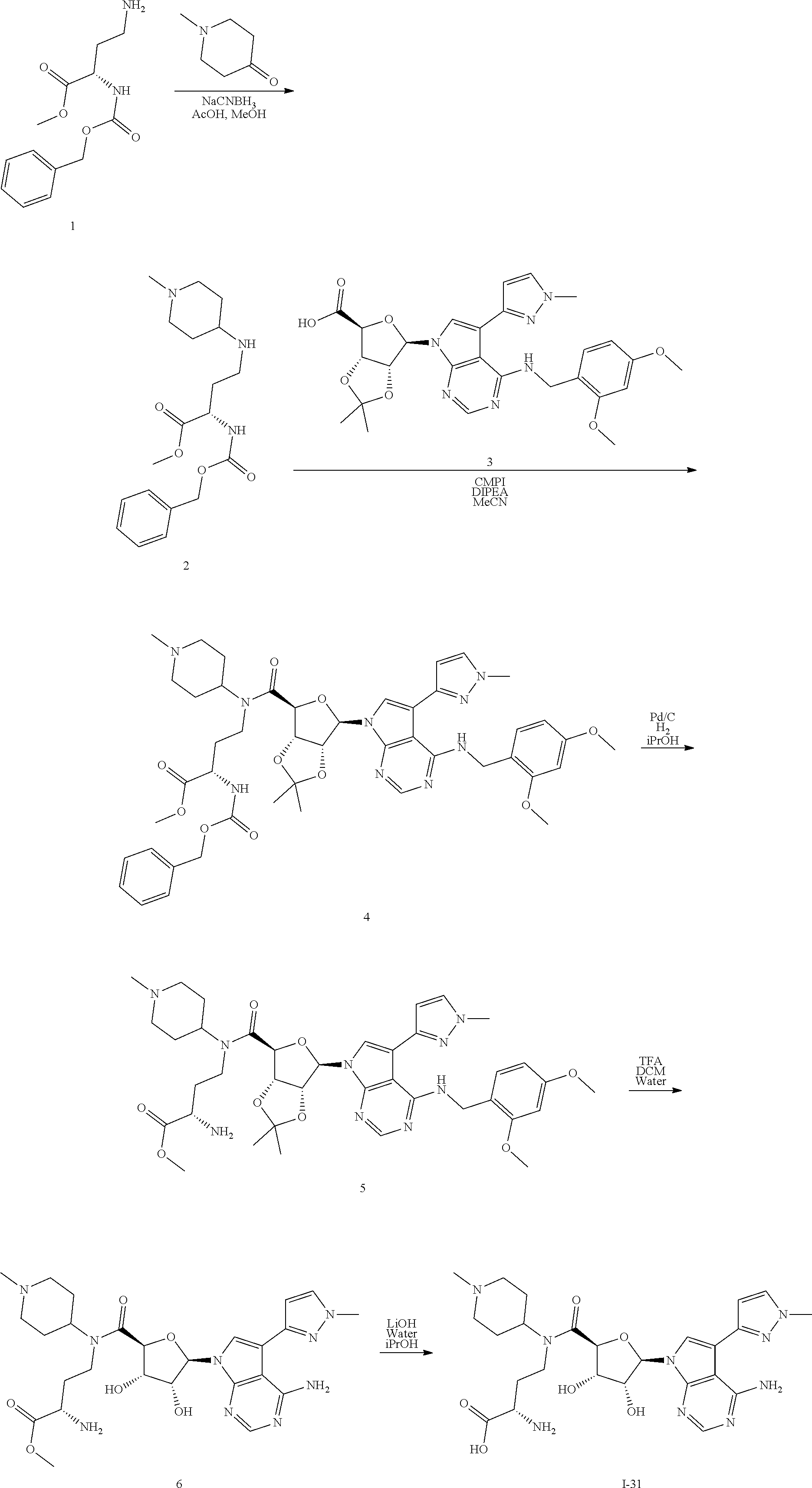

### 4.1 Synthesis of Compound 2

To a mixture of methyl (2S)-4-amino-2-{[(benzyloxy)carbonyl]amino}butanoate (230 mg, 863 μmol) in MeOH (5 mL) was added 1-methylpiperidin-4-one (315 mg, 2.78 mmol) and AcOH (0.1 mL). The mixture was stirred at room temperature for 30 min. Sodium cyanoborohydride (215 mg, 3.42 mmol) was added and the mixture stirred at room temperature for 1 hour. The solvent was removed under reduced pressure and the residue was diluted with water (50 mL) and extracted with ethyl acetate (50 mL). The organic layer was separated, washed with water (50 mL×2) and brine (20 mL), and dried (Na₂SO₄). The solvent was removed under reduced pressure to give crude methyl (2S)-2-{1[(benzyloxy)carbonyl]amino}-4-[(1-methylpiperidin-4-yl)amino]butanoate 2 (180 mg, 495 μmol).

### 4.2 Synthesis of Compound 4

To a solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylic acid (125 mg, 227 μmol) in MeCN (22 mL) was added methyl (2S)-2-{[(benzyloxy)carbonyl]amino}-4-[(1-methylpiperidin-4-yl)amino]butanoate 2 (180 mg, 495 μmol), N,N-diisopropylethylamine (350 mg, 2.70 mmol) and 2-chloro-1-methylpyridin-1-ium iodide (115 mg, 450 μmol) at room temperature. The reaction mixture was stirred at 80° C. for 2 hours. The solvent was removed and the residue was diluted with water (100 mL) and extracted with ethyl acetate (100 mL). The organic layer was separated, washed with water (100 mL×3) and brine (50 mL), and dried (Na₂SO₄). The solvent was removed under reduced pressure to give crude methyl (2S)-4-{1-[(3aS,4S,6R,6aR)-6-(4-{1[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]-N-(1-methylpiperidin-4-yl)formamido}-2-{1[(benzyloxy)carbonyl]amino}butanoate 4 (160 mg, 178 μmol).

### 4.3 Synthesis of Compound 5

To a solution of methyl (2S)-4-{1-[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]-N-(1-methylpiperidin-4-yl)formamido}-2-{[(benzyloxy)carbonyl]amino}butanoate 4 (150 mg, 167 μmol) in isopropyl alcohol (80 mL), was added Pd/C (200 mg, 187 μmol) at room temperature and stirred under a hydrogen atmosphere for 6 hours. The mixture was filtered and washed with isopropyl alcohol (55 mL). The solvent was removed under reduced pressure to give crude methyl (2S)-4-{1-[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]-N-(1-methylpiperidin-4-yl)formamido}-2-aminobutanoate 5 (90.0 mg, 118 μmol).

### 4.4 Synthesis of Compound 6

A solution of methyl (2S)-4-{1-[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]-N-(1-methylpiperidin-4-yl)formamido}-2-aminobutanoate 5 (90 mg, 118 μmol) in TFA (5 mL), water (0.15 mL) and DCM (0.5 mL) was stirred at room temperature for 2 hours. The mixture was concentrated to provide crude methyl (2S)-2-amino-4-{1-[(2S,3S,4R,5R)-5-[4-amino-5-(1-methyl-1H-pyrazol-3-yl)-7H-pyrrolo[2,3-d]pyrimidin-7-yl]-3,4-dihydroxyoxolan-2-yl]-N-(1-methylpiperidin-4-yl)formamido}butanoate 6 (55.0 mg, 96.2 μmol).

### 4.5 Synthesis of Compound I-31

To a solution of methyl (2S)-2-amino-4-{1-[(2S,3S,4R,5R)-5-[4-amino-5-(1-methyl-1H-pyrazol-3-yl)-7H-pyrrolo[2,3-d]pyrimidin-7-yl]-3,4-dihydroxyoxolan-2-yl]-N-(1-methylpiperidin-4-yl)formamido}butanoate 6 (55 mg, 96.2 μmol) in isopropyl alcohol (4 mL), was added water (1.5 mL) and lithium hydroxide hydrate (35 mg, 834 μmol) and stirred at room temperature for 2 hours. The product was purified by Prep-HPLC to afford (S)-2-amino-4-((2S,3S,4R,5R)-5-(4-amino-5-(1-methyl-1H-pyrazol-3-yl)-7H-pyrrolo[2,3-d]pyrimidin-7-yl)-3,4-dihydroxy-N-(1-methylpiperidin-4-yl)tetrahydrofuran-2-carboxamido)butanoic acid (12 mg, 21.5 μmol, 22%). ESI LCMS Calculated for C₂₅H₃₅N₉O₆: [M+H]⁺=558, found 558. ¹H NMR (400 MHz, DMSO-d6) δ ppm 8.97 (br s, 1H), 5.09 (s, 1H), 8.05 (s, 1H), 7.83 (s, 1H), 7.75, dd, J=10.2, 2.2 Hz, 1H), 7.19 (br s, 1H), 6.52 (dd, J=19.8, 2.2 Hz, 1H), 6.29 (dd, J=24.8, 6.4 Hz, 1H), 5.70 (br s, 1H), 4.83 (d, J=35.6 Hz, 1H), 4.41-4.46 (m, 1H), 4.32-4.40 (m, 1H), 4.15 (m, 1H), 3.89 (d, J=3.6 Hz, 3H), 3.66 (m, 1H), 3.20 (m, 1H), 2.73 (m, 1H), 2.14 (d, J=9.2 Hz, 3H), 1.91 (m, 4H), 1.75 (m, 4H), 1.42 (m, 2H).

### Example 5: Synthetic Scheme for Compound I-32

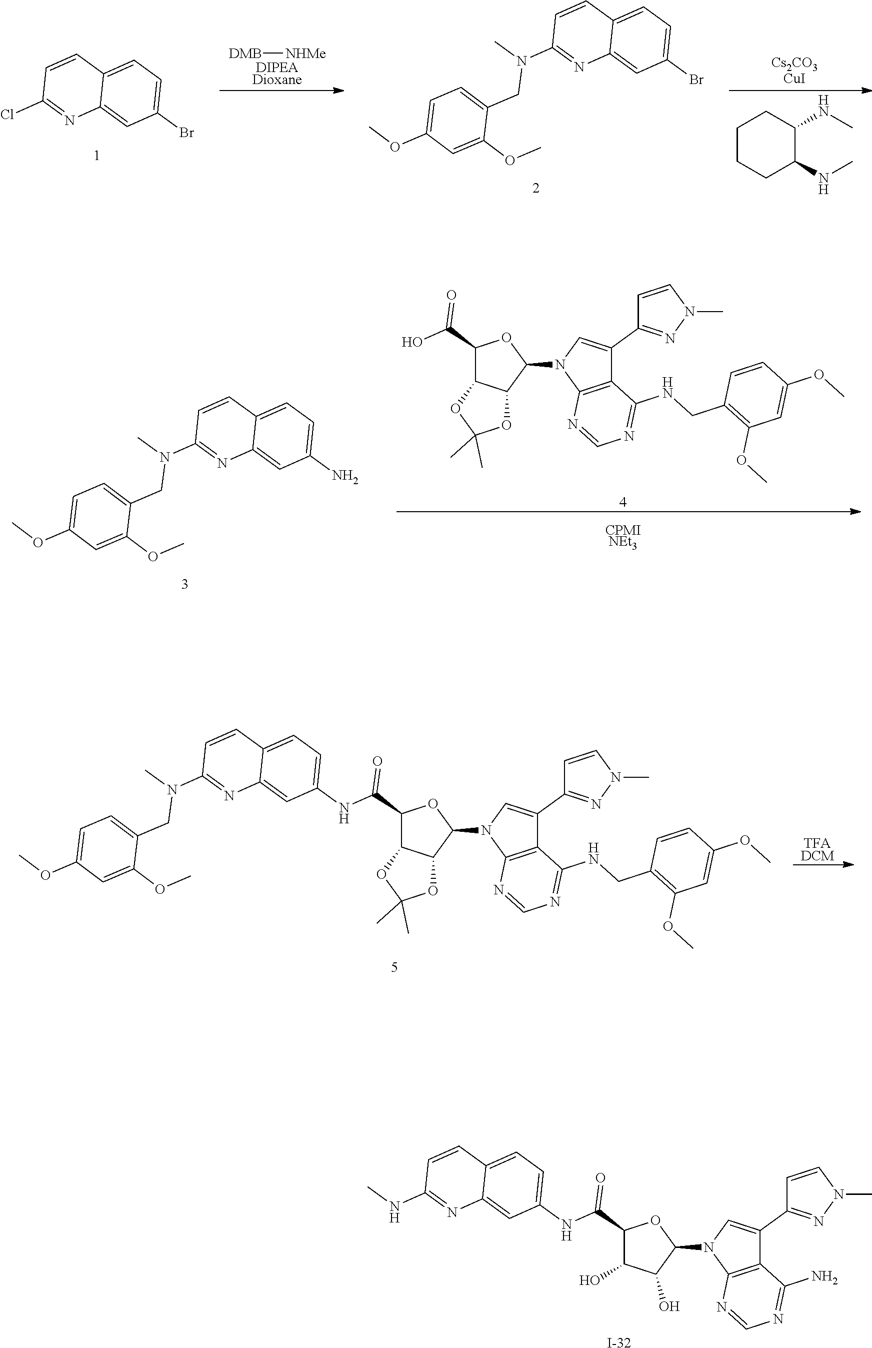

### 5.1 Synthesis of Compound 2

To a solution of 7-bromo-2-chloroquinoline (2 g, 8.24 mmol) in dioxane (5 mL) was added [(2,4-dimethoxyphenyl)methyl](methyl)amine (1.49 g, 8.24 mmol) and N,N-diisopropylethylamine (2.11 g, 16.4 mmol). The mixture was stirred at 120° C. for 6 hours under nitrogen atmosphere. Purification by chromatography on a silica gel column with a gradient of PE/EA (93:7) afforded the 7-bromo-N-[(2,4-dimethoxyphenyl)methyl]-N-methylquinolin-2-amine (2.20 g, 5.68 mmol).

### 5.2 Synthesis of Compound 3

To a solution of 7-bromo-N-[(2,4-dimethoxyphenyl)methyl]-N-methylquinolin-2-amine (400 mg, 1.03 mmol) in NH₃ in MeOH (30%) was added cesium carbonate (671 mg, 2.06 mmol), trans-1,2-Bis(methylamino)cyclohexane (0.2 equiv.) and copper iodide (39.2 mg, 206 μmol). The mixture was stirred at 120° C. for 24 hours. Purification by chromatography on a silica gel column with a gradient of DCM/MeOH (10:1) afforded N2-[(2,4-dimethoxyphenyl)methyl]-N2-methylquinoline-2,7-diamine (100 mg, 309 μmol).

### 5.3 Synthesis of Compound 5

To a solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylic acid (50 mg, 90.8 μmol) in acetonitrile (3 mL) was added N2-[(2,4-dimethoxyphenyl)methyl]-N2-methylquinoline-2,7-diamine (29.3 mg, 90.8 μmol), 2-chloro-1-methylpyridin-1-ium iodide (46.2 mg, 181 μmol) and triethylamine (0.4 mL). The mixture was stirred at 80° C. for 6 hours under nitrogen atmosphere. Purification by chromatography on a silica gel column with a gradient of DCM/MeOH (10:1) afforded (3aS,4S,6R,6aR)-N-(2-{[(2,4-dimethoxyphenyl)methyl](methyl)amino}quinolin-7-yl)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxamide (50.0 mg, 58.4 μmol).

### 5.4 Synthesis of Compound I-32

A mixture of (3aS,4S,6R,6aR)-N-(2-{[(2,4-dimethoxyphenyl)methyl](methyl)amino}quinolin-7-yl)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxamide (50 mg, 58.4 μmol) and TFA (3 mL) in DCM (2 mL) was stirred at room temperature for 5 hours. The mixture was concentrated and neutralized with 7M NH₃ in methanol solution. The mixture was concentrated and the residue was purified by prep-HPLC to afford (2S,3S,4R,5R)-5-[4-amino-5-(1-methyl-1H-pyrazol-3-yl)-7H-pyrrolo[2,3-d]pyrimidin-7-yl]-3,4-dihydroxy-N-[2-(methylamino)quinolin-7-yl]oxolane-2-carboxamide (I-33, 17.0 mg, 32.9 μmol) as a white solid. ESI LC-MS m/z=516.0 [M+H]⁺. ¹H NMR (400 MHz, DMSO-d6) δ ppm 10.42 (br s, 1H), 8.08 (s, 1H), 7.99 (s, 1H), 7.96 (s, 1H), 7.76 (m, 2H), 7.56 (d, J=8.8 Hz, 1H), 7.28 (dd, J=8.8, 2.0 Hz, 2H), 6.98 (m, 1H), 6.65 (d, J=8.8 Hz, 1H), 6.58 (d, J=2.0 Hz, 1H), 6.16 (d, J=6.8 Hz, 1H), 5.74 (d, J=4.8 Hz, 1H), 5.56 (d, J=6.4 Hz, 1H), 4.67 (q, J=6.8 Hz, 1H), 4.54 (d, J=2.0 Hz, 1H), 4.35 (m, 1H), 3.90 (s, 3H), 3.30 (m, 1H), 2.89 (d, J=4.8 Hz, 3H).

Compounds in Table 1 were prepared according to general methods A-B shown above using experimental procedures similar to those described in Examples 1-5. MS and ¹H NMR data are shown below.

TABLE 1

| | Synthetic | MS(ESI) | |
|---|---|---|---|
| Compound No. | Method | [M + H]⁺ | ¹H NMR |
| 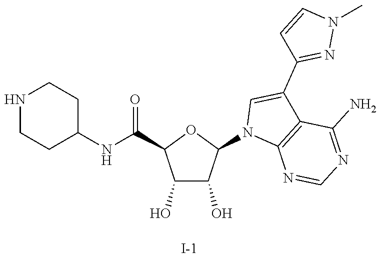 [SMILES: Cn1ccc(-c2cn([C@@H]3O[C@H](C(=O)NC4CCNCC4)[C@@H](O)[C@H]3O)c3ncnc(N)c23)n1 ; [I-]] | A | 443.0 | ¹H NMR (400 MHz, DMSO- d6) δ ppm 9.08 (s, 1H), 8.39 (d, J = 8.2 Hz, 1H), 8.13 (s, 1H), 7.91 (s, 1H), 7.76 (d, J = 2.2 Hz, 1H), 7.30 (s, 2H), 6.56 (d, J = 2.2 Hz, 1H), 6.00 (d, J = 7.5 Hz, 1H), 5.63 (d, J = 4.2 Hz, 1H), 5.44 (d, J = 6.5 Hz, 1H), 4.65 (d, J = 5.0 Hz, 1H), 4.27 (d, J = 1.4 Hz, 1H), 4.13 (s, 1H), 3.89 (s, 3H), 3.72 (s, 2H), 2.91 (s, 2H), 1.70 (dd, J = 42.5, 11.5 Hz, 2H), 1.37-1.21 (m, 2H). |
| 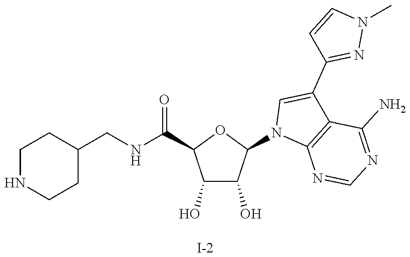 | A | 457.0 | ¹H NMR (400 MHz, DMSO- d6) δ ppm 9.10 (s, 1H), 8.90 (s, 1H), 8.10 (s, 1H), 7.90 (s, 1H), 7.76 (d, J = 2.2 Hz, 1H), 7.33 (s, 1H), 6.57 (d, J = 2.2 Hz, 1H), 5.97 (d, J = 7.7 Hz, 1H), 5.68 (s, 1H), 5.48 (s, 1H), 4.62 (s, 1H), 4.29 (s, 1H), 4.12 (s, 1H), 3.89 (s, 3H), 3.06 (s, 2H), 2.91 (d, J = 11.0 Hz, 2H), 2.40 (s, 3H), 1.54 (s, 3H), 1.02 (d, J = 10.8 Hz, 2H). |
| 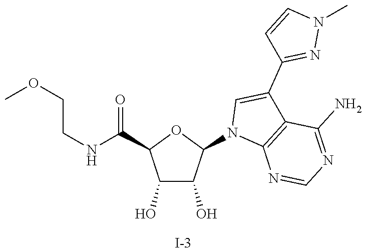 | A | 418.1 | ¹H NMR (400 MHz, DMSO- d6) δ ppm 9.07 (s, 1H), 8.84- 8.72 (m, 1H), 8.11 (s, 1H), 7.89 (s, 1H), 7.76 (d, J = 2.2 Hz, 1H), 7.31 (s, 1H), 6.59 (d, J = 2.3 Hz, 1H), 6.02 (d, J = 7.7 Hz, 1H), 5.68 (s, 1H), 5.47 (s, 1H), 4.68-4.47 (m, 1H), 4.30 (d, J = 1.4 Hz, 1H), 4.14 (d, J = 4.5 Hz, 1H), 3.89 (s, 3H), 3.45- 3.35 (m, 4H), 3.22 (s, 3H). |
| 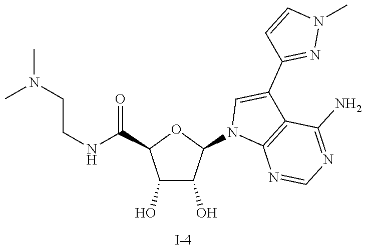 | A | 431.1 | ¹H NMR (400 MHz, DMSO- d6) δ ppm 9.07 (s, 1H), 8.51 (s, 1H), 8.10 (s, 1H), 7.90 (s, 1H), 7.76 (s, J = 2.4 Hz, 1H), 7.31 (s, 1H), 6.6 (d, J = 2.4 Hz, 1H), 6.05 (s, J = 8 Hz, 1H), 5.67 (s, 1H), 5.46 (d, J = 6.8 Hz, 1H), 4.64-4.59 (m, 1H), 4.28 (d, J = 1.2 Hz, 1H), 4.16 (d, J = 2 Hz, 1H), 3.89 (s, 3H), 3.31-3.26 (m, 2H), 2.42-2.38 (m, 2H), 2.14 (s, 6H). |
| 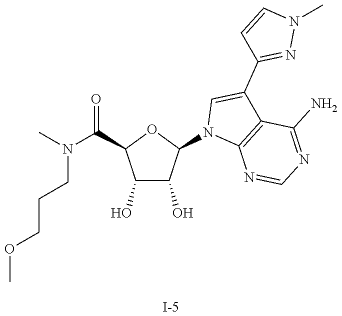 | A | 446.2 | ¹H NMR (400 MHz, DMSO- d6) δ ppm 8.95 (brs, 1H), 8.06 (s, 1H), 7.95 (s, 1H), 7.75 (s, 1H), 7.20 (brs, 1H), 6.51 (dd, J = 5.6 Hz, 2.4 Hz, 1H), 6.27 (t, J = 6.4 Hz, 1H), 5.56 (dd, J = 16 Hz 5.2 Hz, 1H), 6.27 (t, J = 5.2 Hz 1H), 4.76-4.74 (m, 1H), 4.45-4.36 (m, 1H), 4.31-4.26 (m, 1H), 3.84 (s, 3H), 3.41-3.36 (m, 2H), 3.31-3.27 (m, 2H), 3.21 (s, 3H), 3.01-2.86 (m, 3H), 1.77-1.67 (m, 2H) |
| 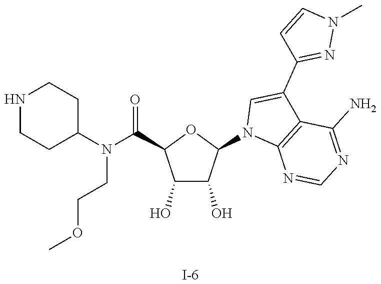 | A | 501.1 | ¹H NMR (400 MHz, DMSO- d6) δ ppm 8.94 (brs, 1H), 8.06 (s, 1H), 7.87-7.74 (m, 2H), 7.19 (brs, 1H), 6.50 (d, J = 4.0 Hz, 1H), 6.26 (d, J = 6.4 Hz, 1H), 5.58-5.48 (m, 2H), 4.80 (s, 1H), 4.50-4.24 (m, 2H), 3.88 (s, 3H), 3.50-3.38 (m, 6H), 3.24- 3.18 (m, 4H), 2.98-2.87 (m, 2H), 1.69-1.38 (m, 4H). |
| 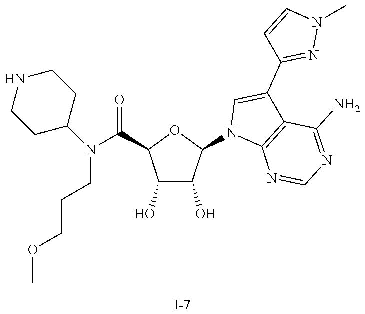 | A | 515.0 | ¹H NMR (400 MHz, DMSO- d6) δ ppm 8.95 (s, 1H), 8.06 (s, 1H), 7.84 (d, J = 37.8 Hz, 1H), 7.76 (t, J = 2.2 Hz, 1H), 7.21 (s, 1H), 6.52-6.44 (m, 1H), 6.32-6.23 (m, 1H), 5.60-5.45 (m, 2H), 4.80-4.65 (m, 1H), 4.54-4.40 (m, 1H), 4.28 (d, J = 31.3 Hz, 1H), 4.15 (s, 1H), 3.89 (s, 3H), 3.78 (s, 1H), 3.23 (s, 6H), 2.99 (s, 2H), 2.91 (s, 1H), 1.87-1.34 (m, 8H). |
| 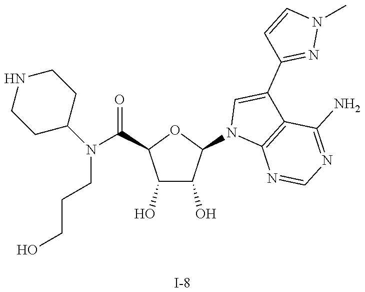 | A | 501.1 | ¹H NMR (400 MHz, DMSO- d6) δ ppm 8.94 (s, 1H), 8.06 (s, 1H), 7.85 (d, J = 48.3 Hz, 1H), 7.76 (dd, J = 4.2 Hz, 2.3 Hz, 1H), 7.20 (s, 1H), 6.49 (d, J = 2.2 Hz, 1H), 6.34-6.21 (m, 1H), 5.53 (s, 2H), 4.84-4.69 (m, 1H), 4.51 (t, J = 41.2 Hz, 2H), 4.27 (d, J = 35.8 Hz, 2H), 3.89 (d, J = 1.3 Hz, 3H), 3.78 (s, 1H), 3.42 (s, 5H), 2.99 (s, 2H), 1.90- 1.30 (m, 7H). |
| 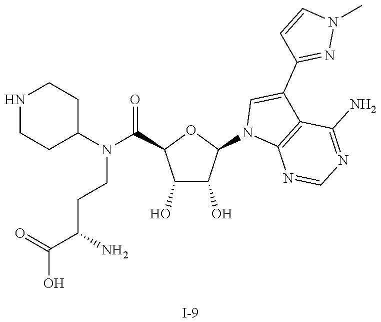 | A | 544.0 | ¹H NMR (400 MHz, DMSO- d6) δ ppm 8.95 (s, 1H), 8.31 (s, 2H), 8.06 (s, 1H), 7.93 (d, J = 66.3 Hz, 1H), 7.77-7.75 (m, 1H), 7.20 (s, 1H), 6.56-6.49 (m, 1H), 6.33-6.27 (m, 1H), 4.82 (d, J = 37.6 Hz, 1H), 4.52-4.39 (m, 1H), 4.32 (s, 1H), 4.23 (s, 1H), 3.97 (s, 1H), 3.89 (d, J = 1.5 Hz, 3H), 3.57-3.49 (m, 3H), 3.35 (s, 2H), 3.25-3.18 (m, 4H), 2.83-2.77 (m, 2H), 1.95 (s, 4H), 1.59-1.56 (m, 1H). |
| 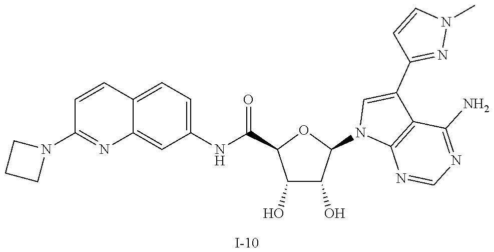 | A | 541.2 | ¹H NMR (400 MHz, DMSO- d6) δ ppm 10.48 (s, 1H), 9.06 (brs, 1H), 8.07 (s, 1H), 8.02 (s, 1H), 7.99 (s, 1H), 7.94 (d, J = 8.8 Hz, 1H), 7.75 (s, 1H), 7.65 (d, J = 8.2 Hz, 1H), 7.32 (d, J = 10.8 Hz, 2H), 6.61-6.57 (m, 2H), 6.165 (d, J = 7.2 Hz, 1H), 5.75 (d, J = 4.8 Hz, 1 H), 5.56 (d, J = 6.4 Hz, 1H), 4.67-4.66 (m, 1H), 4.55 (s, 1H), 4.36 (s, 1H), 4.09 (t, J = 7.2 Hz, 3H) 3.90 (s, 3H), 2.38-2.34 (m, 2H). |
| 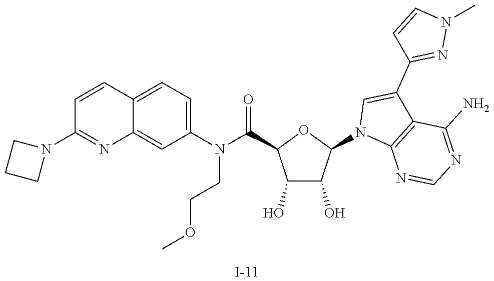 | A | 600.3 | ¹H NMR (400 MHz, DMSO- d6) δ ppm 8.95 (brs, 1H), 8.15 (s, 1H), 8.06-8.02 (m, 3H), 7.79-7.76 (m, 2H), 7.45 (s, 1H), 7.20-7.08 (m, 2H), 6.74 (d, J = 8.8 Hz, 1H), 6.54 (s, 1H), 6.16 (d, J = 6.4 Hz, 1H), 5.44 (brs, 1H), 5.30 (brs, 1 H), 4.53 (brs, 1H), 4.28 (s, 1H), 4.12-4.08 (m, 4H), 4.03-4.00 (m, 1H), 3.91 (s, 3H), 3.88-3.83 (m, 2H), 3.44- 3.41 (m, 1H), 3.18 (s, 3H), 2.38-2.32 (m, 2H). |
| 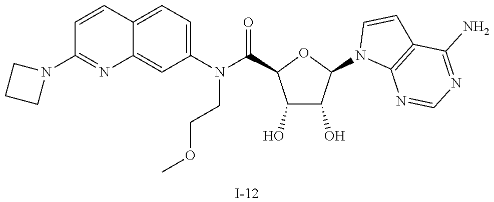 | A | 520.2 | ¹H NMR (400 MHz, DMSO- d6) δ ppm 8.14 (s, 1H), 8.7 (s, 2H), 7.80 (s, 1H), 7.65 (s, 1H), 7.46 (s, 1H), 7.24 (s, 2H), 7.12 (s, 1H), 6.77 (d, J = 8.8 Hz, 1H), 6.71 (s, 1H), 6.14 (s, 1H), 5.40 (s, 1H), 5.31 (s, 1H), 4.49 (s, 1H), 4.27-4.24 (m, 2H), 4.12 (d, J = 7.6 Hz, 4H), 3.99 (s, 1H), 3.44-3.42 (m, 2H), 3.21 (s, 3H), 2.39-2.33 (m, 2H). |
| 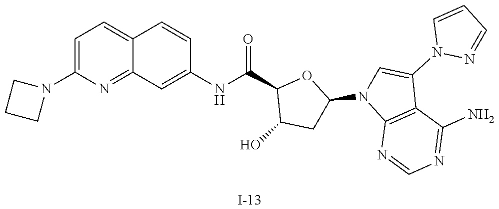 | B | 512.1 | ¹H NMR (400 MHz, DMSO- d6) δ ppm 10.21 (s, 1H), 8.27 (d, J = 2.4 Hz, 1H), 8.14 (s, 1H), 8.05 (s, 1H), 8.01 (s, 1H), 7.91 (d, J = 2.0 Hz, 1H), 7.85 (d, J = 1.6 Hz, 1H), 7.60 (d, J = 4.4 Hz, 1H), 7.28 (dd, J = 2.0 Hz 4.4 Hz, 1H), 6.78 (q, J = 5.2 Hz 8.8 Hz, 1H), 6.58 (d, J = 9.2 Hz, 1H), 6.55 (t, J = 2.4 Hz, 1H), 5.82 (d, J = 4.4 Hz, 1H), 4.67 (m, 1H), 4.51 (d, J = 1.2 Hz, 1H), 4.07 (t, J = 7.6 Hz, |
| | | | 4H), 2.79-2.71 (m, 1H), 2.38- |
| | | | 2.30 (m, 4H). |
| 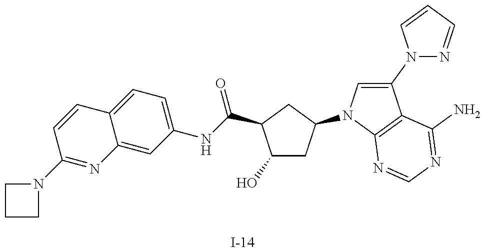 | B | 255.6 | ¹H NMR (400 MHz, DMSO- d6) δ ppm 10.28 (s, 1H), 8.43 (d, J = 2.3 Hz, 2H), 8.13 (s, 2H), 7.96 (s, 2H), 7.83 (d, J = 1.7 Hz, 1H), 7.65 (d, J = 8.3 Hz, 1H), 7.40 (d, J = 8.7 Hz, 1H), 6.69-6.54 (m, 2H), 5.58- 5.31 (m, 2H), 4.58 (s, 1H), 4.12 (s, 4H), 3.06-2.92 (m, 1H), 2.48-2.25 (m, 4H), 2.17 (dd, J = 22.4, 9.9 Hz, 3H). |
| 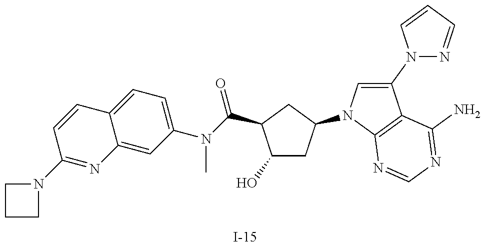 | B | 526.1 | ¹H NMR (400 MHz, DMSO- d6) δ ppm 8.34 (s, 1H), 8.11 (d, J = 6.4 Hz, 1H), 8.04 (d, J = 8.8 Hz, 1H), 7.87 (s, 1H), 7.77 (d, J = 7.6 Hz, 1H), 7.48 (d, J = 1.6 Hz, 1H), 7.13 (dd, J = 8.4 Hz, 2 Hz, 1H), 6.73 (d, J = 8.8 Hz, 1H), 6.60 (s, 1H), 5.40 (s, 1H), 4.58 (s, 1H), 4.37 (s, 1H), 4.10 (t, J = 7.6 Hz, 4H), 3.28 (s, 3H), 2.67-2.63 (m, 1H), 2.40-2.33 (m, 2H), 2.30-2.26 (m, 1H). |
| 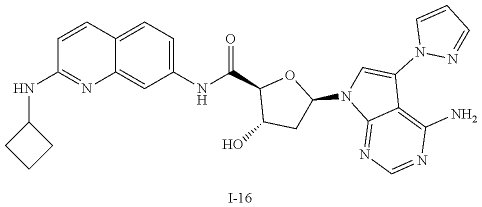 | B | 526.0 | ¹H NMR (400 MHz, DMSO- d6) δ ppm 10.16 (s, 1H), 8.28 (d, J = 2.4 Hz, 1H), 8.15 (s, 1H), 8.05 (s, 1H), 7.91 (d, J = 2.0 Hz, 1H), 7.85 (d, J = 1.6 Hz, 1H), 7.75 (d, J = 4.4 Hz, 1H), 7.51 (d, J = 4.4 Hz, 1H), 7.26-7.21 (m, 2H), 6.78 (q, J = 5.2 Hz 8.8 Hz, 1H), 6.60 (d, J = 9.2 Hz, 1H), 6.54 (t, J = 2.4 Hz, 1H), 5.82 (d, J = 4.4 Hz, 1H), 4.67 (s, 1H), 4.54-4.48 (m, 2H), 3.30 (s, 2H), 2.79-2.72 (m, |
| | | | 1H), 2.35-2.28 (m, 3H), 1.93- |
| | | | 1.66 (m, 4H). |
| 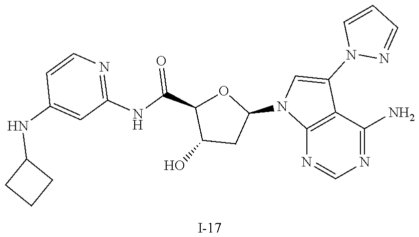 | B | 476.0 | ¹H NMR (400 MHz, DMSO- d6) δ ppm 10.13 (s, 1H), 8.31 (d, J = 8.4 Hz, 1H), 8.00 (s, 1H), 7.86 (d, J = 1.6 Hz, 1H), 7.79 (s, 1H), 7.36 (s, 1H), 6.97 (d, J = 5.6 Hz, 1H), 6.67-6.64 (m, 1H), 6.58 (s, 1H), 6.24 (s, 1H), 5.83 (d, J = 2.8 Hz, 1H), 4.60 (s, 1H), 4.48 (s, 1H), 3.86- 3.82 (m, 1H), 3.28 (s, 1H), 2.77-2.75 (m, 1H), 2.33-2.24 (m, 4H), 1.89-1.70 (m, 4H). |
| 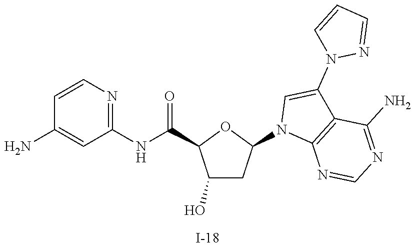 | B | 422.1 | ¹H NMR (400 MHz, DMSO- d6) δ ppm 10.11 (s, 1H), 8.33 (d, J = 2 Hz, 1H), 8.30 (s, 1H), 7.99 (s, 1H), 7.86 (d, J = 2 Hz, 1H), 7.73 (d, J = 5.6 Hz, 1H), 7.38 (s, 1H), 6.67-6.64 (m, 1H), 6.59 (t, J = 2 Hz, 1H), 6.26 (dd, J = 5.6 Hz 1.6 Hz, 1H), 6.15 (s, 1H), 5.84 (d, J = 4 Hz, 1H), 4.60 (t, J = 4.8 Hz, 1H), 4.47 (d, J = 0.8 Hz, 1H), 2.79-2.73 (m, 1H), 2.28-2.24 (m, 1H). |
| 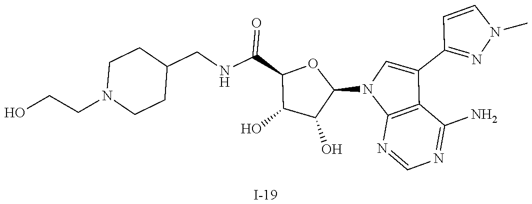 | A | 501.0 | ¹H NMR (400 MHz, DMSO- d6) δ ppm 9.08 (brs, 1H), 8.87 (t, J = 6.0 Hz, 1H), 8.11 (s, 1H), 7.88 (s, 1H), 7.76 (d, J = 2.4 Hz, 1H), 7.32 (brs, 1H), 6.57 (d, J = 2.0 Hz, 1H), 5.97 (d, J = 7.6 Hz, 1H), 5.65 (d, J = 4.4 Hz, 1H), 5.46 (d, J = 6.8 Hz, 1H), 4.66-4.61 (m, 1H), 4.32-4.31 (m, 1H), 4.29 (d, J = 1.6 Hz, 1H), 4.13-4.10 (m, 1H), 3.89 (s, 3H), 3.47-3.40 (m, 2H), 3.08 (t, J = 6.0 Hz, 2H), 2.81- |
| | | | 2.78 (m, 2H), 2.32 (t, J = 6.0 |
| | | | Hz, 2H), 1.88-1.83 (m, 2H), |
| | | | 1.57-1.54 (m, 2H), 1.46-1.39 |
| | | | (m, 1H), 1.18-1.08 (m, 2H). |
| 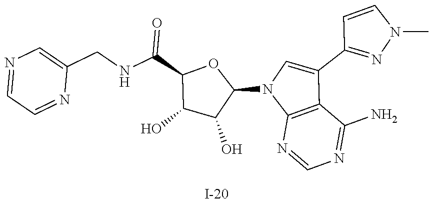 | A | 452.0 | ¹H NMR (400 MHz, DMSO- d6) δ ppm 9.57 (t, J = 5.6 Hz, 1H), 9.06 (s, 1H), 8.62 (d, J = 1.6 Hz, 1H), 8.56-8.52 (m, 2H), 7.92 (s, 1H), 7.88 (s, 1H), 7.73 (d, J = 2.0 Hz, 1H), 7.29 (m, 1H), 6.50 (d, J = 2.4 Hz, 1H), 6.02 (d, J = 7.6 Hz, 1H), 5.70 (d, J = 2.4 Hz, 1H), 5.48 (d, J = 6.8 Hz, 1H), 4.67-4.57 (m, 3H), 4.39 (d, J = 1.2 Hz, 1H), 4.19 (td, J = 1.6 Hz 4.4 Hz, 1H), 3.87 (s, 3H). |
| 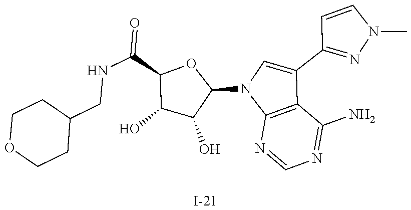 | A | 458.0 | ¹H NMR (400 MHz, DMSO- d6) δ ppm 9.08 (s, 1H), 8.90 (t, J = 4.2 Hz, 1H), 8.12 (s, 1H), 7.89 (s, 1H), 7.76 (d, J = 2.4 Hz, 1H), 7.32 (s, 1H), 6.56 (d, J = 3.2 Hz, 1H), 5.97 (d, J = 8 Hz, 1H), 5.65 (d, J = 4.8 Hz, 1H), 5.44 (d, J = 2 Hz, 1H), 4.65- 4.60 (m, 1H), 4.29 (d, J = 1.6 Hz, 1H), 4.14-4.12 (m, 1H), 3.89 (s, 3H), 3.82-3.78 (m, 1H), 3.24-3.04 (m, 4H), 1.72-1.66 (m, 1H), 1.51-1.17 (m, 4H). |
| 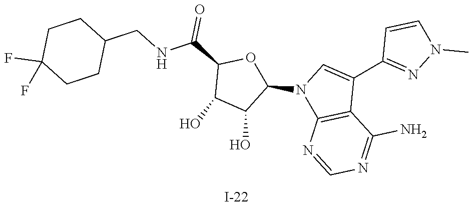 | A | 492.0 | ¹H NMR (400 MHz, DMSO- d6) δ ppm 9.18-9.05 (br, 1H), 8.96 (t, J = 5.9 Hz, 1H), 8.12 (s, 1H), 7.90 (s, 1H), 7.76 (d, J = 2.2 Hz, 1H), 7.40-7.25 (br, 1H), 6.57 (d, J = 2.2 Hz, 1H), 5.98 (d, J = 7.6 Hz, 1H), 5.66 (d, J = 4.1 Hz, 12H), 5.46 (d, J = 6.5 Hz, 1H), 4.66-4.61 (m, 1H), 4.30 (s, 1H), 4.17-4.11 (m, 1H), 3.89 (s, 3H), 3.11 (t, J = 6.3 Hz, 2H), 2.03-1.90 (m, 2H), 1.82-1.54 (m, 5H), 1.24-1.10 |
| | | | (m, 2H). |
| 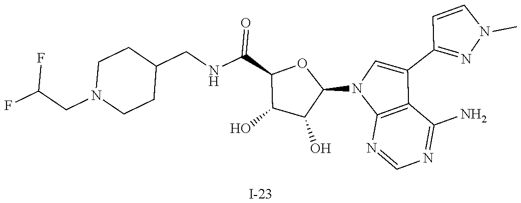 | A | 520.9 | ¹H NMR (400 MHz, DMSO- d6) δ ppm 9.09 (brs, 1H), 8.89 (t, J = 6.0 Hz, 1H), 8.11 (s, 1H), 7.89 (s, 1H), 7.76 (d, J = 2.0 Hz, 1H), 7.32 (brs, 1H), 6.57 (d, J = 2.4 Hz, 1H), 6.08 (tt, J = 56.0, 4.4 Hz, 1H), 5.97 (d, J = 7.6 Hz, 1H), 5.65 (d, J = 4.4 Hz, 1H), 5.45 (d, J = 6.8 Hz, 1H), 4.66-4.61 (m, 1H), 4.29 (d, J = 1.6 Hz, 1H), 4.13- 4.11 (m, 1H), 3.89 (s, 3H), 3.08 (t, J = 6.4 Hz, 2H), 2.85-2.82 |
| | | | (m, 2H), 2.70-2.61 (m, 2H), |
| | | | 2.08-2.03 (m, 2H), 1.57-1.54 |
| | | | (m, 2H), 1.47-1.40 (m, 1H), |
| | | | 1.19-1.09 (m, 2H). |
| 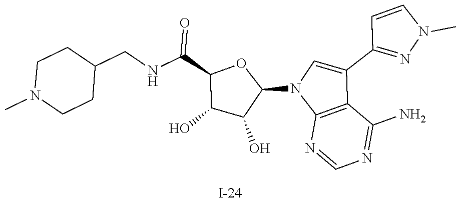 | A | 471.0 | ¹H NMR (400 MHz, DMSO- d6) δ ppm 9.08 (s, 1H), 8.95- 8.81 (m, 1H), 8.10 (s, 1H), 7.88 (s, 1H), 7.76 (d, J = 2.2 Hz, 1H), 7.31 (s, 1H), 6.57 (d, J = 2.3 Hz, 1H), 5.97 (d, J = 7.7 Hz, 1H), 5.64 (d, J = 4.3 Hz, 1H), 5.45 (d, J = 6.6 Hz, 1H), 4.70-4.55 (m, 1H), 4.29 (d, J = 4 Hz, 1H), 4.14-4.10 (m, 1H), 3.89 (s, 3H), 3.15-3.05 (m, 2H), 2.80-2.60 (m, 2H), 2.12 (s, 3H), 1.90-1.70 (m, 2H), 1.65-1.50 |
| | | | (m, 2H), 1.45-1.30 (m, 1H), |
| | | | 1.25-1.05 (m, 2H). |
| 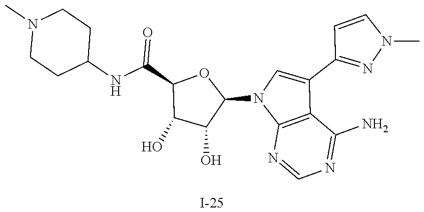 | A | 457.0 | ¹H NMR (400 MHz, DMSO- d6) δ ppm 9.07 (s, 1H), 8.32 (d, J = 8.1 Hz, 1H), 8.09 (s, 1H), 7.92 (s, 1H), 7.75 (d, J = 2.2 Hz, 1H), 7.29 (s, 1H), 6.56 (d, J = 2.3 Hz, 1H), 6.03 (d, J = 7.5 Hz, 1H), 5.62 (d, J = 4.4 Hz, 1H), 5.44 (d, J = 6.6 Hz, 1H), 4.70-4.60 (m, 1H), 4.27 (d, J = 1.6 Hz, 1H), 4.19-4.03 (m, 1H), 3.89 (s, 3H), 3.74-3.42 (m, 1H), 2.91-2.58 (m, 2H), 2.15 (s, 3H), 2.02-1.85 (m, 2H), 1.83-1.73 |
| | | | (m, 1H), 1.72-1.62 (m, 1H), |
| | | | 1.55-1.35 (m, 2H). |
| 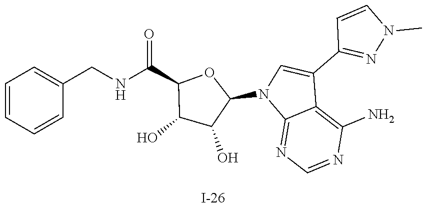 | A | 450.1 | ¹H NMR (400 MHz, DMSO- d6) δ ppm 9.55 (t, J = 6 Hz, 1H), 9.09 (s, 1H), 7.86 (s, 1H), 7.74 (s, 1H), 7.73 (s, 1H), 7.28- 7.26 (m, 6H), 6.52 (d, J = 2 Hz, 1H), 5.96 (d, J = 7.6 Hz, 1H), 5.70 (d, J = 4.4 Hz, 1H), 5.47 (d, J = 6.8 Hz, 1H), 4.67- 4.66 (m, 1H), 4.48-4.46 (m, 3H). |
| 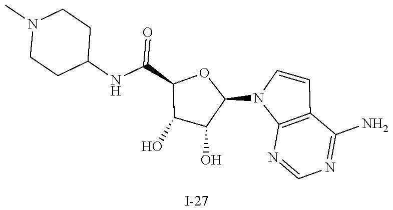 | A | 377.0 | ¹H NMR (400 MHz, DMSO- d6) δ ppm 8.47 (d, J = 8.1 Hz, 1H), 8.09 (s, 1H), 7.41 (d, J = 3.6 Hz, 1H), 7.13 (s, 2H), 6.60 (d, J = 3.6 Hz, 1H), 5.97 (d, J = 7.6 Hz, 1H), 5.61 (d, J = 4.0 Hz, 1H), 5.36 (d, J = 6.6 Hz, 1H), 4.70-4.55 (m, 1H), 4.26 (d, J = 1.4 Hz, 1H), 4.13-4.03 (m, 1H), 3.73-3.45 (m, 1H), 2.81-2.63 (m, 2H), 2.15 (s, 3H), 2.00-1.85 (m, 2H), 1.81-1.72 (m, 1H), 1.71-1.63 (m, 1H), |
| | | | 1.56-1.36 (m, 2H). |
| 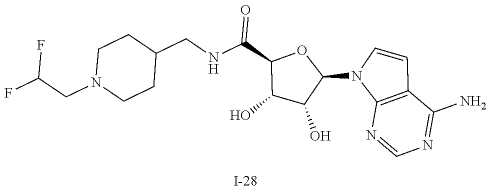 | A | 441.0 | ¹H NMR (400 MHz, DMSO- d6) δ ppm 9.06 (t, J = 6.0 Hz, 1H), 8.11 (s, 1H), 7.37 (d, J = 3.6 Hz, 1H), 7.14 (s, 2H), 6.60 (d, J = 3.6 Hz, 1H), 6.09 (tt, J = 56.0, 4.4 Hz, 1H), 5.92 (d, J = 8.0 Hz, 1H), 5.64 (d, J = 4.0 Hz, 1H), 5.37 (d, J = 6.4 Hz, 1H), 4.62-4.57 (m, 1H), 4.28 (s, 1H), 4.07 (t, J = 4.0 Hz, 1H), 3.11-3.04 (m, 2H), 2.87-2.84 (m, 2H), 2.71-2.62 (m, 2H), |
| | | | 2.10-2.04 (m, 2H), 1.58-1.54 |
| | | | (m, 2H), 1.45-1.38 (m, 1H), |
| | | | 1.19-1.11 (m, 2H). |
| 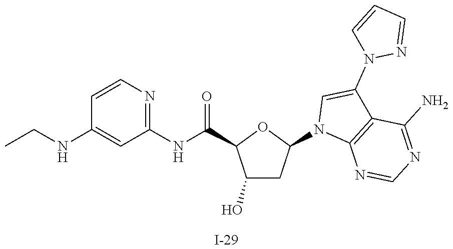 | B | 450.0 | ¹H NMR (400 MHz, DMSO- d6) δ ppm 10.12 (s, 1H), 8.33 (d, J = 2.4 Hz, 1H), 8.31 (s, 1H), 7.99 (s, 1H), 7.86 (d, J = 1.6 Hz, 1H), 7.79 (d, J = 5.8 Hz, 1H), 7.39 (s, 1H), 6.70- 6.62 (m, 2H), 6.61-6.56 (m, 1H), 6.28 (dd, J = 5.8 Hz 2.2 Hz, 1H), 5.83 (d, J = 4.1 Hz, 1H), 4.60 (s, 1H), 4.47 (s, 1H), 3.14-2.99 (m, 2H), 2.79-2.75 (m, 1H), 2.36-2.21 (m, 1H), 1.15 (t, J = 7.2 Hz, 3H). |
| 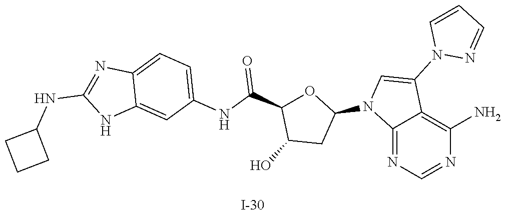 | B | 514.9 | ¹H NMR (400 MHz, DMSO- d6) δ ppm 10.65 (d, 1H), 9.95 (d, 1H), 8.26 (d, 1H), 8.12 (d, 1H), 8.06 (s, 1H), 7.85 (s, 1H), 7.62 (m, 1H), 7.02-6.87 (m, 3H), 6.74-6.70 (m, 1H), 6.56 (d, 1H), 5.79 (d, 1H), 4.61 (s, 1H), 4.44 (s, 1H), 4.18-4.14 (m, 1H), 2.78-2.71 (m, 1H), 2.32- 2.25 (m, 3H), 1.97-1.60 (m, 4H). |
| 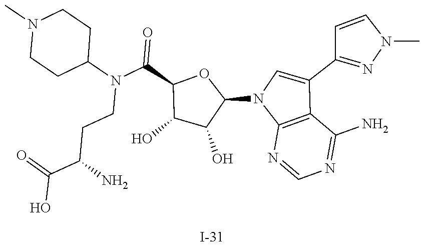 | A | 558.0 | ¹H NMR (400 MHz, DMSO- d6) δ ppm 8.97 (br s, 1H), 5.09 (s, 1H), 8.05 (s, 1H), 7.83 (s, 1H), 7.75, dd, J = 10.2, 2.2 Hz, 1H), 7.19 (br s, 1H), 6.52 (dd, J = 19.8, 2.2 Hz, 1H), 6.29 (dd, J = 24.8, 6.4 Hz, 1H), 5.70 (br s, 1H), 4.83 (d, J = 35.6 Hz, 1H), 4.41-4.46 (m, 1H), 4.32-4.40 (m, 1H), 4.15 (m, 1H), 3.89 (d, J = 3.6 Hz, 3H), 3.66 (m, 1H), 3.20 (m, 1H), 2.73 (m, 1H), 2.14 (d, J = 9.2 Hz, 3H), 1.91 (m, 4H), 1.75 (m, 4H), 1.42 (m, 2H) |
| 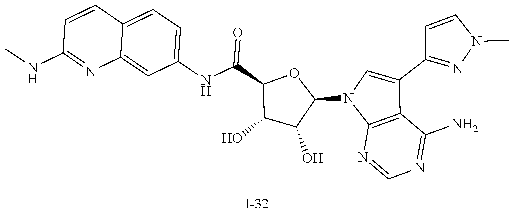 | A | 516.0 | ¹H NMR (400 MHz, DMSO- d6) δ ppm 10.42 (br s, 1H), 8.08 (s, 1H), 7.99 (s, 1H), 7.96 (s, 1H), 7.76 (m, 2H), 7.56 (d, J = 8.8 Hz, 1H), 7.28 (dd, J = 8.8, 2.0 Hz, 2H), 6.98 (m, 1H), 6.65 (d, J = 8.8 Hz, 1H), 6.58 (d, J = 2.0 Hz, 1H), 6.16 (d, J = 6.8 Hz, 1H), 5.74 (d, J = 4.8 Hz, 1H), 5.56 (d, J = 6.4 Hz, 1H), 4.67 (q, J = 6.8 Hz, 1H), 4.54 (d, J = 2.0 Hz, 1H), 4.35 (m, 1H), 3.90 (s, 3H), 3.30 (m, |
| | | | 1H), 2.89 (d, J = 4.8 Hz, 3H) |

### Example 6: Synthetic Scheme for Compounds I-33 and I-34

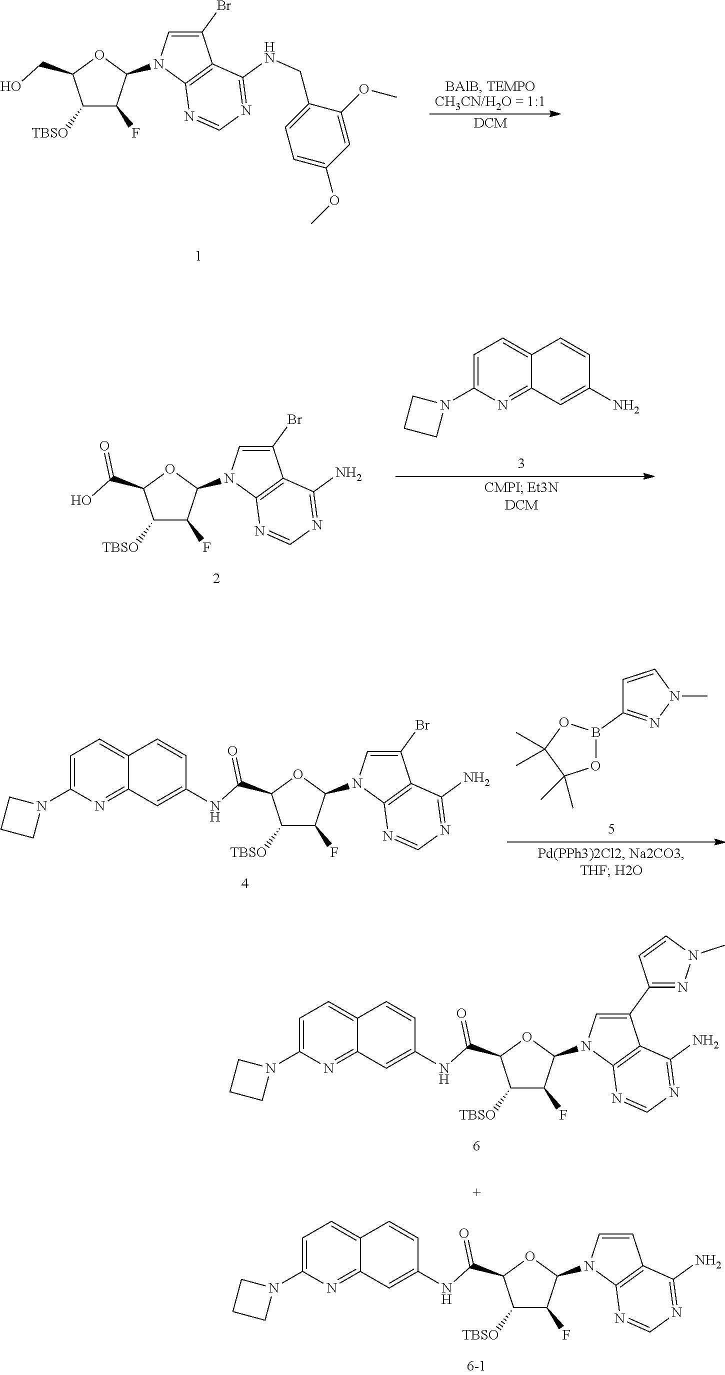

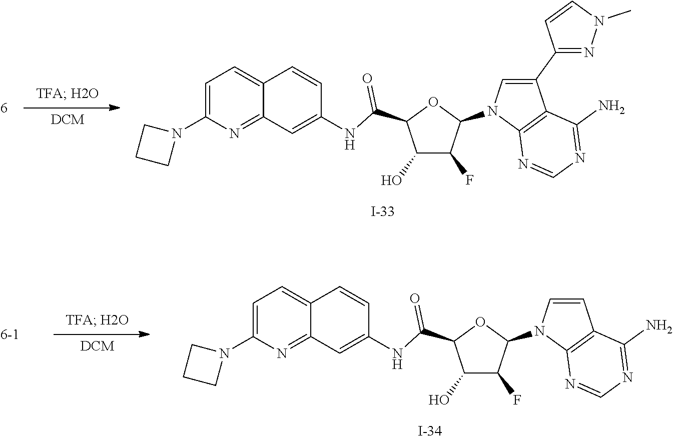

### 6.1 Synthesis of Compound 2

To a solution of [(2R,3R,4S,5R)-5-(5-bromo-4-{[(2,4-dimethoxyphenyl)methyl]amino}-7H-pyrrolo[2,3-d]pyrimidin-7-yl)-3-[(tert-butyldimethylsilyl)oxy]-4-fluorooxolan-2-yl]methanol (300 mg, 490 μmol) in DCM (4 mL), was added (acetyloxy)(phenyl)-lambda3-iodanyl acetate (426 mg, 1.32 mmol), TEMPO (24 mg, 153 μmol), MeCN (0.3 mL) and H₂O (0.3 mL) at and stirred at room temperature for 18 hours. The mixture was diluted with water (40 ml) and DCM (40 mL). The organic layer was separated, washed with H₂O (40 mL) and brine (30 mL), and dried (Na₂SO₄). The solvent was removed under reduced pressure and the residue washed by PE (20 mL×2). The solid was dissolved in THF (3 mL), then PE (40 mL) was added. The solid was separated out, filtered, washed by PE (5 mL×2) to give (2S,3R,4S,5R)-5-{4-amino-5-bromo-7H-pyrrolo[2,3-d]pyrimidin-7-yl}-3-[(tert-butyldimethylsilyl)oxy]-4-fluorooxolane-2-carboxylic acid (133 mg, 279 μmol).

### 6.2 Synthesis of Compound 3

To a solution of (2S,3R,4S,5R)-5-{4-amino-5-bromo-7H-pyrrolo[2,3-d]pyrimidin-7-yl}-3-[(tert-butyldimethylsilyl)oxy]-4-fluorooxolane-2-carboxylic acid (133 mg, 279 μmol) in DCM (20 mL), was added 2-(azetidin-1-yl)quinolin-7-amine (65 mg, 326 μmol), triethylamine (235 mg, 2.32 mmol) and 2-chloro-1-methylpyridin-1-ium iodide (170 mg, 665 μmol) at room temperature and stirred at 50° C. for 2.5 hours. The mixture was diluted with water (40 ml) and DCM (40 mL). The organic layer was separated, washed with H₂O (50 mL×2) and brine (50 mL), and dried (Na₂SO₄). The solvent was removed under reduced pressure and the residue purified by silica gel column chromatography (70% EA) to give (2S,3R,4S,5R)-5-{4-amino-5-bromo-7H-pyrrolo[2,3-d]pyrimidin-7-yl}-N-[2-(azetidin-1-yl)quinolin-7-yl]-3-[(tert-butyldimethylsilyl)oxy]-4-fluorooxolane-2-carboxamide (135 mg, 205 μmol).

### 6.3 Synthesis of Compound 4 and 5

To a solution of (2S,3R,4S,5R)-5-{4-amino-5-bromo-7H-pyrrolo[2,3-d]pyrimidin-7-yl}-N-[2-(azetidin-1-yl)quinolin-7-yl]-3-[(tert-butyldimethylsilyl)oxy]-4-fluorooxolane-2-carboxamide (130 mg, 197 μmol) in THF (22 mL), was added 1-methyl-3-(4,4,5,5-tetramethyl-1,3,2-dioxaborolan-2-yl)-1H-pyrazole (75 mg, 360 μmol), sodium carbonate (60 mg, 566 μmol), H₂O (2.2 mL) and Pd(PPh₃)₂Cl₂ (45 mg, 64.1 μmol) at rt under N₂ and stirred at 75° C. for 18 hours. The solvent was removed under reduced pressure and the residue purified by silica gel column chromatography (MeOH 4%) to give (2S,3R,4S,5R)-5-[4-amino-5-(1-methyl-1H-pyrazol-3-yl)-7H-pyrrolo[2,3-d]pyrimidin-7-yl]-N-[2-(azetidin-1-yl)quinolin-7-yl]-3-[(tert-butyldimethylsilyl)oxy]-4-fluorooxolane-2-carboxamide (55.0 mg, 83.6 μmol). Also obtained (2S,3R,4S,5R)-5-{4-amino-7H-pyrrolo[2,3-d]pyrimidin-7-yl}-N-[2-(azetidin-1-yl)quinolin-7-yl]-3-[(tert-butyldimethylsilyl)oxy]-4-fluorooxolane-2-carboxamide (17 mg).

### 6.4 Synthesis of Compound I-33

A solution of (2S,3R,4S,5R)-5-[4-amino-5-(1-methyl-1H-pyrazol-3-yl)-7H-pyrrolo[2,3-d]pyrimidin-7-yl]-N-[2-(azetidin-1-yl)quinolin-7-yl]-3-[(tert-butyldimethylsilyl)oxy]-4-fluorooxolane-2-carboxamide (55 mg, 83.6 μmol) in TFA (5 mL), H₂O (0.5 mL) and DCM (0.5 mL) was stirred at 55° C. for 4 hours. The mixture was concentrated, neutralized by 4 ml sat aq NaHCO₃ and filtered. The product was dissolved in MeOH and THF, purified by Prep-HPLC to afford (2S,3R,4S,5R)-5-[4-amino-5-(1-methyl-1H-pyrazol-3-yl)-7H-pyrrolo[2,3-d]pyrimidin-7-yl]-N-[2-(azetidin-1-yl)quinolin-7-yl]-4-fluoro-3-hydroxyoxolane-2-carboxamide (30 mg, 66%). ESI LCMS m/z=544 [M+1]; ¹H NMR (400 MHz, DMSO-d6) δ ppm 10.48 (s, 1H), 9.05 (brs, 1H), 8.11 (s, 1H), 8.04 (d, J=1.6 Hz, 1H), 7.94 (d, J=8.4 Hz, 1H), 7.87 (d, J=2.0 Hz, 1H), 7.74 (d, J=2.4 Hz, 1H), 7.65 (d, J=8.4 Hz, 1H), 7.42 (dd, J=8.8, 2.0 Hz, 1H), 7.30 (brs, 1H), 6.75 (dd, J=20.8, 3.2 Hz, 1H), 6.60 (d, J=8.8 Hz, 1H), 6.49 (d, J=2.4 Hz, 1H), 6.41 (d, J=4.8 Hz, 1H), 5.20 (dt, J=52.0, 2.8 Hz, 1H), 4.81-4.75 (m, 1H), 4.58 (d, J=3.2 Hz, 1H), 4.09 (t, J=7.2 Hz, 4H), 3.90 (s, 3H), 2.40-2.32 (m, 2H).

### 6.5 Synthesis of Compound I-34

A solution of (2S,3R,4S,5R)-5-{4-amino-7H-pyrrolo[2,3-d]pyrimidin-7-yl}-N-[2-(azetidin-1-yl)quinolin-7-yl]-3-[(tert-butyldimethylsilyl)oxy]-4-fluorooxolane-2-carboxamide (17 mg, 29.4 μmol) in TFA (3 mL), H₂O (0.5 mL) and DCM (0.5 mL) was stirred at 55° C. for 4 hours. The mixture was concentrated, neutralized by 3 ml NaHCO₃ sat aq, filtered. The product was dissolved in MeOH and THF, purified by Prep-HPLC to afford (2S,3R,4S,5R)-5-{4-amino-7H-pyrrolo[2,3-d]pyrimidin-7-yl}-N-[2-(azetidin-1-yl)quinolin-7-yl]-4-fluoro-3-hydroxyoxolane-2-carboxamide (5 mg, 37%). ESI LCMS m/z=464 [M+1]; ¹H NMR (400 MHz, DMSO-d6) δ ppm 10.52 (s, 1H), 8.11 (s, 1H), 8.02 (d, J=1.6 Hz, 1H), 7.93 (d, J=8.8 Hz, 1H), 7.65 (d, J=8.8 Hz, 1H), 7.48-7.46 (m, 1H), 7.39 (dd, J=8.4, 2.0 Hz, 1H), 7.13 (brs, 2H), 6.72 (dd, J=21.2, 3.2 Hz, 1H), 6.68 (d, J=3.6 Hz, 1H), 6.60 (d, J=8.8 Hz, 1H), 6.39 (d, J=4.8 Hz, 1H), 5.14 (dt, J=51.6, 2.8 Hz, 1H), 4.77-4.71 (m, 1H), 4.54 (d, J=2.8 Hz, 1H), 4.08 (t, J=7.6 Hz, 4H), 2.40-2.32 (m, 2H).

### Example 7: Synthetic Scheme for Compound I-35

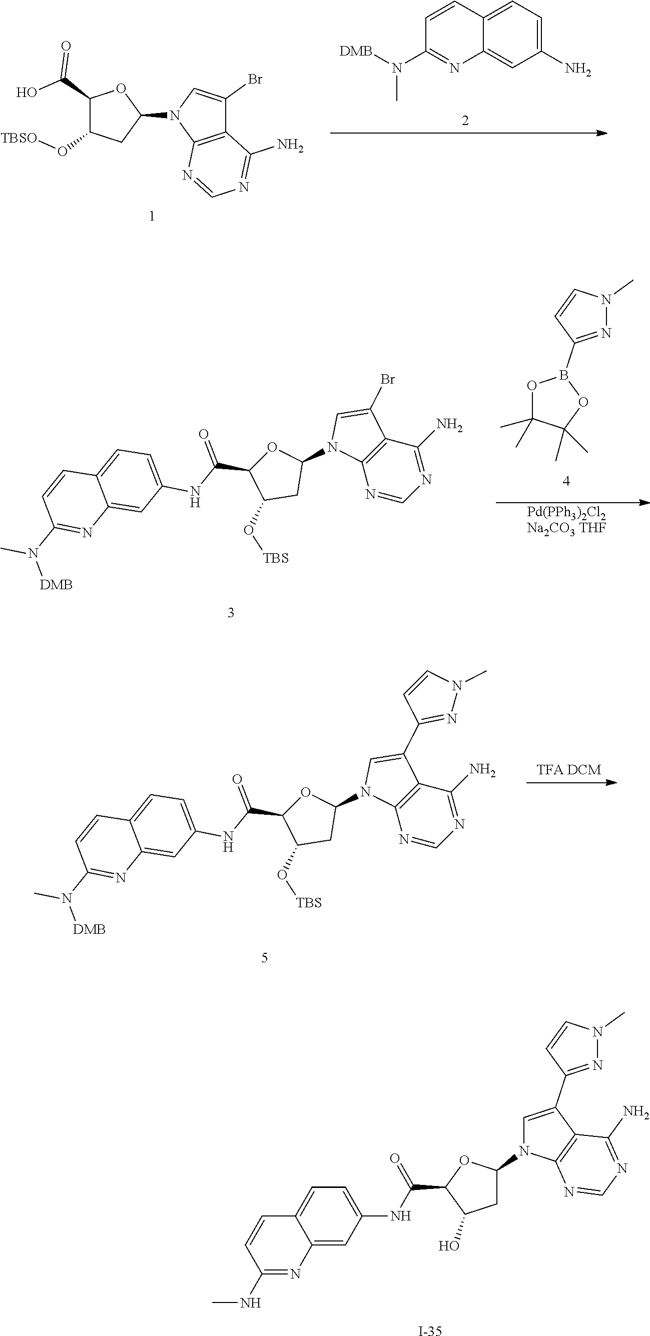

### 7.1 Synthesis of Compound 3

N₂-[(2,4-Dimethoxyphenyl)methyl]-N₂-methylquinoline-2,7-diamine (141 mg, 437 μmol), (2S,3S,5R)-5-{4-amino-5-bromo-7H-pyrrolo[2,3-d]pyrimidin-7-yl}-3-[(tert-butyldimethylsilyl)oxy]oxolane-2-carboxylicacid (200 mg, 437 μmol), CMPI (334 mg, 1.31 mmol), DIEA (168 mg, 1.31 mmol) were mixed in MeCN (10 mL). The mixture was heated to 80° C. and stirred for 12 h. The mixture was diluted with water (80 ml) and extracted with EA (80 ml×2). Combined organic layers were washed with H₂O (100 ml×2) and brine (60 ml), and dried (Na₂SO₄). The solvent was removed under reduced pressure to afford target compound. (200 mg, yield 60.0%). LC-MS m/z=761.9 [M+H]⁺.

### 7.2 Synthesis of Compound 5

(2S,3S,5R)-5-{4-Amino-5-bromo-7H-pyrrolo[2,3-d]pyrimidin-7-yl}-3-[(tert-butyldimethylsilyl)oxy]-N-(2-{[(2,4-dimethoxyphenyl)methyl](methyl)amino}quinolin-7-yl)oxolane-2-carboxamide (200 mg, 262 μmol), 1-methyl-3-(4,4,5,5-tetramethyl-1,3,2-dioxaborolan-2-yl)-1H-pyrazole (136 mg, 655 μmol), Na₂CO₃ (83.3 mg, 786 μmol), Pd(PPh₃)₂Cl₂ (18.3 mg, 26.2 μmol) were mixed in THE (25 mL) and H₂O (3 mL). The mixture was heated to 75° C. and stirred for 12 h under N₂ atmosphere. The reaction was concentrated under pressure to afford the crude. The crude was purified by fish chromatography (MeOH˜DCM: 0˜20%) to afford compound 5 (150 mg, yield 74.9%). LC-MS m/z=763.8 [M+H]⁺.

### 7.3 Synthesis of Compound I-35

(2S,3S,5R)-5-[4-Amino-5-(1-methyl-1H-pyrazol-3-yl)-7H-pyrrolo[2,3-d]pyrimidin-7-yl]-3-[(tert-butyldimethylsilyl)oxy]-N-(2-{[(2,4-dimethoxyphenyl)methyl](methyl)amino}quinolin-7-yl)oxolane-2-carboxamide (150 mg, 196 μmol) was added to TFA (5 mL) and DCM (2 mL). The mixture was stirred at rt for 5 hours. Then the mixture was concentrated to afford crude, neutralized by 4 mL 7M NH₃ in MeOH. The product was purified by Prep-HPLC to afford target compound (66 mg, yield, 67.4%). LC-MS m/z=500.0 [M+H]⁺. ¹H NMR (400 MHz, DMSO-d6) δ 10.26 (s, 1H), 9.04 (s, 1H), 8.08 (s, 1H), 8.00 (s, 1H), 7.95 (d, J=1.7 Hz, 1H), 7.75 (d, J=8.8 Hz, 1H), 7.73 (d, J=2.2 Hz, 1H), 7.54 (d, J=8.6 Hz, 1H), 7.40-7.20 (m, 2H), 7.10-6.90 (m, 1H), 6.69 (dd, J=9.4, 5.4 Hz, 1H), 6.65 (d, J=8.9 Hz, 1H), 6.53 (d, J=2.3 Hz, 1H), 5.82 (d, J=4.1 Hz, 1H), 4.70-4.62 (m, 1H), 4.48 (d, J=1.2 Hz, 1H), 3.89 (s, 3H), 2.89 (d, J=4.7 Hz, 3H), 2.82-2.71 (m, 1H), 2.37-2.17 (m, 1H).

### Example 8: Synthetic Scheme for Compound I-36

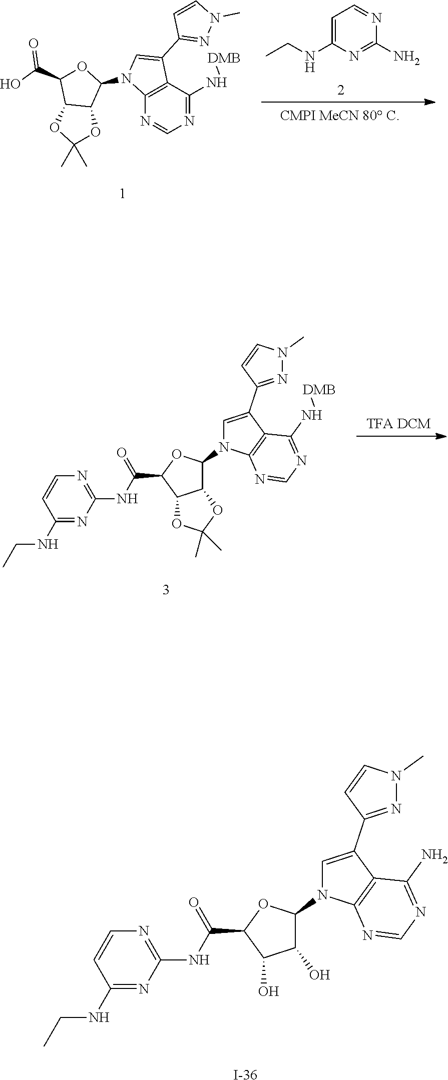

### 8.1 Synthesis of Compound 3

(3aS,4S,6R,6aR)-6-(4-{[(2,4-Dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylicacid (50.0 mg, 90.8 μmol), N4-ethylpyrimidine-2,4-diamine (25.0 mg, 181 μmol), DIEA (35.0 mg, 272 μmol), CMPI (46.1 mg, 181 μmol) were mixed in MeCN (5 mL). The mixture was heated to 80° C. and stirred for 12 h. The mixture was diluted with water (80 mL) and extracted with EA (80 mL×2). Combined organic layers were washed with H₂O (100 mL×2) and brine (60 mL), and dried (Na₂SO₄). The solvent was removed under reduced pressure to afford target compound (50 mg, yield 57.4%). LC-MS m/z=671.0 [M+H]⁺.

### 8.2 Synthesis of Compound I-36

(3aS,4S,6R,6aR)-6-(4-{[(2,4-Dimethoxyphenyl)methyl]amino}-5-(1-methyl-1H-pyrazol-3-yl)-7H-pyrrolo[2,3-d]pyrimidin-7-yl)-N-[4-(ethylamino)pyrimidin-2-yl]-2,2-dimethyl-tetrahydro-2H-furo[3,4-d][1,3]dioxole-4-carboxamide (30.0 mg, 44.7 μmol) was added to TFA (5 mL) and DCM (2 mL). The mixture was stirred at room temperature for 2 hours, then concentrated to afford crude which was neutralized by 4 ml 7M NH₃ in MeOH. The product was purified by Prep-HPLC to afford compound. (4.5 mg, yield, 21.0%). LC-MS m/z=480.9 [M+H]⁺. ¹H NMR (400 MHz, DMSO-d6) δ 10.70 (s, 0.5H), 9.05 (s, 1H), 8.84 (s, 0.5H), 8.22 (s, 0.5H), 8.10 (s, 0.5H), 8.05-7.80 (m, 2H), 7.76 (s, 1H), 7.55-7.15 (m, 2H), 6.70-6.55 (m, 1H), 6.40-6.20 (m, 1H), 6.20-6.00 (m, 1H), 5.74-5.60 (m, 1H), 5.60-5.48 (m, 1H), 5.17-4.79 (m, 1H), 4.75-4.52 (m, 1H), 4.50-4.20 (m, 1H), 3.90 (s, 3H), 1.12 (t, J=6.0 Hz, 3H).

### Example 9: Synthetic Scheme for Compound I-37

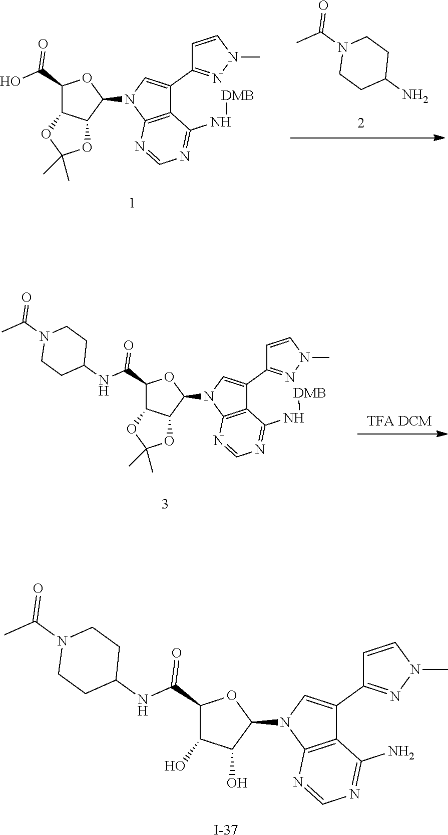

### 9.1 Synthesis of Compound 3

Ethylbis(propan-2-yl)amine (35.1 mg, 272 μmol), (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylic acid (50.0 mg, 90.8 μmol), 1-(4-aminopiperidin-1-yl)ethan-1-one (38.6 mg, 272 μmol), BOP (80.0 mg, 181 μmol) were mixed in MeCN (5 mL). The mixture was stirred at rt for 4 h. The mixture was diluted with water (80 mL) and extracted with EA (80 mL×2). Combined organic layers were washed with H₂O (100 mL×2) and brine (60 mL), and dried (Na₂SO₄). The solvent was removed under reduced pressure to afford target compound (50 mg, yield 57.1%). LC-MS m/z=675.0 [M+H]⁺.

### 9.2 Synthesis of Compound I-37

To a solution of (3aS,4S,6R,6aR)-N-(1-acetylpiperidin-4-yl)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxamide (50.0 mg, 74.1 μmol) in DCM (2 mL) was added TFA (5 mL). The mixture was stirred at rt for 5 h, then the mixture was concentrated to afford crude, neutralized by 4 ml 7M NH₃ in MeOH. The product was purified by Prep-HPLC to afford target compound (5 mg, yield 13.9%). LC-MS m/z=485.2 [M+H]⁺. ¹H NMR (400 MHz, DMSO-d6) δ 9.06 (s, 1H), 8.32 (t, J=9.0 Hz, 1H), 8.04 (d, J=4.0 Hz, 1H), 7.93 (s, 1H), 7.75 (d, J=2.2 Hz, 1H), 7.27 (s, 1H), 6.56 (d, J=2.3 Hz, 1H), 6.06 (d, J=7.4 Hz, 1H), 5.62 (s, 1H), 5.45 (s, 1H), 4.70-4.55 (m, 1H), 4.35-4.20 (m, 2H), 4.17 (d, J=3.3 Hz, 1H), 3.81-3.70 (m, 1H), 3.15-3.05 (m, 2H), 2.75-2.60 (m, 1H), 1.99 (s, 3H), 1.93-1.64 (m, 2H), 1.46-1.08 (m, 2H).

### Example 10: Synthetic Scheme for Compound I-38

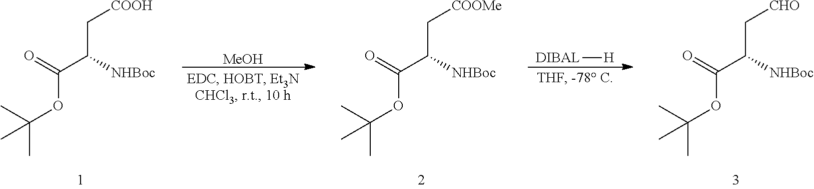

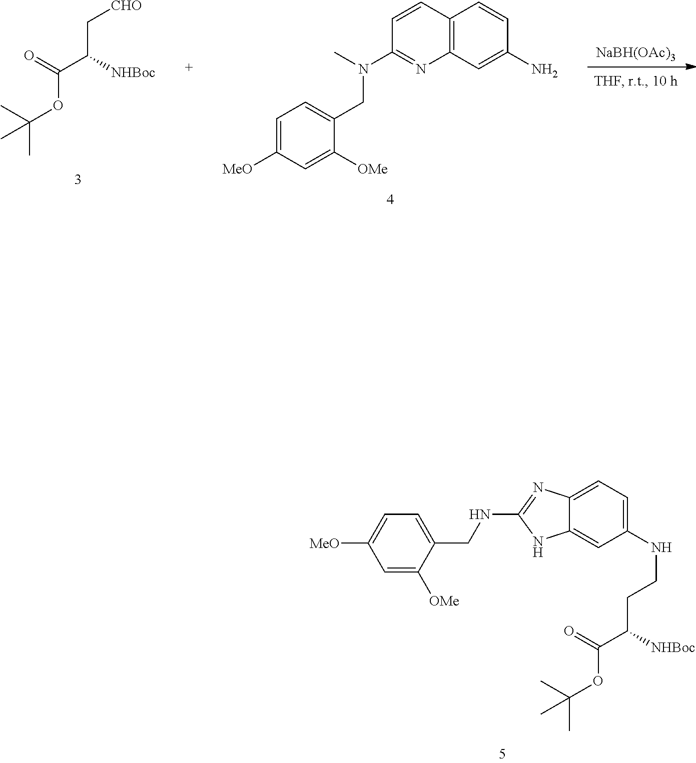 [SMILES: CC(C)(C)OC(=O)N[C@@H](CC=O)C(=O)OC(C)(C)C ; COc1ccc(CN(C)c2ccc3ccc(N)cc3n2)c(OC)c1 ; "THF, r.t., 10 h" ; COc1ccc(CNc2nc3ccc(NCC[C@H](NC(=O)OC(C)(C)C)C(=O)OC(C)(C)C)cc3[nH]2)c(OC)c1 ; "NaBH(OAc)3"]

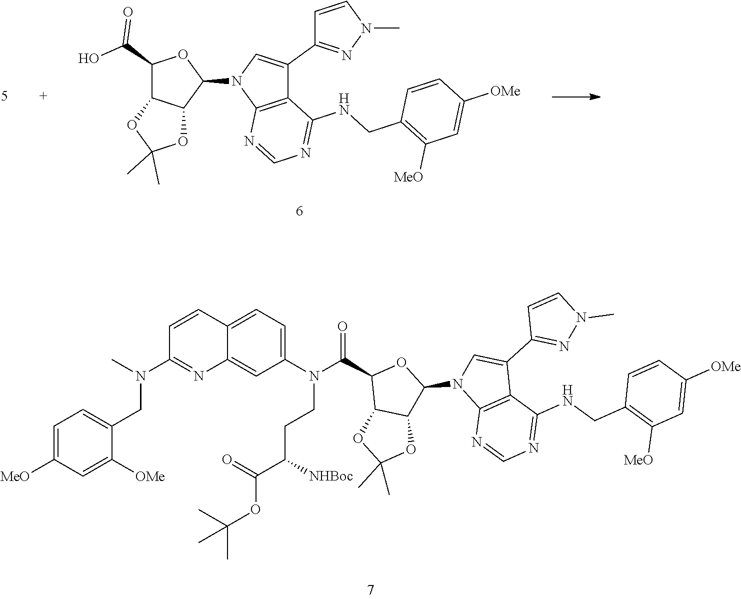 [SMILES: COc1ccc(CNc2ncnc3c2c(-c2ccn(C)n2)cn3[C@@H]2O[C@H](C(=O)O)[C@H]3OC(C)(C)O[C@H]32)c(OC)c1 ; COc1ccc(CNc2ncnc3c2c(-c2ccn(C)n2)cn3[C@@H]2O[C@H](C(=O)N(CC[C@H](NC(=O)OC(C)(C)C)C(=O)OC(C)(C)C)c3ccc4ccc(N(C)Cc5ccc(OC)cc5OC)nc4c3)[C@H]3OC(C)(C)O[C@H]32)c(OC)c1]

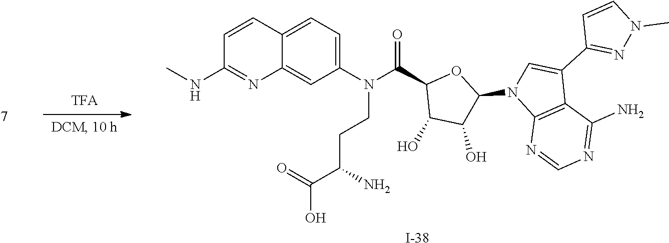

### 10.1 Synthesis of Compound 2

To the mixture of (4S)-5-(tert-butoxy)-4-{[(tert-butoxy)carbonyl]amino}-5-oxopentanoic acid (2 g, 6.59 mmol), (3-{[(ethylimino)methylidene]amino}propyl)dimethylamine hydrochloride (1.64 g, 8.56 mmol), hydroxybenzotriazole (1.15 g, 8.56 mmol) and methanol (631 mg, 19.7 mmol) in chloroform (150 mL), triethylamine (1.65 g, 16.4 mmol) was added dropwise at 0° C. The reaction mixture was stirred at 0° C. for 10 hours. Chloroform (50 mL) and saturated aqueous NH₄Cl (50 mL) was added. The water phase was extracted by chloroform (2×50 mL). The combined organic phase was washed with water (30 mL) and brine (30 mL), and then dried over Na₂SO₄, filtrated, evaporated to dryness. The residue was purified by silica gel flash chromatography (PE:EA=10:1) to give the desired product 1-tert-butyl 5-methyl (2S)-2-{[(tert-butoxy)carbonyl]amino}pentanedioate (1.40 g, 4.41 mmol). LC-MS: no mass.

### 10.2 Synthesis of Compound 3

To the solution of 1-tert-butyl 4-methyl (2S)-2-{[(tert-butoxy)carbonyl]amino}butanedioate (1.4 g, 4.61 mmol) in tetrahydrofuran (30 mL), the solution of bis(isobutyl group) aluminium hydrogen (786 mg, 5.53 mmol) in toluene (3.5 mL) was added dropwise at −78° C. under N₂. The reaction mixture was stirred at −78° C. for 20 minutes under N₂. H₂O (1 mL) was added to quench the reaction, and the mixture was stirred at −78° C. for 30 minutes and then warmed to 0° C. Na₂SO₄ was added, and the mixture was filtered through a pad of Celite. The solvent was evaporated, and the residue was purified by silica gel flash chromatography to afford tert-butyl (2S)-2-{[(tert-butoxy)carbonyl]amino}-4-oxobutanoate (400 mg, 1.46 mmol). LC-MS: no mass.

### 10.3 Synthesis of Compound 5

To a solution of tert-butyl (2S)-2-{[(tert-butoxy)carbonyl]amino}-4-oxobutanoate (400 mg, 1.46 mmol) and N₂-[(2,4-dimethoxyphenyl)methyl]-N₂-methylquinoline-2,7-diamine (611 mg, 1.89 mmol) in tetrahydrofuran (20 mL), sodium triacetoxyborohydride (370 mg, 1.75 mmol) was added in portions at 0° C., and then the reaction mixture was allowed to warm to 20° C. and stirred at 20° C. for 10 hours. The reaction was terminated by the addition of water (25 mL). EA (100 mL) was added, and the water phase was extracted by EA (50 mL×3). The combined organic layer was dried over anhydrous Na₂SO₄ and evaporated to dryness. The residue was purified by C18 reversed-phase chromatography to give the desired product tert-butyl (2S)-2-{[(tert-butoxy)carbonyl]amino}-4-[(2-{[(2,4-dimethoxyphenyl)methyl](methyl)amino}quinolin-7-yl)amino]butanoate (200 mg, 344 μmol). LC-MS m/z=581.1 [M+H]⁺.

### 10.4 Synthesis of Compound 7

To the solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylic acid (150 mg, 272 μmol) and tert-butyl (2S)-2-{[(tert-butoxy)carbonyl]amino}-4-[(2-{[(2,4-dimethoxyphenyl)methyl](methyl)amino}quinolin-7-yl)amino]butanoate (173 mg, 299 μmol) in tetrahydrofuran (3 mL), tripropyl-1,3,5,2lambda5,4lambda5,6lambda5-trioxatriphosphinane-2,4,6-trione (95.1 mg, 299 μmol) and N,N-diisopropylethylamine (175 mg, 1.36 mmol) were added. The reaction mixture was stirred at 20° C. for 12 hours. Water (40 mL) and EA (100 mL) were added, and the water phase was extracted by EA (100 mL×3). The combined organic phase was evaporated to dryness. The residue was purified by C18 reversed-phase chromatography to give the desired product tert-butyl (2S)-4-{1-[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]-N-(2-{[(2,4-dimethoxyphenyl)methyl](methyl)amino}quinolin-7-yl)formamido}-2-{[(tert-butoxy)carbonyl]amino}butanoate (10.0 mg, 8.98 μmol). LC-MS m/z=557.2 [M/2+H]⁺.

### 10.5 Synthesis of Compound I-38

A solution of tert-butyl (2S)-4-{1-[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]-N-(2-{[(2,4-dimethoxyphenyl)methyl](methyl)amino}quinolin-7-yl)formamido}-2-{[(tert-butoxy)carbonyl]amino}butanoate (10 mg, 8.98 μmol) in trifluoroacetic acid (1 mL) and methylene chloride (4 mL) was stirred at 20° C. for 12 h. Then the mixture was concentrated under reduced pressure, the residue was neutralized by 3 ml 7M NH₃ in MeOH to pH=8. The solvents were evaporated, and the residue was purified by Prep-HPLC to afford (2S)-2-amino-4-{1-[(2S,3S,4R,5R)-5-[4-amino-5-(1-methyl-1H-pyrazol-3-yl)-7H-pyrrolo[2,3-d]pyrimidin-7-yl]-3,4-dihydroxyoxolan-2-yl]-N-[2-(methylamino)quinolin-7-yl]formamido}butanoic acid (1.50 mg, 2.43 μmol) as a pale yellow solid. LC-MS m/z=617.0 [M+H]. ¹H NMR (400 MHz, MeOD) δ 8.11 (m, 1H), 8.02 (m, 1H), 7.88-7.81 (m, 2H), 7.74-7.72 (m, 1H), 7.67-7.64 (m, 1H), 7.60 (m, 2H), 7.18-7.16 (m, 1H), 6.82-6.80 (m, 1H), 6.74-6.72 (m, 1H), 6.65-6.64 (m, 1H), 6.26-6.25 (m, 1H), 4.53-4.52 (m, 3H), 3.97-3.93 (m, 6H), 3.24-3.18 (m, 2H), 3.00 (s, 3H).

### Example 11: Synthetic Scheme for Compound I-39

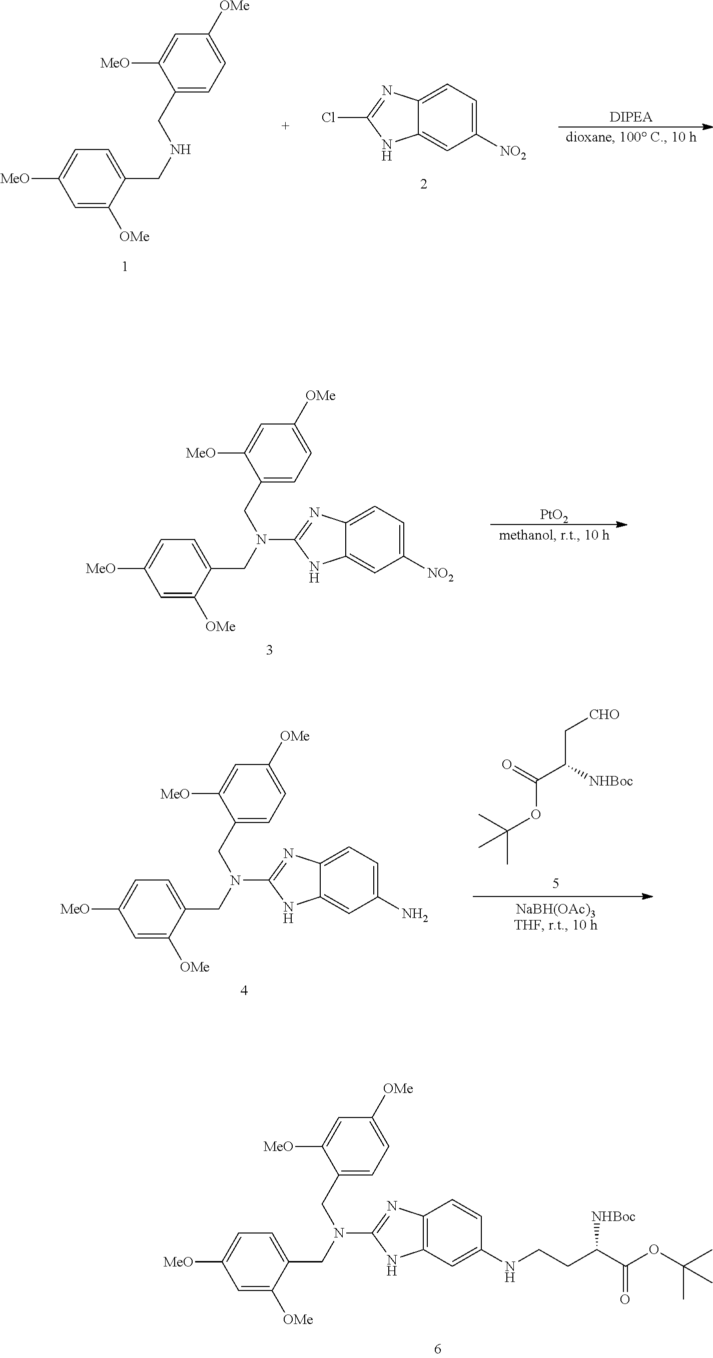

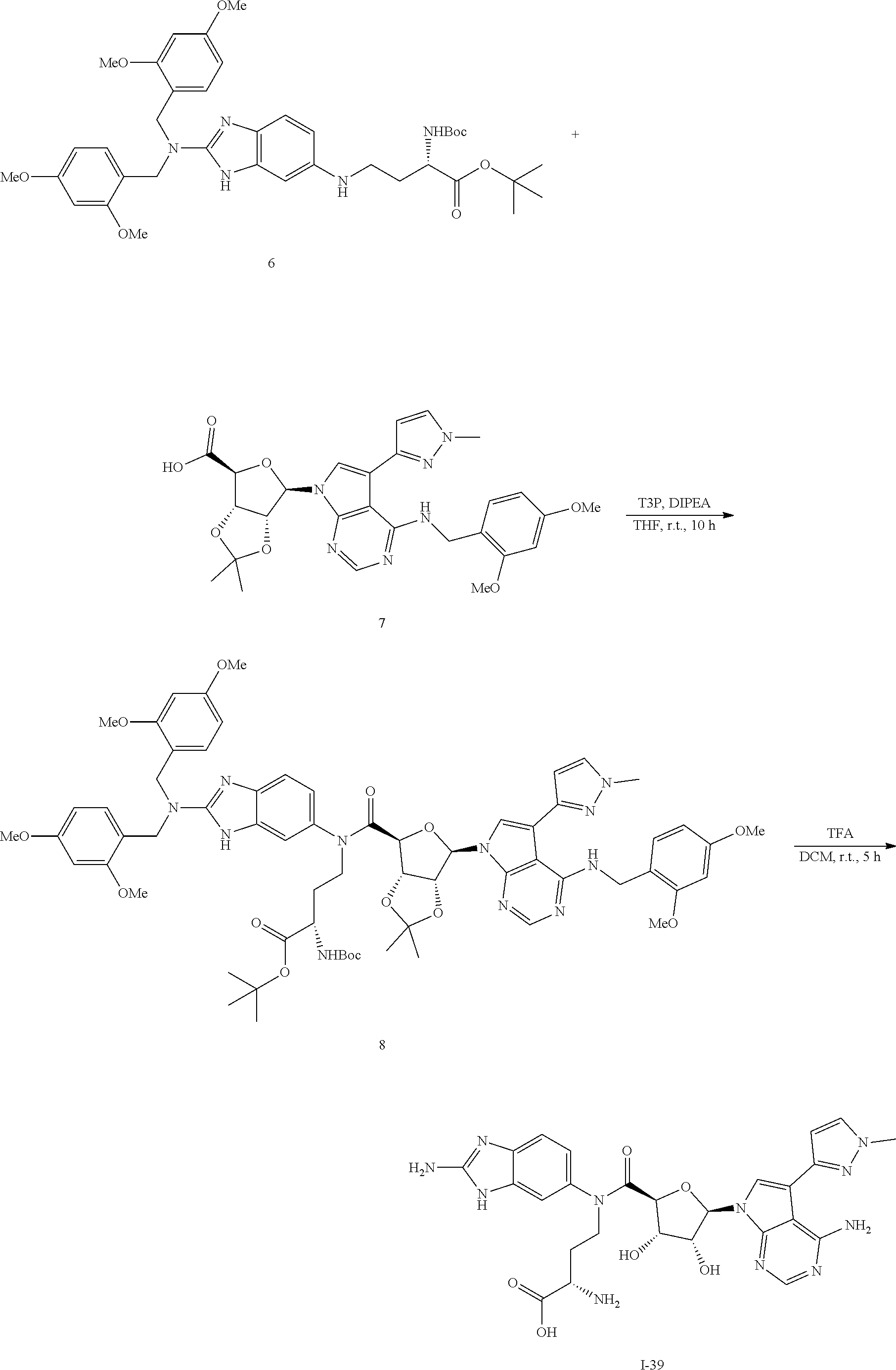

### 11.1 Synthesis of Compound 3

bis[(2,4-Dimethoxyphenyl)methyl]amine (799 mg, 2.52 mmol), 2-chloro-6-nitro-1H-1,3-benzodiazole (250 mg, 1.26 mmol) and N,N-diisopropylethylamine (488 mg, 3.78 mmol) were mixed in dioxane (5 mL). The mixture was stirred at 100° C. for 12 hours. The mixture was evaporated to dryness. The residue was purified by silica gel flash chromatography (PE:EA=3:2) to give N,N-bis[(2,4-dimethoxyphenyl)methyl]-6-nitro-1H-1,3-benzodiazol-2-amine (550 mg, 1.14 mmol). LC-MS m/z=479.3 [M+H]⁺.

### 11.2 Synthesis of Compound 4

A mixture of N,N-bis[(2,4-dimethoxyphenyl)methyl]-6-nitro-1H-1,3-benzodiazol-2-amine (550 mg, 1.14 mmol) and Adams' catalyst (51.7 mg, 228 μmol) in methanol (10 mL) was stirred for 16 h at 20° C. in a round bottom flask under H₂. The reaction mixture was filtered through a pad of Celite with EtOAc, and the combined organics were concentrated in vacuo to dryness. The residue was purified by silica gel flash chromatography (DCM:methanol=20:1) to give the desired product N₂,N₂-bis[(2,4-dimethoxyphenyl)methyl]-1H-1,3-benzodiazole-2,6-diamine (210 mg, 468 μmol). LC-MS m/z=449.0 [M+H]⁺.

### 11.3 Synthesis of Compound 6

To a solution of tert-butyl (2S)-2-{[(tert-butoxy)carbonyl]amino}-4-oxobutanoate (95 mg, 347 μmol) and N₂,N₂-bis[(2,4-dimethoxyphenyl)methyl]-1H-1,3-benzodiazole-2,6-diamine (202 mg, 451 μmol) in tetrahydrofuran (5 mL), sodium triacetoxyborohydride (88.1 mg, 416 μmol) was added in portions at 0° C., and then the reaction mixture was allowed to warm to 20° C. and stirred at 20° C. for 5 hours. The reaction was terminated by the addition of water (25 mL). EA (100 mL) was added, and the water phase was extracted by EA (50 mL×3). The combined organic layer was dried over anhydrous Na₂SO₄ and evaporated to dryness. The residue was purified by silica gel flash chromatography (DCM:NH₃ in MeOH (7.0 M)=20:1) to give the desired product tert-butyl (2S)-4-[(2-{bis[(2,4-dimethoxyphenyl)methyl]amino}-1H-1,3-benzodiazol-6-yl)amino]-2-{[(tert-butoxy)carbonyl]amino}butanoate (200 mg, 283 μmol). LC-MS m/z=706.0 [M+H]⁺.

### 11.4 Synthesis of Compound 8

To the solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylic acid (50 mg, 90.8 μmol) and tert-butyl (2S)-4-[(2-{bis[(2,4-dimethoxyphenyl)methyl]amino}-1H-1,3-benzodiazol-6-yl)amino]-2-{[(tert-butoxy)carbonyl]amino}butanoate (70.4 mg, 99.8 μmol) in tetrahydrofuran (2 mL), tripropyl-1,3,5,2lambda5,4lambda5,6lambda5-trioxatriphosphinane-2,4,6-trione (37.5 mg, 118 μmol) and N,N-diisopropylethylamine (58.6 mg, 454 μmol) were added. The reaction mixture was stirred at 20° C. for 10 h, until the reaction was complete as indicated by LCMS. Water (40 mL) and EA (100 mL) was added, and the water phase was extracted by EA (100 mL×3). The combined organic phase was washed by sat. NH₄Cl (40 mL×2) and brine (40 mL), and evaporated to dryness. The residue was purified by prep-HPLC to give the desired product tert-butyl (2S)-4-{1-[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]-N-(2-{bis[(2,4-dimethoxyphenyl)methyl]amino}-1H-1,3-benzodiazol-6-yl)formamido}-2-{[(tert-butoxy)carbonyl]amino}butanoate (30.0 mg, 24.2 μmol). LC-MS m/z=619.7 [M/2+H]⁺.

### 11.5 Synthesis of Compound I-39

A solution of tert-butyl (2S)-4-{1-[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]-N-(2-{bis[(2,4-dimethoxyphenyl)methyl]amino}-1H-1,3-benzodiazol-6-yl)formamido}-2-{[(tert-butoxy)carbonyl]amino}butanoate (30 mg, 24.2 μmol) in trifluoroacetic acid (1.5 mL) and methylene chloride (6 mL) was stirred at 20° C. for 12 h. Then the mixture was concentrated under reduced pressure, the residue was neutralized by 3 ml 7M NH₃ in MeOH to pH 8. The solvents were evaporated, and the residue was purified by Prep-HPLC to afford (2S)-2-amino-4-[N-(2-amino-1H-1,3-benzodiazol-6-yl)-1-[(2S,3S,4R,5R)-5-[4-amino-5-(1-methyl-1H-pyrazol-3-yl)-7H-pyrrolo[2,3-d]pyrimidin-7-yl]-3,4-dihydroxyoxolan-2-yl]formamido]butanoic acid (8.10 mg, 13.6 μmol) as a pale yellow solid. LC-MS m/z=592.0 [M+H]⁺. ¹H NMR (400 MHz, MeOD) δ 7.23 (d, J=5.0 Hz, 1H), 7.18 (d, J=5.0 Hz, 2H), 6.79 (br, 1H), 6.69 (m, 1H), 6.36-6.34 (m, 2H), 6.29-6.26 (m, 2H), 5.92-5.90 (m, 1H), 5.86-5.81 (m, 1H), 5.68-5.62 (m, 1H), 5.31 (m, 1H), 4.91 (d, J=6.1 Hz, 1H), 4.72 (br, 1H), 3.29-3.26 (m, 1H), 3.15-3.13 (m, 1H), 2.74-2.69 (m, 1H), 2.61 (s, 3H), 2.54-2.49 (m, 1H), 2.35-2.32 (m, 1H), 2.26-2.22 (m, 1H), 2.14-2.10 (m, 2H).

### Example 12: Synthetic Scheme for Compound I-40

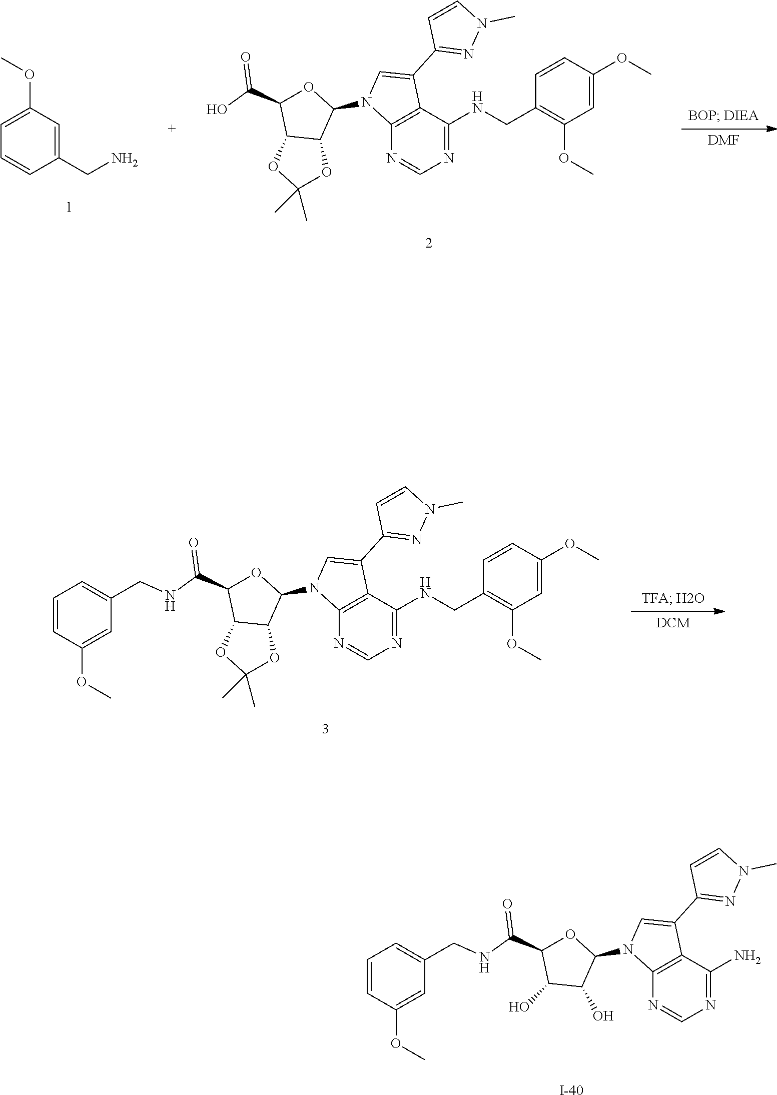

### 12.1 Synthesis of Compound 3

To a solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylic acid (2, 47 mg, 85.3 μmol) in DMF (2 mL), was added 1-(3-methoxyphenyl)methanamine (1, 60 mg, 437 μmol), N,N-diisopropylethylamine (95 mg, 735 μmol) and BOP (79 mg, 178 μmol) at rt and stirred at rt for 16 hours. The solvent was removed under reduced pressure and the residue was diluted with water (50 mL) and EA (50 mL). The organic layer was separated, washed with H₂O (50 mL×3) and brine (20 mL), and dried (Na₂SO₄). The solvent was removed under reduced pressure to give crude (3aS,4S,6R,6aR)-6-(4-{1[(2,4-dimethoxyphenyl)methyl]amino}-5-(1-methyl-1H-pyrazol-3-yl)-7H-pyrrolo[2,3-d]pyrimidin-7-yl)-N-[(3-methoxyphenyl)methyl]-2,2-dimethyl-tetrahydro2H-furo[3,4-d][1,3]dioxole-4-carboxamide (3, 50.0 mg, 74.6 μmol, yield 87.5%). LC-MS m/z=669.8 [M+H]⁺.

### 12.2 Synthesis of Compound I-40

A solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-N-[(3-methoxyphenyl)methyl]-2,2-dimethyl-tetrahydro-2H-furo[3,4-d][1,3]dioxole-4-carboxamide (50 mg, 74.6 μmol) in TFA (4 mL), H₂O (0.15 mL) and DCM (0.5 mL) was stirred at rt for 1.5 hours. Then the mixture was concentrated to afford crude, neutralized by 3 ml 7M NH₃ in MeOH, filtered. The product was purified by Prep-HPLC to afford the compound (16 mg, yield 44.8%) as a white solid. LC-MS m/z=480.0 [M+H]⁺. ¹H NMR (400 MHz, DMSO-d6) δ ppm 9.57 (t, J=5.6 Hz, 1H), 9.08 (brs, 1H), 7.86 (s, 1H), 7.76 (s, 1H), 7.74 (d, J=2.0 Hz, 1H), 7.31 (brs, 1H), 7.23 (t, J=8.0 Hz, 1H), 6.88-6.80 (m, 3H), 6.52 (d, J=2.4 Hz, 1H), 5.95 (d, J=7.6 Hz, 1H), 5.71 (d, J=4.4 Hz, 1H), 5.49 (d, J=6.8 Hz, 1H), 4.68-4.63 (m, 1H), 4.47-4.34 (m, 3H), 4.18-4.16 (m, 1H), 3.88 (s, 3H), 3.70 (s, 3H).

### Example 13: Synthetic Scheme for Compound I-41

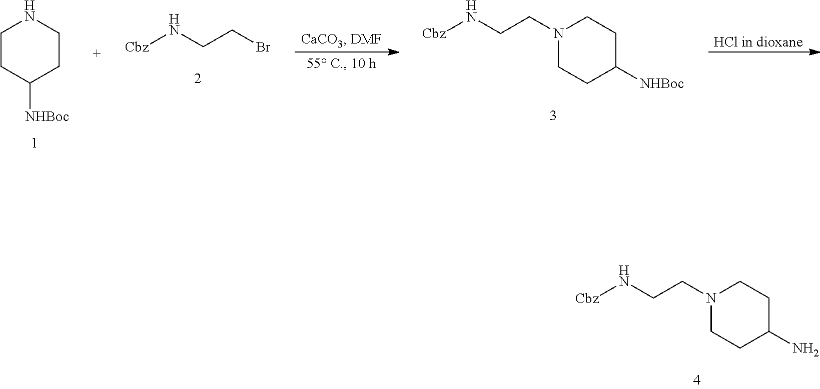

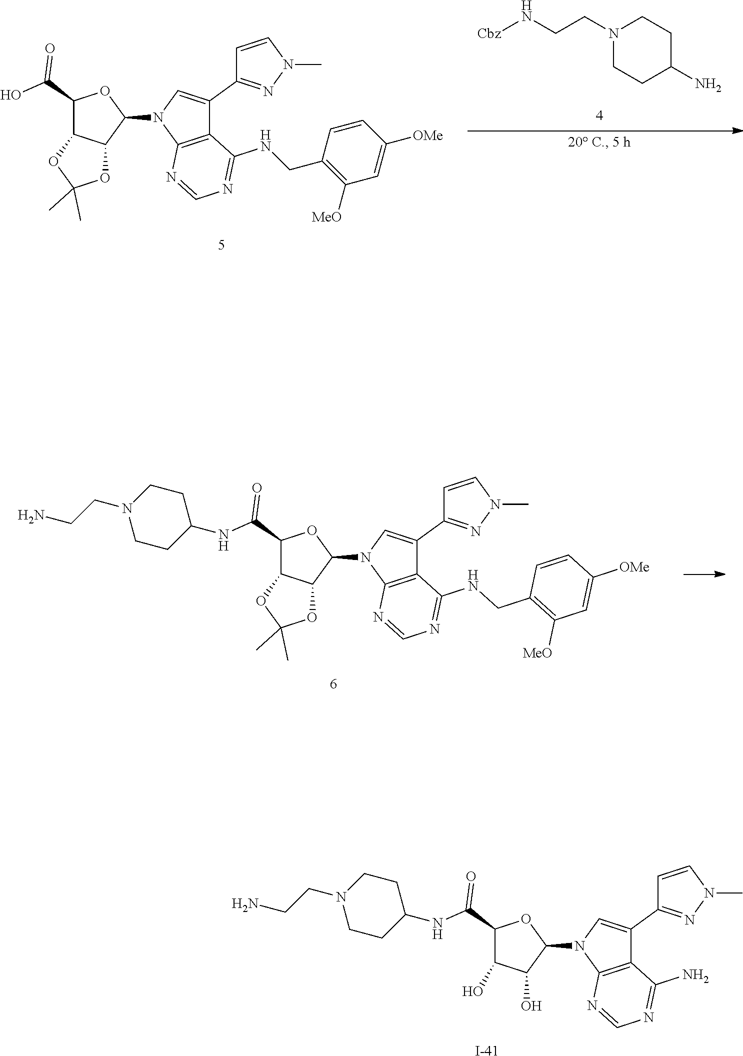

### 13.1 Synthesis of Compound 3

The reaction mixture of tert-butyl N-(piperidin-4-yl)carbamate (350 mg, 1.74 mmol), benzyl N-(2-bromoethyl)carbamate (583 mg, 2.26 mmol) and caesium carbonate (1.70 g, 5.22 mmol) in dimethylformamide (10 mL) was stirred at 55° C. for 10 hours. After the mixture was filtered, DMF was evaporated. And the residue was purified by prep-HPLC to give the desired product benzyl N-[2-(4-{[(tert-butoxy)carbonyl]amino}piperidin-1-yl)ethyl]carbamate (500 mg, yield 28.6%). LCMS (M+H)+=378.0.

### 13.2 Synthesis of Compound 4

The reaction mixture of benzyl N-[2-(4-{[(tert-butoxy)carbonyl]amino}piperidin-1-yl)ethyl]carbamate (500 mg, 1.32 mmol) in HCl in dioxane (4.0 (10 mL) was stirred at 20° C. for 10 hours. The solvent was evaporated to give the crude product benzyl N-[2-(4-aminopiperidin-1-yl)ethyl]carbamate (360 mg, yield 98.1%). LCMS (M+H)⁺=278.1.

### 13.3 Synthesis of Compound 6

To the solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylic acid (30 mg, 54.4 μmol) and benzyl N-[2-(4-aminopiperidin-1-yl)ethyl]carbamate (19.6 mg, 70.7 μmol) in dimethylformamide (2 mL), (1,2,3-benzotriazol-1-yloxy)tris(dimethylamino)phosphanium; hexafluoro-lambda5-phosphanuide (36.0 mg, 81.6 μmol) and N,N-diisopropylethylamine (35.1 mg, 272 μmol) were added. The reaction mixture was stirred at 20° C. for 5 hours.

### 13.4 Synthesis of Compound I-41

A solution of (3aS,4S,6R,6aR)-N-[1-(2-aminoethyl)piperidin-4-yl]-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxamide (30 mg, 44.3 μmol) in trifluoroacetic acid (1.5 mL) and methylene chloride (2 mL) was stirred at 20° C. for 4 hours. Then the mixture was concentrated under reduced pressure, the residue was neutralized by 3 ml 7M NH₃ in MeOH to pH=8. The solvents were evaporated, and the residue was purified by Prep-HPLC to afford (2S,3S,4R,5R)-5-[4-amino-5-(1-methyl-1H-pyrazol-3-yl)-7H-pyrrolo[2,3-d]pyrimidin-7-yl]-N-[1-(2-aminoethyl)piperidin-4-yl]-3,4-dihydroxyoxolane-2-carboxamide (2.00 mg, yield 9.3% 1) as a white solid. ¹H NMR (400 MHz, DMSO-d6) δ 9.08-9.00 (br, 1H), 8.34-8.29 (m, 1H), 8.09 (s, 1H), 7.92 (s, 1H), 7.76-7.75 (m, 1H), 7.33-7.22 (br, 1H), 6.56 (m, 1H), 6.04-6.02 (m, 1H), 5.62-5.61 (m, 1H), 5.45-5.43 (m, 1H), 4.65-4.61 (m, 1H), 4.27 (m, 1H), 4.17-4.12 (m, 1H), 3.89 (s, 3H), 3.67-3.57 (m, 1H), 2.86-2.78 (m, 2H), 2.36-2.32 (m, 4H), 2.18-2.14 (m, 4H), 2.04-1.97 (m, 2H), 1.80-1.64 (m, 2H).

### Example 14: Synthetic Scheme for Compounds I-42 and I-43

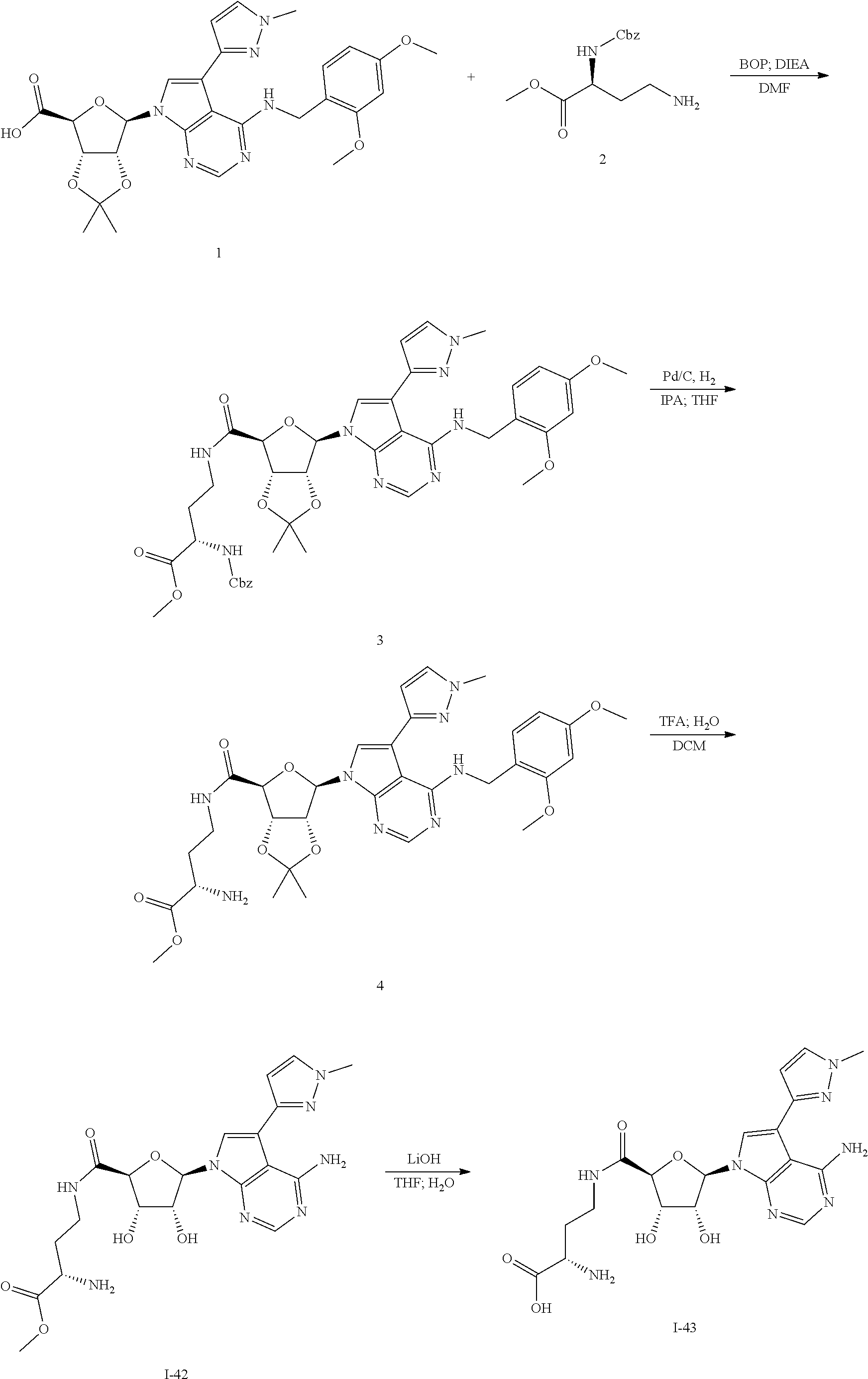

### 14.1 Synthesis of Compound 3

To a solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylic acid (120 mg, 217 μmol) in DMF (7 mL), was added methyl (2S)-4-amino-2-{[(benzyloxy)carbonyl]amino}butanoate (180 mg, 675 μmol), N,N-diisopropylethylamine (755 mg, 5.84 mmol) and (1,2,3-benzotriazol-1-yloxy)tris(dimethylamino)phosphanium; hexafluoro-lambda5-phosphanuide (205 mg, 463 μmol) at rt and stirred at rt for 2 hours. The mixture was diluted with water (100 mL) and EA (100 mL). The organic layer was separated, washed with H₂O (100 mL×3) and brine (30 mL), and dried (Na₂SO₄). The solvent was removed under reduced pressure to give crude methyl (2S)-4-{[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]formamido}-2-{[(benzyloxy)carbonyl]amino}butanoate (180 mg, 225 μmol, yield 104%). LC-MS m/z=798.8 [M+H]⁺.

### 14.2 Synthesis of Compound 4

To a solution of methyl (2S)-4-{[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]formamido}-2-{[(benzyloxy)carbonyl]amino}butanoate (50 mg, 62.5 μmol) in IPA (55 mL) and THF (7 mL), was added Pd/C (125 mg, 117 μmol) at rt and stirred at rt under H2 for 5 hours. LC-MS analysis indicated that reaction was well. The mixture was filtered and washed with THE (20 mL). The solvent was removed under reduced pressure to give crude methyl (2S)-4-{[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]formamido}-2-aminobutanoate (35.0 mg, 52.6 μmol). LC-MS m/z=664.8 [M+H]⁺.

### 14.3 Synthesis of Compound I-42

A solution of methyl (2S)-4-{[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]formamido}-2-aminobutanoate (35 mg, 52.6 μmol) in TFA (4 mL), H₂O (0.15 mL) and DCM (0.5 mL) was stirred at rt for 2 hours. Then the mixture was concentrated to afford crude, neutralized by 2 ml 7M NH₃ in MeOH, filtered. The product was purified by Prep-HPLC to afford the compound (15 mg, yield 60.2%) as a white solid. LC-MS m/z=475.0 [M+H]⁺. ¹H NMR (400 MHz, DMSO-d6) δ ppm 9.09 (brs, 1H), 8.89 (t, J=6.4 Hz, 1H), 8.13 (s, 1H), 7.90 (s, 1H), 7.76 (d, J=2.0 Hz, 1H), 7.32 (brs, 1H), 6.58 (d, J=2.4 Hz, 1H), 6.00 (d, J=7.6 Hz, 1H), 5.66 (d, J=4.0 Hz, 1H), 5.45 (d, J=6.8 Hz, 1H), 4.63-4.60 (m, 1H), 4.26 (d, J=1.2 Hz, 1H), 4.13-4.12 (m, 1H), 3.89 (s, 3H), 3.57 (s, 3H), 3.31-3.23 (m, 2H), 1.90-1.84 (m, 1H), 1.62-1.53 (m, 1H).

### 14.4 Synthesis of Compound I-43

To a solution of methyl (2S)-2-amino-4-{[(2S,3S,4R,5R)-5-[4-amino-5-(1-methyl-1H-pyrazol-3-yl)-7H-pyrrolo[2,3-d]pyrimidin-7-yl]-3,4-dihydroxyoxolan-2-yl]formamido}butanoate (50 mg, 105 μmol) in THF (2 mL), was added H₂O (2 mL) and lithium hydroxide hydrate (38 mg, 905 μmol) at rt and stirred at rt for 1 hour. The product was purified by Prep-HPLC to afford the compound (21 mg, yield 43.4%) as yellow solid. LC-MS m/z=461.0 [M+H]⁺. ¹H NMR (400 MHz, DMSO-d6) δ ppm 9.09 (brs, 1H), 9.07-8.97 (m, 1H), 8.17 (s, 1H), 8.11 (s, 1H), 7.73 (d, J=2.0 Hz, 1H), 7.70 (brs, 2H), 7.29 (brs, 1H), 6.70 (s, 1H), 6.02 (d, J=7.2 Hz, 1H), 5.87 (brs, 1H), 5.53 (brs, 1H), 4.59 (t, J=6.0 Hz, 1H), 4.27 (s, 1H), 4.16 (d, J=3.6 Hz, 1H), 3.89 (s, 3H), 3.28-3.20 (m, 2H), 1.97-1.92 (m, 1H), 1.84-1.79 (m, 1H).

### Example 15: Synthetic Scheme for Compound I-44

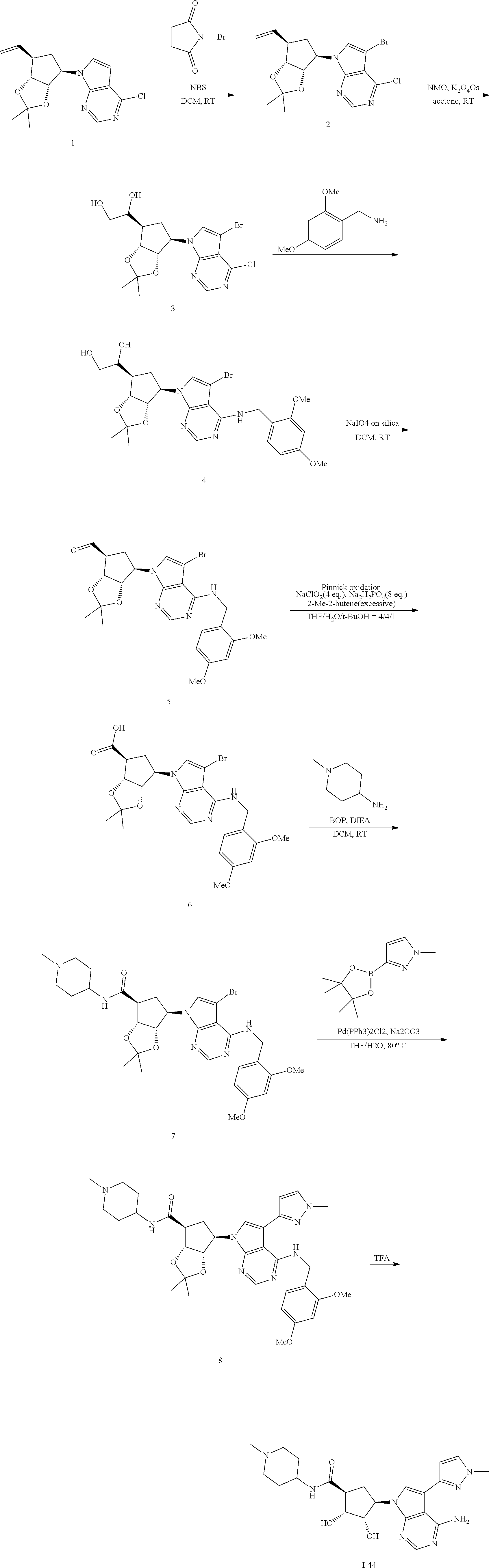

### 15.1 Synthesis of Compound 2

A mixture of 7-[(3aS,4R,6R,6aR)-6-ethenyl-2,2-dimethyl-hexahydrocyclopenta[d][1,3]dioxol-4-yl]-4-chloro-7H-pyrrolo[2,3-d]pyrimidine (2 g, 6.25 mmol) in methylene chloride (10 mL) was added 1-bromopyrrolidine-2,5-dione (1.33 g, 7.50 mmol) slowly, the reaction mixture was stirred at room temperature for 2 h. Then washed with water, extracted with EA (100 mL), the organic layer was dried with Na₂SO₄, filtered and concentrated, the residue was purified by flash chromatography, eluent: (PE\EA:20\1), afford 7-[(3aS,4R,6R,6aR)-6-ethenyl-2,2-dimethyl-hexahydrocyclopenta[d][1,3]dioxol-4-yl]-5-bromo-4-chloro-7H-pyrrolo[2,3-d]pyrimidine (520 mg, 1.30 mmol). MS(ESI): 398.1 [M+H]⁺.

### 15.2 Synthesis of Compound 3

A mixture of 7-[(3aS,4R,6R,6aR)-6-ethenyl-2,2-dimethyl-hexahydrocyclopenta[d][1,3]dioxol-4-yl]-5-bromo-4-chloro-7H-pyrrolo[2,3-d]pyrimidine (500 mg, 1.25 mmol) and N-methylmorpholine N-oxide (219 mg, 1.87 mmol) in acetone (10 mL) was added potassium osmate dihydrate (92.6 mg, 250 μmol), the mixture was stirred for 3 h, then purified by flash chromatography, eluent: (DCM\MeOH\:20\1), afford 1-[(3aR,4R,6R,6aS)-6-{5-bromo-4-chloro-7H-pyrrolo[2,3-d]pyrimidin-7-yl}-2,2-dimethyl-hexahydrocyclopenta[d][1,3]dioxol-4-yl]ethane-1,2-diol (240 mg, 554 μmol). MS(ESI): 431.1 [M+H]⁺.

### 15.3 Synthesis of Compound 4

A mixture of 1-[(3aR,4R,6R,6aS)-6-{5-bromo-4-chloro-7H-pyrrolo[2,3-d]pyrimidin-7-yl}-2,2-dimethyl-hexahydrocyclopenta[d][1,3]dioxol-4-yl]ethane-1,2-diol (400 mg, 924 μmol) and 1-(2,4-dimethoxyphenyl)methanamine (183 mg, 1.10 mmol) in dioxane (3 mL) was heated to 80° C., for 12 h. Then cooled to room temperature, washed with water (10 mL), extracted with EA (20 mL), the organic layer was dried with Na₂SO₄, filtered and concentrated, the residue was purified by flash chromatography, eluent; (DCM\MeOH:10\1), afford 1-[(3aR,4R,6R,6aS)-6-(5-bromo-4-{[(2,4-dimethoxyphenyl)methyl]amino}-7H-pyrrolo[2,3-d]pyrimidin-7-yl)-2,2-dimethyl-hexahydrocyclopenta[d][1,3]dioxol-4-yl]ethane-1,2-diol (255 mg, 452 μmol). MS(ESI): 563.1 [M+H]⁺.

### 15.4 Synthesis of Compound 5

A mixture of (R)-1-((3aR,4R,6R,6aS)-6-(5-bromo-4-((2,4-dimethoxybenzyl)amino)-7H-pyrrolo[2,3-d]pyrimidin-7-yl)-2,2-dimethyltetrahydro-3aH-cyclopenta[d][1,3]dioxol-4-yl)ethane-1,2-diol (300 mg, 0.53 mmol) in DCM (40 mL) was added 1 g of NaIO₄ on silica, the mixture was stirred at room temperature for 3 h then filtered and concentrated, the residue was purified by prep-TLC, eluent: (1:1 PE:EA), to afford the desired product. MS(ESI): 531.1 [M+H]⁺.

### 15.5 Synthesis of Compound 6

A mixture of (3aR,4S,6R,6aS)-6-(5-bromo-4-{[(2,4-dimethoxyphenyl)methyl]amino}-7H-pyrrolo[2,3-d]pyrimidin-7-yl)-2,2-dimethyl-hexahydrocyclopenta[d][1,3]dioxole-4-carbaldehyde (300 mg, 564 μmol) and sodium chlorite (203 mg, 2.25 mmol) and NaH₂PO₄ (541 mg, 4.51 mmol) and isobutylene (628 mg, 11.2 mmol) in tetrahydrofuran (4 mL) and water (4 mL) and 2-methyl-2-propanol (1 mL) was stirred at room temperature for 12 h, then washed with water (20 mL), extracted with EA (50 mL), the organic layer was dried with Na₂SO₄, filtered and concentrated, the residue was purified by prep-HPLC afford (3aR,4S,6R,6aS)-6-(5-bromo-4-{[(2,4-dimethoxyphenyl)methyl]amino}-7H-pyrrolo[2,3-d]pyrimidin-7-yl)-2,2-dimethyl-hexahydrocyclopenta[d][1,3]dioxole-4-carboxylic acid (67.5 mg, 123 μmol). MS(ESI): 547.1 [M+H]⁺.

### 15.6 Synthesis of Compound 7

A mixture (3aR,4S,6R,6aS)-6-(5-bromo-4-{[(2,4-dimethoxyphenyl)methyl]amino}-7H-pyrrolo[2,3-d]pyrimidin-7-yl)-2,2-dimethyl-hexahydrocyclopenta[d][1,3]dioxole-4-carboxylic acid (100 mg, 182 μmol) and 1-methylpiperidin-4-amine (41.5 mg, 364 μmol) and (1,2,3-benzotriazol-1-yloxy)tris(dimethylamino)phosphanium; hexafluoro-lambda5-phosphanuide (160 mg, 364 μmol) and N,N-diisopropylethylamine (70.5 mg, 546 μmol) in dimethylformamide (2 mL) was stirred at room temperature for 12 h. Then quenched with water (10 mL), extracted with EA (20 mL), the organic layer was dried with Na₂SO₄, filtered and concentrated, the residue was purified by flash chromatography, eluent: (DCM\MeOH:10\1), afford (3aR,4S,6R,6aS)-6-(5-bromo-4-{[(2,4-dimethoxyphenyl)methyl]amino}-7H-pyrrolo[2,3-d]pyrimidin-7-yl)-2,2-dimethyl-N-(1-methylpiperidin-4-yl)-hexahydrocyclopenta[d][1,3]dioxole-4-carboxamide (56.0 mg, 87.0 μmol). MS(ESI): 643.2 [M+H]⁺.

### 15.7 Synthesis of Compound 8

A mixture of (3aR,4S,6R,6aS)-6-(5-bromo-4-{[(2,4-dimethoxyphenyl)methyl]amino}-7H-pyrrolo[2,3-d]pyrimidin-7-yl)-2,2-dimethyl-N-(1-methylpiperidin-4-yl)-hexahydrocyclopenta[d][1,3]dioxole-4-carboxamide (70 mg, 108 μmol) and 1-methyl-3-(4,4,5,5-tetramethyl-1,3,2-dioxaborolan-2-yl)-1H-pyrazole (26.8 mg, 129 μmol) and Pd(PPh₃)₂Cl₂ (22.7 mg, 32.4 μmol) and disodium hydrate carbonate (40.1 mg, 324 μmol) in tetrahydrofuran (5 mL) and water (0.5 mL) was heated to 80° C., under N₂, for 12 h, then cooled to room temperature, washed with water (20 mL), extracted with EA (50 mL), the organic layer was dried with Na₂SO₄, filtered and concentrated, the residue was purified by prep-TLC, eluent: 9DCM\MeOH:10\1), afford (3aR,4S,6R,6aS)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-N-(1-methylpiperidin-4-yl)-hexahydrocyclopenta[d][1,3]dioxole-4-carboxamide (32.0 mg, 49.6 μmol). MS(ESI): 645.3 [M+H]⁺.

### 15.8 Synthesis of Compound I-44

A mixture of (3aR,4S,6R,6aS)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-N-(1-methylpiperidin-4-yl)-hexahydrocyclopenta[d][1,3]dioxole-4-carboxamide (40 mg, 62.0 μmol) in trifluoroacetic acid (4 mL) was stirred at room temperature, for 4 h, then concentrated, the residue was neutralized with LiOH solution to PH:8, then concentrated, the residue was dissolved in 7M NH₃ in MeOH, purified prep-HPLC to afford (1S,2R,3S,4R)-4-[4-amino-5-(1-methyl-1H-pyrazol-3-yl)-7H-pyrrolo[2,3-d]pyrimidin-7-yl]-2,3-dihydroxy-N-(1-methylpiperidin-4-yl)cyclopentane-1-carboxamide (2.60 mg, 5.72 μmol). MS(ESI): 456.1 [M+H]⁺. ¹H NMR (400 MHz, DMSO-d6) δ ppm 8.96 (br, 1H), 8.03-8.02 (m, 1H), 7.93-7.91 (m, 1H), 7.83 (s, 1H), 7.73-7.72 (m, 2H), 7.12 (br, 1H), 6.61 9d, J=2.4 Hz, 1H), 5.01-4.87 (m, 3H), 4.22-4.17 (m, 1H), 4.08-4.05 (m, 1H), 3.87 (s, 3H), 3.57-3.55 (m, 1H), 2.78-2.67 (m, 3H), 2.33-2.19 (m, 4H), 2.09-2.02 (m, 2H), 1.99-1.80 (m, 1H), 1.75-1.72 (m, 2H), 1.68-1.39 (m, 2H).

### Example 16: Synthetic Scheme for Compound I-45

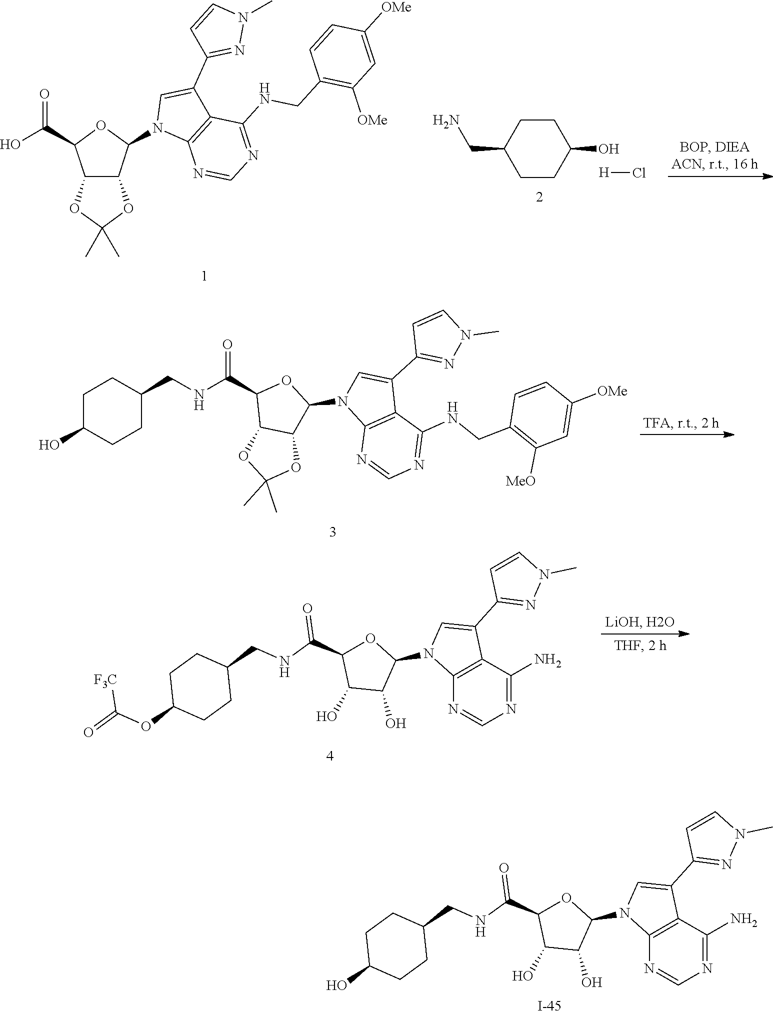

### 16.1 Synthesis of Compound 3

A solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylic acid (50 mg, 90.8 μmol) in acetonitrile (3 mL) was added (1s,4s)-4-(aminomethyl)cyclohexan-1-ol hydrochloride (29.9 mg, 181 μmol), (1,2,3-benzotriazol-1-yloxy)tris(dimethylamino)phosphanium; hexafluoro-lambda5-phosphanuide (60.1 mg, 136 μmol) and N,N-diisopropylethylamine (35.1 mg, 272 μmol). The mixture was stirred at 80° C. overnight. The mixture was diluted with water (80 mL) and extracted with EA (80 mL×2). Combined organic layers were washed with H₂O (100 mL×2) and brine (60 mL), and dried (Na₂SO₄). The solvent was removed under reduced pressure and the crude was used directly for the next step without further purification.

### 16.2 Synthesis of Compound 4

A solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-N-{[(1s,4s)-4-hydroxycyclohexyl]methyl}-tetrahydro-2H-furo[3,4-d][1,3]dioxole-4-carboxamide (40 mg, 60.4 μmol) in trifluoroacetic acid (3 mL) was stirred at room temperature overnight. Then the mixture was concentrated to afford crude, neutralized by aq. Na₂CO₃. The crude was concentrated and used for the next step directly.

### 16.3 Synthesis of Compound I-45

A solution of (1s,4s)-4-({[(2S,3S,4R,5R)-5-[4-amino-5-(1-methyl-1H-pyrazol-3-yl)-7H-pyrrolo[2,3-d]pyrimidin-7-yl]-3,4-dihydroxyoxolan-2-yl]formamido}methyl)cyclohexyl 2,2,2-trifluoroacetate (40 mg, 70.4 μmol) in tetrahydrofuran (3 mL) was added lithium hydroxide (8.43 mg, 352 μmol) and 1 mL H₂O. The mixture was stirred at room temperature overnight. The mixture was purified by prep. HPLC to afford product (2S,3S,4R,5R)-5-[4-amino-5-(1-methyl-1H-pyrazol-3-yl)-7H-pyrrolo[2,3-d]pyrimidin-7-yl]-3,4-dihydroxy-N-{[(1s,4s)-4-hydroxycyclohexyl]methyl}oxolane-2-carboxamide (28 mg, yield 84.5%). LC-MS m/z=472.0 [M+H]⁺. ¹H NMR (400 MHz, DMSO-d6) δ 9.09 (s, 1H), 8.86 (t, J=6.2 Hz, 1H), 8.09 (s, 1H), 7.89 (s, 1H), 7.76 (d, J=2.2 Hz, 1H), 7.31 (s, 1H), 6.57 (d, J=2.3 Hz, 1H), 5.97 (d, J=7.6 Hz, 1H), 5.65 (d, J=3.9 Hz, 1H), 5.45 (d, J=6.3 Hz, 1H), 4.65-4.62 (m, 1H), 4.29 (d, J=1.5 Hz, 1H), 4.25 (d, J=3.4 Hz, 1H), 4.11 (s, 1H), 3.89 (s, 3H), 3.71 (s, 1H), 3.07 (t, J=6.4 Hz, 2H), 1.57-1.48 (m, 3H), 1.36-1.34 (m, 6H).

### Example 17: Synthetic Scheme for Compound I-46

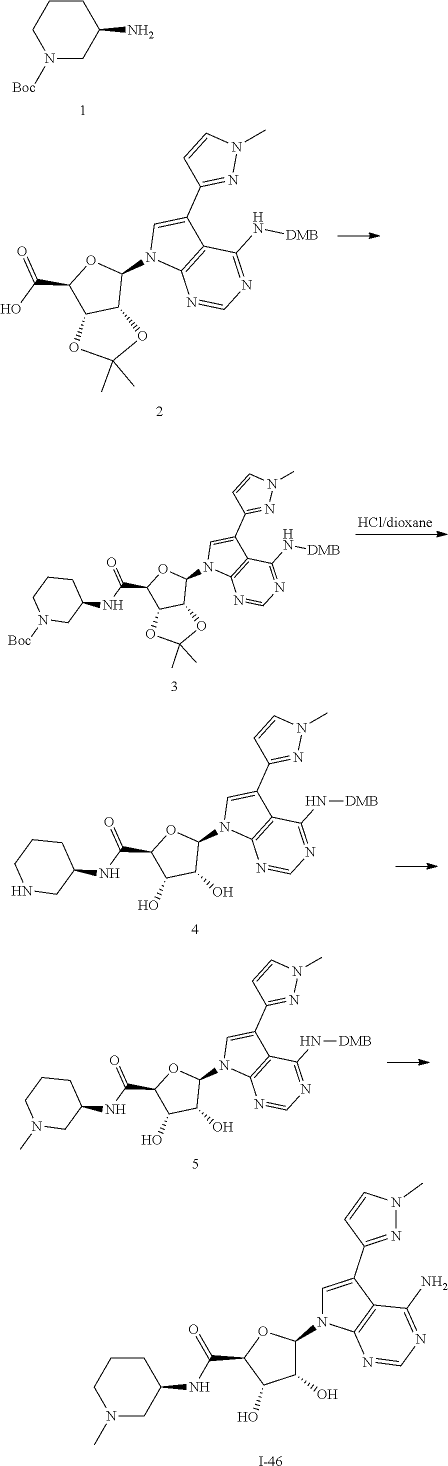

### 17.1 Synthesis of Compound 3

To a solution of tert-butyl (3R)-3-aminopiperidine-1-carboxylate (35 mg, 174 μmol) in ACN (2 mL) was added (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylic acid (95.7 mg, 174 μmol), [(1,2,3-benzotriazol-1-yloxy)bis(dimethylamino)-lambda5-phosphanyl]dimethylamine pentafluoro-lambda5-phosphane hydrofluoride (77.3 mg, 174 μmol) and N,N-diisopropylethylamine (22.4 mg, 174 μmol). The mixture was stirred at 80° C. for 6 h under nitrogen atmosphere and progress of reaction was monitored by LCMS. LCMS detected the starting material consumed. Purification by chromatography on a silica gel column with a gradient of DCM/MeOH (10:1) afforded the desired pure tert-butyl (3R)-3-[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-amido]piperidine-1-carboxylate (100 mg, 78.8%). LC-MS m/z=733 [M+H]⁺.

### 17.2 Synthesis of Compound 4

To a solution of tert-butyl (3S)-3-[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-amido]piperidine-1-carboxylate (75 mg, 102 μmol) in Dioxane (4M in HCl) (2 mL). The mixture was stirred at 25° C. for 2 h under nitrogen atmosphere and progress of reaction was monitored by LCMS. LCMS detected the starting material consumed and the mixture was concentrated in vacuo to give (2S,3S,4R,5R)-5-(4-{[(2,4-dimethoxyphenyl)methyl]amino}-5-(1-methyl-1H-pyrazol-3-yl)-7H-pyrrolo[2,3-d]pyrimidin-7-yl)-3,4-dihydroxy-N-[(3S)-piperidin-3-yl]oxolane-2-carboxamide (50.0 mg, 82.7%).

### 17.3 Synthesis of Compound 5

To a solution of (2S,3S,4R,5R)-5-(4-{[(2,4-dimethoxyphenyl)methyl]amino}-5-(1-methyl-1H-pyrazol-3-yl)-7H-pyrrolo[2,3-d]pyrimidin-7-yl)-3,4-dihydroxy-N-[(3R)-piperidin-3-yl]oxolane-2-carboxamide (50 mg, 84.3 μmol) in MeOH (2 mL) was added formaldehyde (2.53 mg, 84.3 μmol) and sodium cyanoboranuide (5.29 mg, 84.3 μmol). The mixture was stirred at 25° C. for 2 h under nitrogen atmosphere and progress of reaction was monitored by LCMS. LCMS detected the starting material consumed. Filter the mixture and the filtrate was concentrated under reduced pressure to give (2S,3S,4R,5R)-5-(4-{[(2,4-dimethoxyphenyl)methyl]amino}-5-(1-methyl-1H-pyrazol-3-yl)-7H-pyrrolo[2,3-d]pyrimidin-7-yl)-3,4-dihydroxy-N-[(3R)-1-methylpiperidin-3-yl]oxolane-2-carboxamide (20.0 mg, 39.1%).

### 17.4 Synthesis of Compound I-46

A mixture of (2S,3S,4R,5R)-5-(4-{[(2,4-dimethoxyphenyl)methyl]amino}-5-(1-methyl-1H-pyrazol-3-yl)-7H-pyrrolo[2,3-d]pyrimidin-7-yl)-3,4-dihydroxy-N-[(3R)-1-methylpiperidin-3-yl]oxolane-2-carboxamide (20 mg, 32.9 μmol) and TFA (3 mL) in DCM (3 mL) was stirred at room temperature for 3 h. Then the mixture was concentrated, the residue was neutralized with 7M NH₃ in methanol solution. The mixture was concentrated, the residue was purified by prep-HPLC to afford (2S,3S,4R,5R)-5-[4-amino-5-(1-methyl-1H-pyrazol-3-yl)-7H-pyrrolo[2,3-d]pyrimidin-7-yl]-3,4-dihydroxy-N-[(3R)-1-methylpiperidin-3-yl]oxolane-2-carboxamide (2.80 mg, 6.13 μmol) as a white solid. ESI LC-MS m/z=457 [M+H]⁺. ¹H NMR (400 MHz, CD₃OD) δ ppm 9.06 (s, 1H), 8.10 (s, 1H), 8.02 (d, J=8.4 Hz, 1H), 7.92 (s, 1H), 7.75 (d, J=2.4 Hz, 1H), 7.26 (s, 1H), 6.61 (d, J=2.4 Hz 1H), 6.08 (d, J=7.2 Hz, 1H), 5.63 (d, J=4.4 Hz, 1H), 5.45 (d, J=5.2 Hz, 1H), 4.65-4.61 (m, 1H), 4.28 (d, J=1.6 Hz, 1H), 4.20-4.17 (m, 1H), 3.88 (s, 3H), 3.86-3.79 (m, 1H), 2.67-2.62 (m, 1H), 2.43-2.32 (m, 1H), 2.06 (s, 3H), 1.95-1.89 (m, 2H), 1.58-1.20 (m, 5H).

### Example 18: Synthetic Scheme for Compound I-47

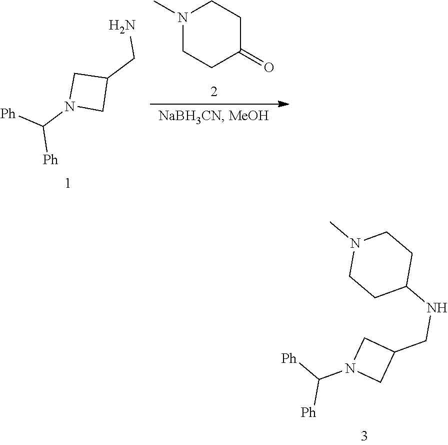

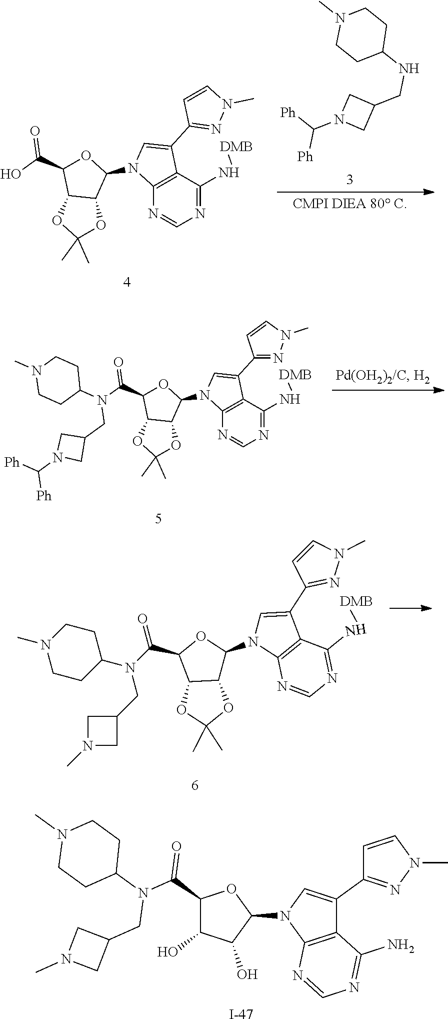

### 18.1 Synthesis of Compound 3

1-[1-(Diphenylmethyl)azetidin-3-yl]methanamine (300 mg, 1.18 mmol), 1-methylpiperidin-4-one (267 mg, 2.36 mmol) were mixed in MeOH (5 mL). the mixture was stirred at rt for 30 min. NaBH₃CN (365 mg, 5.90 mmol) was added and stirred at for 20 h. TLC show reaction completed. The reaction solution concentrated under reduced pressure to afford the crude. The crude was purified by TLC (MeOH/DCM=1/10) to afford the target compound (180 mg, yield 43.6%). LC-MS m/z=350.1 [M+H]⁺.

### 18.2 Synthesis of Compound 5

(3aS,4S,6R,6aR)-6-(4-{[(2,4-Dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylic acid (157 mg, 286 μmol), N-{[1-(diphenylmethyl)azetidin-3-yl]methyl}-1-methylpiperidin-4-amine (100 mg, 286 μmol), CMPI (145 mg, 572 μmol), DIEA (110 mg, 858 μmol) were mixed in MeCN (5 mL). The mixture was heated to 80° C. and stirred for 12 h. The mixture was diluted with water (80 mL) and extracted with EA (80 mL×2). Combined organic layers were washed with H₂O (100 mL×2) and brine (60 mL), and dried (Na₂SO₄). The solvent was removed under reduced pressure to afford crude. The crude was purified by fish chromatography (MeOH˜DCM: 0˜20%) to afford the target compound (150 mg, yield 59.5%). LC-MS m/z=441.9 [M+H]⁺.

### 18.3 Synthesis of Compound 6

(3aS,4S,6R,6aR)-6-(4-{[(2,4-Dimethoxyphenyl)methyl]amino}-5-(1-methyl-1H-pyrazol-3-yl)-7H-pyrrolo[2,3-d]pyrimidin-7-yl)-N-{[1-(diphenylmethyl)azetidin-3-yl]methyl}-2,2-dimethyl-N-(1-methylpiperidin-4-yl)-tetrahydro-2H-furo[3,4-d][1,3]dioxole-4-carboxamide (150 mg, 170 μmol), Pd(OH)₂/C (118 mg, 850 μmol) were mixed in MeOH (15 mL). The mixture was stirred at rt for 20 h under H₂ atmosphere. The reaction solution was filtrated and concentrated under reduced pressure to afford the crude. The crude was purified by prep HPLC to afford target compound as a solid (40 mg, yield 32.2%). LC-MS m/z=729.8 [M+H]⁺.

### 18.4 Synthesis of Compound I-47

To a solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-N-[(1-methylazetidin-3-yl)methyl]-N-(1-methylpiperidin-4-yl)-tetrahydro-2H-furo[3,4-d][1,3]dioxole-4-carboxamide (40.0 mg, 54.8 μmol) in DCM (2 mL) was added TFA (5 mL). The mixture was stirred at rt for 5 hours. Then the mixture was concentrated to afford crude, neutralized by 4 ml 7M NH₃ in MeOH. The product was purified by Prep-HPLC to afford compound as a yellow solid (4 mg, yield 13.5%). LC-MS m/z=540.2 [M+H]⁺. ¹H NMR (400 MHz, DMSO-d6) δ 8.96 (s, 1H), 8.07 (s, 1H), 7.91-7.81 (m, 1H), 7.76 (s, 1H), 7.21 (s, 1H), 6.68-6.40 (m, 1H), 6.30-6.20 (m, 1H), 4.82-4.69 (m, 1H), 4.55-4.46 (m, 1H), 4.46-4.35 (m, 1H), 4.32-4.25 (m, 2H), 3.89 (s, 3H), 3.81-3.63 (m, 3H), 3.61-3.50 (m, 2H), 3.42-3.30 (m, 1H), 3.25-3.15 (m, 1H), 2.96-2.66 (m, 3H), 2.56 (s, 2H), 2.37 (s, 1H), 2.30-2.10 (m, 3H), 2.12-2.05 (m, 1H), 2.03-1.94 (m, 1H), 1.91-1.55 (m, 3H), 1.55-1.35 (m, 1H).

### Example 19: Synthetic Scheme for Compound I-48

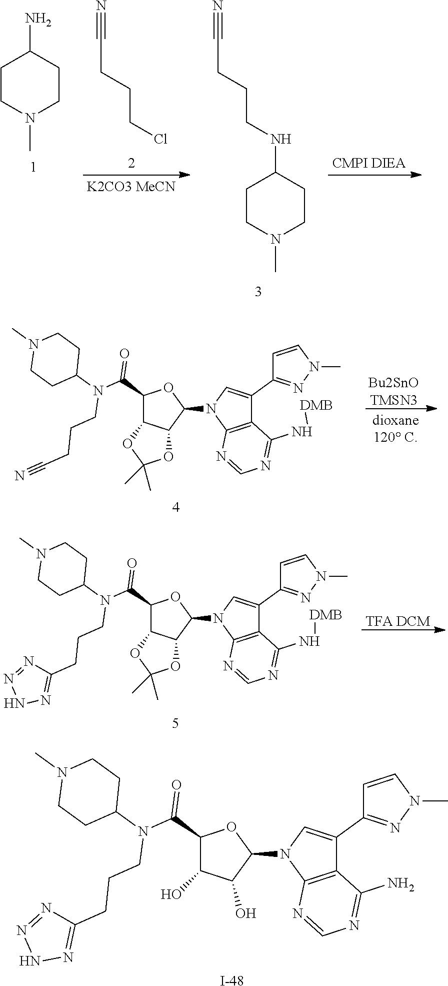

### 19.1 Synthesis of Compound 3

1-Methylpiperidin-4-amine (50.0 mg, 437 μmol), 4-chlorobutanenitrile (90.5 mg, 874 μmol), iodopotassium (7.25 mg, 43.7 μmol), K₂CO₃ (180 mg, 1.31 mmol) were mixed in MeCN (5 mL). The mixture was heated to 80° C. and stirred at for 4 h. TLC show reaction completed. Reaction solution was concentrated to afford the crude. The crude was purified by TLC (MeOH/DCM=1/10) to afford target compound as a liquid. 4-[(1-methylpiperidin-4-yl)amino]butanenitrile (100 mg, yield 12.6%). LC-MS m/z=182.2 [M+H]⁺.

### 19.2 Synthesis of Compound 4

(3aS,4S,6R,6aR)-6-(4-{[(2,4-Dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylic acid (10.0 mg, 18.1 μmol), 4-[(1-methylpiperidin-4-yl)amino]butanenitrile (6.56 mg, 36.2 μmol), CMPI (9.23 mg, 36.2 μmol), DIEA (7.00 mg, 54.3 μmol) were mixed in MeCN (5 mL). The mixture was heated to 80° C. and stirred for 2 h. The mixture was diluted with water (80 mL) and extracted with EA (80 mL×2). Combined organic layers were washed with H₂O (100 mL×2) and brine (60 mL), and dried (Na₂SO₄). The solvent was removed under reduced pressure to afford crude. The crude was purified by flash chromatography (MeOH˜DCM: 0˜20%) to afford the target compound as a solid (100 mg, yield 86.2%). LC-MS m/z=714.2 [M+H]⁺.

### 19.3 Synthesis of Compound 6

(3aS,4S,6R,6aR)-N-(3-Cyanopropyl)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-N-(1-methylpiperidin-4-yl)-tetrahydro-2H-furo[3,4-d][1,3]dioxole-4-carboxamide (100 mg, 140 μmol), dibutylstannanone (69.7 mg, 280 μmol), azidotrimethylsilane (160 mg, 1.39 mmol) were mixed in dioxane (1 mL). The mixture was sealed and stirred at 120° C. for 16 h. The reaction solution was concentrated to afford crude. The crude was purified by prep HPLC to afford the target compound as a solid. (50 mg, yield 47.6%). LC-MS m/z=757.5 [M+H]⁺.

### 19.4 Synthesis of Compound I-48

To a solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-N-(1-methylpiperidin-4-yl)-N-[3-(2H-1,2,3,4-tetrazol-5-yl)propyl]-tetrahydro-2H-furo[3,4-d][1,3]dioxole-4-carboxamide (60.0 mg, 79.2 μmol) in DCM (2 mL) was added TFA (5 mL) The mixture was stirred at rt for 5 hours. Then the mixture was concentrated to afford crude, neutralized by 4 mL 7M NH₃ in MeOH. The product was purified by Prep-HPLC to afford compound (16.4 mg, yield 36.68%). LC-MS m/z=567.2 [M+H]⁺. ¹H NMR (400 MHz, DMSO-d6) δ 8.94 (s, 1H), 8.06 (d, J=2.2 Hz, 1H), 7.98 (s, 0.5H), 7.77 (s, 0.5H), 7.76 (d, J=2.2 Hz, 0.5H), 7.71 (d, J=2.2 Hz, 0.5H), 7.19 (s, 1H), 6.52-6.44 (m, 1H), 6.34-6.24 (m, 1H), 4.89-4.63 (m, 1H), 4.52-4.39 (m, 1H), 4.35-4.20 (m, 1H), 4.15-4.02 (m, 1H), 3.89 (d, J=4.5 Hz, 3H), 3.75-3.68 (m, 1H), 3.40-3.20 (m, 3H), 2.96-2.72 (m, 4H), 2.25-2.15 (m, 2H), 2.12-1.33 (m, 9H).

### Example 20: Synthetic Scheme for Compound I-49

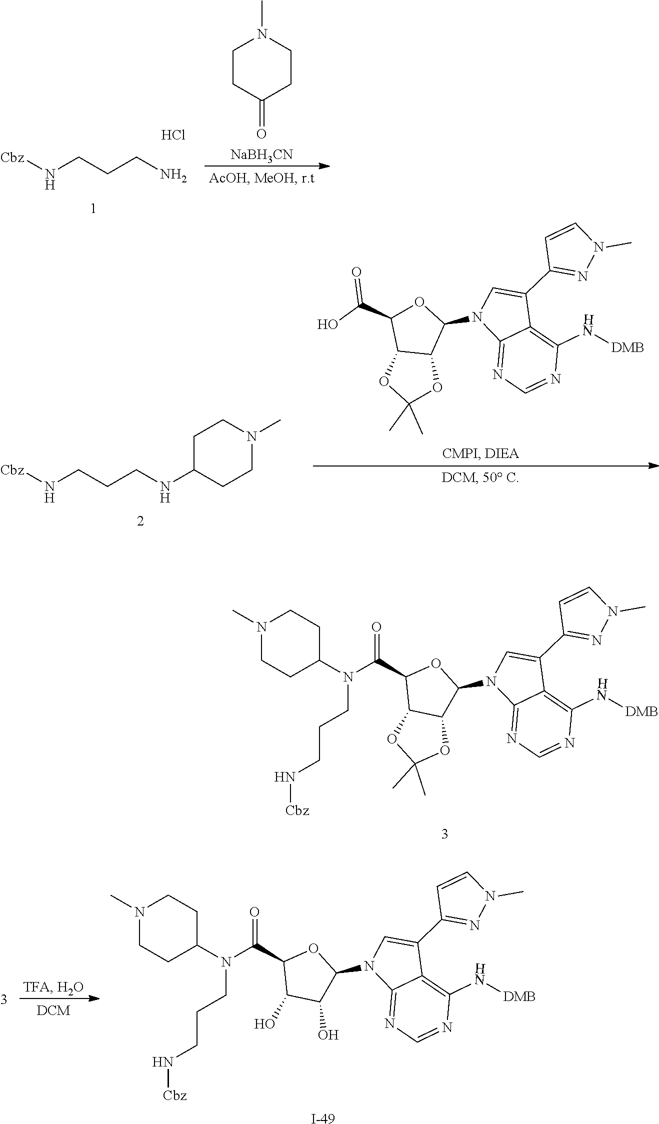

### 20.1 Synthesis of Compound 2

A solution of benzyl N-(3-aminopropyl)carbamate (1 g, 4.80 mmol), 1-methylpiperidin-4-one (1.08 g, 9.60 mmol) and acetic acid (576 mg, 9.60 mmol) in methanol (10 mL) was stirred 3 minutes at room temperature. To this mixture was added sodium cyanoboranuide (603 mg, 9.60 mmol) was stirred for 16 hours. The mixture was charged with water (50 mL) and extracted with ethyl acetate (50 mL×3). The combined organic layers were dried (Na₂SO₄). The solvent was removed under reduced pressure to get crude product. The mixture was purified by silica gel column to get benzyl N-{3-[(1-methylpiperidin-4-yl)amino]propyl}carbamate (850 mg, yield 58.2%). LC-MS m/z=306.3 [M+H]⁺.

### 20.2 Synthesis of Compound 3

A solution of benzyl N-{3-[(1-methylpiperidin-4-yl)amino]propyl}carbamate (300 mg, 982 μmol), 2-chloro-1-methylpyridin-1-ium iodide (751 mg, 2.94 mmol), N,N-diisopropylethylamine (379 mg, 2.94 mmol), 2-chloro-1-methylpyridin-1-ium iodide (751 mg, 2.94 mmol) and (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylic acid (540 mg, 982 μmol) in DCM (10 mL) was stirred at 50° C. for 16 hours. The mixture was charged with water (50 mL) and extracted with ethyl acetate (50 mL×3). The combined organic layers were dried (Na₂SO₄) The solvent was removed under reduced pressure to get crude product. The mixture was purified by silica gel column to get benzyl N-(3-{1-[(3aS,4S,6R,6aR)-6-(4-{1[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]-N-(1-methylpiperidin-4-yl)formamido}propyl)carbamate (600 mg, yield 72.9%). LC-MS m/z=838.5 [M+H]⁺.

### 20.3 Synthesis of Compound I-49

To a solution of benzyl N-(3-{1-[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]-N-(1-methylpiperidin-4-yl)formamido}propyl)carbamate (40 mg, 47.7 μmol) in DCM (1 mL) was added TFA (4 ml) and water (0.2 mL). The mixture was stirred at room temperature for 5 hours. The mixture was concentrated to obtain crude product, which was neutralized by 4 mL 7M NH₃ in MeOH. The methanol was removed under reduced pressure. The product was purified by Prep-HPLC to afford benzyl N-(3-{1-[(2S,3S,4R,5R)-5-[4-amino-5-(1-methyl-1H-pyrazol-3-yl)-7H-pyrrolo[2,3-d]pyrimidin-7-yl]-3,4-dihydroxyoxolan-2-yl]-N-(1-methylpiperidin-4-yl)formamido}propyl)carbamate (6.70 mg, yield 24.6%) as white solid. LC-MS m/z=648.0 [M+H]⁺. ¹H NMR (400 MHz, DMSO-d6) δ ppm 8.95 (s, 1H), 8.06 (s, 1H), 7.85 (d, J=48 Hz, 1H), 7.77-7.70 (m, 1H), 7.39-7.28 (m, 6H), 7.21 (s, 1H), 6.49 (d, J=2.4 Hz, 1H), 6.30-6.25 (m, 1H), 5.56-5.55 (m, 1H), 5.55-5.51 (m, 1H), 5.02 (s, 1H), 4.76-4.50 (m, 1H), 4.40-4.50 (m, 1H), 4.31-4.22 (m, 1H), 3.88 (d, J=6.4 Hz, 3H), 3.22-3.18 (m, 2H), 3.03-2.99 (m, 2H), 2.13 (d, J=10.4 Hz, 3H), 1.94-1.46 (m, 8H).

### Example 21: Synthetic Scheme for Compound I-50

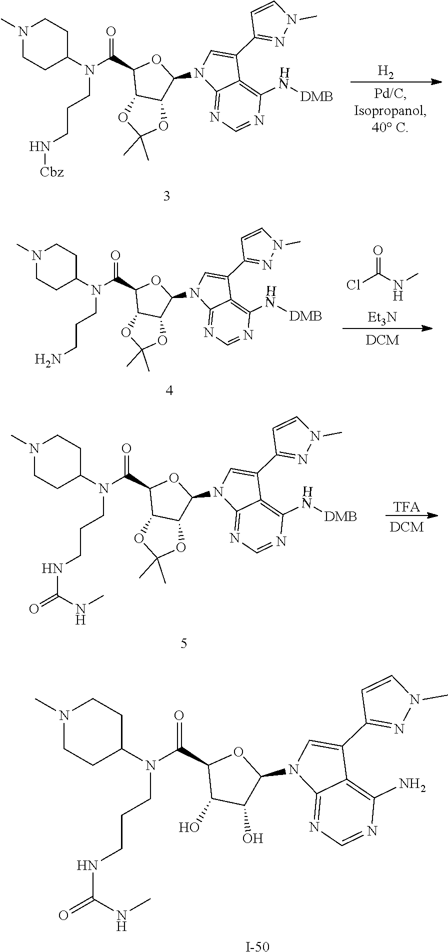

### 21.1 Synthesis of Compound 4

Benzyl N-(3-{1-[(3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxol-4-yl]-N-(1-methylpiperidin-4-yl)formamido}propyl)carbamate (compound 3, prepared as in Example 20, 520 mg, 620 μmol) and Pd/C (100 mg) were mixed with Isopropanol (10 mL) and attached to a hydrogenation apparatus. The system was evacuated and then refilled with hydrogen. The reaction was stirred at room temperature for 16 hours. Pd/C was filtered off and the solvent was removed under reduced pressure to afford (3aS,4S,6R,6aR)-N-(3-aminopropyl)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-N-(1-methylpiperidin-4-yl)-tetrahydro-2H-furo[3,4-d][1,3]dioxole-4-carboxamide (210 mg, 298 μmol, 48.1% yield). LC-MS m/z=704.2 [M+H]⁺.

### 21.2 Synthesis of Compound 5

To a solution of (3aS,4S,6R,6aR)-N-(3-aminopropyl)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-N-(1-methylpiperidin-4-yl)-tetrahydro-2H-furo[3,4-d][1,3]dioxole-4-carboxamide (70 mg, 99.4 μmol) and N-methylcarbamoyl chloride (18.5 mg, 198 μmol) in DCM (3 mL) was added triethylamine (30.1 mg, 298 μmol). The reaction mixture was stirred at room temperature for 30 minutes. The solvent was removed under reduced pressure to afford (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-N-{3-[(methylcarbamoyl)amino]propyl}-N-(1-methylpiperidin-4-yl)-tetrahydro-2H-furo[3,4-d][1,3]dioxole-4-carboxamide (70.0 mg, 91.9 μmol), yield: 92.5%. LC-MS m/z=762.5 [M+H]⁺.

### 21.3 Synthesis of Compound I-50

To a solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-N-{3-[(methylcarbamoyl)amino]propyl}-N-(1-methylpiperidin-4-yl)-tetrahydro-2H-furo[3,4-d][1,3]dioxole-4-carboxamide (70 mg, 91.9 μmol) in DCM (1 mL) was added TFA (6 mL) and H₂O (0.1 mL). The mixture was stirred at room temperature for 2 h. The mixture was concentrated to afford crude, neutralized by 4 ml 7M NH₃ in MeOH. The MeOH was removed under reduced pressure to afford crude. Crude was dissolved in MeOH. The product was purified by Prep-HPLC to afford (2S,3S,4R,5R)-5-[4-amino-5-(1-methyl-1H-pyrazol-3-yl)-7H-pyrrolo[2,3-d]pyrimidin-7-yl]-3,4-dihydroxy-N-{3-[(methylcarbamoyl)amino]propyl}-N-(1-methylpiperidin-4-yl)oxolane-2-carboxamide (21.6 mg, 37.8 μmol) as white solid, yield: 41.2%. LC-MS m/z=572.0 [M+H]⁺. ¹H NMR (400 MHz, DMSO-d6) δ ppm 8.94 (s, 1H), 8.06 (d, 1H), 7.85 (d, J=12 Hz, 1H), 7.75 (dd, J=9.2 Hz, 2.4 Hz, 1H), 7.19 (s, 1H), 6.50-6.48 (m, 1H), 6.29-6.25 (m, 1H), 6.29-6.27 (m, 1H), 5.97-5.73 (m, 1H), 5.55-5.50 (m, 1H), 4.70 (dd, J=8 Hz, J=2 Hz, 1H), 4.50-4.39 (m, 1H), 4.39-4.21 (m, 1H), 4.06 (s, 3H), 3.26-3.13 (m, 1H), 3.00-3.26 (m, 2H), 2.82-2.74 (m, 2H), 2.67 (d, J=2 Hz, 3H), 2.13 (d, J=9.2 Hz, 3H). 2.09-1.45 (m, 9H).

### Example 22: Synthetic Scheme for Compound I-51

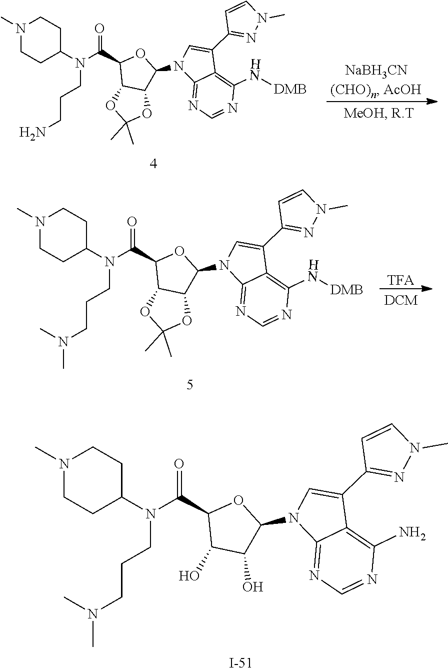

### 22.1 Synthesis of Compound 5

To a solution of (3aS,4S,6R,6aR)-N-(3-aminopropyl)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-N-(1-methylpiperidin-4-yl)-tetrahydro-2H-furo[3,4-d][1,3]dioxole-4-carboxamide (compound 4, prepared as in Example 21, 60 mg, 85.2 μmol), acetic acid (10.2 mg, 170 μmol) and formaldehyde (25.5 mg, 852 μmol) in MeOH (3 mL) was stirred at room temperature for 30 minutes. Then added sodium cyanoborohydride (10.6 mg, 170 μmol). The reaction mixture was stirred at room temperature for 16 hours. The mixture was added with water (50 mL) and extracted with EA (50 mL×3). The combined organic layers were dried (Na₂SO₄). The solvent was removed under reduced pressure to afford crude. The mixture was purified by silica gel column (DCM:MeOH=10:1) to afford (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-N-[3-(dimethylamino)propyl]-2,2-dimethyl-N-(1-methylpiperidin-4-yl)-tetrahydro-2H-furo[3,4-d][1,3]dioxole-4-carboxamide (45.0 mg, 61.4 μmol), yield 72.2%. LC-MS m/z=732.0 [M+H]⁺.

### 22.2 Synthesis of Compound I-51

To a solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-N-[3-(dimethylamino)propyl]-2,2-dimethyl-N-(1-methylpiperidin-4-yl)-tetrahydro-2H-furo[3,4-d][1,3]dioxole-4-carboxamide (45 mg, 61.4 μmol) in DCM (1 mL) was added TFA (6 mL) and H₂O (0.1 mL). The mixture was stirred at room temperature for 2 hours. Then the mixture was concentrated to afford crude, neutralized by 4 ml 7M NH₃ in MeOH. The MeOH was removed under reduced pressure to afford crude. Then crude is dissolved using MeOH. The product was purified by Prep-HPLC to afford (2S,3S,4R,5R)-5-[4-amino-5-(1-methyl-1H-pyrazol-3-yl)-7H-pyrrolo[2,3-d]pyrimidin-7-yl]-N-[3-(dimethylamino)propyl]-3,4-dihydroxy-N-(1-methylpiperidin-4-yl)oxolane-2-carboxamide (5.10 mg, 9.41 μmol) as white solid. LC-MS m/z=542.3 [M+H]⁺. ¹H NMR (400 MHz, DMSO-d6) δ ppm 8.2 (d, J=2.4 Hz, 1H), 8.95 (d, J=8 Hz, 1H), 7.64 (s, 1H), 6.63 (s, 1H), 6.44-6.40 (m, 1H), 3.49 (s, 1H), 3.13-3.06 (m, 1H), 4.41-4.11 (m, 1H), 3.98 (s, 3H), 3.67-3.38 (m, 4H), 3.29-3.09 (m, 1H), 2.92 (s, 6H), 2.85 (d, J=14.4 Hz, 3H), 2.28-1.83 (m, 6H).

### Example 23: Synthetic Scheme for Compound I-52

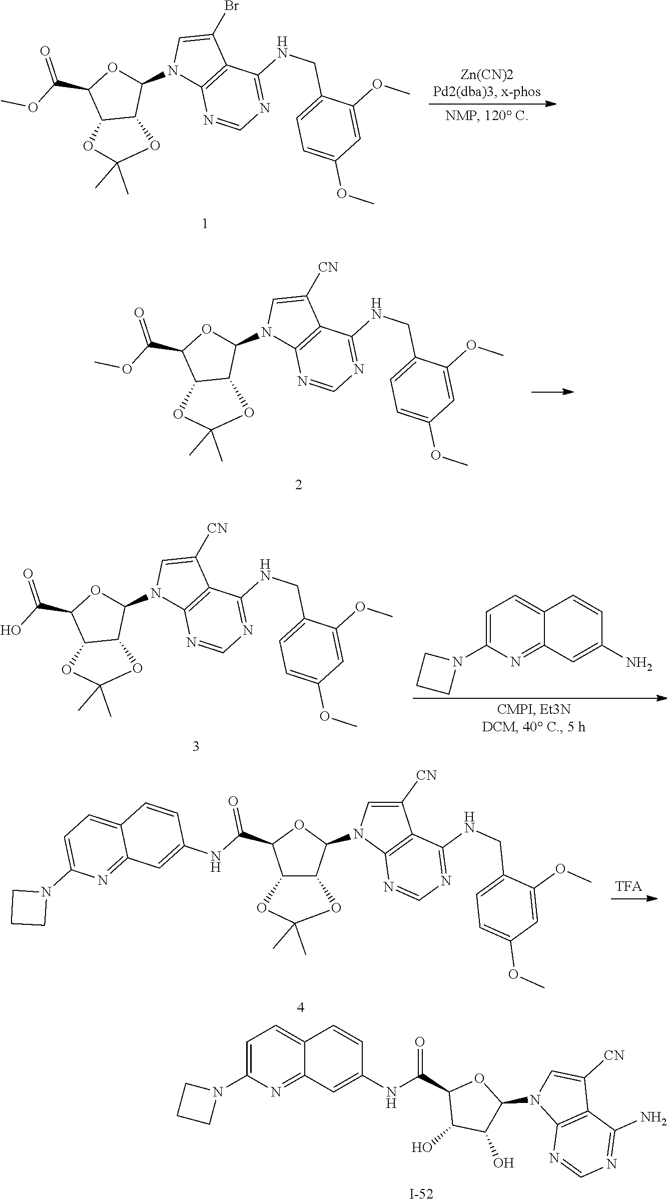

### 23.1 Synthesis of Compound 3

A mixture of methyl (3aS,4S,6R,6aR)-6-(5-bromo-4-{[(2,4-dimethoxyphenyl)methyl]amino}-7H-pyrrolo[2,3-d]pyrimidin-7-yl)-2,2-dimethyl-tetrahydro-2H-furo[3,4-d][1,3]dioxole-4-carboxylate (150 mg, 266 μmol) and Pd₂dba₃ (48.7 mg, 53.2 μmol) and XPhos (25.3 mg, 53.2 μmol) and bis(cyano) zinc (93.7 mg, 798 μmol) in N-methylpyrrolidone (3 mL) was heated to 120° C., under N₂, for 12 h. Then cooled to room temperature, washed with water (20 mL), extracted with EA (60 mL), the organic layer was dried with Na₂SO₄, filtered and concentrated, the residue was purified by prep-HPLC afford (3aS,4S,6R,6aR)-6-(5-cyano-4-{[(2,4-dimethoxyphenyl)methyl]amino}-7H-pyrrolo[2,3-d]pyrimidin-7-yl)-2,2-dimethyl-tetrahydro-2H-furo[3,4-d][1,3]dioxole-4-carboxylic acid (25.0 mg, 50.4 μmol). MS(ESI): 496.1 [M+H]⁺.

### 23.2 Synthesis of Compound 4

A mixture of (3aS,4S,6R,6aR)-6-(5-cyano-4-{[(2,4-dimethoxyphenyl)methyl]amino}-7H-pyrrolo[2,3-d]pyrimidin-7-yl)-2,2-dimethyl-tetrahydro-2H-furo[3,4-d][1,3]dioxole-4-carboxylic acid (25 mg, 50.4 μmol) and 2-(azetidin-1-yl)quinolin-7-amine (12.0 mg, 60.4 μmol) and 2-chloro-1-methylpyridin-1-ium (12.8 mg, 100 μmol) and triethylamine (20.3 mg, 201 μmol) in methylene chloride (4 mL) was heated to 40° C., under N₂, for 4 h, then washed with water (20 mL), extracted with EA (50 mL), the organic layer was dried with Na₂SO₄, filtered and concentrated. The residue was used directly for the next step.

### 23.3 Synthesis of Compound I-52

A mixture of (3aS,4S,6R,6aR)-N-[2-(azetidin-1-yl)quinolin-7-yl]-6-(5-cyano-4-{[(2,4-dimethoxyphenyl)methyl]amino}-7H-pyrrolo[2,3-d]pyrimidin-7-yl)-2,2-dimethyl-tetrahydro-2H-furo[3,4-d][1,3]dioxole-4-carboxamide (25 mg, 36.9 μmol) in trifluoroacetic acid (4 mL) was stirred at room temperature for 12 h, then concentrated under reduced pressure, then neutralized with saturated NaHCO₃ solution to pH 8, then filtered and solid was collected, dissolved in 3 mL of MeOH, then purified by prep-HPLC to afford (2S,3S,4R,5R)-5-{4-amino-5-cyano-7H-pyrrolo[2,3-d]pyrimidin-7-yl}-N-[2-(azetidin-1-yl)quinolin-7-yl]-3,4-dihydroxyoxolane-2-carboxamide (9.00 mg, 18.4 μmol). MS(ESI): 526.7 [M+H]⁺. ¹H NMR (400 MHz, DMSO-d6) δ ppm 8.43 (d, J=2.4 Hz, 1H), 8.12 (s, 1H), 7.87-7.77 (m, 3H), 7.52 (d, J=8.0 Hz, 1H), 7.36 (s, 1H), 7.19 (d, J=8.4 Hz, 1H), 7.05 (d, J=8.4 Hz, 1H), 6.64 (d, J=8.8 Hz, 1H), 6.56-6.55 (m, 1H), 6.64 (d, J=4.8 Hz, 1H), 6.56-6.55 (m, 1H), 5.48 (d, J=4.8 Hz, 1H), 5.36-5.19 (m, 1H), 4.91-4.77 (m, 1H), 4.59-4.46 (m, 1H), 3.95-3.87 (m, 1H), 2.78-2.75 (m, 2H), 2.44-2.41 (m, 2H), 2.33-2.31 (m, 2H), 2.13-2.07 (m, 2H), 1.93-1.88 (m, 3H), 1.73-1.67 (m, 2H).

### Example 24: Synthetic Scheme for Compound I-53

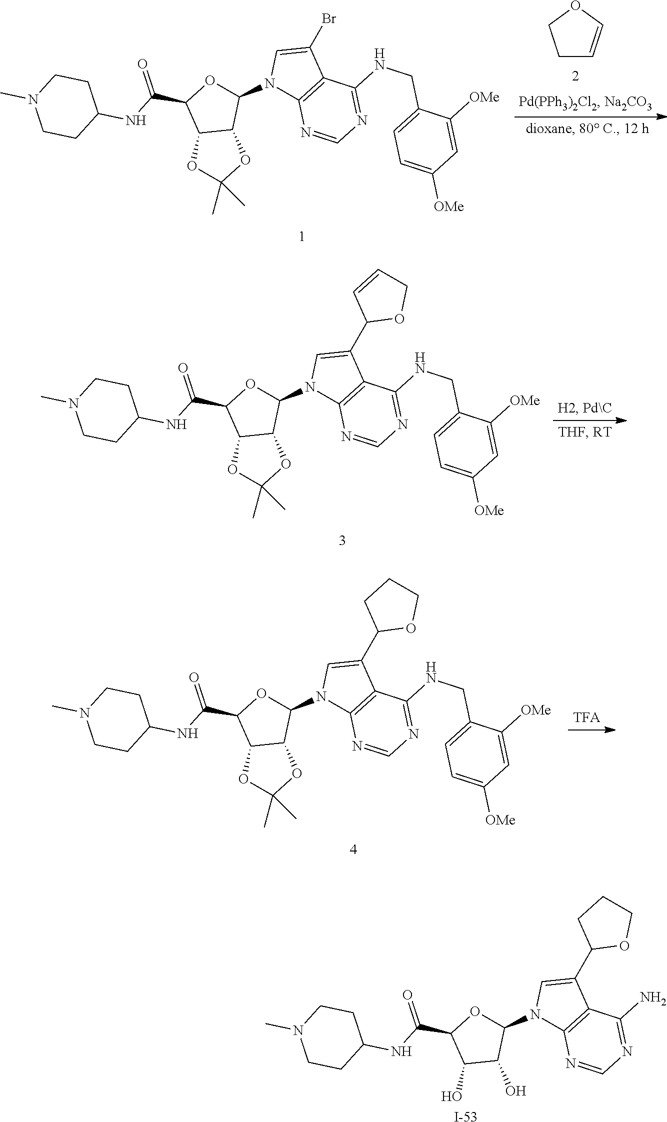

### 24.1 Synthesis of Compound 3

A mixture of (3aS,4S,6R,6aR)-6-(5-bromo-4-{[(2,4-dimethoxyphenyl)methyl]amino}-7H-pyrrolo[2,3-d]pyrimidin-7-yl)-2,2-dimethyl-N-(1-methylpiperidin-4-yl)-tetrahydro-2H-furo[3,4-d][1,3]dioxole-4-carboxamide (100 mg, 154 μmol) and Pd(PPh3)₂Cl2 (10.8 mg, 15.4 μmol) and disodium hydrate carbonate (57.2 mg, 462 μmol) and 1-methyl-5-(4,4,5,5-tetramethyl-1,3,2-dioxaborolan-2-yl)pyrazole (80.1 mg, 385 μmol) in tetrahydrofuran (4 mL) and water (0.4 mL) was heated to 80° C., under N₂ for 12 h, then cooled to room temperature, washed with water (10 mL), extracted with EA (20 mL), the organic layer was dried with Na₂SO₄, filtered and concentrated, the residue was purified by prep-TLC, afford (3aS,4S,6R,6aR)-6-[5-(2,5-dihydrofuran-2-yl)-4-{[(2,4-dimethoxyphenyl)methyl]amino}-7H-pyrrolo[2,3-d]pyrimidin-7-yl]-2,2-dimethyl-N-(1-methylpiperidin-4-yl)-tetrahydro-2H-furo[3,4-d][1,3]dioxole-4-carboxamide (64.0 mg, 100 μmol). MS(ESI): 635.3 [M+H]⁺.

### 24.2 Synthesis of Compound 4

A mixture of (3aS,4S,6R,6aR)-6-[5-(2,5-dihydrofuran-2-yl)-4-{[(2,4-dimethoxyphenyl)methyl]amino}-7H-pyrrolo[2,3-d]pyrimidin-7-yl]-2,2-dimethyl-N-(1-methylpiperidin-4-yl)-tetrahydro-2H-furo[3,4-d][1,3]dioxole-4-carboxamide (64.0 mg, 100 μmol) and 20 mg of Pd\C in 6 mL of THF, was stirred at room temperature, under H2, for 6 h, then filtered and concentrated, the residue was used directly for the next step. MS(ESI): 637.3 [M+H]⁺.

### 24.3 Synthesis of Compound I-53

A mixture of (3aS,4S,6R,6aR)-6-(4-{[(2,4-dimethoxyphenyl)methyl]amino}-5-(oxolan-2-yl)-7H-pyrrolo[2,3-d]pyrimidin-7-yl)-2,2-dimethyl-N-(1-methylpiperidin-4-yl)-tetrahydro-2H-furo[3,4-d][1,3]dioxole-4-carboxamide (20 mg, 31.4 μmol) in trifluoroacetic acid (3 mL) was stirred at room temperature, for 4 h, then concentrated under reduced pressure, the residue was neutralized with saturated Na₂CO₃ solution to Ph 8, concentrated under reduced pressure, the residue was purified by prep-HPLC to afford (2S,3S,4R,5R)-5-[4-amino-5-(oxolan-2-yl)-7H-pyrrolo[2,3-d]pyrimidin-7-yl]-3,4-dihydroxy-N-(1-methylpiperidin-4-yl)oxolane-2-carboxamide (8.00 mg, 17.9 μmol). MS(ESI): 447.2 [M+H]⁺. ¹H NMR (400 MHz, DMSO-d6) δ ppm 8.35 (t, J=7.6 Hz, 1H), 8.10 (s, 1H), 7.43 (s, 1H), 6.92 (br, 2H), 5.99 (dd, J=7.6, 2.4 Hz, 1H), 5.61 (d, J=4.0 Hz, 1H), 5.39 (d, J=6.8 Hz, 1H), 4.96 (t, J=7.2 Hz, 1H), 4.58-4.56 (m, 1H), 4.25 (d, J=1.6 Hz, 1H), 4.09-4.07 (m, 1H), 3.96-3.94 (m, 1H), 3.87-3.44 (m, 1H), 3.63-3.54 (m, 1H), 2.72-2.69 (m, 2H), 2.24-2.20 (m, 1H), 2.15 (s, 3H), 2.03-1.40 (m, 9H).

### Example 25: Synthetic Scheme for Compound I-54

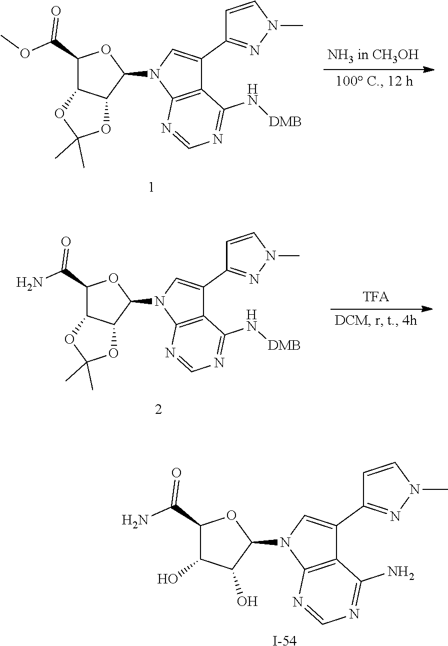

### 25.1 Synthesis of Compound 2

To the solution of methyl (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxylate (100 mg, 177 μmol) and NH₃ in CH₃OH (10 ml). The reaction mixture was stirred at 100° C. for 12 h, until the reaction was complete as indicated by LCMS. Water (40 mL) and EA (100 mL) was added, and the water phase was extracted by EA (100 mL×3). The combined organic phase was washed by sat. NH₄Cl (40 mL×2) and brine (40 mL), and evaporated to dryness. The residue was purified by C18 reversed-phase chromatography to give the desired product (45.0 mg, yield 46.2%). LC-MS m/z=550.3 [M+H]⁺.

### 25.2 Synthesis of Compound I-54

A solution of (3aS,4S,6R,6aR)-6-(4-{[(2,4-dimethoxyphenyl)methyl]amino}-5-(1-methyl-1H-pyrazol-3-yl)-7H-pyrrolo[2,3-d]pyrimidin-7-yl)-2,2-dimethyl-tetrahydro-2H-furo[3,4-d][1,3]dioxole-4-carboxamide (45 mg, 81.8 μmol) in trifluoroacetic acid (1.5 mL) and methylene chloride (5 mL) was stirred at 20° C. for 4 h. Then the mixture was concentrated under reduced pressure, the residue was neutralized by 3 mL 7M NH₃ in MeOH to pH 8. The solvents were evaporated, and the residue was purified by Prep-HPLC to afford the compound (27.4 mg, yield 93.1%) as a white solid. LC-MS m/z=360.0 [M+H]⁺. ¹H NMR (400 MHz, DMSO-d6) δ 9.12-9.00 (br, 1H), 8.11 (s, 1H), 8.03 (s, 1H), 7.94 (s, 1H), 7.75 (d, J=2.2 Hz, 1H), 7.40 (s, 1H), 7.33-7.23 (br, 1H), 6.57 (d, J=2.2 Hz, 1H), 6.03 (d, J=7.5 Hz, 1H), 5.61 (d, J=4.5 Hz, 1H), 5.44 (d, J=6.7 Hz, 1H), 4.59 (dd, J=12.0, 6.9 Hz, 1H), 4.23 (d, J=1.7 Hz, 1H), 4.14 (d, J=4.6 Hz, 1H), 3.89 (s, 3H).

### Example 26: Synthetic Scheme for Compound I-55

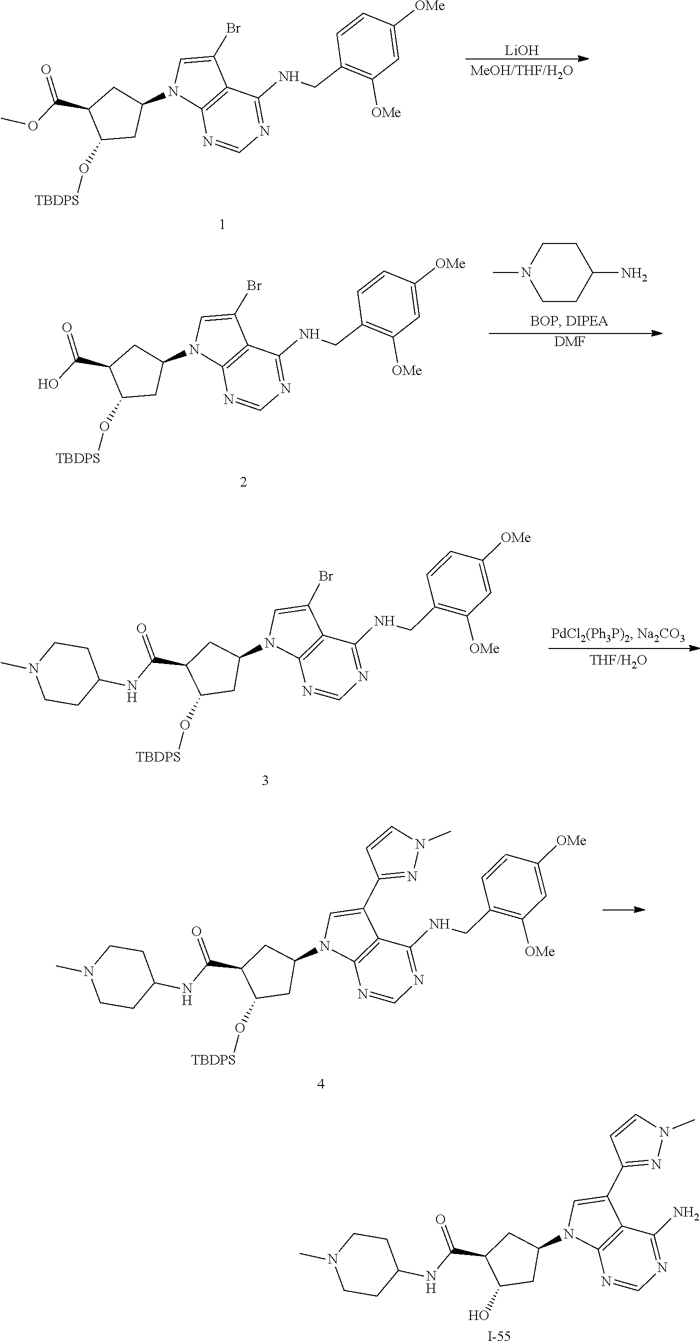

### 26.1 Synthesis of Compound 2

Lithium hydroxide (48.1 mg, 2.01 mmol) was added to methyl (1S,2S,4R)-4-(5-bromo-4-{[(2,4-dimethoxyphenyl)methylamino-7H-pyrrolo[2,3-d]pyrimidin-7-yl)-2-(tert-butyldiphenylsilyl)oxy]cyclopentane-1-carboxylate (500 mg, 672 s mow) in MeOH/THF (10 mL). The mixture was stirred at room temperature overnight. The mixture was concentrated in vacuum and diluted with water (30 mL), extracted with DCM (30 mL) twice. Combined organic layers were washed with brine (40 mL), and dried with anhydrous Na₂SO₄. The solvent was removed under reduced pressure to afford (1S,2S,4R)-4-(5-bromo-4-{[(2,4-dimethoxyphenyl)methyl]amino}-7H-pyrrolo[2,3-d]pyrimidin-7-yl)-2-[(tert-butyldiphenylsilyl)oxy]cyclopentane-1-carboxylic acid (400 mg) as a white solid. LC-MS m/z=728.9 [M+H]⁺.

### 26.2 Synthesis of Compound 3

(1S,2S,4R)-4-(5-Bromo-4-((2,4-dimethoxybenzyl)amino)-7H-pyrrolo[2,3-d]pyrimidin-7-yl)-2-((tert-butyldiphenylsilyl)oxy)cyclopentane-1-carboxylic acid (400 mg, 548 μmol) and 1-methylpiperidin-4-amine (125 mg, 1.1 mmol) were mixed in DMF (8 mL), then BOP (484 mg, 1.1 mmol) and DIPEA (212 mg, 1.1 mmol) were added. The mixture was stirred at 40° C. for 4 hours. The crude was purified by silica gel column chromatography (12 g, DCM/MeOH: 0˜20%) to afford the target (1S,2S,4R)-4-(5-bromo-4-{[(2,4-dimethoxyphenyl)methyl]amino}-7H-pyrrolo[2,3-d]pyrimidin-7-yl)-2-[(tert-butyldiphenylsilyl)oxy]-N-(1-methylpiperidin-4-yl)cyclopentane-1-carboxamide (250 mg, 302 μmol) as a yellow solid. LC-MS m/z=413.1 [M/2+H]⁺.

### 26.3 Synthesis of Compound 4

(1S,2S,4R)-4-(5-Bromo-4-{1[(2,4-dimethoxyphenyl)methyl]amino}-7H-pyrrolo[2,3-d]pyrimidin-7-yl)-2-[(tert-butyldiphenylsilyl)oxy]-N-(1-methylpiperidin-4-yl)cyclopentane-1-carboxamide (250 mg, 302 μmol), 1-methyl-3-(4,4,5,5-tetramethyl-1,3,2-dioxaborolan-2-yl)-1H-pyrazole (125 mg, 604 μmol), Na₂CO₃ (96.0 mg, 906 μmol) and Na₂CO₃ (96.0 mg, 906 μmol) were mixed in THF/H₂O (10 mL), the reaction mixture was stirred at 75° C. for 12 hours under nitrogen atmosphere. The crude was purified by silica gel column chromatography (12 g, DCM/MeOH: 0˜20%) to afford the target (1S,2S,4R)-2-[(tert-butyldiphenylsilyl)oxy]-4-(4-{[(2,4-dimethoxyphenyl)methyl]amino}-5-(1-methyl-1H-pyrazol-3-yl)-7H-pyrrolo[2,3-d]pyrimidin-7-yl)-N-(1-methylpiperidin-4-yl)cyclopentane-1-carboxamide (200 mg) as a yellow solid.

### 26.4 Synthesis of Compound I-55

Into a solution of (1S,2S,4R)-2-[(tert-butyldiphenylsilyl)oxy]-4-(4-{[(2,4-dimethoxyphenyl)methyl]amino}-5-(1-methyl-1H-pyrazol-3-yl)-7H-pyrrolo[2,3-d]pyrimidin-7-yl)-N-(1-methylpiperidin-4-yl)cyclopentane-1-carboxamide (200 mg, 241 μmol) in DCM (5 mL) was added TFA (0.5 mL). The reaction mixture was stirred at 25° C. for 3 h. Then the mixture was concentrated under reduced pressure, the residue was neutralized by 3 mL 7M NH₃ in MeOH to pH 8, and then filtered. The filtrate was purified by Prep-HPLC to provide (1S,2S,4R)-4-(4-amino-5-(1-methyl-1H-pyrazol-3-yl)-7H-pyrrolo[2,3-d]pyrimidin-7-yl)-2-hydroxy-N-(1-methylpiperidin-4-yl)cyclopentane-1-carboxamide (30 mg, 68.41 μmol) as a white solid. (1S,2S,4R)-4-(4-amino-5-(1-methyl-1H-pyrazol-3-yl)-7H-pyrrolo[2,3-d]pyrimidin-7-yl)-2-hydroxy-N-(1-methylpiperidin-4-yl)cyclopentane-1-carboxamide (30 mg). LC-MS m/z=439.0 [M+H]⁺. ¹H NMR (400 MHz, DMSO-d6) δ 8.96 (s, 1H), 8.04 (s, 1H), 7.91-7.80 (m, 2H), 7.72 (d, J=2.3 Hz, 1H), 7.10 (s, 1H), 6.63 (d, J=2.3 Hz, 1H), 5.35-5.19 (m, 1H), 5.09 (d, J=4.7 Hz, 1H), 4.47-4.29 (m, 1H), 3.87 (s, 3H), 3.64-3.45 (m, 1H), 2.67 (ddd, J=13.8, 11.6, 7.3 Hz, 3H), 2.40-2.28 (m, 1H), 2.25-2.07 (m, 4H), 2.06-1.86 (m, 4H), 1.70 (dd, J=19.1, 10.3 Hz, 2H), 1.51-1.30 (m, 2H).

### Example 27: Synthetic Scheme for Compound I-56

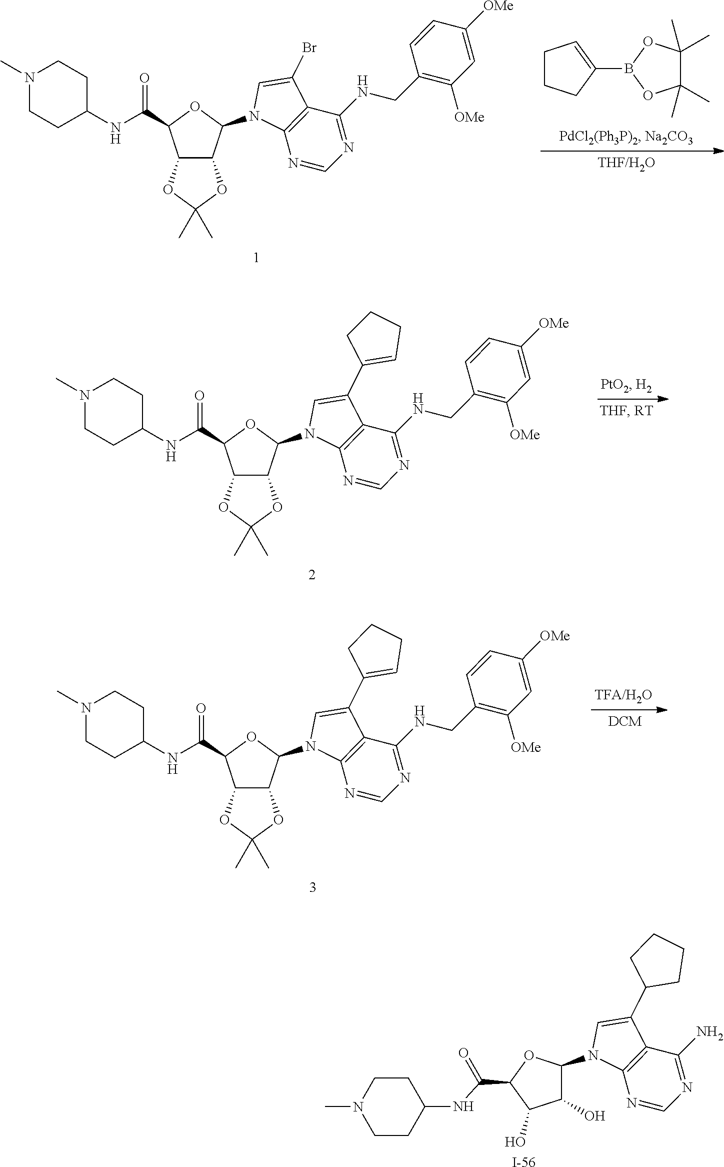

### 27.1 Synthesis of Compound 2

(3aS,4S,6R,6aR)-6-(5-Bromo-4-{1[(2,4-dimethoxyphenyl)methyl]amino}-7H-pyrrolo[2,3-d]pyrimidin-7-yl)-2,2-dimethyl-N-(1-methylpiperidin-4-yl)-tetrahydro-2H-furo[3,4-d][1,3]dioxole-4-carboxamide (100 mg, 154 μmol), 2-(cyclopent-1-en-1-yl)-4,4,5,5-tetramethyl-1,3,2-dioxaborolane (59.7 mg, 308 μmol), Pd(PPh₃)₂Cl₂ (10.8 mg, 15.4 μmol) and Na₂CO₃ (32.6 mg, 308 μmol) were mixed in THF/H₂O (8 mL), the reaction mixture was stirred at 75° C. for 12 hours under nitrogen atmosphere. The mixture was concentrated in vacuum to afford crude product. The residue was purified by silica gel column chromatography (12 g, DCM/MeOH: 0˜20%)) to afford the target compound (3aS,4S,6R,6aR)-6-[5-(cyclopent-1-en-1-yl)-4-{[(2,4-dimethoxyphenyl)methyl]amino}-7H-pyrrolo[2,3-d]pyrimidin-7-yl]-2,2-dimethyl-N-(1-methylpiperidin-4-yl)-tetrahydro-2H-furo[3,4-d][1,3]dioxole-4-carboxamide (40.0 mg) as a yellow oil. LC-MS m/z=633.0 [M+H]⁺.

### 27.2 Synthesis of Compound 3

PtO₂ (2.86 mg, 12.6 μmol) was added to the solution of (3aS,4S,6R,6aR)-6-[5-(cyclopent-1-en-1-yl)-4-{[(2,4-dimethoxyphenyl)methyl]amino}-7H-pyrrolo[2,3-d]pyrimidin-7-yl]-2,2-dimethyl-N-(1-methylpiperidin-4-yl)-tetrahydro-2H-furo[3,4-d][1,3]dioxole-4-carboxamide (40 mg, 63.2 μmol) in THF (4 mL). The mixture was stirred at room temperature under H₂ for 2 hours. The mixture was filtered and filtrate was concentrated in vacuum to afford crude (3aS,4S,6R,6aR)-6-(5-cyclopentyl-4-{[(2,4-dimethoxyphenyl)methyl]amino}-7H-pyrrolo[2,3-d]pyrimidin-7-yl)-2,2-dimethyl-N-(1-methylpiperidin-4-yl)-tetrahydro-2H-furo[3,4-d][1,3]dioxole-4-carboxamide (40.0 mg) as a yellow oil. LC-MS m/z=635.3 [M+H]⁺.

### 27.3 Synthesis of Compound I-56

Into a solution of (3aS,4S,6R,6aR)-6-(5-cyclopentyl-4-{[(2,4-dimethoxyphenyl)methyl]amino}-7H-pyrrolo[2,3-d]pyrimidin-7-yl)-2,2-dimethyl-N-(1-methylpiperidin-4-yl)-tetrahydro-2H-furo[3,4-d][1,3]dioxole-4-carboxamide (40.0 mg, 63.0 μmol) in DCM (3 mL) was added TFA (0.5 mL). The reaction mixture was stirred at 25° C. for 3 h. Then the mixture was concentrated under reduced pressure, the residue was neutralized by 3 mL 7M NH₃ in MeOH to pH=8, and then filtered. The filtrate was purified by Prep-HPLC to afford (2S,3S,4R,5R)-5-(4-amino-5-cyclopentyl-7H-pyrrolo[2,3-d]pyrimidin-7-yl)-3,4-dihydroxy-N-(1-methylpiperidin-4-yl)tetrahydrofuran-2-carboxamide (10 mg, 22.5 μmol) as a white solid. LC-MS m/z=445.2 [M+H]⁺. ¹H NMR (400 MHz, DMSO-d6) δ 8.33 (d, J=8.1 Hz, 1H), 8.05 (s, 1H), 7.22 (s, 1H), 6.63 (s, 2H), 5.97 (d, J=7.6 Hz, 1H), 5.58 (d, J=4.3 Hz, 1H), 5.34 (d, J=6.7 Hz, 1H), 4.57 (dd, J=11.9, 7.1 Hz, 1H), 4.23 (d, J=1.4 Hz, 1H), 4.08 (t, J=3.7 Hz, 1H), 3.59 (d, J=7.7 Hz, 1H), 2.72 (s, 2H), 2.15 (s, 3H), 2.08-1.15 (m, 15H).

### Example 28: Synthetic Scheme for Compound I-57

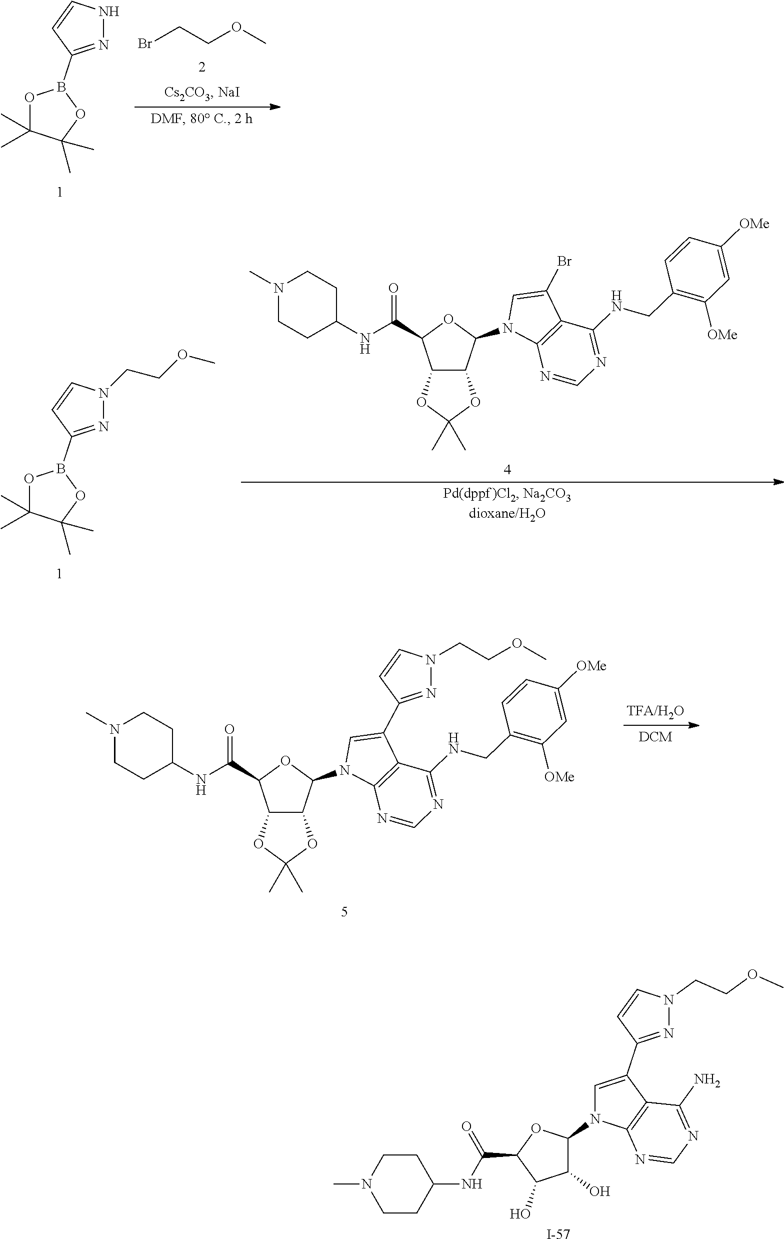

### 28.1 Synthesis of Compound 3

1-Bromo-2-methoxyethane (1.07 g, 7.72 mmol) was added to the solution of 3-(4,4,5,5-tetramethyl-1,3,2-dioxaborolan-2-yl)-1H-pyrazole (1 g, 5.15 mmol) in DMF (10 mL), then caesium carbonate (3.35 g, 10.3 mmol), NaI (771 mg, 5.15 mmol) were added and the mixture was stirred at 80° C. for 2 hours. Crude was purified by silica gel column chromatography (20 g, CH₃CN/MeOH: 0˜10%) to afford 200 mg of product.

### 28.2 Synthesis of Compound 5

1-(2-Methoxyethyl)-3-(4,4,5,5-tetramethyl-1,3,2-dioxaborolan-2-yl)-1H-pyrazole (200 mg, 793 μmol), (3aS,4S,6R,6aR)-6-(5-bromo-4-((2,4-dimethoxybenzyl)amino)-7H-pyrrolo[2,3-d]pyrimidin-7-yl)-2,2-dimethyl-N-(1-methylpiperidin-4-yl)tetrahydrofuro[3,4-d][1,3]dioxole-4-carboxamide (563.3 mg, 872 μmol), Pd(dppf)Cl₂ (58 mg, 79.3 μmol) and Na₂CO₃ (250 mg, 2.37 mmol) were mixed in Dioxane/H₂O (10 mL), the reaction mixture was stirred at 75° C. for 12 hours under nitrogen atmosphere. The mixture was concentrated in vacuum to provide crude product. The residue was purified by silica gel column chromatography (12 g, DCM/MeOH: 0˜20%) to afford the target compound (3aS,4S,6R,6aR)-6-(4-((2,4-dimethoxybenzyl)amino)-5-(1-(2-methoxyethyl)-1H-pyrazol-3-yl)-7H-pyrrolo[2,3-d]pyrimidin-7-yl)-2,2-dimethyl-N-(1-methylpiperidin-4-yl)tetrahydrofuro[3,4-d][1,3]dioxole-4-carboxamide (100 mg, 144.7 μmol) as a yellow oil. LC-MS m/z=691.0 [M+H]⁺.

### 28.3 Synthesis of Compound I-57

Into a solution of (3aS,4S,6R,6aR)-6-(4-((2,4-dimethoxybenzyl)amino)-5-(1-(2-methoxyethyl)-1H-pyrazol-3-yl)-7H-pyrrolo[2,3-d]pyrimidin-7-yl)-2,2-dimethyl-N-(1-methylpiperidin-4-yl)tetrahydrofuro[3,4-d][1,3]dioxole-4-carboxamide (100 mg, 144.7 μmol) in DCM (6 mL) was added TFA (0.5 mL). The reaction mixture was stirred at 25° C. for 3 h. Then the mixture was concentrated under reduced pressure, the residue was neutralized by 3 mL 7M NH₃ in MeOH to pH=8, and then filtered. The filtrate was purified by Prep-HPLC to afford (2S,3S,4R,5R)-5-(4-amino-5-(1-(2-methoxyethyl)-1H-pyrazol-3-yl)-7H-pyrrolo[2,3-d]pyrimidin-7-yl)-3,4-dihydroxy-N-(1-methylpiperidin-4-yl)tetrahydrofuran-2-carboxamide (18 mg) as a white solid. LC-MS m/z=501.2 [M+H]⁺. ¹H NMR (400 MHz, DMSO-d6) δ 9.07 (s, 1H), 8.34 (d, J=8.1 Hz, 1H), 8.09 (s, 1H), 7.91 (s, 1H), 7.78 (d, J=2.2 Hz, 1H), 7.29 (s, 1H), 6.55 (d, J=2.2 Hz, 1H), 6.04 (d, J=7.5 Hz, 1H), 5.64 (d, J=4.4 Hz, 1H), 5.45 (d, J=6.6 Hz, 1H), 4.63 (dd, J=11.7, 7.0 Hz, 1H), 4.37-4.23 (m, 3H), 4.14 (t, J=3.6 Hz, 1H), 3.71 (t, J=5.2 Hz, 2H), 3.67-3.53 (m, 1H), 3.25 (s, 3H), 2.70 (s, 2H), 2.14 (s, 3H), 1.93 (dd, J=14.5, 11.6 Hz, 2H), 1.73 (dd, J=41.0, 11.7 Hz, 2H), 1.56-1.36 (m, 2H).

### Example 29: Synthetic Scheme for Compound I-58

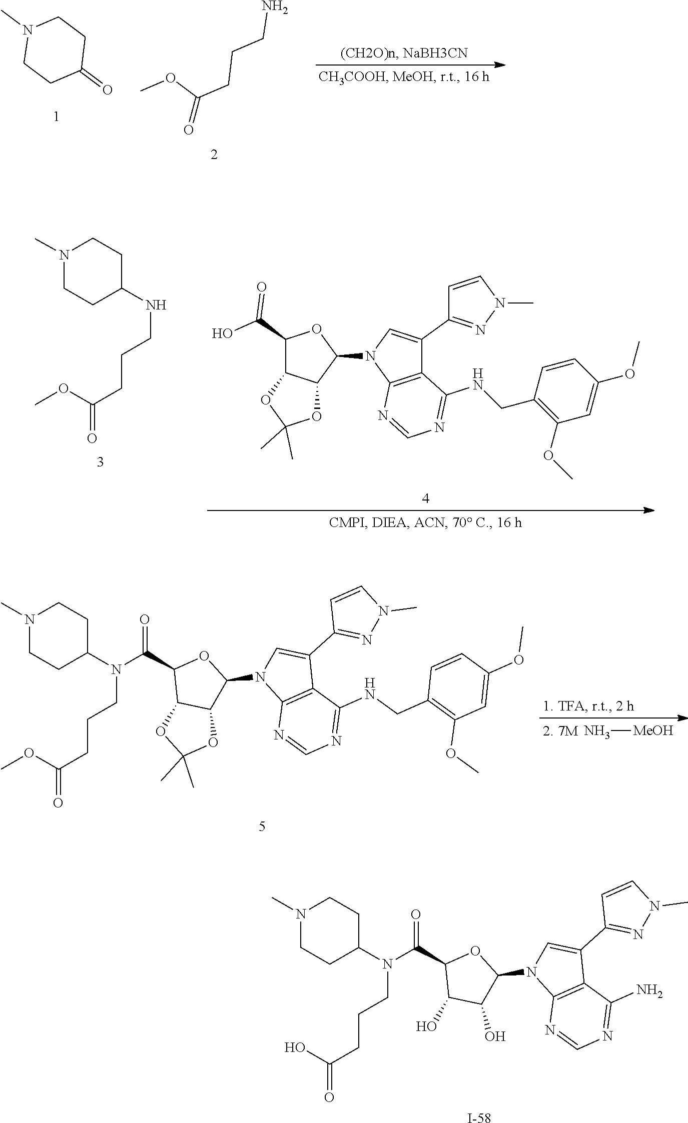

### 29.1 Synthesis of Compound 3

Methyl 4-aminobutanoate (500 mg, 4.26 mmol) was dissolved in the methanol (10 ml). 1-methylpiperidin-4-one (750 mg, 6.62 mmol), acetic acid (0.3 ml, 0.004995 mmol), and sodium cyanoborohydride (400 mg, 6.36 mmol) were added. The reaction mixture was stirred at room temperature overnight. The crude mixture was purified by silica gel chromatography (DCM:MeOH=10:1) to afford methyl 4-[(1-methylpiperidin-4-yl)amino]butanoate (350 mg).

### 29.2 Synthesis of Compound 5

Methyl 4-[(1-methylpiperidin-4-yl)amino]butanoate (350 mg, 1.63 mmol) was dissolved in MeCN (20 mL). (3aS,4S,6R,6aR)-6-(4-((2,4-dimethoxybenzyl)amino)-5-(1-methyl-1H-pyrazol-3-yl)-7H-pyrrolo[2,3-d]pyrimidin-7-yl)-2,2-dimethyltetrahydrofuro[3,4-d][1,3]dioxole-4-carboxylic acid (900 mg, 1.65 mmol), CMPI (42 mg, 0.163 mmol) and DIEA (0.5 mL) were added. The mixture was stirred at 70° C. overnight. The mixture was washed with water and extracted three times with EA. The solvent was removed under reduced pressure to give methyl 4-((3aS,4S,6R,6aR)-6-(4-((2,4-dimethoxybenzyl)amino)-5-(1-methyl-1H-pyrazol-3-yl)-7H-pyrrolo[2,3-d]pyrimidin-7-yl)-2,2-dimethyl-N-(1-methylpiperidin-4-yl)tetrahydrofuro[3,4-d][1,3]dioxole-4-carboxamido)butanoate (31.7 mg). LC-MS m/z=747.3 [M+H]⁺.

### 29.3 Synthesis of Compound I-58

Methyl 4-((3aS,4S,6R,6aR)-6-(4-((2,4-dimethoxybenzyl)amino)-5-(1-methyl-1H-pyrazol-3-yl)-7H-pyrrolo[2,3-d]pyrimidin-7-yl)-2,2-dimethyl-N-(1-methylpiperidin-4-yl)tetrahydrofuro[3,4-d][1,3]dioxole-4-carboxamido)butanoate (31.7 mg, 0.042 mmol) was dissolved in TFA (5 mL). The mixture was stirred at room temperature for 2 hours. The solvent was removed under reduced pressure. Neutralized with saturated solution 4 mL 7M NH₃·MeOH, all the target transformed to P10-1106. The crude was purified by Prep-HPLC to afford 4-{1-[(2S,3S,4R,5R)-5-[4-amino-5-(1-methyl-1H-pyrazol-3-yl)-7H-pyrrolo[2,3-d]pyrimidin-7-yl]-3,4-dihydroxyoxolan-2-yl]-N-(1-methylpiperidin-4-yl)formamido}butanoic acid (6.80 mg, yield 29.5%), as a white solid. LC-MS m/z=543.1 [M+H]⁺. ¹H NMR (400 MHz, DMSO-d6) δ 8.94 (s, 1H), 8.11 (s, 1H), 8.06 (s, 1H), 7.77-7.74 (m, J=8.6, 2.2 Hz, 1H), 7.18 (s, 1H), 6.54-6.50 (m, J=11.6, 2.3 Hz, 1H), 6.32-6.25 (m, J=23.7, 6.6 Hz, 1H), 5.03-4.75 (m, J=113.1, 1.9 Hz, 1H), 4.45-4.23 (m, 1H), 4.19-4.12 (m, 1H), 4.10-4.04 (m, J=11.9 Hz, 1H), 3.90-3.88 (m, J=5.4 Hz, 1H), 3.26-3.18 (m, 1H), 2.79 (s, 1H), 2.14 (s, 1H), 1.93-1.88 (m, J=12.6, 7.5 Hz, 2H), 1.82-1.62 (m, J=51.6, 15.9, 9.7 Hz, 2H), 1.55-1.38 (m, 1H).

### Example 30: Synthetic Scheme for Compound I-59

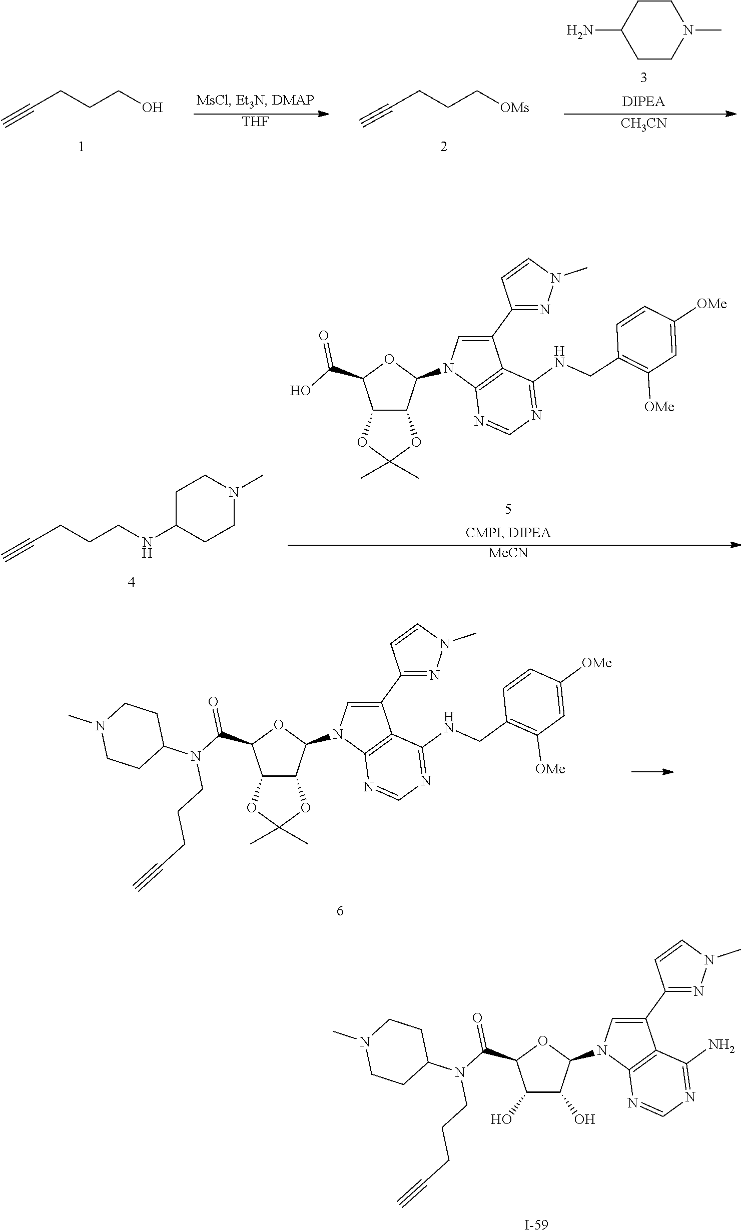

### 30.1 Synthesis of Compound 2

Triethylamine (1.80 g, 17.8 mmol) and 4-dimethylaminopyridine (36.2 mg, 297 μmol) were added to the solution of pent-4-yn-1-ol (500 mg, 5.94 mmol) in THE (15 mL), then methanesulfonyl chloride (1.55 g, 13.6 mmol) was added to solution at 0° C. The mixture was warmed up and stirred at room temperature for 12 hours. The mixture was filtered and filtrate was extracted with EA (20 mL×2). Combined organic layers were washed with brine (30 mL), and dried with anhydrous Na₂SO₄. The solvent was removed under reduced pressure to afford crude pent-4-yn-1-yl methanesulfonate (600 mg, 3.69 mmol) as yellow oil.

### 30.2 Synthesis of Compound 4

N,N-Diisopropylethylamine (317 mg, 2.46 mmol) was added to the mixture of pent-4-yn-1-yl methanesulfonate (200 mg, 1.23 mmol) and 1-methylpiperidin-4-amine (350 mg, 3.07 mmol) in MeCN (4 mL). The mixture was stirred at 80° C. for 12 hours. The crude was purified by silica gel column chromatography (12 g, DCM/NH₃ in MeOH (3M): 0˜15%) to afford 1-methyl-N-(pent-4-yn-1-yl)piperidin-4-amine (40.0 mg) as a gray oil.

### 30.3 Synthesis of Compound 6

(3aS,4S,6R,6aR)-6-(4-((2,4-Dimethoxybenzyl)amino)-5-(1-methyl-1H-pyrazol-3-yl)-7H-pyrrolo[2,3-d]pyrimidin-7-yl)-2,2-dimethyltetrahydrofuro[3,4-d][1,3]dioxole-4-carboxylic acid (122.2 mg, 221 μmol) and 1-methyl-N-(pent-4-yn-1-yl)piperidin-4-amine (40.0 mg, 221 μmol) were mixed in MeCN (3 mL), then CMPI (113 mg, 442 μmol) and DIPEA (58 mg, 442 μmol) were added. The mixture was stirred at 40° C. for 4 hours. The crude was purified by silica gel column chromatography (12 g, DCM/MeOH: 0˜20%) to afford the target (3aS,4S,6R,6aR)-6-(4-((2,4-dimethoxybenzyl)amino)-5-(1-methyl-1H-pyrazol-3-yl)-7H-pyrrolo[2,3-d]pyrimidin-7-yl)-2,2-dimethyl-N-(1-methylpiperidin-4-yl)-N-(pent-4-yn-1-yl)tetrahydrofuro[3,4-d][1,3]dioxole-4-carboxamide (80 mg, 112 μmol) as a yellow oil. LC-MS m/z=357.2 [M/2+H]⁺.

### 30.4 Synthesis of Compound I-59

Into a solution of (3aS,4S,6R,6aR)-6-(4-((2,4-dimethoxybenzyl)amino)-5-(1-methyl-1H-pyrazol-3-yl)-7H-pyrrolo[2,3-d]pyrimidin-7-yl)-2,2-dimethyl-N-(1-methylpiperidin-4-yl)-N-(pent-4-yn-1-yl)tetrahydrofuro[3,4-d][1,3]dioxole-4-carboxamide (80 mg, 112 μmol) in DCM (3 mL) was added TFA (0.2 mL). The reaction mixture was stirred at 25° C. for 3 h. Then the mixture was concentrated under reduced pressure, the residue was neutralized by 1 mL 7M NH₃ in MeOH to pH 8, and then filtered. The filtrate was purified by Prep-HPLC to afford (2S,3S,4R,5R)-5-(4-amino-5-(1-methyl-1H-pyrazol-3-yl)-7H-pyrrolo[2,3-d]pyrimidin-7-yl)-3,4-dihydroxy-N-(1-methylpiperidin-4-yl)-N-(pent-4-yn-1-yl)tetrahydrofuran-2-carboxamide (4 mg) as a white solid. LC-MS m/z=523.2 [M+H]⁺. ¹H NMR (400 MHz, DMSO-d6) δ 8.95 (s, 1H), 8.06 (s, 1H), 7.79 (dd, J=30.9, 27.9 Hz, 2H), 7.21 (s, 1H), 6.55-6.44 (m, 1H), 6.27 (dd, J=9.4, 6.3 Hz, 1H), 5.52 (td, J=9.9, 5.9 Hz, 2H), 4.72 (dd, J=34.5, 2.5 Hz, 1H), 4.52-4.22 (m, 2H), 3.82 (t, J=42.6 Hz, 4H), 3.23 (s, 2H), 2.80 (ddd, J=50.2, 22.7, 6.4 Hz, 3H), 2.18 (dd, J=24.4, 8.3 Hz, 5H), 1.91 (dd, J=42.2, 11.0 Hz, 2H), 1.77-1.35 (m, 6H).

Compounds in Table 2 were prepared according to methods shown above using experimental procedures similar to those described in Examples 6-30. MS and ¹H NMR data are shown below.

TABLE 2

| | Com- | | MS | |
|---|---|---|---|---|
| | pound | Synthetic | (ESI) | |
| Structure | No. | Method | [M + H]⁺ | ¹H NMR |
| 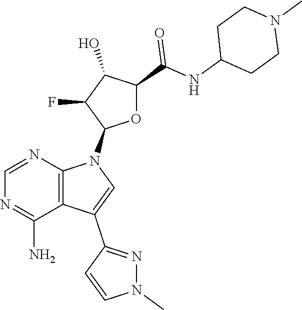 [SMILES: CN1CCC(NC(=O)[C@H]2O[C@@H](n3cc(-c4ccn(C)n4)c4c(N)ncnc43)[C@@H](F)[C@@H]2O)CC1] | I-60 | 6 | 459 | ¹H NMR (400 MHz, DMSO- d6) δ ppm 9.02 (brs, 1H), 8.23 (d, J = 8.0 Hz, 1H), 8.09 (s, 1H), 7.78 (d, J = 2.4 Hz, 1H), 7.77 (d, J = 2.4 Hz, 1H), 7.29 (brs, 1H), 6.65 (dd, J = 22.0, 3.2 Hz, 1H), 6.50 (d, J = 2.4 Hz, 1H), 6.27 (d, J = 4.8 Hz, 1H), 5.09 (dt, J = 51.2, 2.8 Hz, 1H), 4.64-4.58 (m, 1H), 4.30 (d, J = 2.4 Hz, 1H), 3.90 (s, 3H), 3.67-3.59 (m, 1H), 2.72- 2.67 (m, 2H), 2.14 (s, 3H), 1.96-1.90 (m, 2H), 1.70-1.67 (m, 2H), 1.62-1.49 (m, 2H). |
| 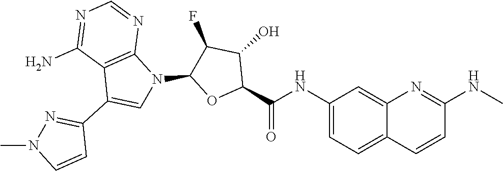 | I-61 | 6 | 518 | ¹H NMR (400 MHz, DMSO- d6) δ ppm 10.45 (s, 1H), 9.05 (brs, 1H), 8.12 (s, 1H), 7.99 (d, J = 2.0 Hz, 1H), 7.88 (d, J = 2.0 Hz, 1H), 7.76 (d, J = 8.8 Hz, 1H), 7.74 (d, J = 2.4 Hz, 1H), 7.57 (d, J = 8.4 Hz, 1H), 7.37 (dd, J = 8.8, 2.0 Hz, 1H), 7.32 (brs, 1H), 7.01-6.97 (m, 1H), 6.75 (dd, J = 21.2, 3.6 Hz, 1H), 6.66 (d, J = 8.8 Hz, |
| | | | | 1H), 6.50 (d, J = 2.4 Hz, 1H), |
| | | | | 6.42 (d, J = 4.4 Hz, 1H), 5.20 |
| | | | | (dt, J = 52.0, 2.8 Hz, 1H), 4.80- |
| | | | | 4.76 (m, 1H), 4.57 (d, J = 2.8 |
| | | | | Hz, 1H), 3.90 (s, 3H), 2.90 (d, |
| | | | | J = 2.8 Hz, 3H). |
| 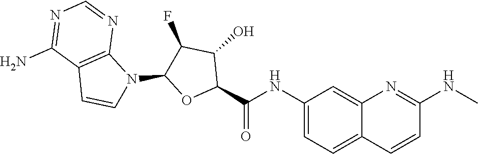 [SMILES: CNc1ccc2ccc(NC(=O)[C@H]3O[C@@H](n4ccc5c(N)ncnc54)[C@@H](F)[C@@H]3O)cc2n1] | I-62 | 6 | 438 | ¹H NMR (400 MHz, DMSO- d6) δ ppm 10.49 (s, 1H), 8.11 (s, 1H), 7.97 (d, J = 1.6 Hz, 1H), 7.76 (d, J = 8.8 Hz, 1H), 7.57 (d, J = 8.4 Hz, 1H), 7.48- 7.46 (m, 1H), 7.34 (dd, J = 8.4, 2.0 Hz, 1H), 7.13 (brs, 2H), 6.98-6.97 (m, 1H), 6.74-6.64 (m, 3H), 6.39 (d, J = 4.8 Hz, 1H), 5.14 (dt, J = 51.2, 2.8 Hz, |
| | | | | 1H), 4.76-4.70 (m, 1H), 4.53 |
| | | | | (d, J = 2.8 Hz, 1H), 2.89 (d, J = |
| | | | | 4.8 Hz, 3H). |
| 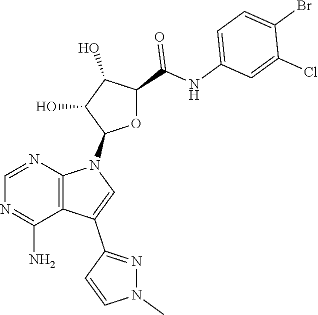 [SMILES: Cn1ccc(-c2cn([C@@H]3O[C@H](C(=O)Nc4ccc(Br)c(Cl)c4)[C@@H](O)[C@H]3O)c3ncnc(N)c23)n1] | I-63 | 8 | 548 | |
| 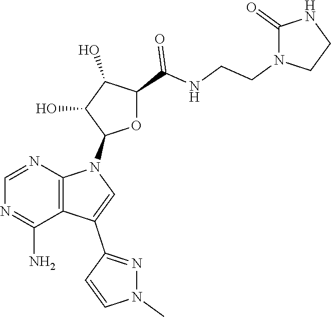 [SMILES: Cn1ccc(-c2cn([C@@H]3O[C@H](C(=O)NCCN4CCNC4=O)[C@@H](O)[C@H]3O)c3ncnc(N)c23)n1] | I-64 | 8 | 472 | |
| 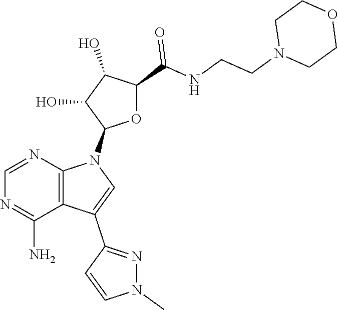 [SMILES: Cn1ccc(-c2cn([C@@H]3O[C@H](C(=O)NCCN4CCOCC4)[C@@H](O)[C@H]3O)c3ncnc(N)c23)n1] | I-65 | 8 | 473 | |
| 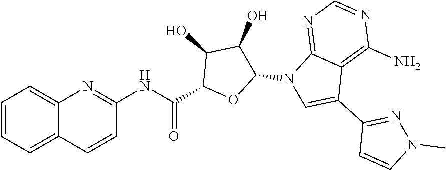 [SMILES: Cn1ccc(-c2cn([C@@H]3O[C@H](C(=O)Nc4ccc5ccccc5n4)[C@@H](O)[C@H]3O)c3ncnc(N)c23)n1] | I-66 | 8 | 487 | ¹H NMR (400 MHz, DMSO- d6) δ 11.82 (s, 1H), 9.16 (s, 1H), 8.57 (s, 1H), 8.41 (dd, J = 19.2, 9.0 Hz, 2H), 8.00-7.90 (m, 2H), 7.88-7.72 (m, 3H), 7.60-7.49 (m, 1H), 7.37 (s, 1H), 6.61 (d, J = 2.3 Hz, 1H), 5.99 (d, J = 7.7 Hz, 1H), 5.86 (s, 1H), 5.59 (s, 1H), 4.81 (s, 1H), 4.61 (d, J = 1.6 Hz, 1H), 4.36 (s, 1H), 3.91 (s, 3H). |
| 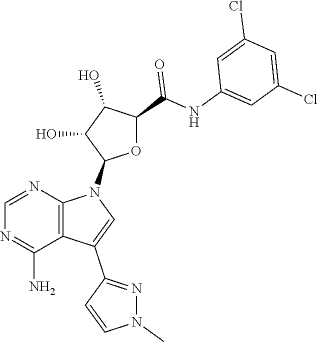 [SMILES: Cn1ccc(-c2cn([C@@H]3O[C@H](C(=O)Nc4cc(Cl)cc(Cl)c4)[C@@H](O)[C@H]3O)c3ncnc(N)c23)n1] | I-67 | 8 | 504 | |
| 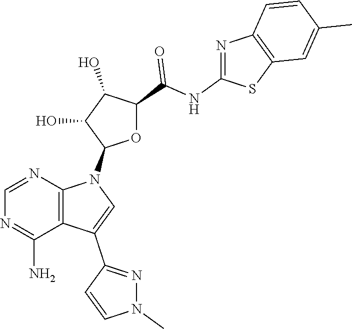 [SMILES: Cc1ccc2nc(NC(=O)[C@H]3O[C@@H](n4cc(-c5ccn(C)n5)c5c(N)ncnc54)[C@H](O)[C@@H]3O)sc2c1] | I-68 | 8 | 507 | |
| 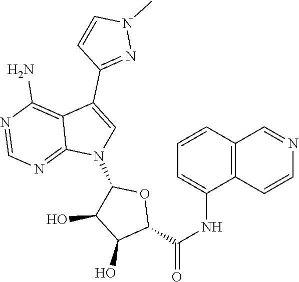 [SMILES: Cn1ccc(-c2cn([C@@H]3O[C@H](C(=O)Nc4cccc5cnccc45)[C@@H](O)[C@H]3O)c3ncnc(N)c23)n1] | I-69 | 8 | 487 | |
| 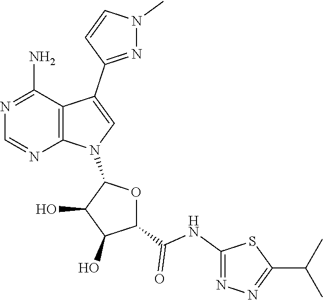 [SMILES: CC(C)c1nnc(NC(=O)[C@H]2O[C@@H](n3cc(-c4ccn(C)n4)c4c(N)ncnc43)[C@H](O)[C@@H]2O)s1] | I-70 | 8 | 486 | |
| 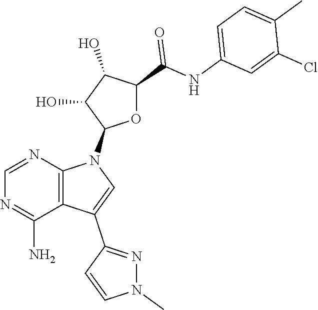 [SMILES: Cc1ccc(NC(=O)[C@H]2O[C@@H](n3cc(-c4ccn(C)n4)c4c(N)ncnc43)[C@H](O)[C@@H]2O)cc1Cl] | I-71 | 8 | 484 | |
| 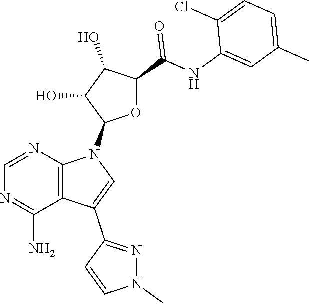 [SMILES: Cc1ccc(Cl)c(NC(=O)[C@H]2O[C@@H](n3cc(-c4ccn(C)n4)c4c(N)ncnc43)[C@H](O)[C@@H]2O)c1] | I-72 | 8 | 484 | |
| 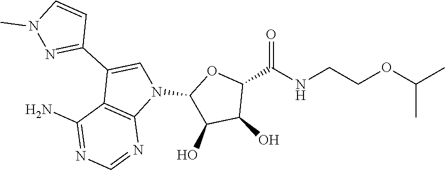 [SMILES: CC(C)OCCNC(=O)[C@H]1O[C@@H](n2cc(-c3ccn(C)n3)c3c(N)ncnc32)[C@H](O)[C@@H]1O] | I-73 | 8 | 446 | |
| 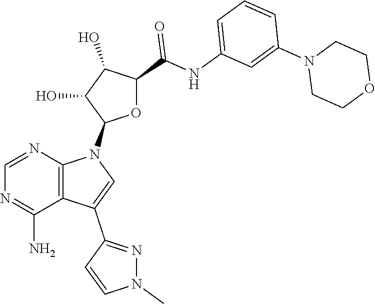 [SMILES: Cn1ccc(-c2cn([C@@H]3O[C@H](C(=O)Nc4cccc(N5CCOCC5)c4)[C@@H](O)[C@H]3O)c3ncnc(N)c23)n1] | I-74 | 8 | 521 | |
| 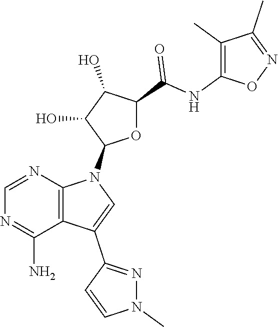 [SMILES: Cc1noc(NC(=O)[C@H]2O[C@@H](n3cc(-c4ccn(C)n4)c4c(N)ncnc43)[C@H](O)[C@@H]2O)c1C] | I-75 | 8 | 455 | |
| 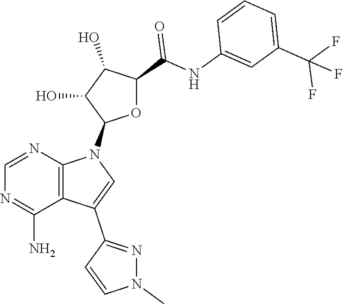 [SMILES: Cn1ccc(-c2cn([C@@H]3O[C@H](C(=O)Nc4cccc(C(F)(F)F)c4)[C@@H](O)[C@H]3O)c3ncnc(N)c23)n1] | I-76 | 8 | 504 | ¹H NMR (400 MHz, DMSO- d6) δ 10.63 (s, 1H), 9.07 (s, 1H), 8.10 (s, 1H), 8.04 (s, 1H), 7.96 (s, 1H), 7.85 (d, J = 8.0 Hz, 1H), 7.75 (d, J = 2.2 Hz, 1H), 7.59 (t, J = 8.0 Hz, 1H), 7.48 (d, J = 7.8 Hz, 1H), 7.29 (s, 1H), 6.55 (d, J = 2.3 Hz, 1H), 6.17 (d, J = 7.1 Hz, 1H), 5.75 (d, J = 4.7 Hz, 1H), 5.56 (d, J = 6.5 Hz, 1H), 4.67 (dd, J = 11.5, 6.8 Hz, 1H), 4.51 (d, J = 2.2 Hz, 1H), 4.43-4.27 (m, 1H), 3.89 (s, 3H). |
| 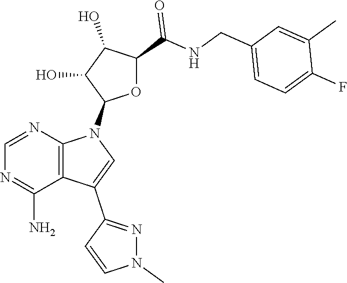 [SMILES: Cc1cc(CNC(=O)[C@H]2O[C@@H](n3cc(-c4ccn(C)n4)c4c(N)ncnc43)[C@H](O)[C@@H]2O)ccc1F] | I-77 | 8 | 482 | |
| 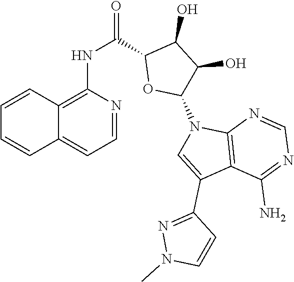 [SMILES: Cn1ccc(-c2cn([C@@H]3O[C@H](C(=O)Nc4nccc5ccccc45)[C@@H](O)[C@H]3O)c3ncnc(N)c23)n1] | I-78 | 8 | 487 | |
| 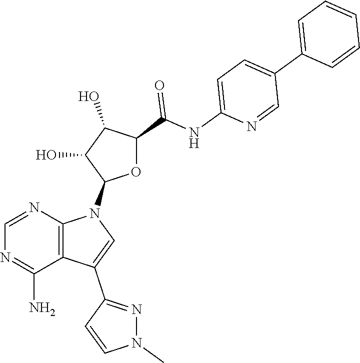 [SMILES: Cn1ccc(-c2cn([C@@H]3O[C@H](C(=O)Nc4ccc(-c5ccccc5)cn4)[C@@H](O)[C@H]3O)c3ncnc(N)c23)n1] | I-79 | 8 | 513 | |
| 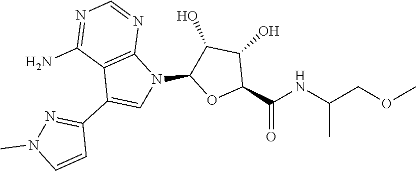 | I-80 | 8 | 432 | |
| 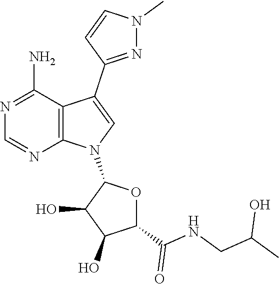 [SMILES: CC(O)CNC(=O)[C@H]1O[C@@H](n2cc(-c3ccn(C)n3)c3c(N)ncnc32)[C@H](O)[C@@H]1O] | I-81 | 8 | 418 | |
| 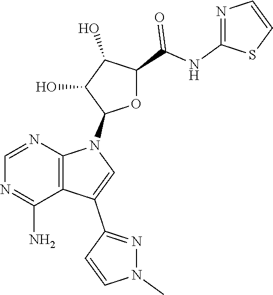 [SMILES: Cn1ccc(-c2cn([C@@H]3O[C@H](C(=O)Nc4nccs4)[C@@H](O)[C@H]3O)c3ncnc(N)c23)n1] | I-82 | 8 | 443 | |
| 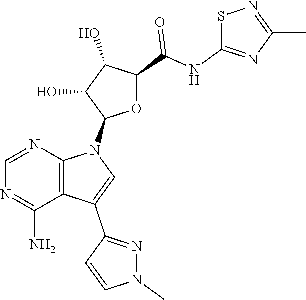 [SMILES: Cc1nsc(NC(=O)[C@H]2O[C@@H](n3cc(-c4ccn(C)n4)c4c(N)ncnc43)[C@H](O)[C@@H]2O)n1] | I-83 | 8 | 458 | |
| 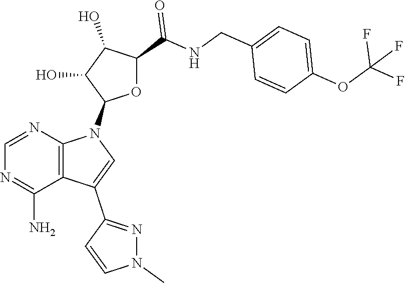 [SMILES: Cn1ccc(-c2cn([C@@H]3O[C@H](C(=O)NCc4ccc(OC(F)(F)F)cc4)[C@@H](O)[C@H]3O)c3ncnc(N)c23)n1] | I-84 | 8 | 534 | |
| 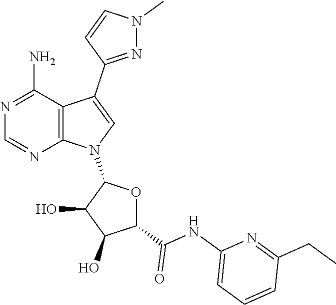 [SMILES: CCc1cccc(NC(=O)[C@H]2O[C@@H](n3cc(-c4ccn(C)n4)c4c(N)ncnc43)[C@H](O)[C@@H]2O)n1] | I-85 | 8 | 465 | |
| 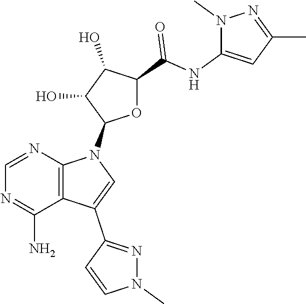 [SMILES: Cc1cc(NC(=O)[C@H]2O[C@@H](n3cc(-c4ccn(C)n4)c4c(N)ncnc43)[C@H](O)[C@@H]2O)n(C)n1] | I-86 | 8 | 454 | ¹H NMR (400 MHz, DMSO- d6) δ ppm 10.71 (s, 1H), 7.96 (d, J = 1.6 Hz, 2H), 7.76 (d, J = 2.0 Hz, 1H), 6.58 (d, J = 2.0 Hz, 1H), 6.13 (d, J = 7.2 Hz, 1H), 6.03 (s, 1H), 5.80 (d, J = 4.8 Hz, 1H), 5.58 (d, J = 6.8 Hz, 1H), 4.66-4.65 (m, 1H), 4.53 (d, J = 2.0 Hz, 1H), 4.33- 4.31 (m, 1H), 3.89 (s, 3H), 3.53 (s, 3H), 2.12 (s, 3H). |
| 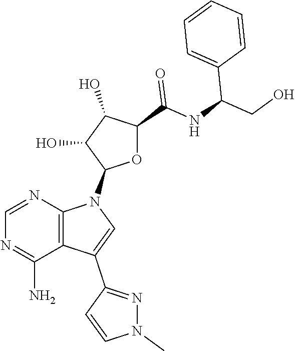 [SMILES: Cn1ccc(-c2cn([C@@H]3O[C@H](C(=O)N[C@H](CO)c4ccccc4)[C@@H](O)[C@H]3O)c3ncnc(N)c23)n1] | I-87 | 8 | 480 | ¹H NMR (400 MHz, DMSO- d6) δ 9.06 (s, 1H), 8.94 (d, J = 8.3 Hz, 1H), 7.88 (d, J = 2.7 Hz, 2H), 7.75 (d, J = 2.3 Hz, 1H), 7.33 (ddd, J = 32.8, 23.8, 7.4 Hz, 6H), 6.51 (d, J = 2.3 Hz, 1H), 6.00 (d, J = 7.6 Hz, 1H), 5.67 (s, 2H), 4.96 (d, J = 5.0 Hz, 2H), 4.72 (dd, J = 7.6, 4.9 Hz, 1H), 4.39 (d, J = 1.5 Hz, 1H), 4.11 (d, J = 3.3 Hz, 1H), 3.89 (s, 3H), 3.68-3.52 (m, 2H). |
| 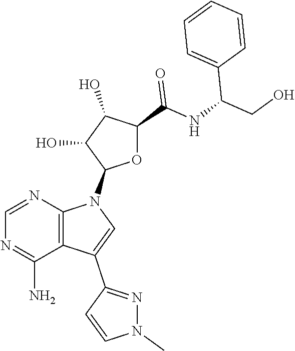 [SMILES: Cn1ccc(-c2cn([C@@H]3O[C@H](C(=O)N[C@@H](CO)c4ccccc4)[C@@H](O)[C@H]3O)c3ncnc(N)c23)n1] | I-88 | 8 | 480 | ¹H NMR (400 MHz, DMSO- d6) δ 9.07 (s, 1H), 8.74 (d, J = 8.4 Hz, 1H), 7.99 (d, J = 14.1 Hz, 2H), 7.75 (d, J = 2.2 Hz, 1H), 7.36-7.13 (m, 6H), 6.61 (d, J = 2.3 Hz, 1H), 6.15 (d, J = 7.7 Hz, 1H), 5.66 (d, J = 3.9 Hz, 1H), 5.47 (d, J = 6.4 Hz, 1H), 5.10-4.92 (m, 2H), 4.57 (s, 1H), 4.38 (d, J = 1.4 Hz, 1H), 4.16 (s, 1H), 3.89 (s, 3H), 3.65 (dd, J = 14.8, 7.5 Hz, 2H). |
| 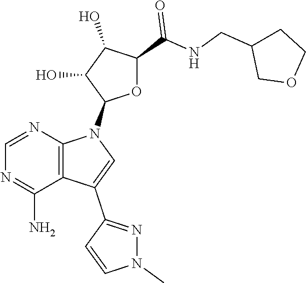 [SMILES: Cn1ccc(-c2cn([C@@H]3O[C@H](C(=O)NCC4CCOC4)[C@@H](O)[C@H]3O)c3ncnc(N)c23)n1] | I-89 | 8 | 444 | |
| 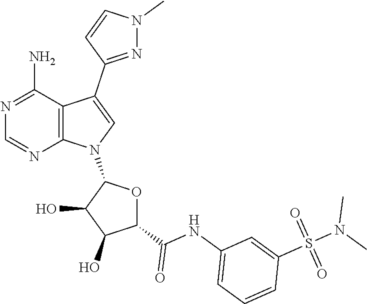 [SMILES: CN(C)S(=O)(=O)c1cccc(NC(=O)[C@H]2O[C@@H](n3cc(-c4ccn(C)n4)c4c(N)ncnc43)[C@H](O)[C@@H]2O)c1] | I-90 | 8 | 543 | |
| 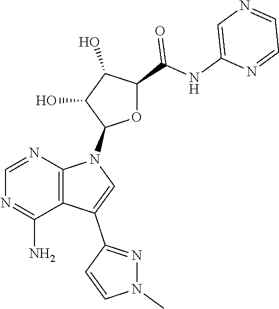 [SMILES: Cn1ccc(-c2cn([C@@H]3O[C@H](C(=O)Nc4cnccn4)[C@@H](O)[C@H]3O)c3ncnc(N)c23)n1] | I-91 | 8 | 438 | |
| 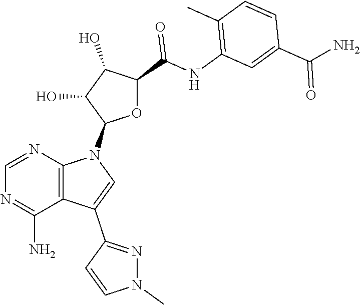 [SMILES: Cc1ccc(C(N)=O)cc1NC(=O)[C@H]1O[C@@H](n2cc(-c3ccn(C)n3)c3c(N)ncnc32)[C@H](O)[C@@H]1O] | I-92 | 8 | 493 | |
| 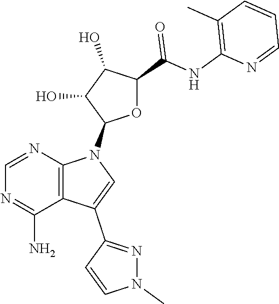 [SMILES: Cc1cccnc1NC(=O)[C@H]1O[C@@H](n2cc(-c3ccn(C)n3)c3c(N)ncnc32)[C@H](O)[C@@H]1O] | I-93 | 8 | 451 | |
| 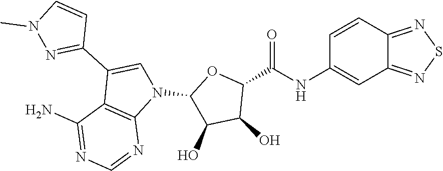 [SMILES: Cn1ccc(-c2cn([C@@H]3O[C@H](C(=O)Nc4ccc5nsnc5c4)[C@@H](O)[C@H]3O)c3ncnc(N)c23)n1] | I-94 | 8 | 494 | |
| 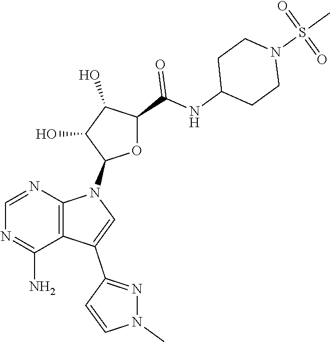 [SMILES: Cn1ccc(-c2cn([C@@H]3O[C@H](C(=O)NC4CCN(S(C)(=O)=O)CC4)[C@@H](O)[C@H]3O)c3ncnc(N)c23)n1] | I-95 | 8 | 521 | ¹H NMR (400 MHz, DMSO- d6) δ ppm 9.08 (brs, 1H), 8.39 (d, J = 8.0 Hz, 1H), 8.09 (s, 1H), 7.94 (s, 1H), 7.75 (d, J = 2.4 Hz, 1H), 7.27 (brs, 1H), 6.57 (d, J = 2.0 Hz, 1H), 6.08 (d, J = 7.2 Hz, 1H), 5.65 (d, J = 4.4 Hz, 1H), 5.49 (d, J = 6.8 Hz, 1H), 4.63-4.60 (m, 1H), 4.29 (d, J = 1.6 Hz, 1H), 4.17- 4.16 (m, 1H), 3.89 (s, 3H), 3.81-3.77 (m, 1H), 3.51-3.45 (m, 2H), 2.86 (s, 3H), 2.86- 2.79 (m, 2H), 1.93-1.91 (m, 1H), 1.84-1.81 (m, 1H), 1.52- 1.44 (m, 2H). |
| 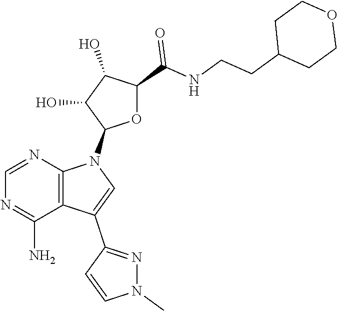 [SMILES: Cn1ccc(-c2cn([C@@H]3O[C@H](C(=O)NCCC4CCOCC4)[C@@H](O)[C@H]3O)c3ncnc(N)c23)n1] | I-96 | 8 | 472 | |
| 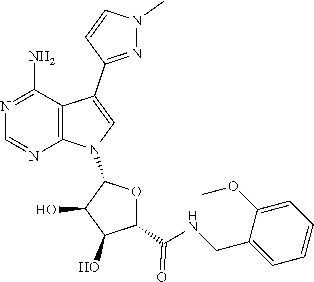 [SMILES: COc1ccccc1CNC(=O)[C@H]1O[C@@H](n2cc(-c3ccn(C)n3)c3c(N)ncnc32)[C@H](O)[C@@H]1O] | I-97 | 8 | 480 | |
| 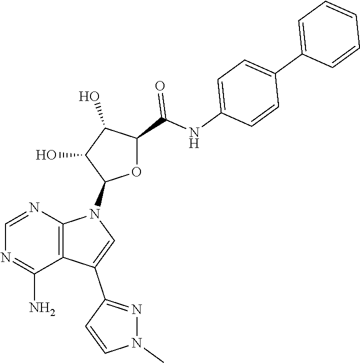 [SMILES: Cn1ccc(-c2cn([C@@H]3O[C@H](C(=O)Nc4ccc(-c5ccccc5)cc4)[C@@H](O)[C@H]3O)c3ncnc(N)c23)n1] | I-98 | 8 | 512 | |
| 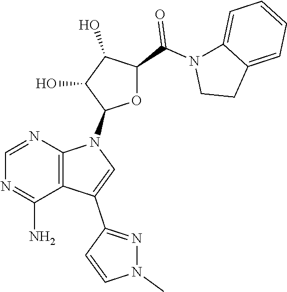 [SMILES: Cn1ccc(-c2cn([C@@H]3O[C@H](C(=O)N4CCc5ccccc54)[C@@H](O)[C@H]3O)c3ncnc(N)c23)n1] | I-99 | 8 | 462 | |
| 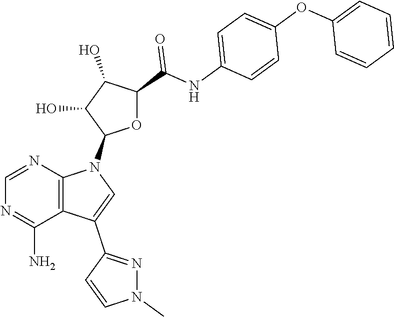 [SMILES: Cn1ccc(-c2cn([C@@H]3O[C@H](C(=O)Nc4ccc(Oc5ccccc5)cc4)[C@@H](O)[C@H]3O)c3ncnc(N)c23)n1] | I-100 | 8 | 528 | |
| 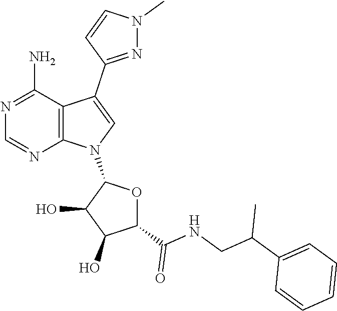 [SMILES: CC(CNC(=O)[C@H]1O[C@@H](n2cc(-c3ccn(C)n3)c3c(N)ncnc32)[C@H](O)[C@@H]1O)c1ccccc1] | I-101 | 8 | 478 | |
| 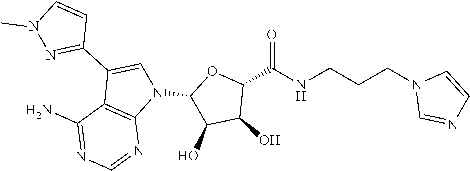 [SMILES: Cn1ccc(-c2cn([C@@H]3O[C@H](C(=O)NCCCn4ccnc4)[C@@H](O)[C@H]3O)c3ncnc(N)c23)n1] | I-102 | 8 | 468 | |
| 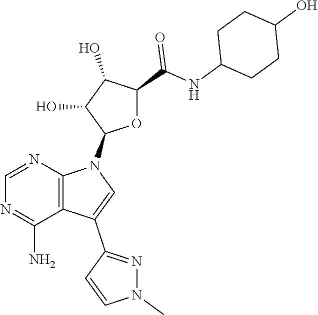 [SMILES: Cn1ccc(-c2cn([C@@H]3O[C@H](C(=O)NC4CCC(O)CC4)[C@@H](O)[C@H]3O)c3ncnc(N)c23)n1] | I-103 | 8 | 458 | ¹H NMR (400 MHz, DMSO- d6) δ ppm 9.09 (s, 1H), 8.20 (d, J = 8.0 Hz, 1H), 8.08 (s, 1H), 7.91 (s, 1H), 7.76 (d, J = 2.4 Hz, 1H), 7.29 (s, 1H), 6.57 (d, J = 2.4 Hz, 1H), 6.03 (d, J = 7.6 Hz, 1H), 5.63 (d, J = 4.4 Hz, 1H), 5.45 (d, J = 6.4 Hz, 1H), 4.64-4.59 (m, 2H), 4.26 (d, J = 1.6 Hz, 1H), 4.14-4.12 (m, 1H), 3.89 (s, 3H), 1.82- 1.72 (m, 4H), 1.25-1.21 (m, 4H). |
| 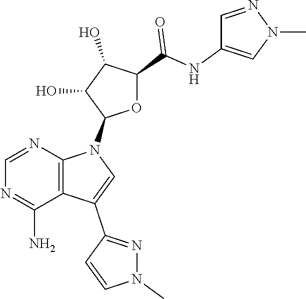 [SMILES: Cn1cc(NC(=O)[C@H]2O[C@@H](n3cc(-c4ccn(C)n4)c4c(N)ncnc43)[C@H](O)[C@@H]2O)cn1] | I-104 | 8 | 440 | |
| 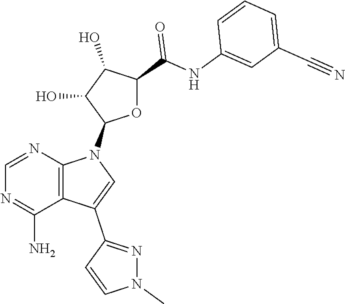 [SMILES: Cn1ccc(-c2cn([C@@H]3O[C@H](C(=O)Nc4cccc(C#N)c4)[C@@H](O)[C@H]3O)c3ncnc(N)c23)n1] | I-105 | 8 | 461 | |
| 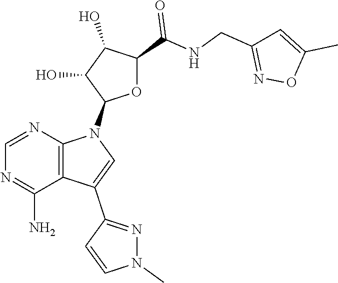 [SMILES: Cc1cc(CNC(=O)[C@H]2O[C@@H](n3cc(-c4ccn(C)n4)c4c(N)ncnc43)[C@H](O)[C@@H]2O)no1] | I-106 | 8 | 455 | |
| 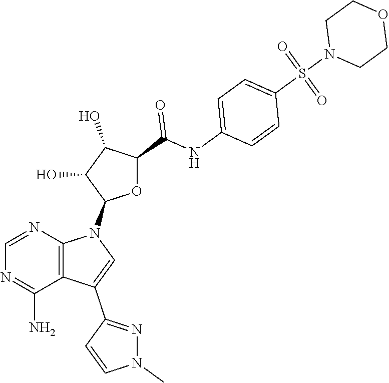 [SMILES: Cn1ccc(-c2cn([C@@H]3O[C@H](C(=O)Nc4ccc(S(=O)(=O)N5CCOCC5)cc4)[C@@H](O)[C@H]3O)c3ncnc(N)c23)n1] | I-107 | 8 | 585 | |
| 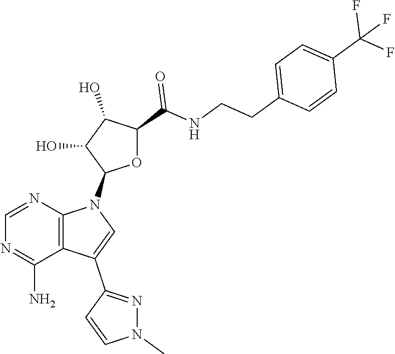 [SMILES: Cn1ccc(-c2cn([C@@H]3O[C@H](C(=O)NCCc4ccc(C(F)(F)F)cc4)[C@@H](O)[C@H]3O)c3ncnc(N)c23)n1] | I-108 | 8 | 532 | ¹H NMR (400 MHz, DMSO- d6) δ ppm 8.98 (t, J = 8.0 Hz, 1H), 7.97 (s, 1H), 7.85 (s, 1H), 7.74 (d, J = 2.0 Hz, 1H), 7.58 (s, 1H), 7.56-7.47 (m, 4H), 6.57 (d, J = 2.0 Hz, 1H), 5.94 (d, J = 8.0 Hz, 1H), 5.75 (s, 1H), 5.65 (d, J = 4.4 Hz, 1H), 5.42 (d, J = 6.8 Hz, 1H), 4.56- 4.53 (m, 1H), 4.25 (d, J = 1.2 Hz, 1H), 4.10-4.05 (m, 1H), 3.89 (s, 3H), 2.90-2.85 (m, 2H), 1.85-1.83 (m, 2H). |
| 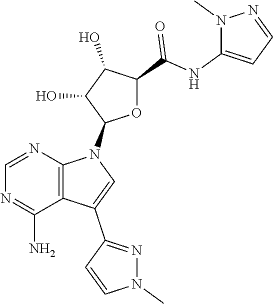 [SMILES: Cn1ccc(-c2cn([C@@H]3O[C@H](C(=O)Nc4ccnn4C)[C@@H](O)[C@H]3O)c3ncnc(N)c23)n1] | I-109 | 8 | 440 | |
| 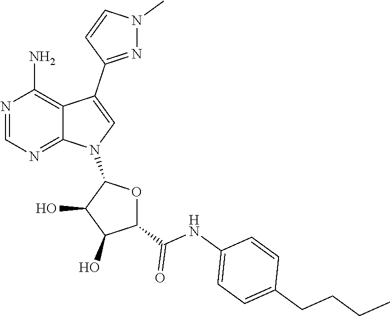 [SMILES: CCCCc1ccc(NC(=O)[C@H]2O[C@@H](n3cc(-c4ccn(C)n4)c4c(N)ncnc43)[C@H](O)[C@@H]2O)cc1] | I-110 | 8 | 492 | |
| 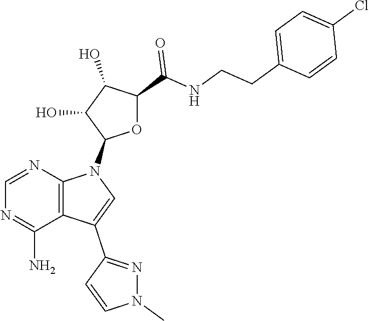 [SMILES: Cn1ccc(-c2cn([C@@H]3O[C@H](C(=O)NCCc4ccc(Cl)cc4)[C@@H](O)[C@H]3O)c3ncnc(N)c23)n1] | I-111 | 8 | 498 | ¹H NMR (400 MHz, DMSO- d6) δ 9.22 (s, 1H), 9.06 (t, J = 5.8 Hz, 1H), 8.12 (s, 1H), 8.01 (s, 1H), 7.89 (d, J = 2.2 Hz, 1H), 7.53-7.39 (m, 3H), 7.37 (d, J = 8.5 Hz, 2H), 6.72 (d, J = 2.3 Hz, 1H), 6.09 (d, J = 7.7 Hz, 1H), 5.78 (d, J = 4.3 Hz, 1H), 5.60 (d, J = 6.7 Hz, 1H), 4.77-4.67 (m, 1H), 4.39 (d, J = 1.4 Hz, 1H), 4.19 (dd, J = 6.6, 2.4 Hz, 1H), 4.03 (s, 3H), 3.61 (dd, J = 13.2, 6.3 Hz, 1H), 2.91 (t, J = 7.1 Hz, 2H). |
| 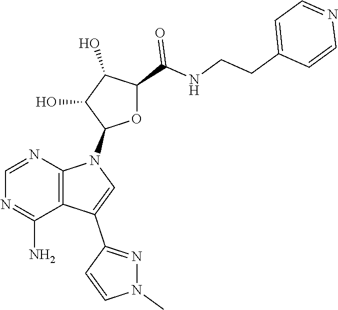 [SMILES: Cn1ccc(-c2cn([C@@H]3O[C@H](C(=O)NCCc4ccncc4)[C@@H](O)[C@H]3O)c3ncnc(N)c23)n1] | I-112 | 8 | 465 | |
| 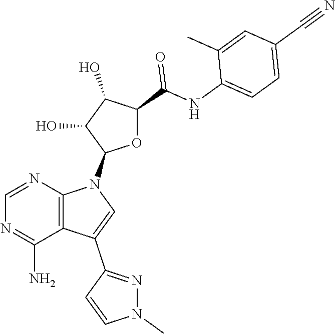 [SMILES: Cc1cc(C#N)ccc1NC(=O)[C@H]1O[C@@H](n2cc(-c3ccn(C)n3)c3c(N)ncnc32)[C@H](O)[C@@H]1O] | I-113 | 8 | 475 | |
| 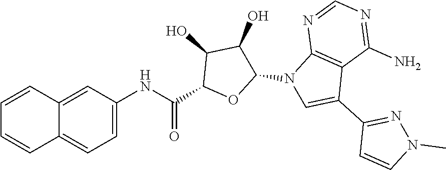 [SMILES: Cn1ccc(-c2cn([C@@H]3O[C@H](C(=O)Nc4ccc5ccccc5c4)[C@@H](O)[C@H]3O)c3ncnc(N)c23)n1] | I-114 | 8 | 486 | |
| 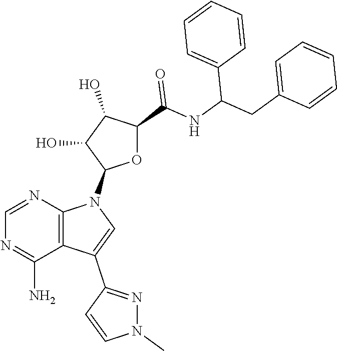 [SMILES: Cn1ccc(-c2cn([C@@H]3O[C@H](C(=O)NC(Cc4ccccc4)c4ccccc4)[C@@H](O)[C@H]3O)c3ncnc(N)c23)n1] | I-115 | 8 | 540 | |
| 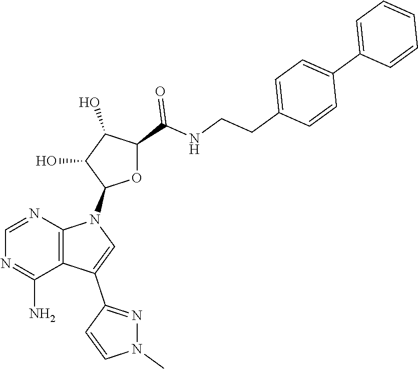 [SMILES: Cn1ccc(-c2cn([C@@H]3O[C@H](C(=O)NCCc4ccc(-c5ccccc5)cc4)[C@@H](O)[C@H]3O)c3ncnc(N)c23)n1] | I-116 | 8 | 540 | |
| 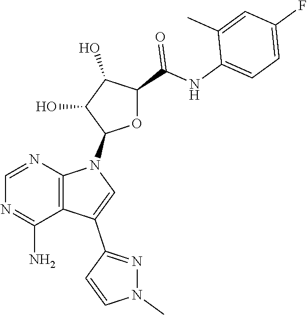 [SMILES: Cc1cc(F)ccc1NC(=O)[C@H]1O[C@@H](n2cc(-c3ccn(C)n3)c3c(N)ncnc32)[C@H](O)[C@@H]1O] | I-117 | 8 | 468 | |
| 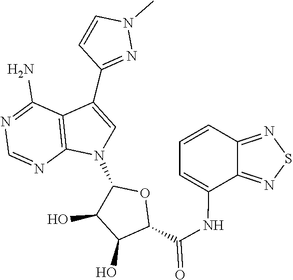 [SMILES: Cn1ccc(-c2cn([C@@H]3O[C@H](C(=O)Nc4cccc5nsnc45)[C@@H](O)[C@H]3O)c3ncnc(N)c23)n1] | I-118 | 8 | 494 | |
| 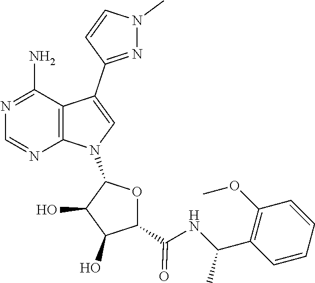 [SMILES: COc1ccccc1[C@H](C)NC(=O)[C@H]1O[C@@H](n2cc(-c3ccn(C)n3)c3c(N)ncnc32)[C@H](O)[C@@H]1O] | I-119 | 8 | 494 | |
| 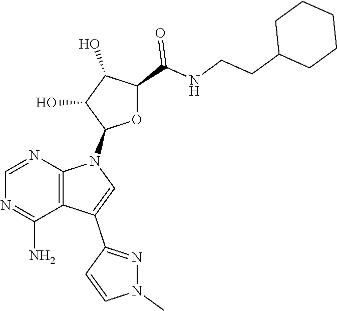 [SMILES: Cn1ccc(-c2cn([C@@H]3O[C@H](C(=O)NCCC4CCCCC4)[C@@H](O)[C@H]3O)c3ncnc(N)c23)n1] | I-120 | 8 | 470 | ¹H NMR (400 MHz, CD3OD) δ ppm 8.08 (s, 1H), 7.70 (s, 1H), 7.56 (d, J = 4.0 Hz, 1H), 6.59 (d, J = 4.0 Hz, 1H), 6.03 (d, J = 8.0 Hz, 1H), 4.82-4.78 (m, 1H), 4.43 (d, J = 1.6 Hz, 1H), 4.32-4.30 (m, 1H), 3.93 (s, 3H), 3.36-3.33 (m, 2H), 1.76-1.63 (m, 6H), 1.48-1.42 (m, 2H), 1.33-1.15 (m, 6H), 0.95-0.90 (m, 2H). |
| 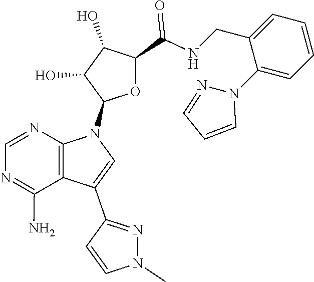 [SMILES: Cn1ccc(-c2cn([C@@H]3O[C@H](C(=O)NCc4ccccc4-n4cccn4)[C@@H](O)[C@H]3O)c3ncnc(N)c23)n1] | I-121 | 8 | 516 | ¹H NMR (400 MHz, CD3OD) δ ppm 7.84 (d, J = 1.6 Hz, 1H), 7.61-7.36 (m, 8H), 6.48 (d, J = 2.0 Hz, 1H), 6.43 (d, J = 1.6 Hz, 1H), 5.99 (d, J = 8.8 Hz, 1H), 4.74-4.71 (m, 1H), 4.58-4.54 (m, 1H), 4.42-4.35 (m, 2H), 4.28 (d, J = 4.4 Hz, 1H), 3.91 (s, 3H). |
| 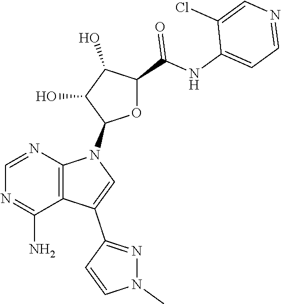 [SMILES: Cn1ccc(-c2cn([C@@H]3O[C@H](C(=O)Nc4ccncc4Cl)[C@@H](O)[C@H]3O)c3ncnc(N)c23)n1] | I-122 | 8 | 471 | |
| 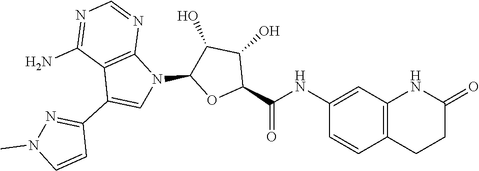 [SMILES: Cn1ccc(-c2cn([C@@H]3O[C@H](C(=O)Nc4ccc5c(c4)NC(=O)CC5)[C@@H](O)[C@H]3O)c3ncnc(N)c23)n1] | I-123 | 8 | 505 | |
| 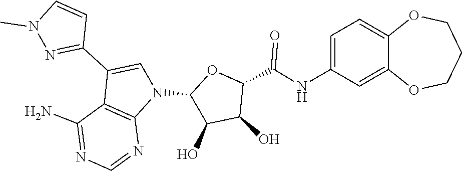 [SMILES: Cn1ccc(-c2cn([C@@H]3O[C@H](C(=O)Nc4ccc5c(c4)OCCCO5)[C@@H](O)[C@H]3O)c3ncnc(N)c23)n1] | I-124 | 8 | 508 | |
| 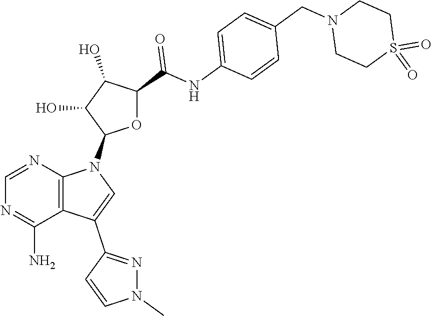 [SMILES: Cn1ccc(-c2cn([C@@H]3O[C@H](C(=O)Nc4ccc(CN5CCS(=O)(=O)CC5)cc4)[C@@H](O)[C@H]3O)c3ncnc(N)c23)n1] | I-125 | 8 | 583 | |
| 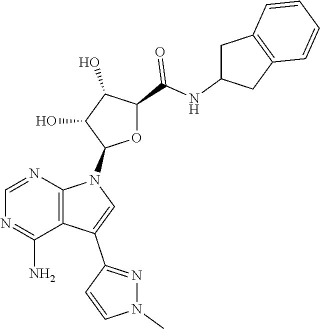 [SMILES: Cn1ccc(-c2cn([C@@H]3O[C@H](C(=O)NC4Cc5ccccc5C4)[C@@H](O)[C@H]3O)c3ncnc(N)c23)n1] | I-126 | 8 | 476 | |
| 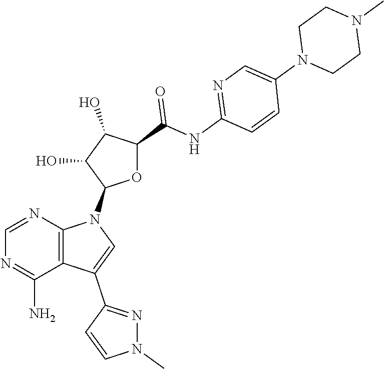 [SMILES: CN1CCN(c2ccc(NC(=O)[C@H]3O[C@@H](n4cc(-c5ccn(C)n5)c5c(N)ncnc54)[C@H](O)[C@@H]3O)nc2)CC1] | I-127 | 8 | 535 | |
| 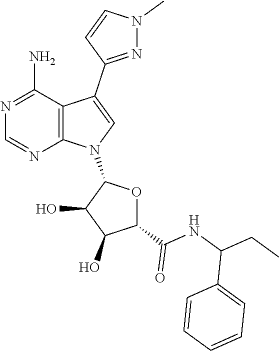 [SMILES: CCC(NC(=O)[C@H]1O[C@@H](n2cc(-c3ccn(C)n3)c3c(N)ncnc32)[C@H](O)[C@@H]1O)c1ccccc1] | I-128 | 8 | 468 | |
| 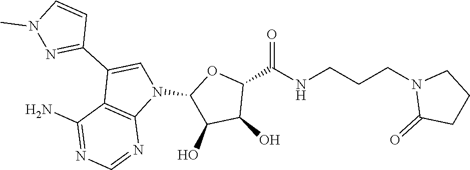 [SMILES: Cn1ccc(-c2cn([C@@H]3O[C@H](C(=O)NCCCN4CCCC4=O)[C@@H](O)[C@H]3O)c3ncnc(N)c23)n1] | I-129 | 8 | 485 | |
| 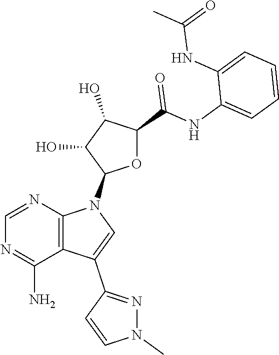 [SMILES: CC(=O)Nc1ccccc1NC(=O)[C@H]1O[C@@H](n2cc(-c3ccn(C)n3)c3c(N)ncnc32)[C@H](O)[C@@H]1O] | I-130 | 8 | 493 | |
| 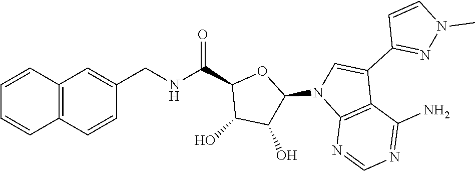 [SMILES: Cn1ccc(-c2cn([C@@H]3O[C@H](C(=O)NCc4ccc5ccccc5c4)[C@@H](O)[C@H]3O)c3ncnc(N)c23)n1] | I-131 | 8 | 500 | |
| 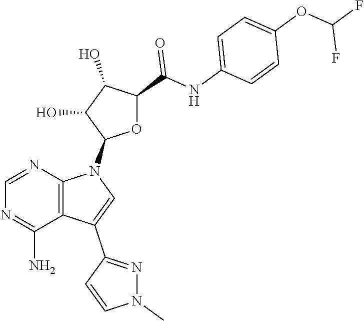 [SMILES: Cn1ccc(-c2cn([C@@H]3O[C@H](C(=O)Nc4ccc(OC(F)F)cc4)[C@@H](O)[C@H]3O)c3ncnc(N)c23)n1] | I-132 | 8 | 502 | |
| 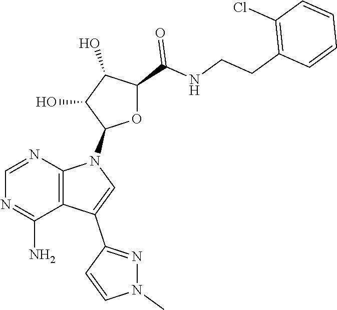 [SMILES: Cn1ccc(-c2cn([C@@H]3O[C@H](C(=O)NCCc4ccccc4Cl)[C@@H](O)[C@H]3O)c3ncnc(N)c23)n1] | I-133 | 8 | 498 | ¹H NMR (400 MHz, MeOD) δ 6.56 (s, 1H), 6.31 (s, 1H), 6.26 (d, J = 2.1 Hz, 1H), 6.10-5.99 (m, 1H), 6.00-5.93 (m, 1H), 5.90-5.80 (m, 2H), 5.27 (d, J = 2.2 Hz, 1H), 4.62 (d, J = 7.8 Hz, 1H), 3.44 (dd, J = 7.8, 4.9 Hz, 1H), 3.12 (s, 1H), 2.95 (d, J = 4.8 Hz, 1H), 2.63 (s, 3H), 2.31 (dtd, J = 20.3, 13.5, 6.9 Hz, 2H), 1.84-1.62 (m, 2H). |
| 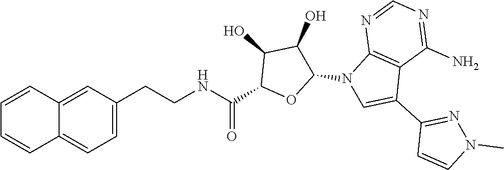 [SMILES: Cn1ccc(-c2cn([C@@H]3O[C@H](C(=O)NCCc4ccc5ccccc5c4)[C@@H](O)[C@H]3O)c3ncnc(N)c23)n1] | I-134 | 8 | 514 | |
| 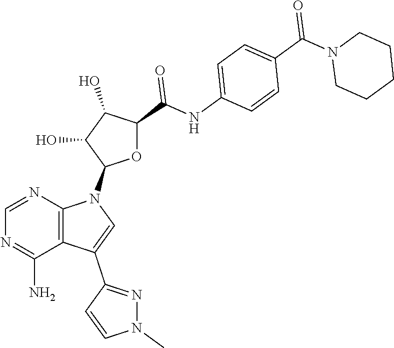 [SMILES: Cn1ccc(-c2cn([C@@H]3O[C@H](C(=O)Nc4ccc(C(=O)N5CCCCC5)cc4)[C@@H](O)[C@H]3O)c3ncnc(N)c23)n1] | I-135 | 8 | 547 | |
| 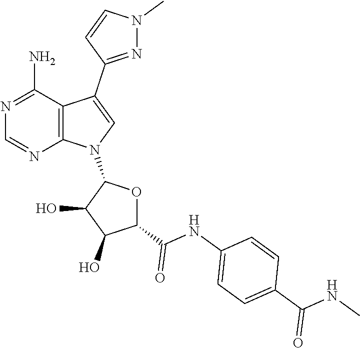 [SMILES: CNC(=O)c1ccc(NC(=O)[C@H]2O[C@@H](n3cc(-c4ccn(C)n4)c4c(N)ncnc43)[C@H](O)[C@@H]2O)cc1] | I-136 | 8 | 493 | |
| 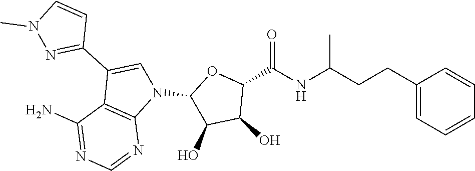 [SMILES: CC(CCc1ccccc1)NC(=O)[C@H]1O[C@@H](n2cc(-c3ccn(C)n3)c3c(N)ncnc32)[C@H](O)[C@@H]1O] | I-137 | 8 | 492 | |
| 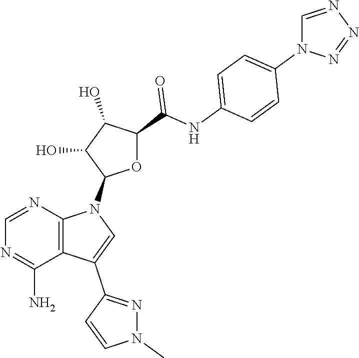 [SMILES: Cn1ccc(-c2cn([C@@H]3O[C@H](C(=O)Nc4ccc(-n5cnnn5)cc4)[C@@H](O)[C@H]3O)c3ncnc(N)c23)n1] | I-138 | 8 | 504 | |
| 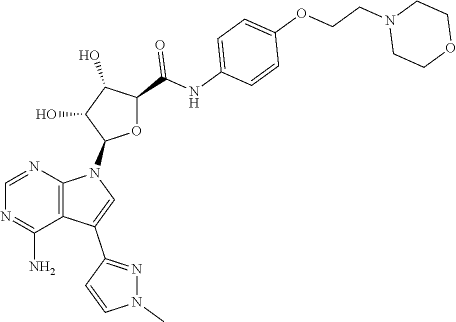 [SMILES: Cn1ccc(-c2cn([C@@H]3O[C@H](C(=O)Nc4ccc(OCCN5CCOCC5)cc4)[C@@H](O)[C@H]3O)c3ncnc(N)c23)n1] | I-139 | 8 | 565 | ¹H NMR (400 MHz, DMSO- d6) δ ppm 10.34 (s, 1H), 9.09 (brs, 1H), 8.03 (s, 1H), 7.98 (s, 1H), 7.75 (d, J = 2.0 Hz, 1H), 7.51 (d, J = 8.8 Hz, 2H), 7.29 (brs, 1H), 6.93 (d, J = 9.2 Hz, 2H), 6.55 (d, J = 2.0 Hz, 1H), 6.11 (d, J = 7.2 Hz, 1H), 5.71 (d, J = 4.8 Hz, 1H), 5.53 (d, J = 6.4 Hz, 1H), 4.67-4.63 (m, 1H), 4.46 (d, J = 2.0 Hz, 1H), 4.30-4.29 (m, 1H), 4.06 (t, J = 6.4 Hz, 2H), 3.88 (s, 3H), 3.58 (t, J = 4.4 Hz, 4H), 2.68 (t, J = 7.0 Hz, 2H), 2.48-2.45 (m, 4H). |
| 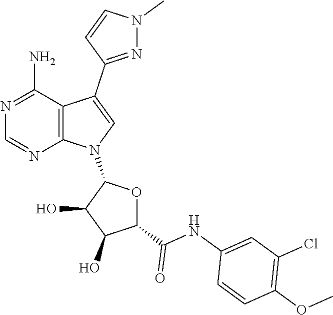 [SMILES: COc1ccc(NC(=O)[C@H]2O[C@@H](n3cc(-c4ccn(C)n4)c4c(N)ncnc43)[C@H](O)[C@@H]2O)cc1Cl] | I-140 | 8 | 500 | |
| 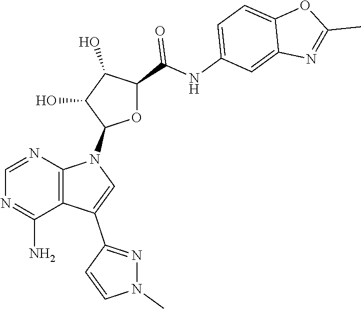 [SMILES: Cc1nc2cc(NC(=O)[C@H]3O[C@@H](n4cc(-c5ccn(C)n5)c5c(N)ncnc54)[C@H](O)[C@@H]3O)ccc2o1] | I-141 | 8 | 491 | |
| 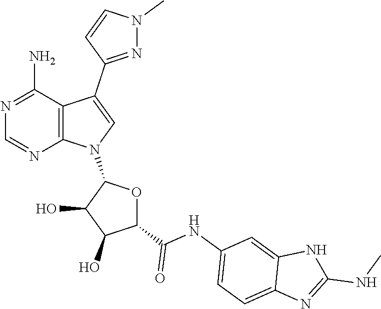 [SMILES: CNc1nc2ccc(NC(=O)[C@H]3O[C@@H](n4cc(-c5ccn(C)n5)c5c(N)ncnc54)[C@H](O)[C@@H]3O)cc2[nH]1] | I-142 | 8 | 505 | ¹H NMR (400 MHz, CD3OD) δ ppm 10.65 (d, 1H), 9.95 (d, 1H), 8.26 (d, 1H), 8.12 (d, 1H), 8.06 (s, 1H), 7.85 (s, 1H), 7.62 (s, 0.6H), 7.62 (s, 0.5H), 7.02- 6.87 (m, 3H), 6.74-6.70 (m, 1H), 6.56 (d, 1H), 5.79 (d, 1H), 4.61 (s, 1H), 4.44 (s, 1H), 4.18-4.14 (m, 1H), 2.78-2.71 (m, 1H), 2.32-2.25 (m, 3H), 1.97-1.60 (m, 4H). |
| 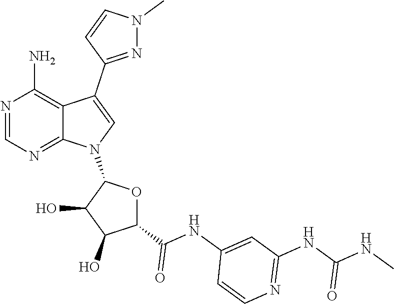 [SMILES: CNC(=O)Nc1cc(NC(=O)[C@H]2O[C@@H](n3cc(-c4ccn(C)n4)c4c(N)ncnc43)[C@H](O)[C@@H]2O)ccn1] | I-143 | 8 | 509 | ¹H NMR (400 MHz, DMSO- d6) δ ppm 9.30 (s, 1H), 9.04 (b, 1H), 8.22 (b, 1H), 8.11 (s, 1H), 7.07 (d, J = 5.6 Hz, 2H), 7.99 (s, 1H), 7.765 (s, 1H), 7.62 (s, 1H), 7.28 (b, 1H), 7.205 (d, J = 6.0 Hz, 1H), 6.575 (s, 1H), 6.16 (d, J = 6.8 Hz, 1H), 5.745 (d, J = 4.8 Hz, 1H), 5.585 (d, J = 6.4 Hz, 1H), 4.60-4.57 (m, 1H), 4.52 (s, 1H), 4.32 (s, 1H), 3.90 (s, 3H), 2.73 (d, J = 4.4 Hz, 3H). |
| 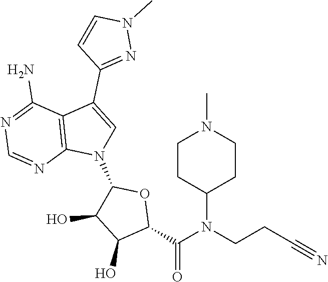 [SMILES: CN1CCC(N(CCC#N)C(=O)[C@H]2O[C@@H](n3cc(-c4ccn(C)n4)c4c(N)ncnc43)[C@H](O)[C@@H]2O)CC1] | I-144 | 8 | 510 | ¹H NMR (400 MHz, DMSO) δ 9.04-8.90 (br, 1H), 8.06 (s, 1H), 7.86-7.84 (m, 1H), 7.77- 7.75 (m, 1H), 7.26-7.15 (br, 1H), 6.56-6.51 (m, 1H), 6.29- 6.27 (m, 1H), 5.64-5.58 (m, 1H), 5.53 (d, J = 6.6 Hz, 1H), 4.81-4.74 (m, 1H), 4.51- 4.39 (m, 1H), 4.34-4.29 (m, 1H), 3.89 (s, 3H), 3.71-3.60 (m, 1H), 3.54-3.42 (m, 2H), 3.30-3.28 (m, 2H), 2.82-2.67 (m, 4H), 2.19- 2.14 (m, 3H), 1.99-1.67 (m, 4H). |
| 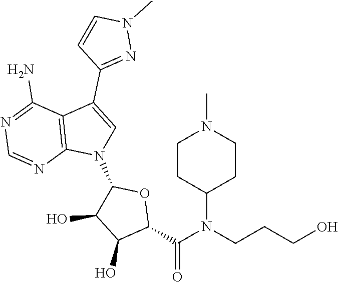 [SMILES: CN1CCC(N(CCCO)C(=O)[C@H]2O[C@@H](n3cc(-c4ccn(C)n4)c4c(N)ncnc43)[C@H](O)[C@@H]2O)CC1] | I-145 | 8 | 515.2 | |
| 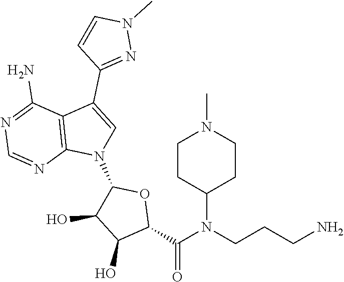 [SMILES: CN1CCC(N(CCCN)C(=O)[C@H]2O[C@@H](n3cc(-c4ccn(C)n4)c4c(N)ncnc43)[C@H](O)[C@@H]2O)CC1] | I-146 | 8 | 514.2 | ¹H NMR (400 MHz, DMSO- d6) δ 9.02-8.89 (br, 1H), 8.06 (d, J = 1.2 Hz, 1H), 7.96-7.83 (m, 1H), 7.78-7.74 (m, 1H), 7.26-7.16 (br, 1H), 6.54-6.49 (m, 1H), 6.30-6.26 (m, 1H), 5.76-5.47 (br, 2H), 4.79- 4.65 (m, 1H), 4.51-4.39 (m, 1H), 4.34-4.19 (m, 1H), 4.11- 3.63 (m, 4H), 3.53-3.44 (m, 1H), 3.26-3.23 (m, 2H), 3.13- 2.92 (m, 2H), 2.83-2.78 (m, 1H), 2.72-2.62 (m, 2H), 2.15- 2.12 (m, 3H), 1.99-1.39 (m, 8H). |
| 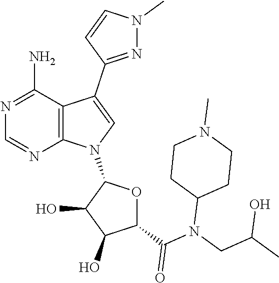 [SMILES: CC(O)CN(C(=O)[C@H]1O[C@@H](n2cc(-c3ccn(C)n3)c3c(N)ncnc32)[C@H](O)[C@@H]1O)C1CCN(C)CC1] | I-147 | 8 | 515.2 | |
| 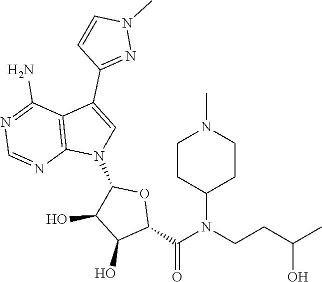 [SMILES: CC(O)CCN(C(=O)[C@H]1O[C@@H](n2cc(-c3ccn(C)n3)c3c(N)ncnc32)[C@H](O)[C@@H]1O)C1CCN(C)CC1] | I-148 | 8 | 529 | ¹H NMR (400 MHz, DMSO- d6) δ 8.94 (s, 1H), 8.06 (s, 1H), 7.94-7.70 (m, 2H), 7.19 (s, 1H), 6.55-6.45 (m, 1H), 6.27 (t, J = 6.8 Hz, 1H), 5.60-5.32 (m, 2H), 4.79-4.74 (m, 0.5H), 4.72-4.69 (m, 0.5H), 4.62-4.58 (m, 0.5H), 4.55-4.48 (m, 1H), 4.46-4.38 (m, 0.5H), 4.35- 4.30 (m, 0.5H), 4.25-4.20 (m, 0.5H), 4.15-4.00 (m, 0.5H), 3.89 (d, J = 1.9 Hz, 3H), 3.80- 3.55 (m, 1.5H), 3.24-3.14 (m, 1H), 2.95-2.65 (m, 2H), 2.14 (d, J = 4.0 Hz, Hz, 3H), 2.05- 1.24 (m, 9H), 1.11-0.99 (m, 3H). |
| 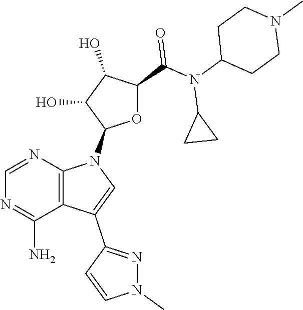 [SMILES: CN1CCC(N(C(=O)[C@H]2O[C@@H](n3cc(-c4ccn(C)n4)c4c(N)ncnc43)[C@H](O)[C@@H]2O)C2CC2)CC1] | I-149 | 8 | 497 | ¹H NMR (400 MHz, DMSO- d6) δ ppm 8.34 (s, 1H), 8.11 (d, J = 6.4 Hz, 1H), 8.04 (d, J = 8.8 Hz, 1H), 7.87 (s, 1H), 7.77 (d, J = 7.6 Hz, 1H), 7.48 (d, J = 1.6 Hz, 1H), 7.13 (dd, J = 8.4 Hz, 2 Hz, 1H), 6.73 (d, J = 8.8 Hz, 1H), 6.60 (s, 1H), 5.40 (s, 1H), 4.58 (s, 1H), 4.37 (s, 1H), 4.10 (t, J = 7.6 Hz, 4H), 3.28 (s, 3H), 2.67-2.63 (m, 1H), 2.40- 2.33 (m, 2H), 2.30-2.26 (m, 1H). |
| 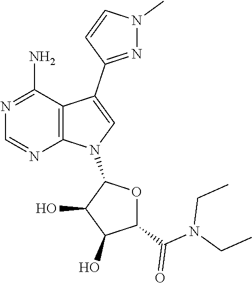 [SMILES: CCN(CC)C(=O)[C@H]1O[C@@H](n2cc(-c3ccn(C)n3)c3c(N)ncnc32)[C@H](O)[C@@H]1O] | I-150 | 8 | 416.1 | |
| 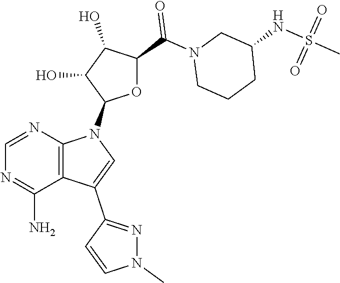 [SMILES: Cn1ccc(-c2cn([C@@H]3O[C@H](C(=O)N4CCC[C@@H](NS(C)(=O)=O)C4)[C@@H](O)[C@H]3O)c3ncnc(N)c23)n1] | I-151 | 8 | 520.8 | ¹H NMR (400 MHz, DMSO- d6) δ 8.96 (s, 1H), 8.06 (s, 1H), 7.92 (d, J = 26.1 Hz, 1H), 7.75 (s, 1H), 7.26-7.24 (m, 2H), 6.54 (d, J = 2.2 Hz, 1H), 6.26 (d, J = 6.5 Hz, 1H), 5.59-5.39 (m, 2H), 4.77 (d, J = 15.3 Hz, 1H), 4.40-4.35 (m, 2H), 4.23- 4.20 (m, 0.6H), 3.89 (s, 3.6H), 3.80-3.66 (m, 2H), 3.21-3.12 (m, 0.6H), 3.03 (s, 1H), 2.94 (d, J = 16.5 Hz, 3.3H), 2.81- 2.71 (m, 0.5H), 1.90-1.84 (m, 1H), 1.70-1.65 (m, 1H), 1.44- 1.35 (m, 2H). |
| 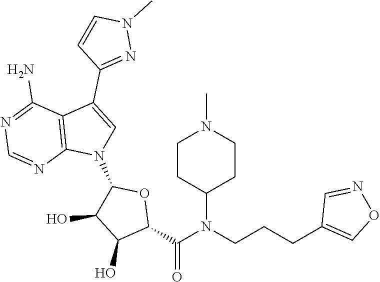 [SMILES: CN1CCC(N(CCCc2cnoc2)C(=O)[C@H]2O[C@@H](n3cc(-c4ccn(C)n4)c4c(N)ncnc43)[C@H](O)[C@@H]2O)CC1] | I-152 | 8 | 566 | ¹H NMR (400 MHz, DMSO- d6) δ 9.03-8.88 (br, 1H), 8.75- 8.72 (m, 1H), 8.55-8.52 (m, 1H), 8.06 (m, 1H), 7.91-7.71 (m, 2H), 7.27-7.15 (br, 1H), 6.50-6.44 (m, 1H), 6.29-6.25 (m, 1H), 5.56-5.50 (m, 2H), 4.77-4.65 (m, 1H), 4.51-4.25 (m, 2H), 4.05-3.66 (m, 4H), 3.26-3.15 (m, 2H), 2.79- 2.68 (m, 2H), 2.44 (t, J = 7.6 Hz, 2H), 2.15-2.12 (m, 3H), 1.96-1.83 (m, 2H), 1.77- 1.58 (m, 4H), 1.54-1.41 (m, 2H). |
| 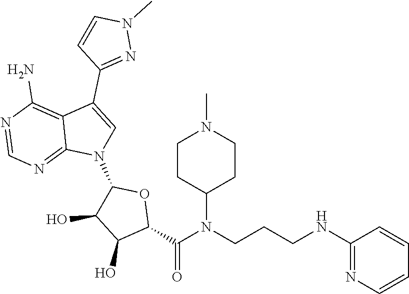 [SMILES: CN1CCC(N(CCCNc2ccccn2)C(=O)[C@H]2O[C@@H](n3cc(-c4ccn(C)n4)c4c(N)ncnc43)[C@H](O)[C@@H]2O)CC1] | I-153 | 8 | 591 | ¹H NMR (400 MHz, CD3OD) δ ppm 8.94 (brs, 1H), 8.06 (d, J = 1.2 Hz, 1H), 7.96-7.71 (m, 2H), 7.36-7.20 (m, 2H), 6.53- 6.42 (m, 4H), 6.29-6.25 (m, 1H), 5.58-5.50 (m, 2H), 4.79- 4.69 (m, 1H), 4.49-4.23 (m, 2H), 4.06-3.67 (m, 4H), 4.28- 4.21 (m, 4H), 2.80-2.67 (m, 2H), 2.14 (d, J = 10.8 Hz, 1H), 1.98-1.41 (m, 9H). |
| 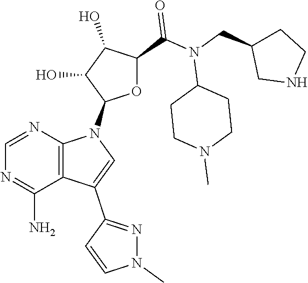 [SMILES: CN1CCC(N(C[C@H]2CCNC2)C(=O)[C@H]2O[C@@H](n3cc(-c4ccn(C)n4)c4c(N)ncnc43)[C@H](O)[C@@H]2O)CC1] | I-154 | 8 | 540 | ¹H NMR (400 MHz, CD3OD) δ 8.10 (s, 1H), 7.86 (d, J = 8.8 Hz, 1H), 7.57 (d, J = 8.8 Hz, 1H), 7.34 (d, J = 3.6 Hz, 1H), 7.03 (d, J = 2 Hz, 1H), 6.96 (dd, J = 8.8, 2.4 Hz, 1H), 6.69- 6.46 (m, 2H), 5.03-5.10 (m, 1H), 4.47-4.44 (m, 1H), 4.24 (d, J = 5.2 Hz, 1H), 4.19-4.17 (m, 1H), 3.84-3.81 (m, 1H), 2.51-2.63 (m, 2H), 1.89-1.97 (m, 1H). |
| 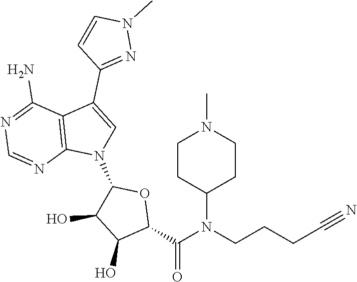 [SMILES: CN1CCC(N(CCCC#N)C(=O)[C@H]2O[C@@H](n3cc(-c4ccn(C)n4)c4c(N)ncnc43)[C@H](O)[C@@H]2O)CC1] | I-155 | 8 | 524 | ¹H NMR (400 MHz, DMSO- d6) δ 8.95 (s, 1H), 8.10-8.04 (m, 1H), 7.90-7.80 (m, 1H), 7.78-7.72 (m, 1H), 7.21 (s, 1H), 6.54-6.48 (m, 1H), 6.32- 6.22 (m, 1H), 5.72-5.48 (m, 2H), 4.80-4.65 (m, 1H), 4.57- 4.37 (m, 1H), 4.35-4.20 (m, 1H), 4.05-4.00 (m, 1H), 3.89 (s, 3H), 3.75-3.60 (m, 1H), 3.25 (t, J = 7.9 Hz, 2H), 2.85- 2.65 (m, 2H), 2.59-2.52 (m, 1H), 2.14 (d, J = 10.8 Hz, 3H), 2.01-1.33 (m, 9H). |
| 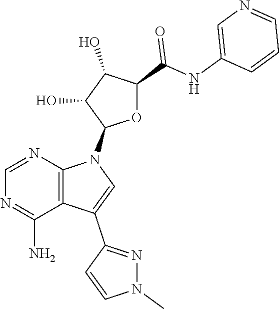 [SMILES: Cn1ccc(-c2cn([C@@H]3O[C@H](C(=O)Nc4cccnc4)[C@@H](O)[C@H]3O)c3ncnc(N)c23)n1] | I-156 | 8 | 437 | ¹H NMR (400 MHz, DMSO- d6) δ ppm 10.57 (s, 1H), 9.06 (brs, 1H), 8.77 (d, J = 2.4 Hz, 1H), 8.33 (d, J = 4.8, 1.6 Hz, 1H), 8.08-8.05 (m, 1H), 8.04 (s, 1H), 7.99 (s, 1H), 7.75 (d, J = 2.4 Hz, 1H), 7.41-7.38 (m, 1H), 7.29 (brs, 1H), 6.56 (d, J = 2.4 Hz, 1H), 6.18 (d, J = 7.2 Hz, 1H), 5.75 (d, J = 4.4 Hz, 1H), 5.57 (d, J = 6.8 Hz, 1H), 4.68-4.63 (m, 1H), 4.52 (d, J = 2.0 Hz, 1H), 4.38-4.35 (m, 1H), 3.89 (s, 3H). |
| 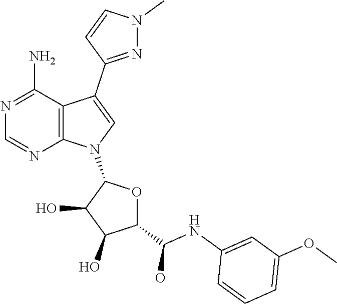 | I-157 | 8 | 466 | ¹H NMR (400 MHz, DMSO- d6) δ ppm 10.32 (s, 1H), 9.06 (brs, 1H), 8.06 (s, 1H), 7.97 (s, 1H), 7.75 (d, J = 2.4 Hz, 1H), 7.29-7.18 (m, 4H), 6.72-6.69 (m, 1H), 6.56 (d, J = 2.0 Hz, 1H), 6.14 (d, J = 6.8 Hz, 1H), 5.71 (d, J = 4.4 Hz, 1H), 5.54 (d, J = 6.4 Hz, 1H), 4.68-4.63 (m, 1H), 4.48 (d, J = 2.4 Hz, 1H), 4.34-4.31 (m, 1H), 3.89 (s, 3H), 3.73 (s, 3H). |
| 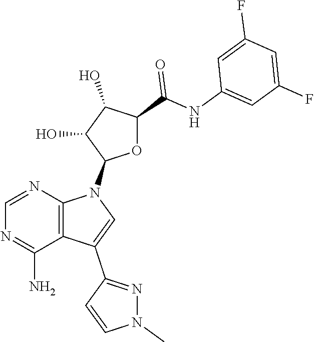 [SMILES: Cn1ccc(-c2cn([C@@H]3O[C@H](C(=O)Nc4cc(F)cc(F)c4)[C@@H](O)[C@H]3O)c3ncnc(N)c23)n1] | I-158 | 8 | 472 | ¹H NMR (400 MHz, DMSO- d6) δ 10.57 (s, 1H), 9.05 (s, 1H), 8.05 (s, 1H), 7.95 (s, 1H), 7.75 (d, J = 2.3 Hz, 1H), 7.44- 7.36 (m, 2H), 7.35-7.15 (m, 1H), 7.05-6.87 (m, 1H), 6.55 (d, J = 2.0 Hz, 1H), 6.21 (d, J = 6.9 Hz, 1H), 5.74 (d, J = 4.8 Hz, 1H), 5.57 (d, J = 6.4 Hz, 1H), 4.75-4.25 (m, 1H), 4.49 (d, J = 2.4 Hz, 1H), 4.42-4.32 (m, 1H), 3.89 (s, 3H). |
| 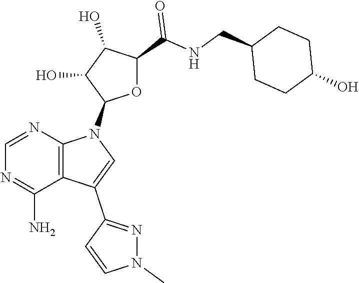 [SMILES: Cn1ccc(-c2cn([C@@H]3O[C@H](C(=O)NC[C@H]4CC[C@H](O)CC4)[C@@H](O)[C@H]3O)c3ncnc(N)c23)n1] | I-159 | 8 | 472 | |
| 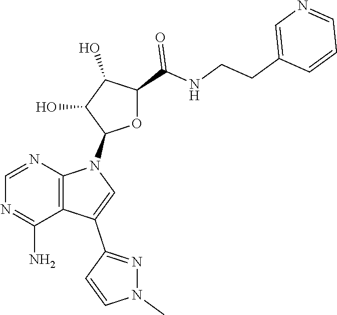 [SMILES: Cn1ccc(-c2cn([C@@H]3O[C@H](C(=O)NCCc4cccnc4)[C@@H](O)[C@H]3O)c3ncnc(N)c23)n1] | I-160 | 8 | 465 | ¹H NMR (400 MHz, DMSO- d6) δ 9.08 (s, 1H), 8.97 (s, 1H), 8.42 (d, J = 2.0 Hz, 1H), 8.37 (d, J = 3.3 Hz, 1H), 7.97 (s, 1H), 7.86 (s, 1H), 7.75 (d, J = 2.3 Hz, 1H), 7.67-7.60 (m, 1H), 7.38-7.22 (m, 2H), 6.59 (d, J = 2.3 Hz, 1H), 5.94 (d, J = 7.7 Hz, 1H), 5.64 (d, J = 4.3 Hz, 1H), 5.43 (d, J = 6.7 Hz, 1H), 4.65-4.45 (m, 1H), 4.24 (s, 1H), 4.10-4.04 (m, 1H), 3.89 (s, 3H), 3.55-3.45 (m, 1H), 3.45-3.35 (m, 1H), 2.84-2.77 (m, 2H). |
| 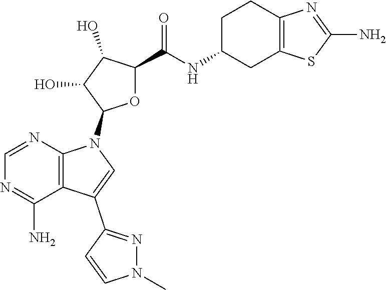 [SMILES: Cn1ccc(-c2cn([C@@H]3O[C@H](C(=O)N[C@@H]4CCc5nc(N)sc5C4)[C@@H](O)[C@H]3O)c3ncnc(N)c23)n1] | I-161 | 8 | 511.9 | ¹H NMR (400 MHz, DMSO- d6) δ ppm 9.03 (s, 1H), 8.34 (d, J = 8 Hz, 1H), 8.14 (s, 1H), 8.05 (s, 1H), 7.95 (s, 1H), 7.74 (d, J = 2 Hz, 1H), 7.26 (s, 1H), 6.67 (s, 2H), 6.58 (d, J = 2.4 Hz, 1H), 6.11 (d, J = 7.2 Hz, 1H), 5.64 (s, 1H), 5.47 (s, 1H), 4.62 (s, 1H), 4.32 (d, J = 1.6 Hz, 1H), 4.19 (d, J = 3.6 Hz, 1H), 4.06-4.02 (m, 1H), 3.89 (s, 3H), 2.89-2.81 (m, 1H), 2.49-2.32 (m, 3H), 1.84-1.69 (m, 2H). |
| 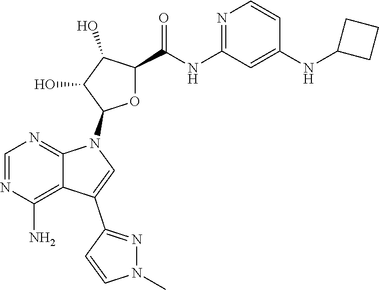 [SMILES: Cn1ccc(-c2cn([C@@H]3O[C@H](C(=O)Nc4cc(NC5CCC5)ccn4)[C@@H](O)[C@H]3O)c3ncnc(N)c23)n1] | I-162 | 8 | 506 | |
| 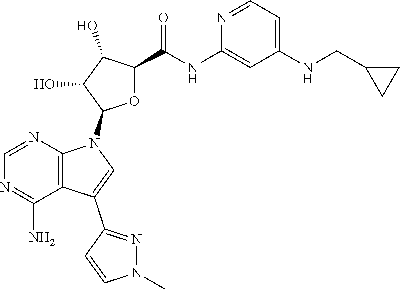 [SMILES: Cn1ccc(-c2cn([C@@H]3O[C@H](C(=O)Nc4cc(NCC5CC5)ccn4)[C@@H](O)[C@H]3O)c3ncnc(N)c23)n1] | I-163 | 8 | 506 | |
| 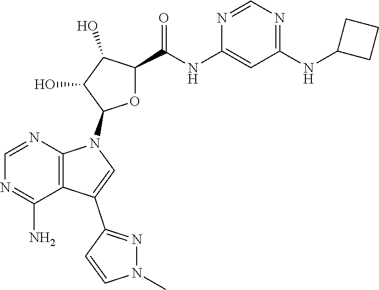 [SMILES: Cn1ccc(-c2cn([C@@H]3O[C@H](C(=O)Nc4cc(NC5CCC5)ncn4)[C@@H](O)[C@H]3O)c3ncnc(N)c23)n1] | I-164 | 8 | 507 | |
| 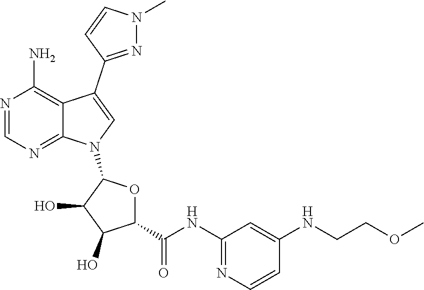 [SMILES: COCCNc1ccnc(NC(=O)[C@H]2O[C@@H](n3cc(-c4ccn(C)n4)c4c(N)ncnc43)[C@H](O)[C@@H]2O)c1] | I-165 | 8 | 510 | ¹H NMR (400 MHz, DMSO- d6) δ ppm 10.83 (s, 1H), 9.09- 9.01 (br, 1H), 8.33 (s, 1H), 7.86 (s, 1H), 7.83 (d, J = 5.6 Hz, 1H), 7.76 (s, 1H), 7.44 (s, 1H), 7.31-7.24 (br, 1H), 6.76- 6.73 (m, 1H), 6.58 (s, 1H), 6.36-6.32 (m, 1H), 5.98 (d, J = 7.6 Hz, 1H), 5.75 (d, J = 4.4 Hz, 1H), 5.53 (d, J = 7.6 Hz, 1H), 4.73-4.68 (m, 1H), 4.49 (s, 1H), 4.27-4.24 (m, 1H), 3.89 (s, 3H), 3.50-3.47 (m, 2H), 3.27 (s, 3H), 3.25-3.23 (m, 2H). |
| 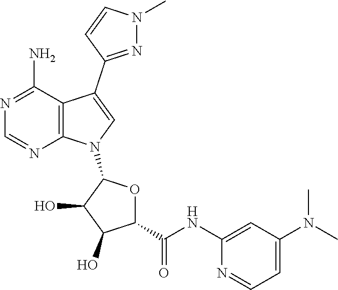 [SMILES: CN(C)c1ccnc(NC(=O)[C@H]2O[C@@H](n3cc(-c4ccn(C)n4)c4c(N)ncnc43)[C@H](O)[C@@H]2O)c1] | I-166 | 8 | 480 | |
| 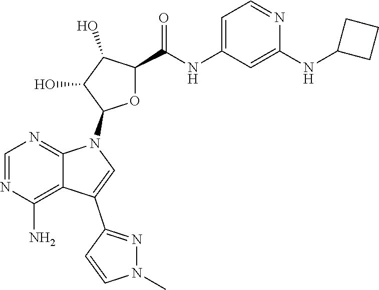 [SMILES: Cn1ccc(-c2cn([C@@H]3O[C@H](C(=O)Nc4ccnc(NC5CCC5)c4)[C@@H](O)[C@H]3O)c3ncnc(N)c23)n1] | I-167 | 8 | 506 | ¹H NMR (400 MHz, DMSO- d6) δ ppm 9.12-8.85 (br, 1H), 8.05 (s, 2H), 7.95 (d, J = 3.2 Hz, 1H), 7.81 (s, 1H), 7.32- 7.14 (br, 1H), 6.54 (s, 2H), 6.37-6.29 (m, 2H), 6.22 (d, J = 7.2 Hz, 1H), 5.42 (d, J = 7.2 Hz, 1H), 5.38 (s, 1H), 4.84- 4.76 (m, 1H), 4.59-4.47 (m, 1H), 4.15 (d, J = 14.4 Hz, 2H), 3.87 (s, 3H), 2.15-1.98 (m, 2H), 1.85-1.76 (m, 2H), 1.69- 1.43 (m, 2H). |
| 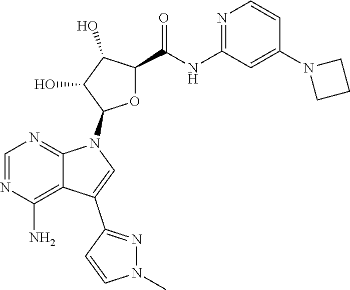 [SMILES: Cn1ccc(-c2cn([C@@H]3O[C@H](C(=O)Nc4cc(N5CCC5)ccn4)[C@@H](O)[C@H]3O)c3ncnc(N)c23)n1] | I-168 | 8 | 492 | ¹H NMR (400 MHz, DMSO- d6) δ ppm 10.95 (s, 1H), 9.15- 9.00 (br, 1H), 8.33 (s, 1H), 7.93 (d, J = 6 Hz, 1H), 7.87 (s, 1H), 7.15 (s, 1H), 7.34-7.27 (br, 1H), 7.22 (s, 1H), 6.59 (s, 1H), 6.14 (d, J = 6 Hz, 1H), 5.97 (d, J = 7.6 Hz, 1H), 5.745 (d, J = 4.4 Hz, 1H), 5.51 (d, J = 6.8 Hz, 1H), 4.76-4.72 (m, 1H), 4.49 (s, 1H), 4.27-4.25 (m, 1H), 3.94-3.90 (m, 7H), 2.44- 2.35 (m, 2H). |
| 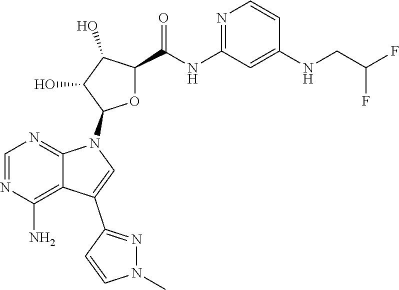 [SMILES: Cn1ccc(-c2cn([C@@H]3O[C@H](C(=O)Nc4cc(NCC(F)F)ccn4)[C@@H](O)[C@H]3O)c3ncnc(N)c23)n1] | I-169 | 8 | 516 | ¹H NMR (400 MHz, DMSO- d6) δ ppm 10.97 (s, 1H), 9.12- 9.15 (br, 1H), 8.33 (s, 1H), 7.9 (t, J = 3.2 Hz, 1H), 7.76 (s, 1H), 7.54 (s, 1H), 7.32-7.25 (br, 1H), 7.02-6.99 (m, 1H), 6.59 (s, 1H), 6.47 (d, J = 5.6 Hz, 1H), 6.10-6.14 (m, 0.5H), 5.98 (d, J = 7.6 Hz, 1H), ), 5.75 (d, J = 4.4 Hz, 1H), 5.63-5.60 (m, 0.5H), 5.53 (d, J = 6.4 Hz, 1H), 4.72-4.70 (m, 2H), 4.50 (s, 1H), 4.27-4.14 (m, 1H), 3.90 (s, 3H), 3.57-3.54 (m, 2H). |
| 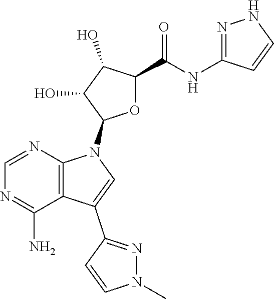 [SMILES: Cn1ccc(-c2cn([C@@H]3O[C@H](C(=O)Nc4cc[nH]n4)[C@@H](O)[C@H]3O)c3ncnc(N)c23)n1] | I-170 | 8 | 426 | ¹H NMR (400 MHz, CD3OD) δ ppm 10.65 (d, 1H), 9.95 (d, 1H), 8.26 (d, 1H), 8.12 (d, 1H), 8.06 (s, 1H), 7.85 (s, 1H), 7.62 (s, 0.6H), 7.62 (s, 0.5H), 7.02- 6.87 (m, 3H), 6.74-6.70 (m, 1H), 6.56 (d, 1H), 5.79 (d, 1H), 4.61 (s, 1H), 4.44 (s, 1H), 4.18-4.14 (m, 1H), 2.78-2.71 (m, 1H), 2.32-2.25 (m, 3H), 1.97-1.60 (m, 4H). |
| 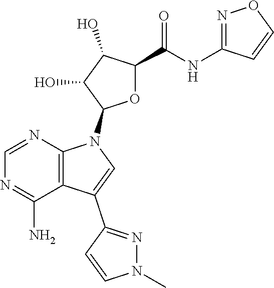 [SMILES: Cn1ccc(-c2cn([C@@H]3O[C@H](C(=O)Nc4ccon4)[C@@H](O)[C@H]3O)c3ncnc(N)c23)n1] | I-171 | 8 | 427 | |
| 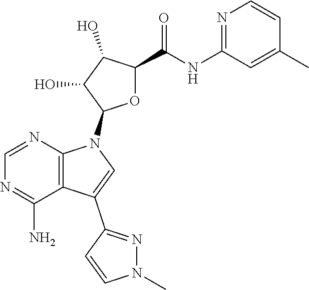 [SMILES: Cc1ccnc(NC(=O)[C@H]2O[C@@H](n3cc(-c4ccn(C)n4)c4c(N)ncnc43)[C@H](O)[C@@H]2O)c1] | I-172 | 8 | 451 | |
| 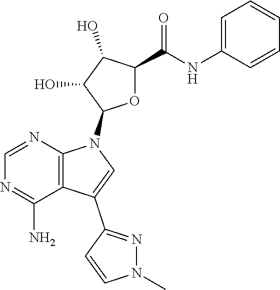 [SMILES: Cn1ccc(-c2cn([C@@H]3O[C@H](C(=O)Nc4ccccc4)[C@@H](O)[C@H]3O)c3ncnc(N)c23)n1] | I-173 | 8 | 436 | ¹H NMR (400 MHz, DMSO- d6) δ 10.39 (s, 1H), 9.07 (s, 1H), 8.05 (s, 1H), 7.98 (s, 1H), 7.76 (d, J = 2.2 Hz, 1H), 7.62 (d, J = 7.7 Hz, 2H), 7.36 (t, J = 7.9 Hz, 2H), 7.32-7.26 (m, 1H), 7.12 (t, J = 7.4 Hz, 1H), 6.56 (d, J = 2.2 Hz, 1H), 6.14 (d, J = 7.0 Hz, 1H), 5.72 (d, J = 4.6 Hz, 1H), 5.55 (d, J = 6.4 Hz, 1H), 4.70-4.60 (m, 1H), 4.50 (d, J = 2.2 Hz, 1H), 4.37-4.19 (m, 1H), 3.89 (s, 3H). |
| 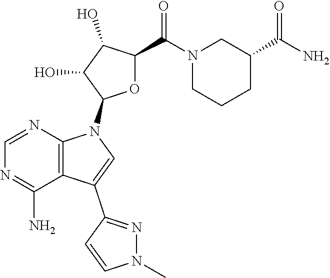 [SMILES: Cn1ccc(-c2cn([C@@H]3O[C@H](C(=O)N4CCC[C@@H](C(N)=O)C4)[C@@H](O)[C@H]3O)c3ncnc(N)c23)n1] | I-174 | 8 | 471 | ¹H NMR (400 MHz, DMSO- d6) δ ppm 8.90 (s, 1H), 8.06- 8.06 (t, J = 1.6 Hz, 1H), 7.96- 7.94 (t, J = 7.2 Hz, 1H), 7.75 (s, 1H), 7.37 (m, 1H), 6.93 (s, 1H), 6.56-6.54 (m, 1H), 6.28-6.26 (m, 1H), 6.03 (s, 2H), 4.85- 4.84 (m, 1H), 4.39- 4.36 (m, 3H), 3.88 (s, 5H), 3.14- 3.11 (m, 1H), 3.00-2.97 (m, 1H), 2.69-2.49 (m, 4H). |
| 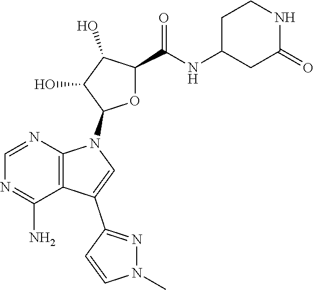 [SMILES: Cn1ccc(-c2cn([C@@H]3O[C@H](C(=O)NC4CCNC(=O)C4)[C@@H](O)[C@H]3O)c3ncnc(N)c23)n1] | I-175 | 9 | 457 | ¹H NMR (400 MHz, DMSO- d6) δ ppm 9.05 (s, 1H), 8.36 (d, J = 6.8 Hz, 1H), 8.07 (t, J = 2.4 Hz, 1H), 7.94 (t, J = 2.4 Hz, 1H), 7.75 (d, J = 2.4 Hz, 1H), 7.54 (s, 1H), 7.25 (s, 1H), 6.57- 6.53 (m, 1H), 6.09 (d, 7.2 Hz, 1H), 5.46 (d, J = 2.4 Hz, 1H), 4.62 (d, J = 2.4 Hz, 1H), 4.62- 4.30 (m, 1H), 4.27 (s, 1H), 4.17-4.13 (m, 1H), 4.02 (s, 1H), 3.86 (s, 3H), 3.22-3.13 (m, 2H), 2.44-2.33 (m, 2H), 1.92-1.63 (m, 2H). |
| 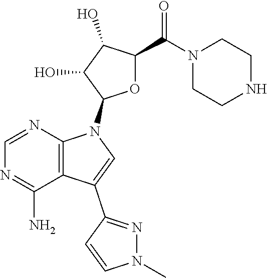 [SMILES: Cn1ccc(-c2cn([C@@H]3O[C@H](C(=O)N4CCNCC4)[C@@H](O)[C@H]3O)c3ncnc(N)c23)n1] | I-176 | 9 | 429 | ¹H NMR (400 MHz, DMSO- d6) δ 8.96 (s, 1H), 8.06 (s, 1H), 7.93 (s, 1H), 7.75 (d, J = 2.4 Hz, 1H), 7.20 (s, 1H), 6.52 (d, J = 2.0 Hz, 1H), 6.27 (d, J = 6.4 Hz, 1H), 5.66-5.56 (m, 1H), 5.54-5.44 (m, 1H), 4.75 (s, 1H), 4.50-4.35 (m, 1H), 4.35- 4.25 (m, 1H), 3.89 (s, 3H), 3.50-3.40 (m, 4H), 2.72-2.56 (m, 4H). |
| 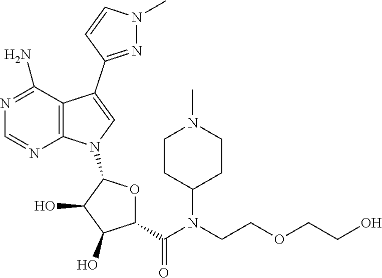 [SMILES: CN1CCC(N(CCOCCO)C(=O)[C@H]2O[C@@H](n3cc(-c4ccn(C)n4)c4c(N)ncnc43)[C@H](O)[C@@H]2O)CC1] | I-177 | 9 | 545 | ¹H NMR (400 MHz, DMSO- d6) δ 8.99-8.85 (br, 1H), 8.06 (s, 1H), 7.85 (d, J = 22.7 Hz, 1H), 7.77-7.75 (m, 1H), 7.29- 7.12 (br, 1H), 6.51 (dd, J = 12.3, 2.2 Hz, 1H), 6.27-6.25 (m, 1H), 5.79-5.54 (br, 1H), 4.84-4.77 (m, 1H), 4.51- 4.40 (m, 1H), 4.33-4.24 (m, 1H), 4.04-3.95 (m, 1H), 3.89 (s, 3H), 3.74-4.57 (m, 1H), 3.49-3.47 (m, 4H), 3.43-3.39 (m, 5H), 2.81-2.79 (m, 2H), 2.68-2.62 (m, 2H), 2.14- 2.11 (m, 5H), 1.92-1.86 (m, 2H). |
| 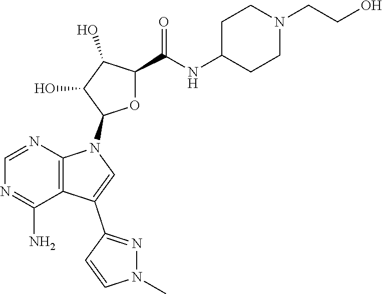 [SMILES: Cn1ccc(-c2cn([C@@H]3O[C@H](C(=O)NC4CCN(CCO)CC4)[C@@H](O)[C@H]3O)c3ncnc(N)c23)n1] | I-178 | 9 | 487 | ¹H NMR (400 MHz, DMSO- d6) δ 9.07 (s, 1H), 8.31 (d, J = 8.1 Hz, 1H), 8.09 (s, 1H), 7.92 (s, 1H), 7.75 (d, J = 2.2 Hz, 1H), 7.28 (s, 1H), 6.56 (d, J = 2.3 Hz, 1H), 6.04 (d, J = 7.5 Hz, 1H), 5.63 (s, 1H), 5.45 (d, J = 6.0 Hz, 1H), 4.70-4.55 (m, 1H), 4.35 (t, J = 5.4 Hz, 1H), 4.28 (d, J = 1.6 Hz, 1H), 4.14 (s, 1H), 3.89 (s, 3H), 3.70-3.55 (m, 1H), 3.50-3.40 (m, 2H), 2.88-2.78 (m, 2H), 2.36 (t, J = 6.3 Hz, 2H), 2.10-1.95 (m, 2H), 1.85-1.75 (m, 1H), 1.70-1.60 (m, 1H), 1.54-1.20 (m, 2H). |
| 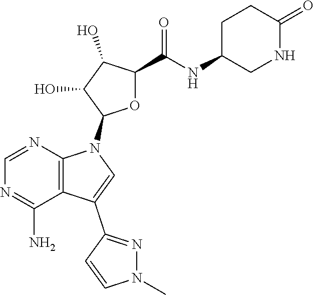 [SMILES: Cn1ccc(-c2cn([C@@H]3O[C@H](C(=O)N[C@H]4CCC(=O)NC4)[C@@H](O)[C@H]3O)c3ncnc(N)c23)n1] | I-179 | 9 | 457 | |
| 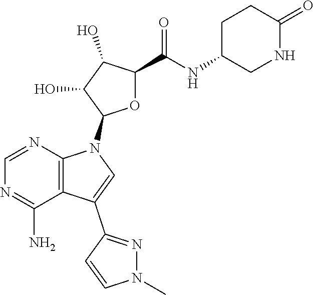 [SMILES: Cn1ccc(-c2cn([C@@H]3O[C@H](C(=O)N[C@@H]4CCC(=O)NC4)[C@@H](O)[C@H]3O)c3ncnc(N)c23)n1] | I-180 | 9 | 457 | ¹H NMR (400 MHz, DMSO- d6) δ ppm 9.19-8.99 (br, 1H), 8.46 (d, J = 7.2 Hz, 1H), 8.09 (s, 1H), 7.94 (s, 1H), 7.75 (s, 1H), 7.44 (s, 1H), 7.31-7.19 (br, 1H), 6.56 (s, 1H), 6.08 (d, J = 7.2 Hz, 1H), 5.62 (s, 1H), 5.45 (d, J = 6.4 Hz, 1H), 4.61- 4.57 (m, 1H), 4.30 (s, 1H), 4.17 (s, 1H), 4.03-3.99 (br, 1H), 3.89 (s, 3H), 3.26-3.23 (m, 1H), 3.05-3.00 (m, 1H), 2.25- 2.17 (m, 2H), 1.87-1.75 (m, 2H). |
| 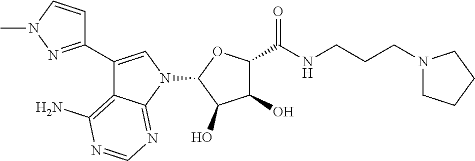 [SMILES: Cn1ccc(-c2cn([C@@H]3O[C@H](C(=O)NCCCN4CCCC4)[C@@H](O)[C@H]3O)c3ncnc(N)c23)n1] | I-181 | 9 | 471 | ¹H NMR (400 MHz, DMSO- d6) δ ppm 9.08-9.06 (br, 1H), 8.26 (t, J = 5.2 Hz, 1H), 8.10 (s, 1H), 7.89 (s, 1H), 7.75 (s, 1H), 7.37-7.25 (br, 1H), 6.59 (s, 1H), 6.00 (d, J = 7.2 Hz, 1H), 5.63 (s, 1H), 5.44 (d, J = 6.8 Hz, 1H), 4.65-4.60 (m, 1H), 4.26 (s, 1H), 4.14 (s, 1H), 3.89 (s, 3H), 4.28-3.16 (m, 2H), |
| | | | | 2.42-2.24 (m, 6H), 1.66-1.48 |
| | | | | (m, 6H). |
| 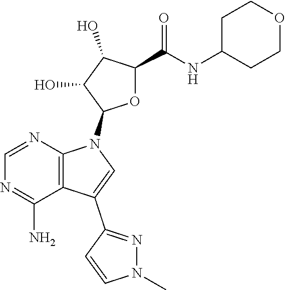 [SMILES: Cn1ccc(-c2cn([C@@H]3O[C@H](C(=O)NC4CCOCC4)[C@@H](O)[C@H]3O)c3ncnc(N)c23)n1] | I-182 | 9 | 444 | |
| 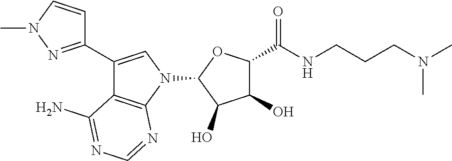 | I-183 | 9 | 445 | ¹H NMR (400 MHz, DMSO- d6) δ ppm 6.86 (s, 1H), 6.56 (s, 1H), 6.19 (s, 1H), 6.01 (d, J = 1.6 Hz, 1H), 5.04 (d, J = 2 Hz, 1H), 4.49 (d, J = 7.6 Hz, 1H), 3.24-3.21 (m, 1H), 2.92 (s, 1H), 2.81 (d, J = 6.8 Hz, 1H), 2.37 (s, 3H), 1.91-1.81 (m, 2H), 1.56 (t, J = 7.6 Hz, 2H), 1.32 (s, 6H), 0.41-0.39 (m, |
| | | | | 2H). |
| 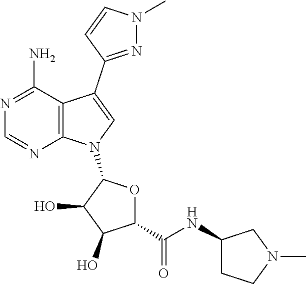 [SMILES: CN1CC[C@@H](NC(=O)[C@H]2O[C@@H](n3cc(-c4ccn(C)n4)c4c(N)ncnc43)[C@H](O)[C@@H]2O)C1] | I-184 | 9 | 443 | ¹H NMR (400 MHz, DMSO- d6) δ 9.11-9.01 (br, 1H), 8.61 (d, J = 7.8 Hz, 1H), 8.15 (s, 1H), 7.91 (s, 1H), 7.76 (d, J = 2.2 Hz, 1H), 7.32-7.24 (br, 1H), 6.59 (d, J = 2.3 Hz, 1H), 6.02 (d, J = 7.5 Hz, 1H), 5.64 (d, J = 4.4 Hz, 1H), 5.45 (d, J = 6.6 Hz, 1H), 4.64 (dd, J = 11.9, 6.9 Hz, 1H), 4.32-4.28 (m, 2H), 4.15-4.13 (m, 1H), 3.89 (s, 1H), 3.31-3.29 (m, 2H), 2.67-2.54 (m, 2H), 2.21 (s, 3H), 2.15-2.10 (m, 1H), 1.56- 1.49 (m, 1H). |
| 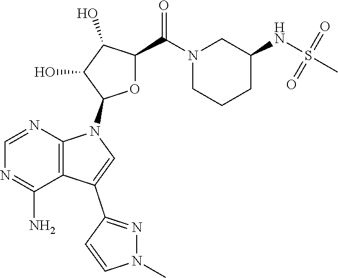 [SMILES: Cn1ccc(-c2cn([C@@H]3O[C@H](C(=O)N4CCC[C@H](NS(C)(=O)=O)C4)[C@@H](O)[C@H]3O)c3ncnc(N)c23)n1] | I-185 | 9 | 521 | ¹H NMR (400 MHz, DMSO- d6) δ 8.96 (s, 1H), 8.06 (s, 1H), 7.93 (d, J = 53.8 Hz, 1H), 7.75 (d, J = 2.2 Hz, 1H), 7.28-7.22 (m, 2H), 6.55-6.53 (m, 1H), 6.25 (d, J = 6.7 Hz, 2H), 5.63 (d, J = 4.9 Hz, 1H), 5.55-5.44 (m, 1H), 4.75 (s, 1H), 4.43- 4.35 (m, 1H), 4.28 (s, 1H), 3.98 (s, 0.5H), 3.89 (s, 3H), 3.83 (s, 0.6H), 3.74-3.71 (m, 0.6H), 3.22-3.20 (m, 0.6H), 3.09-3.02 (m, 1.4H), 2.95 (s, 1.6H), 2.85- 2.83 (m, 0.4H), 2.79 (s, 1.4H), 2.73-2.62 (m, 0.6H), 1.96-1.93 (m, 1H), 1.81-1.65 (m, 1H), 1.44-1.42 (m, 2H). |
| 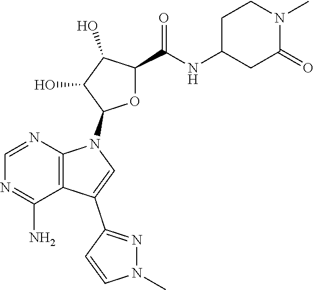 [SMILES: CN1CCC(NC(=O)[C@H]2O[C@@H](n3cc(-c4ccn(C)n4)c4c(N)ncnc43)[C@H](O)[C@@H]2O)CC1=O] | I-186 | 9 | 471 | ¹H NMR (400 MHz, DMSO- d6) δ ppm 8.10 (d, J = 8.4 Hz, 1H), 7.81 (d, J = 8.4 Hz, 1H), 7.59 (t, J = 2 Hz, 1H), 6.62- 6.61 (m, 1H), 6.17-6.12 (m, 1H), 4.86-4.77 (m, 1H), 4.45 (d, J = 2.4 Hz, 1H), 4.42-4.37 (m, 1H), 4.26 (s, 1H), 3.95 (s, 3H), 3.44-3.88 (m, 2H), 2.94 (d, J = 4.8 Hz, 3H), 2.7-2.33 (m, 2H), 2.12-1.89 (m, 2H). |
| 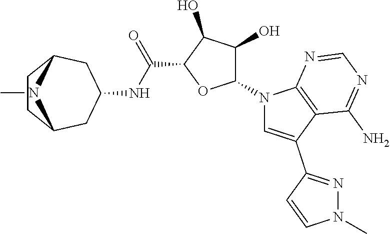 [SMILES: CN1[C@@H]2CC[C@H]1C[C@@H](NC(=O)[C@H]1O[C@@H](n3cc(-c4ccn(C)n4)c4c(N)ncnc43)[C@H](O)[C@@H]1O)C2] | I-187 | 9 | 483 | ¹H NMR (400 MHz, DMSO- d6) δ 9.03 (s, 1H), 8.25 (s, 2H), 8.07 (s, 1H), 7.99 (s, 1H), 7.80- 7.72 (m, 2H), 7.25 (s, 1H), 6.56 (d, J = 2.0 Hz, 1H), 6.15 (d, J = 6.8 Hz, 1H), 4.66-4.54 (m, 1H), 4.35 (s, 1H), 4.22 (s, 1H), 3.89 (s, 3H), 3.84 (s, 1H), 3.29 (s, 1H), 2.32 (s, 3H), 2.17-2.09 (m, 2H), 1.90 (s, 2H), 1.83- 1.63 (m, 5H). |
| 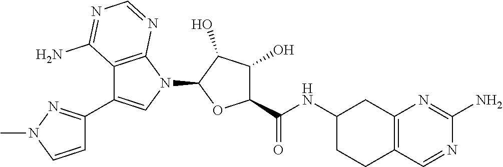 [SMILES: Cn1ccc(-c2cn([C@@H]3O[C@H](C(=O)NC4CCc5cnc(N)nc5C4)[C@@H](O)[C@H]3O)c3ncnc(N)c23)n1] | I-188 | 9 | 507 | ¹H NMR (400 MHz, DMSO- d6) δ 9.06 (s, 1H), 8.50-8.30 (m, 1H), 8.07 (d, J = 6.6 Hz, 1H), 7.96 (d, J = 3.0 Hz, 1H), 7.95-7.87 (m, 1H), 7.80-7.72 (m, 1H), 7.29 (s, 1H), 6.70- 6.55 (m, 1H), 6.30 (s, 2H), 6.09 (t, J = 8.0 Hz, 1H), 5.70-5.58 (m, 1H), 5.54-5.42 (m, 1H), 4.70-4.60 (m, 1H), 4.32 (s, 1H), 4.25-4.15 (m, 1H), 4.10- |
| | | | | 3.95 (m, 1H), 3.89 (s, 3H), |
| | | | | 2.90-2.80 (m, 1H), 2.80-2.70 |
| | | | | (m, 2H), 2.40-2.35 (m, 1H), |
| | | | | 2.11-1.53 (m, 3H). |
| 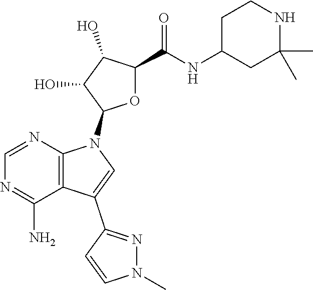 [SMILES: Cn1ccc(-c2cn([C@@H]3O[C@H](C(=O)NC4CCNC(C)(C)C4)[C@@H](O)[C@H]3O)c3ncnc(N)c23)n1] | I-189 | 9 | 471 | |
| 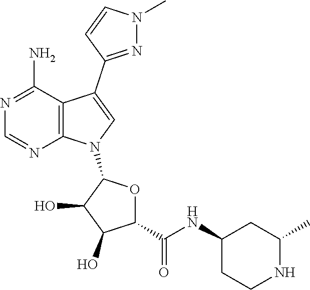 [SMILES: C[C@H]1C[C@H](NC(=O)[C@H]2O[C@@H](n3cc(-c4ccn(C)n4)c4c(N)ncnc43)[C@H](O)[C@@H]2O)CCN1] | I-190 | 9 | 457 | ¹H NMR (400 MHz, DMSO- d6) δ ppm 9.02 (s, 1H), 8.08 (s, 1H), 7.98 (s, 1H), 7.83 (d, J = 6.4 Hz, 1H), 7.75 (d, J = 2.4 Hz, 1H), 7.25 (s, 1H), 6.57 (s, J = 2.4 Hz, 1H), 6.12 (d, J = 7.2 Hz, 1H), 5.59 (d, J = 4.4 Hz, 1H), 5.47 (d, J = 6.8 Hz, 1H), 4.67-4.62 (m, 1H), 5.35 (s, 1H), 4.18 (s, 1H), 4.02 (s, 1H), 3.88 (s, 3H), 2.70-2.61 (m, 3H), 1.61-1.24 (m, 4H), 0.86 (d, J = 6 Hz, 3H). |
| 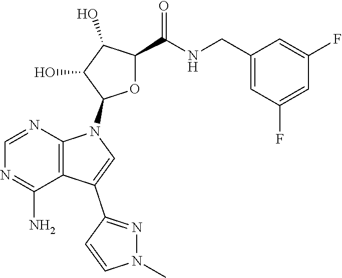 [SMILES: Cn1ccc(-c2cn([C@@H]3O[C@H](C(=O)NCc4cc(F)cc(F)c4)[C@@H](O)[C@H]3O)c3ncnc(N)c23)n1] | I-191 | 9 | 486 | ¹H NMR (400 MHz, DMSO- d6) δ 9.59 (t, J = 6.0 Hz, 1H), 9.08 (s, 1H), 7.87 (d, J = 3.9 Hz, 2H), 7.74 (d, J = 2.3 Hz, 1H), 7.31 (s, 1H), 7.13-7.06 (m, 1H), 7.02 (d, J = 6.2 Hz, 2H), 6.53 (d, J = 2.3 Hz, 1H), 5.98 (d, J = 7.6 Hz, 1H), 5.70 (d, J = 4.4 Hz, 1H), 5.49 (d, J = 6.6 Hz, 1H), 4.68-4.57 (m, 1H), 4.44 (d, J = 6.1 Hz, 2H), 4.38 (d, J = 1.7 Hz, 1H), 4.20-4.17 (m, 1H), 3.88 (s, 3H). |
| 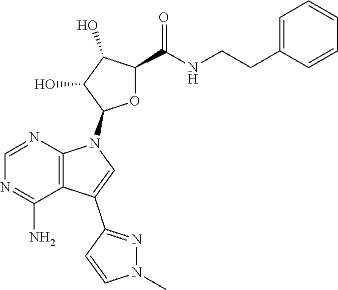 [SMILES: Cn1ccc(-c2cn([C@@H]3O[C@H](C(=O)NCCc4ccccc4)[C@@H](O)[C@H]3O)c3ncnc(N)c23)n1] | I-192 | 9 | 464 | ¹H NMR (400 MHz, DMSO- d6) δ ppm 9.07 (s, 1H), 8.88 (d, J = 5.6 Hz, 1H), 7.99 (s, 1H), 7.88 (s, 1H), 7.95 (d, J = 2.4 Hz, 1H), 7.29-7.13 (m, 6H), 6.58 (d, J = 2 Hz, 1H), 5.95 (d, J = 7.6 Hz, 1H), 5.63 (d, J = 4.4 Hz, 1H), 5.42 (d, J = 9.2 Hz, 1H), 4.60-4.56 (m, 1H), 4.25 (s, 1H), 4.08-4.05 (m, 1H), 3.88 (s, 3H), 3.50-3.34 (m, 2H), 2.77 (t, J = 7.2 Hz, 2H). |
| 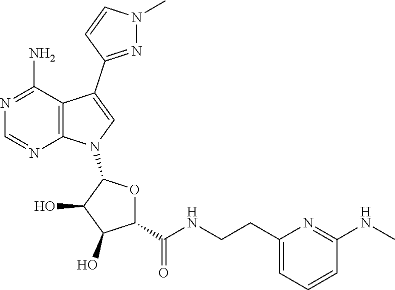 [SMILES: CNc1cccc(CCNC(=O)[C@H]2O[C@@H](n3cc(-c4ccn(C)n4)c4c(N)ncnc43)[C@H](O)[C@@H]2O)n1] | I-193 | 9 | 494 | ¹H NMR (400 MHz, DMSO- d6) δ ppm 9.06 (br, 1H), 8.85- 8.82 (m, 1H), 8.04 (s, 1H), 7.89 (s, 1H), 7.74 (d, J = 2.4 Hz, 1H), 7.29 (br, 1H), 7.23-7.19 (m, 1H), 6.58 (d, J = 2.0 Hz, 1H), 6.29 (d, J = 6.0 Hz, 1H), 6.26-6.22 (m, 1H), 6.19 (d, J = 8.4 Hz, 1H), 5.63 (d, J = 4.4 Hz, 1H), 5.41 (d, J = 6.8 Hz, 1H), 4.62-4.57 (m, 1H), 4.26 (d, J = 1.2 Hz, 1H), 4.15-4.13 (m, 1H), 3.88 (s, 3H), 3.55- 3.50 (m, 1H), 3.48-3.41 (m, 1H), 2.72-2.68 (m, 1H), 2.65 (d, J = 4.8 Hz, 3H) |
| 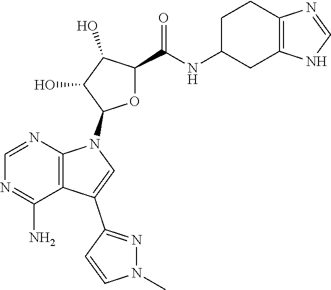 [SMILES: Cn1ccc(-c2cn([C@@H]3O[C@H](C(=O)NC4CCc5nc[nH]c5C4)[C@@H](O)[C@H]3O)c3ncnc(N)c23)n1] | I-194 | 9 | 480 | ¹H NMR (400 MHz, DMSO- d6) δ 9.06 (s, 1H), 8.52-8.36 (m, 1H), 8.02 (d, J = 7.0 Hz, 1H), 7.95 (d, J = 6.4 Hz, 1H), 7.80-7.75 (m, 1H), 7.42 (d, J = 3.3 Hz, 1H), 7.27 (s, 1H), 6.60- 6.55 (m, 1H), 6.07 (d, J = 7.3 Hz, 1H), 4.75-4.55 (m, 2H), 4.40-4.28 (m, 1H), 4.23-4.15 (m, 2H), 4.16-4.00 (s, 2H), 3.89 (s, 3H), 2.91-2.75 (m, 1H), 2.60-2.55 (m, 1H), 2.44- 2.31 (m, 1H), 2.03-1.85 (m, 1H), 1.81-1.71 (m, 1H). |
| 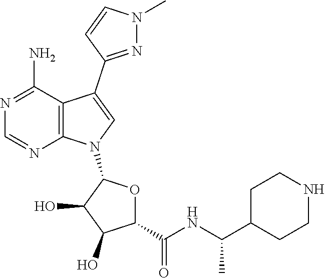 [SMILES: C[C@H](NC(=O)[C@H]1O[C@@H](n2cc(-c3ccn(C)n3)c3c(N)ncnc32)[C@H](O)[C@@H]1O)C1CCNCC1] | I-195 | 9 | 471 | ¹H NMR (400 MHz, DMSO- d6) δ 9.06 (s, 1H), 8.21 (d, J = 9.2 Hz, 1H), 8.08 (s, 1H), 7.94 (s, 1H), 7.75 (d, J = 2.4 Hz, 1H), 7.27 (s, 1H), 6.55 (d, J = 6.4 Hz, 1H), 6.02 (d, J = 6.8 Hz, 1H), 5.65 (s, 1H), 5.46 (s, 1H), 4.63 (s, 1H), 4.30 (s, 1H), 4.12 (d, J = 3.2 Hz, 1H), 3.89 (s, 3H), 3.72-3.15 (m, 1H), 2.91-2.83 (m, 2H), 2.43-2.27 (m, 2H), 1.51 (t, J = 9.2 Hz, 2H), 1.34 (s, 1H), 1.07 (d, J = 6.8 Hz, 3H), 1.06-0.89 (m, 2H). |
| 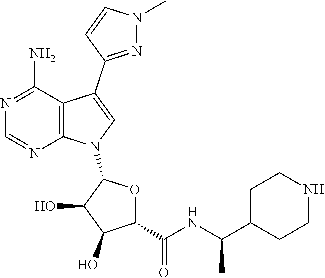 [SMILES: C[C@@H](NC(=O)[C@H]1O[C@@H](n2cc(-c3ccn(C)n3)c3c(N)ncnc32)[C@H](O)[C@@H]1O)C1CCNCC1] | I-196 | 9 | 471 | ¹H NMR (400 MHz, DMSO- d6) δ ppm 8.41 (s, 1H), 8.12 (s, 1H), 7.80 (s, 1H), 7.59 (s, 1H), 6.61 (d, J = 1.2 Hz, 1H), 6.11 (d, J = 6.4 Hz, 1H), 4.83- 4.80 (m, 2H), 4.47 (s, 1H), 4.40 (d, J = 2.4 Hz, 1H), 3.92 (s, 3H), 3.92-3.90 (m, 1H), 3.38- 3.34 (m, 1H), 3.33 (s, 1H), 2.98-2.86 (m, 1H), 2.01-1.92 (m, 2H), 1.76-1.74 (m, 2H), 1.54-1.45 (m, 2H), 1.20 (d, J = 6.4 Hz, 2H). |
| 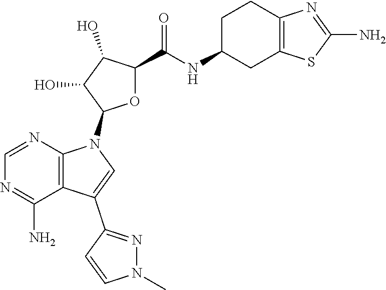 [SMILES: Cn1ccc(-c2cn([C@@H]3O[C@H](C(=O)N[C@H]4CCc5nc(N)sc5C4)[C@@H](O)[C@H]3O)c3ncnc(N)c23)n1] | I-197 | 9 | 512 | |
| 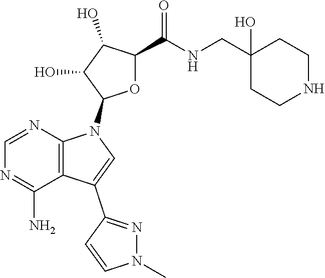 [SMILES: Cn1ccc(-c2cn([C@@H]3O[C@H](C(=O)NCC4(O)CCNCC4)[C@@H](O)[C@H]3O)c3ncnc(N)c23)n1] | I-198 | 9 | 473 | ¹H NMR (400 MHz, DMSO- d6) δ ppm 9.06 (s, 1H), 8.49 (s, 1H), 8.14 (s, 1H), 7.89 (s, 1H), 7.75 (d, J = 2.4 Hz, 1H), 7.27 (s, 1H), 6.60 (d, J = 2.4 Hz, 1H), 6.04 (d, J = 7.6 Hz, 1H), 5.68 (s, 1H), 5.48 (s, 1H), 4.59 (t, J = 7.2 Hz, 1H), 4.41 (s, 1H), 4.34 (s, 1H), 4.14 (d, J = 4.4 Hz, 1H), 3.87 (s, 3H), 3.16 (d, J = 5.6 Hz, 1H), 2.74-2.64 (m, 3H), 2.50-2.32 (m, 2H), 1.37-1.32 (m, 4H) |
| 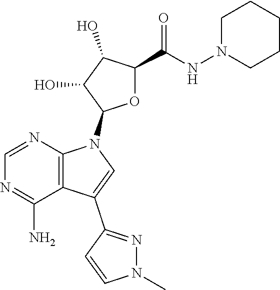 [SMILES: Cn1ccc(-c2cn([C@@H]3O[C@H](C(=O)NN4CCCCC4)[C@@H](O)[C@H]3O)c3ncnc(N)c23)n1] | I-199 | 9 | 443 | ¹H NMR (400 MHz, DMSO- d6) δ 9.38 (s, 1H), 9.06 (s, 1H), 8.10 (s, 1H), 7.95 (s, 1H), 7.76 (d, J = 2.2 Hz, 1H), 6.56 (d, J = 2.2 Hz, 1H), 6.06 (d, J = 7.3 Hz, 1H), 5.62 (d, J = 4.5 Hz, 1H), 5.47 (d, J = 6.6 Hz, 1H), 4.61 (d, J = 5.1 Hz, 1H), 4.23 (s, 1H), 4.15 (s, 1H), 3.89 (s, 3H), 2.70-2.67 (m, 4H), 1.59- 1.55 (m, 5H), 1.35 (s, 2H). |
| 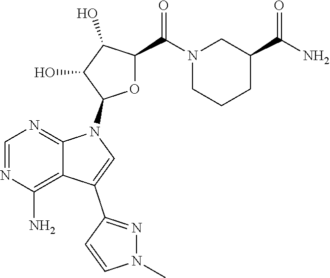 [SMILES: Cn1ccc(-c2cn([C@@H]3O[C@H](C(=O)N4CCC[C@H](C(N)=O)C4)[C@@H](O)[C@H]3O)c3ncnc(N)c23)n1] | I-200 | 9 | 471 | ¹H NMR (400 MHz, DMSO- d6) δ 8.95 (br, 1H), 8.06 (s, 1H), 7.99 (d, J = 2.8 Hz, 1H), 7.76-7.74 (m, 1H), 7.38-7.31 (m, 1H), 7.18 (br, 1H), 6.89 (s, 1H), 6.54-6.50 (m, 1H), 6.28 (t, J = 6 Hz, 1H), 5.61-5.59 (m, 1H), 5.50-5.46 (m, 1H), 4.49- 4.47 (m, 1H), 4.37-4.33 (m, 1H), 4.29-4.24 (m, 1H), 3.89-3.88 (m, 5H), 3.17-2.6 (m, 2H), 2.22-2.20 (m, 1H), 1.89- 1.30 (m, 4H). |
| 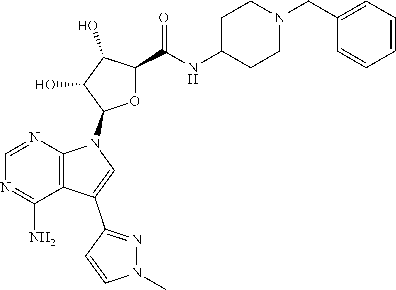 [SMILES: Cn1ccc(-c2cn([C@@H]3O[C@H](C(=O)NC4CCN(Cc5ccccc5)CC4)[C@@H](O)[C@H]3O)c3ncnc(N)c23)n1] | I-201 | 9 | 533 | ¹H NMR (400 MHz, DMSO- d6) δ ppm 9.60 (s, 1H), 8.33- 8.31 (t, J = 8 Hz, 1H, 1H), 8.10 (s, 1H), 7.92 (s, 1H), 7.74 (s, 1H), 7.34-7.24 (m, 6H), 6.56 (s, 1H), 6.04-6.02 (t, J = 7.6 Hz, 1H), 5.63-5.62 (m, 1H), 5.46-5.742 (m, 1H), 4.29-4.28 (m, 1H), 4.27 (s, 1H), 4.14 (s, 1H), 4.13 (m, 3H), 3.44 (s, 1H), 3.42 (m, 2H), 2.75 (s, 2H), 2.49- 2.49 (m, 2H), 2.01-2.00 (m, 2H), 1.99-1.98 (m, 2H). |
| 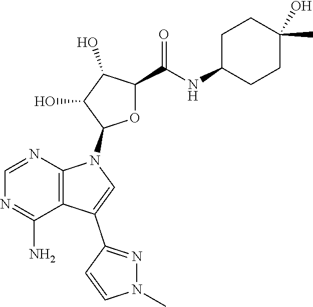 [SMILES: Cn1ccc(-c2cn([C@@H]3O[C@H](C(=O)N[C@H]4CC[C@](C)(O)CC4)[C@@H](O)[C@H]3O)c3ncnc(N)c23)n1] | I-202 | 9 | 474 | ¹H NMR (400 MHz, DMSO- d6) δ ppm 9.10 (s, 1H), 8.67- 8.65 (t, J = 8.4 Hz, 1H), 8.25 (s, 1H), 8.13 (s, 1H), 7.89 (s, 1H), 7.75 (s, 1H), 7.30 (s, 1H), 6.57 (s, 1H), 5.92- 5.90 (m, 1H), 5.66- 5.65 (m, 1H), 5.43- 5.42 (m, 1H), 4.69-4.67 (m, 1H), 4.26 (s, 1H), 4.15 (s, 1H), 4.08-4.07 (m, 1H), 4.056 (s, 3H), 3.89- 3.63 (m, 1H), 1.72-1.62 (m, 11H). |
| 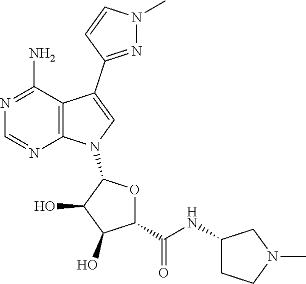 [SMILES: CN1CC[C@H](NC(=O)[C@H]2O[C@@H](n3cc(-c4ccn(C)n4)c4c(N)ncnc43)[C@H](O)[C@@H]2O)C1] | I-203 | 12 | 443 | ¹H NMR (400 MHz, DMSO- d6) δ 9.13-9.02 (br, 1H), 8.65 (d, J = 7.6 Hz, 1H), 8.14 (s, 1H), 7.91 (s, 1H), 7.76 (d, J = 2.2 Hz, 1H), 7.33-7.24 (br, 1H), 6.57 (d, J = 2.3 Hz, 1H), 6.00 (d, J = 7.6 Hz, 1H), 5.63 (d, J = 4.5 Hz, 1H), 5.45 (d, J = 6.6 Hz, 1H), 4.66- 4.60 (m, 1H), 4.34-4.28 (m, 2H), 4.14-4.11 (m, 1H), 3.89 (s, 3H), 3.33- 3.28 (m, 2H), 2.69-2.63 (m, 1H), 2.39-2.28 (m, 2H), 2.21 (s, 3H), 1.65-1.56 (m, 1H). |
| 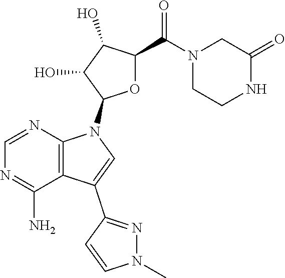 [SMILES: Cn1ccc(-c2cn([C@@H]3O[C@H](C(=O)N4CCNC(=O)C4)[C@@H](O)[C@H]3O)c3ncnc(N)c23)n1] | I-204 | 12 | 443 | ¹H NMR (400 MHz, DMSO- d6) δ 9.02-8.90 (br, 1H), 8.16- 8.11 (m, 1H), 8.06-8.05 (m, 2H), 7.98 (s, 1H), 7.91 (s, 1H), 7.75 (d, J = 2.2 Hz, 1H), 7.24- 7.12 (br, 1H), 6.55-6.53 (m, 1H), 6.27-6.25 (m, 1H), 4.81- 4.76 (m, 1H), 4.41-4.31 (m, 2H), 4.11 (s, 1H), 3.99 (d, J = 2.2 Hz, 1H), 3.89 (s, 3H), 3.69- 3.65 (m, 2H), 3.20-3.16 (m, 2H). |
| 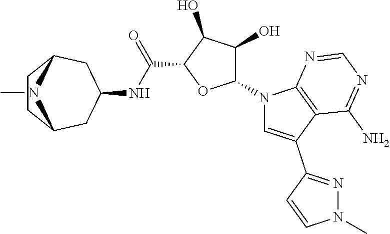 [SMILES: CN1[C@@H]2CC[C@H]1C[C@H](NC(=O)[C@H]1O[C@@H](n3cc(-c4ccn(C)n4)c4c(N)ncnc43)[C@H](O)[C@@H]1O)C2] | I-205 | 12 | 483 | ¹H NMR (400 MHz, DMSO- d6) δ 9.10 (s, 1H), 8.49 (d, J = 8.6 Hz, 1H), 8.18 (s, 1H), 7.88 (s, 1H), 7.76 (d, J = 2.3 Hz, 1H), 7.30 (s, 1H), 6.57 (d, J = 2.3 Hz, 1H), 5.97 (d, J = 7.6 Hz, 1H), 5.57 (s, 1H), 4.64- 4.61 (m, J = 7.6, 4.6 Hz, 1H), 4.24 (d, J = 1.3 Hz, 1H), 4.10 (d, J = 4.6 Hz, 1H), 3.98 (s, 1H), 3.89 (s, 3H), 3.07 (s, 2H), 2.17 (s, 3H), 1.96 (s, 2H), 1.65- 1.51 (m, J = 18.5, 14.5 Hz, 6H). |
| 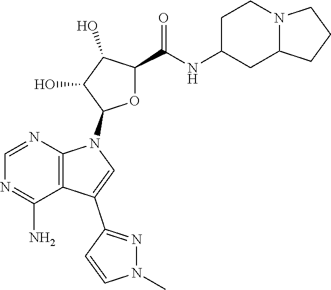 [SMILES: Cn1ccc(-c2cn([C@@H]3O[C@H](C(=O)NC4CCN5CCCC5C4)[C@@H](O)[C@H]3O)c3ncnc(N)c23)n1] | I-206 | 12 | 483 | |
| 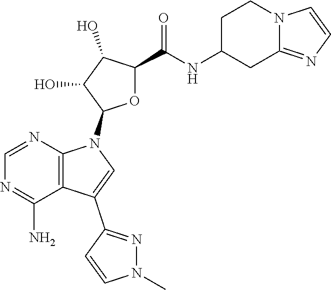 [SMILES: Cn1ccc(-c2cn([C@@H]3O[C@H](C(=O)NC4CCn5ccnc5C4)[C@@H](O)[C@H]3O)c3ncnc(N)c23)n1] | I-207 | 12 | 480.2 | ¹H NMR (400 MHz, MeOD) δ 6.76 (d, J = 12.6 Hz, 1H), 6.70 (s, 1H), 6.10 (s, 1H), 5.94- 5.88 (m, 2H), 5.14 (m, 1H), 4.83-4.82 (m, 1H), 3.16-3.11 (m, 1H), 3.06-2.96 (m, 2H), 2.91-2.87 (m, 1H), 2.81- 2.69 (m, 2H), 2.00-1.90 (m, 1H), 1.77 (s, 3H), 1.56-1.45 (dt, J = 17.3, 7.2 Hz, 1H), 0.84- 0.64 (m, 2H). |
| 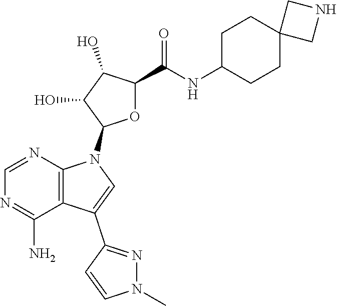 [SMILES: Cn1ccc(-c2cn([C@@H]3O[C@H](C(=O)NC4CCC5(CC4)CNC5)[C@@H](O)[C@H]3O)c3ncnc(N)c23)n1] | I-208 | 12 | 483 | ¹H NMR (400 MHz, DMSO- d6) δ ppm 9.07 (br, 1H), 8.19- 8.17 (m, 1H), 8.06-8.03 (m, 1H), 7.94 (s, 1H), 7.76 (d, J = 2 Hz, 1H), 7.29 (br, 1H), 6.56 (d, J = 2.4 Hz, 1H), 6.01 (d, J = 7.6 Hz, 2H), 5.68 (s, 1H), 4.62 (s, 1H), 4.26 (s, 1H), 4.13 (s, 1H), 3.88 (s, 3H), 3.62-3.56 (m, 1H), 3.23-3.15 (m, 4H), 1.92-1.88 (m, 1H), 1.80-1.60 (m, 4H), 1.46-1.21 (m, 4H). |
| 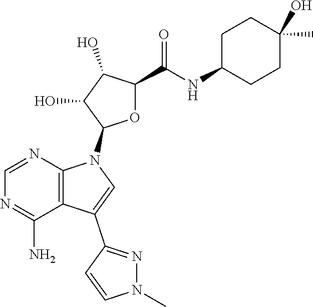 [SMILES: Cn1ccc(-c2cn([C@@H]3O[C@H](C(=O)N[C@H]4CC[C@@](C)(O)CC4)[C@@H](O)[C@H]3O)c3ncnc(N)c23)n1] | I-209 | 12 | 472 | ¹H NMR (400 MHz, DMSO- d6) δ ppm 9.11 (br, 1H), 8.655 (d, J = 8.4 Hz, 1H), 8.29 (s, 1H), 7.89 (s, 1H), 7.75 (d, J = 2 Hz, 1H), 7.31 (br, 1H), 6.57 (s, 1H), 5.91 (d, J = 7.6 Hz, 1H), 5.66 (d, J = 4 Hz, 1H), 5.43 (d, J = 6.8 Hz, 1H), 4.71-4.67 (m, 1H), 4.26 (s, 1H), 4.15 (s, 1H), 4.09-4.07 (m, 1H), 3.89 (s, 3H), 3.63 (br, 1H), 1.70-1.32 (m, 8H), 1.11 (s, 3H). |
| 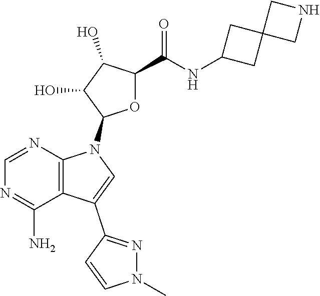 [SMILES: Cn1ccc(-c2cn([C@@H]3O[C@H](C(=O)NC4CC5(CNC5)C4)[C@@H](O)[C@H]3O)c3ncnc(N)c23)n1] | I-210 | 12 | 455 | ¹H NMR (400 MHz, DMSO- d6) δ ppm 9.08 (brs, 1H), 8.66 (d, J = 7.6 Hz, 1H), 8.06 (s, 1H), 7.92 (s, 1H), 7.76 (d, J = 2.4 Hz, 1H), 7.30 (brs, 1H), 6.56 (d, J = 2.0 Hz, 1H), 6.02 (d, J = 7.2 Hz, 1H), 5.63 (brs, 2H), 4.60-4.57 (m, 1H), 4.23 (d, J = 1.6 Hz, 1H), 4.13-4.12 (m, 2H), 3.89 (s, 3H), 3.65- 3.51 (m, 4H), 2.47-2.38 (m, 2H), 2.07-1.96 (m, 2H). |
| 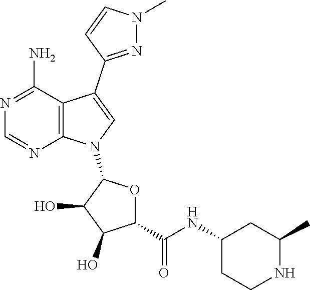 [SMILES: C[C@@H]1C[C@@H](NC(=O)[C@H]2O[C@@H](n3cc(-c4ccn(C)n4)c4c(N)ncnc43)[C@H](O)[C@@H]2O)CCN1] | I-211 | 12 | 457 | ¹H NMR (400 MHz, DMSO- d6) δ ppm 9.04 (s, 1H), 8.08 (s, 1H), 7.97 (s, 1H), 7.91 (d, J = 6 Hz, 1H), 7.76 (d, J = 2 Hz, 1H), 7.24 (s, 1H), 6.55 (d, J = 2 Hz, 1H), 6.12 (d, J = 6.4 Hz, 1H), 5.61 (d, J = 4.4 Hz, 1H), 5.47 (d, J = 6.4 Hz, 1H), 4.66- 4.62 (m, 1H), 4.35 (d, J = 1.6 Hz, 1H), 4.17-4.16 (m, 1H), 4.05-4.03 (m, 1H), 3.89 (s, 3H), 2.79-2.65 (m, 2H), 1.58- 1.24 (m, 4H), 0.93 (d, J = 6.4 Hz, 3H). |
| 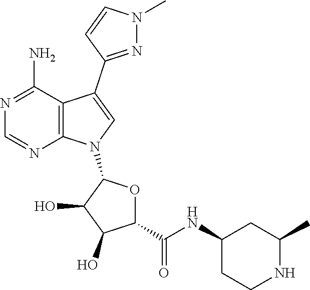 [SMILES: C[C@@H]1C[C@H](NC(=O)[C@H]2O[C@@H](n3cc(-c4ccn(C)n4)c4c(N)ncnc43)[C@H](O)[C@@H]2O)CCN1] | I-212 | 12 | 457 | ¹H NMR (400 MHz, DMSO- d6) δ 9.09 (s, 1H), 8.36 (d, J = 8.4 Hz, 1H), 8.12 (s, 1H), 7.90 (s, 1H), 7.76 (d, J = 2.2 Hz, 1H), 7.48-7.10 (m, 1H), 6.57 (d, J = 2.2 Hz, 1H), 6.00 (d, J = 7.6 Hz, 1H), 5.65 (d, J = 4.2 Hz, 1H), 5.45 (d, J = 6.6 Hz, 1H), 4.70-4.60 (m, 1H), 4.26 (s, 1H), 4.20-4.10 (m, 1H), 3.89 (s, 3H), 3.82-3.60 (m, 1H), 2.97-2.80 (m, 1H), 2.60- 2.50 (m, 2H), 1.79 (d, J = 12.1 Hz, 1H), 1.62 (d, J = 10.2 Hz, 1H), 1.25-1.15 (m, 1H), 0.98 (d, J = 6.3 Hz, 3H), 0.96-0.87 (m, 2H). |
| 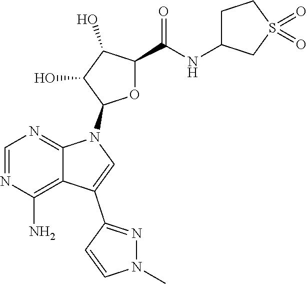 [SMILES: Cn1ccc(-c2cn([C@@H]3O[C@H](C(=O)NC4CCS(=O)(=O)C4)[C@@H](O)[C@H]3O)c3ncnc(N)c23)n1] | I-213 | 12 | 478 | ¹H NMR (400 MHz, DMSO- d6) δ ppm 9.04 (s, 1H), 8.69 (t, J = 6.8 Hz, 1H), 8.10 (d, J = 2.8 Hz, 1H), 7.91 (d, J = 6.8 Hz, 1H), 7.76-7.73 (m, 1H), 7.21 (s, 1H), 6.60 (dd, J = 15.6 Hz, 2.4 Hz, 1H), 6.13 (d, J = 7.6, 1H), 5.65 (d, J = 2.4 Hz, 1H), 5.49-5.34 (m, 1H), 4.59- 4.53 (m, 2H), 4.52 (s, 1H), 4.23 (s, 1H), 3.89 (s, 3H), 3.53-3.32 (m, 1H), 3.28-2.94 (m, 3H), 2.41-2.10 (m, 2H). |
| 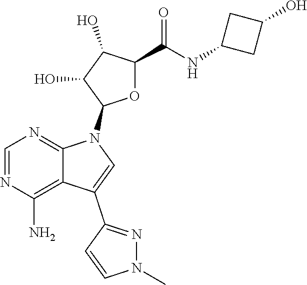 [SMILES: Cn1ccc(-c2cn([C@@H]3O[C@H](C(=O)N[C@H]4C[C@@H](O)C4)[C@@H](O)[C@H]3O)c3ncnc(N)c23)n1] | I-214 | 12 | 430 | ¹H NMR (400 MHz, DMSO- d6) δ 9.09 (s, 1H), 8.81 (d, J = 7.7 Hz, 1H), 8.21 (s, 1H), 7.91 (s, 1H), 7.76 (d, J = 2.3 Hz, 1H), 7.31 (s, 1H), 6.57 (d, J = 2.3 Hz, 1H), 5.97 (d, J = 7.5 Hz, 1H), 5.58 (s, 2H), 5.15 (s, 1H), 4.64-4.61 (m, J = 7.6, 4.7 Hz, 1H), 4.25 (d, J = 1.6 Hz, 1H), 4.10 (d, J = 4.9 Hz, 1H), 3.89 (s, 3H), 3.87-3.73 (m, 2H), 2.71-2.52 (m, 2H), 1.88-1.67 (m, 2H). |
| 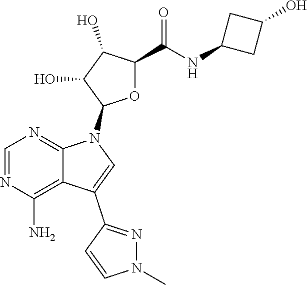 [SMILES: Cn1ccc(-c2cn([C@@H]3O[C@H](C(=O)N[C@H]4C[C@H](O)C4)[C@@H](O)[C@H]3O)c3ncnc(N)c23)n1] | I-215 | 12 | 430 | |
| 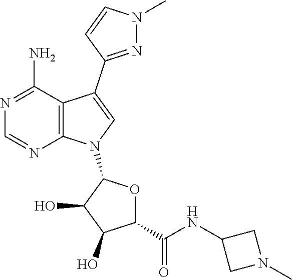 [SMILES: CN1CC(NC(=O)[C@H]2O[C@@H](n3cc(-c4ccn(C)n4)c4c(N)ncnc43)[C@H](O)[C@@H]2O)C1] | I-216 | 12 | 429.1 | |
| 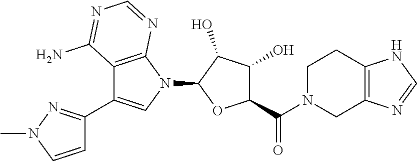 [SMILES: Cn1ccc(-c2cn([C@@H]3O[C@H](C(=O)N4CCc5[nH]cnc5C4)[C@@H](O)[C@H]3O)c3ncnc(N)c23)n1] | I-217 | 12 | 466 | ¹H NMR (400 MHz, DMSO- d6) δ 12.00-11.75 (m, 1H), 8.95 (s, 1H), 8.06 (d, J = 2.8 Hz, 1H), 7.95-7.80 (m, 1H), 7.80-7.60 (m, 1H), 7.60-7.40 (m, 1H), 7.20 (s, 1H), 6.70- 6.50 (m, 1H), 6.45-6.35 (m, 1H), 6.30-6.25 (m, 1H), 5.70- 5.55 (m, 1H), 5.54-5.40 (m, 1H), 4.90-4.85 (m, 1H), 4.60- |
| | | | | 4.45 (m, 2H), 4.44-4.35 (m, |
| | | | | 2H), 3.88 (d, J = 4.1 Hz, 3H), |
| | | | | 3.85-3.65 (m, 2H), 2.70-2.55 |
| | | | | (m, 2H). |
| 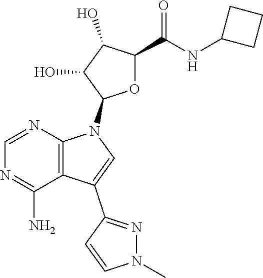 [SMILES: Cn1ccc(-c2cn([C@@H]3O[C@H](C(=O)NC4CCC4)[C@@H](O)[C@H]3O)c3ncnc(N)c23)n1] | I-218 | 12 | 414 | ¹H NMR (400 MHz, DMSO- d6) δ ppm 9.08 (br, 1H), 8.71 (d, J = 8.0 Hz, 1H), 8.10 (s, 1H), 7.92 (s, 1H), 7.75 (d, J = 4.8 Hz, 1H), 7.31 (br, 1H), 6.56 (d, J = 2.0 Hz, 1H), 6.02 (d, J = 7.6 Hz, 1H), 5.63 (d, J = 4.4 Hz, 1H), 5.46 (d, J = 6.8 Hz, 1H), 5.64-4.59 (m, 1H), 4.32-4.26 (m, 1 H), 4.24 (s, H), 4.21-4.11 (m, 1H), 3.89 (s, 3H) 2.26-2.16 (m, 2H). 1.71-1.57 (m, 2H). |
| 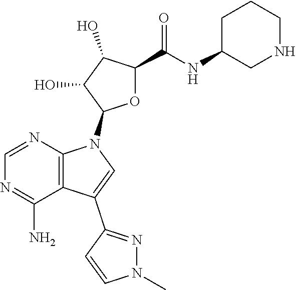 [SMILES: Cn1ccc(-c2cn([C@@H]3O[C@H](C(=O)N[C@H]4CCCNC4)[C@@H](O)[C@H]3O)c3ncnc(N)c23)n1] | I-219 | 12 | 443 | ¹H NMR (400 MHz, CD3OD) δ ppm 9.07 (brs, 1H), 8.12 (s, 1H), 8.10 (s, 1H), 7.92 (s ,1H), 7.75 (d, J = 2.0 Hz, 1H), 7.26 (brs, 1H), 6.57 (d, J = 2.4 Hz, 1H), 6.06 (d, J = 7.6 Hz, 1H), 4.63-4.62 (m, 1H), 4.28-4.13 (m, 4H), 3.97 (s, 3H), 2.91-2.84 (m, 2H), 2.42-2.35 (m, 2H), 2.91-2.84 (m, 2H), 1.89-1.65 (m, 4H). |
| 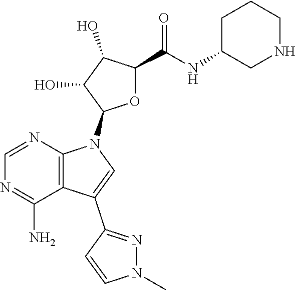 [SMILES: Cn1ccc(-c2cn([C@@H]3O[C@H](C(=O)N[C@@H]4CCCNC4)[C@@H](O)[C@H]3O)c3ncnc(N)c23)n1] | I-220 | 12 | 443 | ¹H NMR (400 MHz, CD3OD) δ ppm 9.07 (brs, 1H), 8.16-7.75 (m, 3H), 7.28 (s, 1H), 6.57 (s, 1H), 6.27-6.04 (m, 2H), 4.62- 4.59 (m, 1H), 4.28-4.13 (m, 4H), 3.89 (s, 1H), 3.68-3.66 (m, 1H), 3.02-2.84 (m, 2H), 2.94-2.32 (m, 2H), 1.75-1.49 (m, 4H). |
| 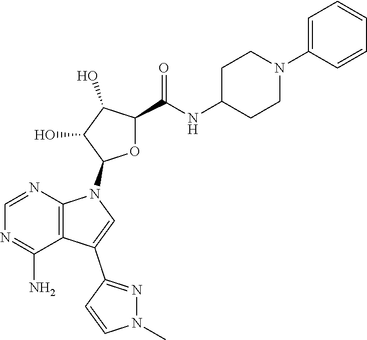 [SMILES: Cn1ccc(-c2cn([C@@H]3O[C@H](C(=O)NC4CCN(c5ccccc5)CC4)[C@@H](O)[C@H]3O)c3ncnc(N)c23)n1] | I-221 | 12 | 519 | ¹H NMR (400 MHz, DMSO- d6) δ ppm 9.06 (br, 1H), 8.39 (d, J = 7.6 Hz, 0.7H), 8.06 (s, 0.3H), 7.99 (s, 0.7H), 7.94-7.93 (m, 1H), 7.76-7.74 (m, 1H), 7.53 (d, J = 8.0 Hz, 0.3H), 7.31 (br, 0.7H), 7.21-7.16 (m, 2.3H), 6.95-6.91 (m, 2H), 6.77-6.73 (m, 1H), 6.63 (d, J = 2.0 Hz, 0.3H), 6.56 (d, J = 2.4 Hz, 0.7H), 6.26 (d, J = 7.2 Hz, 0.3H), 6.04 (d, J = 7.6 Hz, 0.7H), 5.65 (d, J = 4.4 Hz, 0.7H), 5.47-5.44 (m, 1H), 5.37 (d, J = 5.2 Hz, 0.3H), 4.87 (d, J = 2.8 Hz, 0.3H), 4.78- 4.71 (s, 0.3 H), 4.66-4.61 (s, 0.7H), 4.30-4.29 (m, 1H), 4.17- 4.14 (m, J = 0.7H), 3.89 (s, 3H), 3.86-3.83 (m, 1H), 3.72- 3.63 (m, 2H), 3.28-3.27 (m, 2H) |
| | | | | 1.89-1.77 (m, 1.7H), 1.59-1.53 |
| | | | | (m, 2.3H). |
| 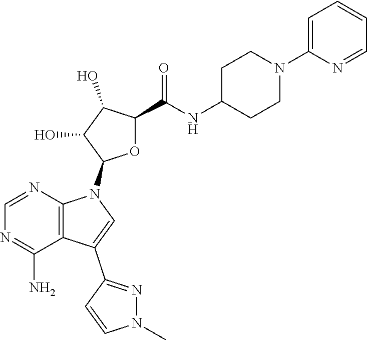 [SMILES: Cn1ccc(-c2cn([C@@H]3O[C@H](C(=O)NC4CCN(c5ccccn5)CC4)[C@@H](O)[C@H]3O)c3ncnc(N)c23)n1] | I-222 | 12 | 520 | ¹H NMR (400 MHz, DMSO- d6) δ ppm 9.05 (br, 1H), 8.38 (d, J = 8 Hz, 1H), 8.11 (d, J = 4.8 Hz, 1H), 7.93-7.90 (m, 1H), 7.75 (d, J = 2.4 Hz, 1H), 7.515 (t, J = 7.2 Hz, 1H), 7.28 (br, 1H), 6.84 (d, J = 8.4 Hz, 1H), 6.63-6.60 (m, 1H), 6.555 (d, J = 2.4 Hz, 1H), 6.03 (d, J = 7.6 Hz, 1H), 5.64 (d, J = 4.4 Hz, 1H), 5.45 (d, J = 6.4 Hz, 1H), 4.65-4.61 (m, 1H), 4.28-4.23 (m, 2H), 4.16- 4.14 (m, 1H), 3.99-3.93 (m, 1H), 3.88 (s, 3H), 2.95-2.81 (m, 2H), 1.91-1.73 (m, 2H), 1.52- 1.38 (m, 2H). |
| 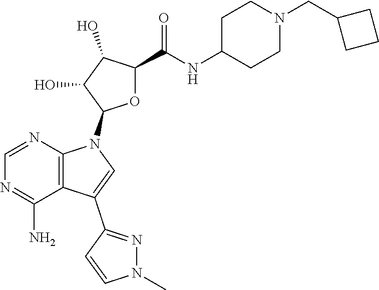 [SMILES: Cn1ccc(-c2cn([C@@H]3O[C@H](C(=O)NC4CCN(CC5CCC5)CC4)[C@@H](O)[C@H]3O)c3ncnc(N)c23)n1] | I-223 | 12 | 511 | |
| 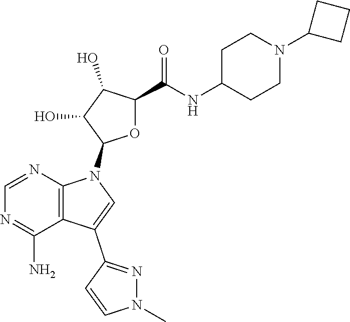 [SMILES: Cn1ccc(-c2cn([C@@H]3O[C@H](C(=O)NC4CCN(C5CCC5)CC4)[C@@H](O)[C@H]3O)c3ncnc(N)c23)n1] | I-224 | 12 | 497 | |
| 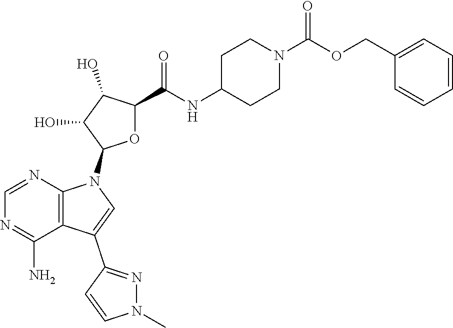 [SMILES: Cn1ccc(-c2cn([C@@H]3O[C@H](C(=O)NC4CCN(C(=O)OCc5ccccc5)CC4)[C@@H](O)[C@H]3O)c3ncnc(N)c23)n1] | I-225 | 12 | 577 | ¹H NMR (400 MHz, DMSO- d6) δ ppm 9.06 (br, 1H), 8.33 (d, J = 6.8 Hz, 1H), 8.05 (s, 1H), 7.94 (s, 1H), 7.75 (s, 1H), 7.40- 7.30 (s, 6H), 6.56 (d, J = 2.4 Hz, 1H), 6.06 (d, J = 7.2 Hz, 1H), 5.65 (d, J = 4.4 Hz, 1H), 5.47 (d, J = 6.4 Hz, 1H), 5.08 (s, 2H), 4.62-4.80 (m, 1H), 4.28 (s, 1H) 4.18-4.15 (m, 1H), 3.97-3.94 (m, 2H), 3.88-3.53 (m, 4H), 2.93 (br, 2H), 1.85-1.71 (m, 2H) 1.34-1.31 (m, 2H). |
| 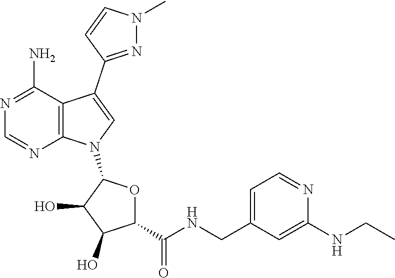 [SMILES: CCNc1cc(CNC(=O)[C@H]2O[C@@H](n3cc(-c4ccn(C)n4)c4c(N)ncnc43)[C@H](O)[C@@H]2O)ccn1] | I-226 | 12 | 494 | ¹H NMR (400 MHz, DMSO- d6) δ 9.35 (t, J = 5.7 Hz, 1H), 9.11-9.04 (br, 1H), 7.92 (d, J = 2.1 Hz, 1H), 7.85 (s, 1H), 7.82 (s, 1H), 7.74 (d, J = 2.3 Hz, 1H), 7.29 (dd, J = 8.6, 2.4 Hz, 2H), 6.54 (d, J = 2.3 Hz, 1H), 6.39-6.37 (m, 2H), 5.94 (d, J = 7.7 Hz, 1H), 5.68 (d, J = 4.3 Hz, 1H), 5.45 (d, J = 6.7 Hz, 1H), 4.63-4.58 (m, 1H), 4.32 (d, J = 1.3 Hz, 1H), 4.32- 4.12 (m, 3H), 3.88 (s, 3H), 3.24- 3.17 (m, 2H), 1.10 (t, J = 7.1 Hz, 3H). |
| 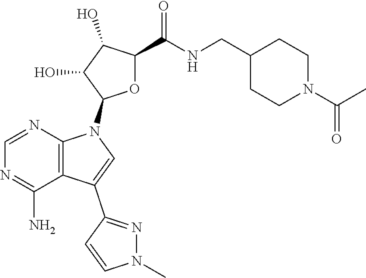 [SMILES: CC(=O)N1CCC(CNC(=O)[C@H]2O[C@@H](n3cc(-c4ccn(C)n4)c4c(N)ncnc43)[C@H](O)[C@@H]2O)CC1] | I-227 | 12 | 499 | ¹H NMR (400 MHz, DMSO- d6) δ 7.07 (s, 1H), 8.94 (t, J = 5.6 Hz, 1H), 8.12 (d, J = 1.2 Hz, 1H), 7.90 (s, 1H), 7.76 (d, J = 2.4 Hz, 1H), 7.31 (s, 1H), 6.57 (d, J = 2.4 Hz, 1H), 5.57 (d, J = 8 Hz, 1H), 5.65 (d, J = 2.8 Hz, 1H), 5.45 (d, J = 6.8 Hz, 1H), 4.65-4.61 (m, 1H), 4.33 (s, 1H), 4.30 (d, J = 1.6 Hz, 2H), 4.13 (s, J = 4.4 Hz, 1H), 3.88 (s, 3H), 3.75 (d, J = 9.6 Hz, 1H), 3.10 (t, J = 6.4 Hz, 2H), 2.93 (t, J = 2.4 Hz, 1H), 2.44-2.32 (m, 2H), 1.95 (s, 3H, 1.71-1.61 (m, 2H), 1.07-0.92 (m, 2H). |
| 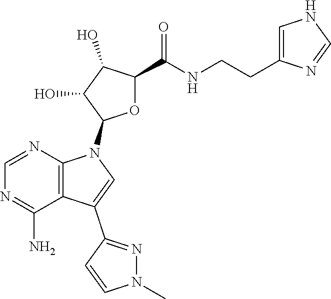 [SMILES: Cn1ccc(-c2cn([C@@H]3O[C@H](C(=O)NCCc4c[nH]cn4)[C@@H](O)[C@H]3O)c3ncnc(N)c23)n1] | I-228 | 12 | 454 | ¹H NMR (400 MHz, DMSO- d6) δ 12.13-11.65 (br, 1H), 9.18-8.76 (m, 3H), 8.05 (s, 1H), 7.96-7.89 (m, 1H), 7.74 (d, J = 2.0 Hz, 1H), 7.48 (s, 1H), 7.34-7.20 (m, 1H), 6.89- 6.74 (m, 1H), 6.59 (d, J = 2.1 Hz, 1H), 6.01 (d, J = 6.7 Hz, 1H), 4.59-4.56 (m, 1H), 4.27 (m, 1H), 4.13-4.12 (m, 2H), 3.89 (s, 3H), 2.78-2.60 (m, 4H). |
| 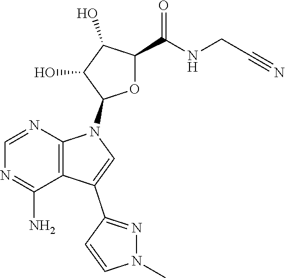 [SMILES: Cn1ccc(-c2cn([C@@H]3O[C@H](C(=O)NCC#N)[C@@H](O)[C@H]3O)c3ncnc(N)c23)n1] | I-229 | 12 | 399 | ¹H NMR (400 MHz, DMSO- d6) δ 9.76 (t, J = 5.5 Hz, 1H), 9.17-9.05 (br, 1H), 8.19 (s, 1H), 7.86 (s, 1H), 7.76 (d, J = 2.2 Hz, 1H), 7.43-7.29 (br, 1H), 6.60 (d, J = 2.3 Hz, 1H), 6.00 (d, J = 7.7 Hz, 1H), 5.76 (d, J = 4.5 Hz, 1H), 5.51 (d, J = 6.6 Hz, 1H), 4.60-4.55 (m, 1H), 4.37 (m, 1H), 4.31-4.30 (m, 2H), 4.18-4.16 (m, 1H), 3.89 (s, 3H). |
| 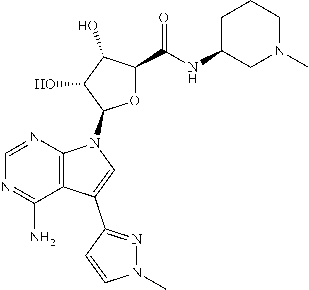 [SMILES: CN1CCC[C@H](NC(=O)[C@H]2O[C@@H](n3cc(-c4ccn(C)n4)c4c(N)ncnc43)[C@H](O)[C@@H]2O)C1] | I-230 | 15 | 457 | ¹H NMR (400 MHz, CD3OD) δ ppm 9.02 (brs, 1H), 8.11-8.07 (s, 1H), 7.92 (s, 1H), 7.75 (d, J = 1.6 Hz, 1H), 7.29 (s, 1H), 6.57 (d, J = 2.4 Hz, 1H), 6.07 (d, J = 7.6 Hz, 1H), 5.63 (d, J = 4.4 Hz, 1H), 5.46 (d, J = 6.8 Hz, 1H) 4.65-4.60 (m, 1H), 4.28 (d, J = 1.6 Hz, 1H), 4.17-4.15 (m, 1H), 3.89 (s, 3H), 3.86- 3.81 (m, 1H), 2.67-2.58 (m, 1H), 2.10 (s, 3H), 1.93-1.23 (m, 8H). |
| 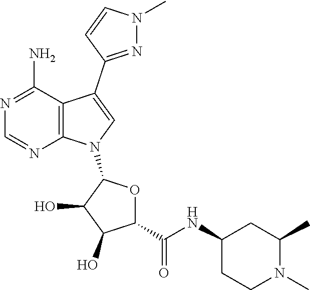 [SMILES: C[C@@H]1C[C@H](NC(=O)[C@H]2O[C@@H](n3cc(-c4ccn(C)n4)c4c(N)ncnc43)[C@H](O)[C@@H]2O)CCN1C] | I-231 | 15 | 471 | ¹H NMR (400 MHz, DMSO- d6) δ 9.09 (s, 1H), 8.32 (s, 1H), 8.20-8.03 (m, 1H), 7.96-7.88 (m, 1H), 7.83-7.66 (m, 1H), 6.72-6.43 (m, 1H), 6.02 (d, J = 7.6 Hz, 1H), 5.65 (d, J = 4.3 Hz, 1H), 5.50-5.40 (m, 1H), 4.70-4.60 (m, 1H), 4.40-4.30 (m, 1H), 4.20-4.10 (m, 1H), 3.89 (d, J = 2.3 Hz, 3H), 3.68 (s, 1H), 2.79 (s, 1H), 2.17 (s, 3H), 1.85-7.75 (m, 1H), 1.72- 1.60 (m, 1H), 1.50-1.36 (m, 1H), 1.34-1.10 (m, 1H), 1.09- 0.80 (m, 5H). |
| 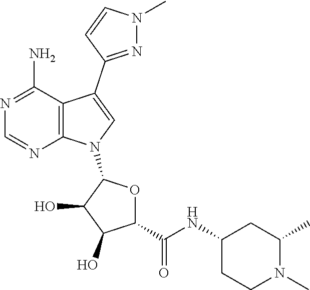 [SMILES: C[C@H]1C[C@@H](NC(=O)[C@H]2O[C@@H](n3cc(-c4ccn(C)n4)c4c(N)ncnc43)[C@H](O)[C@@H]2O)CCN1C] | I-232 | 15 | 471 | |
| 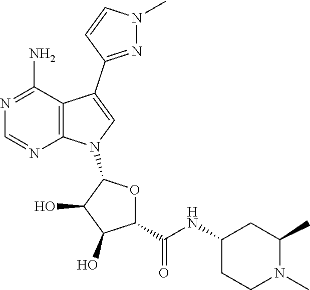 [SMILES: C[C@@H]1C[C@@H](NC(=O)[C@H]2O[C@@H](n3cc(-c4ccn(C)n4)c4c(N)ncnc43)[C@H](O)[C@@H]2O)CCN1C] | I-233 | 15 | 471 | ¹H NMR (400 MHz, DMSO- d6) δ ppm 9.04 (s, 1H), 8.06 (s, 1H), 7.96 (s, 1H), 7.95 (s, 1H), 7.75 (d, J = 2 Hz, 1H), 7.29 (s, 1H), 6.56 (d, J = 2 Hz, 1H), 6.11 (d, J = 7.2 Hz, 1H), 5.61 (d, J = 3.6 Hz, 1H), 5.48 (d, J = 6.4 Hz, 1H), 4.63 (s, 1H), 4.33 (d, J = 2 Hz, 1H), 4.16 (s, 1H), 3.96 (s, 1H), 3.91 (s, 3H), 2.39- 2.16 (m, 3H), 2.13 (s, 3H), 1.75-1.40 (m, 4H), 1.37 (d, J = 3.6 Hz, 3H). |
| 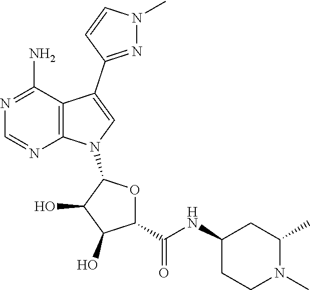 [SMILES: C[C@H]1C[C@H](NC(=O)[C@H]2O[C@@H](n3cc(-c4ccn(C)n4)c4c(N)ncnc43)[C@H](O)[C@@H]2O)CCN1C] | I-234 | 15 | 471 | ¹H NMR (400 MHz, DMSO- d6) δ 9.04 (s, 1H), 8.09 (s, 1H), 7.97 (s, 1H), 7.89 (s, 1H), 7.76 (d, J = 2.4 Hz, 1H), 7.29 (s, 1H), 6.58 (d, J = 2.4 Hz, 1H), 5.60 (d, J = 4.4 Hz, 1H), 5.48 (d, J = 6.4 Hz, 1H), 4.65-4.61 (m, 1H), 4.33 (d, J = 2 Hz, 1H), 4.21-4.18 (m, 1H), 3.94 (s, 1H), 3.93 (s, 3H), 2.37-2.31 (m, 3H), 2.21 (s, 3H), 1.65- 1.51 (m, 4H), 0.92 (d, J = 5.6 Hz, 3H). |
| 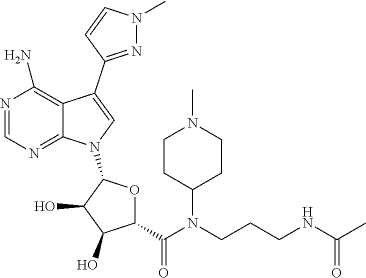 [SMILES: CC(=O)NCCCN(C(=O)[C@H]1O[C@@H](n2cc(-c3ccn(C)n3)c3c(N)ncnc32)[C@H](O)[C@@H]1O)C1CCN(C)CC1] | I-235 | 21 | 556 | ¹H NMR (400 MHz, DMSO- d6) δ ppm 8.94 (s, 1H), 8.06 (d, 1H), 7.85 (d, J = 12 Hz, 1H), 7.75 (dd, J = 9.2 Hz, 2.4 Hz, 1H), 7.19 (s, 1H), 6.50-6.48 (m, 1H), 6.29-6.25 (m, 1H), 6.29-6.27 (m, 1H), 5.97-5.73 (m, 1H), 5.55-5.50 (m, 1H), 4.70 (dd, J = 8 Hz, J = 2 Hz, 1H), 4.50-4.39 (m, 1H), 4.39- 4.21 (m, 1H), 4.06 (s, 3H), 3.26-3.13 (m, 1H), 3.00-3.26 (m, 2H), 2.82-2.74 (m, 2H), 2.67 (d, J = 2 Hz, 3H), 2.13 (d, J = 9.2 Hz, 3H), 2.09-1.45 (m, 9H). |

### Biological Assays

METTL3-14 Standard Enzyme Assay

Assays were performed in a 25 μl-volume in 384-well V-bottom polypropylene microplates (Greiner Bio-One, cat. No. 781280) at ambient temperature. Optimized 1× assay buffer was 20 mM HEPES pH 7.5, 50 mM KCl, 250 μM MgCl2, 1 mM DTT, 0.01% Tween, 0.01% BSG, 0.004 U/μl RNAseOUT (cat. No. 10777019, ThermoFisher Scientific, Waltham, MA). For compound screening METTL3/METTL14 (final concentration, f.c.=2.5 nM) was added using a Multidrop Combi (ThermoFisher Scientific, Waltham, MA) and preincubated for 5 min. Reactions were started by adding 3′ biotinylated RNA (UCUGGACUAAA-biotin) (f.c.=100 nM) and 3H-SAM (f.c.=100 nM) substrates. Reactions proceeded for 30 minutes and were quenched with excess non-radioactive SAM (f.c.=15 μM). The reactions were then transferred to streptavidin-coated flashplates and incubated for 2 hours at 25° C. Following two washing cycles with 0.1% Tween-20, the plates were sealed and read on a TopCount (PerkinElmer, Waltham, MA) plate-based scintillation counter. For determination of kinetic parameters, reaction times were optimized so that measurements were taken during the initial velocity phase of the reaction.

METTL1 Assay

Assays were performed in a 25 μl-volume in 384-well V-bottom polypropylene microplates (Greiner Bio-One, cat. No. 781280) at ambient temperature. Optimized 1× assay buffer was 20 mM HEPES pH 7.5, 50 mM KCl, 250 μM MgCl2, 1 mM DTT, 0.01% Tween, 0.01% BSG, 0.004 U/μl RNAseOUT (cat. No. 10777019, ThermoFisher Scientific, Waltham, MA). For compound screening METTL1/WDR4 (final concentration, f.c.=6.25 nM) was added using a Multidrop Combi (ThermoFisher Scientific, Waltham, MA) and preincubated for 5 min. Reactions were started by adding 3′ biotinylated RNA (GCCGAGAUAGCUCAGUUGGGAGAGCGUUAGACUGAAGAUCUAAAGGUCCCUG GUUCAAUCCCGGGUUUCGGCA-biotin) (f.c.=25 nM) and 3H-SAM (f.c.=60 nM) substrates. Reactions proceeded for 20 minutes and were quenched with excess non-radioactive SAM (f.c.=15 μM). The reactions were then transferred to streptavidin-coated flashplates and incubated for 2 hours at 25° C. Following two washing cycles with 0.1% Tween-20, the plates were sealed and read on a TopCount (PerkinElmer, Waltham, MA) plate-based scintillation counter. For determination of kinetic parameters, reaction times were optimized so that measurements were taken during the initial velocity phase of the reaction.

METTL16 Assay

Assays were performed in a 25 μl-volume in 384-well V-bottom polypropylene microplates (Greiner Bio-One, cat. No. 781280) at ambient temperature. Optimized 1× assay buffer was 20 mM HEPES pH 7.5, 50 mM KCl, 1 mM DTT, 0.01% Tween, 0.01% BSG, 0.004 U/μl RNAseOUT (cat. No. 10777019, ThermoFisher Scientific, Waltham, MA). For compound screening METTL16 (final concentration, f.c.=100 nM) was added using a Multidrop Combi (ThermoFisher Scientific, Waltham, MA) and preincubated for 5 min. Reactions were started by adding 3′ biotinylated RNA (CGAUACAGAGAAGAUUAGCAUACGCAAAUUCGUGAAGCG-biotin) (f.c.=50 nM), 3H-SAM (f.c.=200 nM) and non-radiolabeled SAM (f.c.=800 nM) substrates. Reactions proceeded for 20 minutes and were quenched with excess non-radioactive SAM (f.c.=100 μM). The reactions were then transferred to streptavidin-coated flashplates and incubated for 2 hours at 25° C. Following two washing cycles with 0.1% Tween-20, the plates were sealed and read on a TopCount (PerkinElmer, Waltham, MA) plate-based scintillation counter. For determination of kinetic parameters, reaction times were optimized so that measurements were taken during the initial velocity phase of the reaction.

PRMT5 Assay

Assays were performed in a 25 μl-volume in 384-well V-bottom polypropylene microplates (Greiner Bio-One, cat. No. 781280) at ambient temperature. Optimized 1× assay buffer was 20 mM Tris-HCl pH 8.0, 1 mM DTT, 0.01% Tween, 0.01%. For compound screening PRMT5-MEP50 (final concentration, f.c.=2.5 nM) was added using a Multidrop Combi (ThermoFisher Scientific, Waltham, MA) and preincubated for 5 min. Reactions were started by adding 3′ biotinylated histone H4 peptide acetylated on serine 1 (Ac-SGRGKGGKGLGKGGAKRHRKVGGK-Biotin) (f.c.=100 nM) and 3H-SAM (f.c.=250 nM) substrates. Reactions proceeded for 60 minutes and were quenched with excess non-radioactive SAM (f.c.=15 μM). The reactions were then transferred to streptavidin-coated flashplates and incubated for 2 hours at 25° C. Following two washing cycles with 0.1% Tween-20, the plates were sealed and read on a TopCount (PerkinElmer, Waltham, MA) plate-based scintillation counter. For determination of kinetic parameters, reaction times were optimized so that measurements were taken during the initial velocity phase of the reaction.

m⁶A-mRNA LC-MS/MS Assay

5×10⁶ MOLM-13 (DSMZ) cells were seeded into 10 cm dishes in RPMI 1640 media containing 10% fetal bovine serum and placed in a humidified tissue culture incubator at 37° C. overnight. Compounds were resuspended in 100% DMSO and dosed into each dish at a fixed concentration to comprise an 8-point dose response with a 4-fold serial dilution ranging from 25 μM to 1.5 nM in 0.25% DMSO final and allowed to incubate for 24 hours in a humidified tissue culture incubator at 37° C. Cells were harvested by centrifugation followed by mRNA extraction using DIRECT Dynabeads mRNA DIRECT kit (Life Technologies). mRNA was quantified on NanoDrop spectrophotometer (Thermo Fisher Scientific) and digested into single nucleosides using Nucleoside Digestion Mix (New England Biolabs). Nucleosides are quantified with retention time on a BEH C₁₈ column (Waters) and the nucleoside-to-base ion mass transition of 282.1-150.1 (m⁶A) and 268-136 (A) on an API 6500+ triple quadrupole mass spectrometer. Quantification is performed in comparison with the standard curve, obtained from pure nucleoside standards (Selleck Chemicals) running with the same batch of samples. Percentage m⁶A in cellular mRNA is calculated as 100*(m⁶A/A).

Cell Proliferation Assay

MOLM-13 (DSMZ) cells were seeded at 1000 cells per well in a volume of 44 μL in a Falcon 384-well tissue culture treated clear bottom microplate in RPMI 1640 media containing 10% fetal bovine serum using a Multidrop Combi (ThermoFisher Scientific). Cells were incubated overnight at 37° C. in a humidified tissue culture incubator. The Mosquito® HTS Liquid Handler was used to make a compound/media intermediate plate by aliquoting 1 μL of compound from the initial compound dilution plate (concentrations ranging from 10.0 mM to 38.0 nM in 100% DMSO) into a V bottom 384-well screen matrix plate containing 49 μL of media containing the appropriate serum (50-fold dilution, 2% DMSO). The Apricot liquid handling system was used to transfer 6.2 μL compounds from the intermediate plate into the Falcon 384-well tissue culture plate containing 44 μL cells (10-point, 4-fold dilution spanning concentrations 25.0 μM to 95.1 μM, 0.25% DMSO final), and placed in a humidified tissue culture incubator at 37° C. After 48 hours, 25 μL of Cell Titer-Glo reagent (Promega) was added to each well using a Multidrop Combi. The plate was protected from light and placed on an IKA plate shaker for at 300 rpm for 10 minutes at room temperature. The plate was read on an EnVision plate reader (Perkin Elmer) using the Ultra Sensitive Luminescence protocol. Data analysis was performed by normalizing the raw luminescence units to an average of the positive control values for staurosporine (100% cell death) and the negative control values for DMSO (0% cell death). An IC₅₀ was calculated using a 4-parameter logistic nonlinear regression model in GraphPad Prism.

FLT3/FLT3-ITD LanthaScreen Assays

Assays were performed in a 10 μl-volume in 384-well polypropylene microplates (Perkin Elmer Proxiplate, cat. No. 6059480) at ambient temperature. Optimized 1× assay buffer was 50 mM Hepes pH 7.5, 2 mM DTT, 0.01% Tween, 0.01% BSA, 10 mM MgCl₂. For compound screening FLT3 (final concentration, f.c.=20 μM) or FLT3-ITD (f.c.=120 μM) was added using a Multidrop Combi (ThermoFisher Scientific, Waltham, MA) and preincubated for 10 min. Reactions were started by adding fluorescein-poly GT (f.c.=200 nM) and ATP (f.c.=57 μM) substrates. Reactions proceeded for 40 minutes. The reactions were stopped with a detection mixture of EDTA (f.c.=20 mM) and terbium-conjugated anti-phoso tyrosine antibody pY20 (f.c.=2 nM). The plates were sealed and read on an Envision (PerkinElmer, Waltham, MA) using 340 nM excitation and emission reads at 495 and 520 nM.

FLT3/FLT3-ITD Caliper Assays

Assays were performed in a 20 μl-volume in 384-well polypropylene microplates (Perkin Elmer Proxiplate, cat. No. 6059480) at ambient temperature. Optimized 1× assay buffer was 50 mM Hepes pH 7.5, 2 mM DTT, 0.01% Tween, 0.01% BSA, 10 mM MgCl₂. For compound screening FLT3 (final concentration, f.c.=0.9 nM) or FLT3-ITD (f.c.=7 nM) was added using a Multidrop Combi (ThermoFisher Scientific, Waltham, MA) and preincubated for 10 min. Reactions were started by adding fluorescein-peptide (sequence 5-FAM-EAIYAAPFAKKK-CONH2) (f.c.=300 μM) and ATP (FLT3 f.c.=97 μM FLT3-ITD f.c.=146 μM) substrates. Reactions proceeded for 60 minutes. The reactions were stopped with LabChip ProfilerPro Separation Buffer containing EDTA (f.c.=20 mM). The plates were read on a Caliper microfluidics reader (PerkinElmer, Waltham, MA).

Table 3 shows IC₅₀ values for selected compounds of this invention measured in the METTL3 biochemical assay, PRMT5 biochemical assay, METTL1 biochemical assay, METTL16 biochemical assay, m⁶A cellular assay and MOLM-13 cell proliferation assay described above, wherein each compound number corresponds to the compound numbering set forth in Examples 1-32 of table 1 disclosed above. For METTL3, PRMT5, METTL1 and METTL16 biochemical assays, “A” represents an IC₅₀ of less than 10 nM (i.e., IC₅₀<10 nM); “B” represents an IC₅₀ of equal to or greater than 10 nM and lesser than 100 nM (i.e., 10 nM≤IC₅₀<100 nM); “C” represents an IC₅₀ of equal to or greater than 100 nM and less than 1000 nM (i.e., 100 nM≤IC₅₀<1000 nM); and “D” represents an IC₅₀ of equal to or greater than 1000 nM (i.e., IC₅₀≥1000 nM). For m⁶A cellular assay and MOLM-13 cell proliferation assay, “*” represents an IC₅₀ of equal to or greater than 10 μM (i.e., IC₅₀≥10 μM); “**” represents an IC₅₀ value of equal to or greater than 1 μM and less than 10 μM (i.e., 1 μM≤IC₅₀<10 μM); and “***” represents an IC₅₀ of less than 1 μM (i.e., IC₅₀<1 μM).

TABLE 3

| | | | | | | MOLM- |
|---|---|---|---|---|---|---|
| Com- | | | | | | 13 cell |
| pound | | | | | | prolif- |
| No. | METTL3 | PRMT5 | METTL1 | METTL16 | m⁶A | eration |
| I-1 | D | D | | | | |
| I-2 | C | B | | | | |
| I-3 | D | D | | | | |
| I-4 | D | B | | | | |
| I-5 | D | D | | | | |
| I-6 | D | A | | | | |
| I-7 | D | B | | | | |
| I-8 | C | A | | | | |
| I-9 | C | D | | | | |
| I-10 | A | B | | | *** | |
| I-11 | C | C | | | | |
| I-12 | D | D | | | | |
| I-13 | A | D | | | | |
| I-14 | B | | | | | |
| I-15 | B | | | | | |
| I-16 | A | | | | | |
| I-17 | D | | | | | |
| I-18 | B | D | | | | |
| I-19 | C | B | | | | |
| I-20 | C | D | | | | |
| I-21 | C | D | | | | |
| I-22 | C | C | | | | |
| I-23 | D | C | | | | |
| I-24 | C | B | | | | |
| I-25 | C | B | | | | |
| I-26 | C | D | | | | |
| I-27 | D | C | | | | |
| I-28 | D | D | | | | |
| I-29 | C | D | | | | |
| I-30 | A | C | | | | |
| I-31 | A | A | | | | |
| I-32 | A | A | | | *** | |

Table 4 shows IC₅₀ values for selected compounds of this invention measured in the METTL3 biochemical assay, PRMT5 biochemical assay, FLT3/FLT3-ITD Lanthascreen assay and FLT3/FLT3-ITD Calipher assay described above, wherein each compound number corresponds to the compound numbering set forth in Example 31 of table 1 and Examples 33-235 of table 2 disclosed above. For METTL3, PRMT5, FLT3/FLT3-ITD Lanthascreen assay and FLT3/FLT3-ITD Calipher assays, “A” represents an IC₅₀ of less than 10 nM (i.e., IC₅₀<10 nM); “B” represents an IC₅₀ of equal to or greater than 10 nM and lesser than 100 nM (i.e., 10 nM≤IC₅₀<100 nM); “C” represents an IC₅₀ of equal to or greater than 100 nM and less than 1000 nM (i.e., 100 nM≤IC₅₀<1000 nM); and “D” represents an IC₅₀ of equal to or greater than 1000 nM (i.e., IC₅₀≥1000 nM).

TABLE 4

| | | | | FLT3- | | |
|---|---|---|---|---|---|---|
| | | | FLT3 | ITD | | FLT3- |
| Com- | | | Lantha- | Lantha- | FLT3 | ITD |
| pound | | | screen | screen | Calipher | Calipher |
| No. | METTL3 | PRMT5 | assay | assay | assay | assay |
| I-31 | | | D | D | | |
| I-33 | A | D | | | D | D |
| I-34 | B | D | | | D | D |
| I-35 | A | B | D | D | | |
| I-36 | D | C | | | D | D |
| I-37 | D | D | | | D | D |
| I-38 | A | C | | | | |
| I-39 | C | D | | | | |
| I-40 | C | C | | | D | D |
| I-41 | D | D | | | | |
| I-42 | D | C | D | D | | |
| I-43 | C | D | D | D | | |
| I-44 | C | B | D | D | | |
| I-45 | D | D | D | D | | |
| I-46 | D | B | D | D | | |
| I-47 | D | B | | | | |
| I-48 | D | B | | | | |
| I-49 | C | B | | | | |
| I-50 | D | C | | | | |
| I-51 | C | C | | | | |
| I-52 | B | C | | | C | C |
| I-53 | C | C | D | D | | |
| I-54 | D | C | D | D | | |
| I-55 | D | D | | | | |
| I-56 | C | C | D | D | | |
| I-57 | C | C | D | D | | |
| I-58 | D | B | | | | |
| I-59 | C | A | | | | |
| I-60 | C | D | | | D | D |
| I-61 | A | B | C | C | | |
| I-62 | C | A | D | D | | |
| I-63 | D | | | | | |
| I-64 | D | | | | | |
| I-65 | D | | | | | |
| I-66 | D | | | | | |
| I-67 | D | | | | | |
| I-68 | D | | | | | |
| I-69 | D | | | | | |
| I-70 | D | | | | | |
| I-71 | D | | | | | |
| I-72 | D | | | | | |
| I-73 | D | | | | | |
| I-74 | D | | | | | |
| I-75 | D | | | | | |
| I-76 | D | | | | | |
| I-77 | D | | | | | |
| I-78 | D | | | | | |
| I-79 | D | | | | | |
| I-80 | D | | | | | |
| I-81 | D | | | | | |
| I-82 | D | | | | | |
| I-83 | D | | | | | |
| I-84 | D | | | | | |
| I-85 | D | | | | | |
| I-86 | D | | | | | |
| I-87 | D | | | | | |
| I-88 | D | | | | | |
| I-89 | D | | | | | |
| I-90 | D | | | | | |
| I-91 | D | | | | | |
| I-92 | D | | | | | |
| I-93 | D | | | | | |
| I-94 | C | | | | | |
| I-95 | C | | | | | |
| I-96 | C | | | | | |
| I-97 | C | | | | | |
| I-98 | C | | | | | |
| I-99 | C | | | | | |
| I-100 | C | | | | | |
| I-101 | C | | | | | |
| I-102 | C | | | | | |
| I-103 | C | | | | | |
| I-104 | C | | | | | |
| I-105 | C | | | | | |
| I-106 | C | | | | | |
| I-107 | C | | | | | |
| I-108 | C | | | | | |
| I-109 | C | | | | | |
| I-110 | C | | | | | |
| I-111 | C | | | | | |
| I-112 | C | | | | | |
| I-113 | C | | | | | |
| I-114 | C | | | | | |
| I-115 | C | | | | | |
| I-116 | C | | | | | |
| I-117 | C | | | | | |
| I-118 | C | | | | | |
| I-119 | C | | | | | |
| I-120 | C | | | | | |
| I-121 | C | | | | | |
| I-122 | C | | | | | |
| I-123 | C | | | | | |
| I-124 | C | | | | | |
| I-125 | C | | | | | |
| I-126 | C | | | | | |
| I-127 | C | | | | | |
| I-128 | C | | | | | |
| I-129 | C | | | | | |
| I-130 | C | | | | | |
| I-131 | C | | | | | |
| I-132 | C | | | | | |
| I-133 | C | | | | | |
| I-134 | C | | | | | |
| I-135 | C | | | | | |
| I-136 | C | | | | | |
| I-137 | C | | | | | |
| I-138 | C | | | | | |
| I-139 | C | | | | | |
| I-140 | B | | | | | |
| I-141 | B | | | | | |
| I-142 | A | A | | | D | D |
| I-143 | C | B | D | D | | |
| I-144 | C | B | D | D | | |
| I-145 | C | A | D | D | | |
| I-146 | B | B | D | D | | |
| I-147 | D | A | D | D | | |
| I-148 | D | B | D | D | | |
| I-149 | B | A | D | D | | |
| I-150 | D | C | | | D | D |
| I-151 | D | D | D | D | | |
| I-152 | C | C | | | | |
| I-153 | D | B | | | | |
| I-154 | C | C | | | | |
| I-155 | C | B | | | | |
| I-156 | C | B | | | D | D |
| I-157 | D | C | | | D | D |
| I-158 | D | C | | | D | D |
| I-159 | D | D | D | D | | |
| I-160 | D | B | | | D | D |
| I-161 | C | C | | | D | D |
| I-162 | C | B | | | D | D |
| I-163 | C | B | | | D | D |
| I-164 | D | C | | | D | D |
| I-165 | C | C | | | D | D |
| I-166 | C | B | | | D | D |
| I-167 | C | C | | | D | D |
| I-168 | C | B | | | D | D |
| I-169 | C | C | | | D | D |
| I-170 | D | D | | | C | C |
| I-171 | D | D | | | D | D |
| I-172 | D | D | | | D | D |
| I-173 | C | | | | D | D |
| I-174 | D | D | D | D | | |
| I-175 | D | D | | | D | D |
| I-176 | C | C | | | D | D |
| I-177 | D | B | D | D | | |
| I-178 | C | B | D | D | | |
| I-179 | D | D | | | D | D |
| I-180 | D | D | | | D | D |
| I-181 | D | B | | | D | D |
| I-182 | D | C | | | D | D |
| I-183 | D | B | | | D | D |
| I-184 | C | B | D | D | | |
| I-185 | D | C | D | D | | |
| I-186 | D | D | D | D | | |
| I-187 | C | B | D | D | | |
| I-188 | C | C | D | D | | |
| I-189 | C | B | D | D | | |
| I-190 | D | D | D | D | | |
| I-191 | C | C | | | D | D |
| I-192 | C | C | | | D | D |
| I-193 | C | D | | | D | D |
| I-194 | B | C | D | D | | |
| I-195 | C | C | D | D | | |
| I-196 | D | C | | | D | D |
| I-197 | C | D | | | D | D |
| I-198 | C | B | | | D | D |
| I-199 | D | C | | | D | D |
| I-200 | D | D | | | | |
| I-201 | B | B | | | | |
| I-202 | C | D | D | D | | |
| I-203 | C | B | D | D | | |
| I-204 | D | D | | | D | D |
| I-205 | C | C | D | D | | |
| I-206 | C | C | | | | |
| I-207 | C | C | | | | |
| I-208 | C | C | | | | |
| I-209 | C | D | D | D | | |
| I-210 | C | B | D | D | | |
| I-211 | C | C | D | D | | |
| I-212 | C | C | D | D | | |
| I-213 | D | D | | | | |
| I-214 | D | D | | | | |
| I-215 | D | D | | | | |
| I-216 | C | B | | | | |
| I-217 | C | B | | | | |
| I-218 | C | D | | | | |
| I-219 | C | B | D | D | | |
| I-220 | C | A | D | D | | |
| I-221 | C | C | | | | |
| I-222 | C | D | | | | |
| I-223 | B | B | | | | |
| I-224 | C | C | | | | |
| I-225 | C | C | | | | |
| I-226 | C | C | | | D | D |
| I-227 | D | D | | | D | D |
| I-228 | C | C | | | D | D |
| I-229 | D | D | | | D | D |
| I-230 | D | B | | | | |
| I-231 | C | C | D | D | | |
| I-232 | C | C | | | | |
| I-233 | C | C | D | D | | |
| I-234 | C | C | D | D | | |
| I-235 | D | C | | | | |

In Vivo Studies

The following are various models of AML that will be used to assess the PK/PD relationship and efficacy of compounds in vivo.

### A. Subcutaneous Xenograft* Model:

Several human-derived AML cell lines will be tested in immunocompromised mice to elucidate the PK/PD relationship as well as the efficacy of compounds to inhibit tumor growth. Compounds will be administered to mice using an appropriate route of administration and dosing regimen at various concentrations and samples taken at various timepoints after dosing to evaluate plasma and tumoral exposure (pharmacokinetic measurements) as well as the effect on the m⁶A-mRNA pharmacodynamic biomarker extracted from tumors at varying timepoints. Body weight will be measured daily to assess tolerability.

### B. Disseminated Xenograft* Model:

Studies analogous to the ones described above will be conducted but rather as a disseminated model of disease achieved by tail-vein injection of various human AML cell lines. Cell lines may be luciferized for whole body imaging to assess disease burden at various doses and timepoints following drug administration. A Kaplan-Meier estimate will be used to assess survival over time. Other measurements of disease burden will be taken such as effects on composition of the bone marrow and spleen size. *note: xenograft includes both cell-line derived (CDX) and patient-derived (PDX) models

Various genetically engineered mouse models (GEMMs) of AML in immunocompotent mice will also be used for the in vivo PK/PD/efficacy studies described above.

1. A compound of formula (I′): 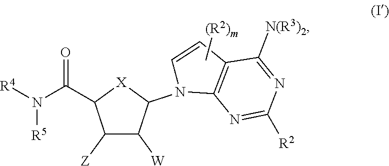
or a pharmaceutically acceptable salt thereof, wherein:
X is selected from O and CH₂;
R¹ is selected from H, C₁₋₆alkyl and —C(═O)—C₁₋₆alkyl;
Z is H and W is —OR¹, or Z is —OR¹ and W is selected from H, halo, —OR¹, C₁₋₆alkyl and —NH₂,
R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl, 5 to 6-membered heteroaryl, halo, —CN, —OR^{2a}, —N(R^{2a})₂, and —C(═O)N(R^{2a})₂, wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, —C₁₋₆alkyl-C₁₋₃alkoxy, C₁₋₆haloalkyl, C₃₋₈cycloalkyl, 4 to 7-membered heterocycloalkyl, phenyl, 5-to 6-membered heteroaryl, halo, —CN, —OR^{2a}, —C(═O)N(R^{2a})₂, and —N(R^{2a})₂;
R^{2a}, for each occurrence, is independently selected from H, C₁₋₆alkyl, C₃₋₈cycloalkyl, and 4 to 6-membered heterocycloalkyl;
R³, for each occurrence, is H or C₁₋₆alkyl optionally substituted with 1 to 3 substituents independently selected from C₃₋₆cycloalkyl, phenyl and halo;
R⁴ is H, C₁₋₆alkyl, C₃₋₈cycloalkyl, 4 to 6-membered heterocycloalkyl or 5 to 6-membered heteroaryl, wherein the C₁₋₆alkyl, C₃₋₈cycloalkyl, 4 to 6-membered heterocycloalkyl and 5 to 6-membered heteroaryl represented by R⁴ are each optionally substituted with 1 to 4 substituents independently selected from C₁₋₆alkyl, —CN, —N(R^{4a})₂, —OR^{4a}, and —C(O)OR^{4a};
R^{4a} is H or C₁₋₄alkyl optionally substituted with —OH or C₁₋₆alkoxy,
R⁵ is H, C₁₋₆alkyl, ring A, or —C₁₋₆alkylene-ring A, each of which is optionally substituted with 1 to 4 R⁶;
or R⁴ and R⁵ together with the N atom from which they are attached form a 4 to 10-membered heterocycloalkyl optionally containing an additional heteroatom selected from O, N and S, wherein the heterocycloalkyl is optionally substituted with 1 to 3 R⁶; or the heterocycloalkyl is optionally fused with a phenyl or a 5 to 6-membered heteroaryl;
ring A is C₃₋₈cycloalkyl, phenyl, 4 to 6-membered heterocycloalkyl, 7 to 10-membered spiro or bridged bicyclic heterocycloalkyl, 5 to 6-membered heteroaryl, or 8 to 10-membered bicyclic heteroaryl, each of which is optionally substituted with 1 to 4 R⁶;
R⁶, for each occurrence, is independently C₁₋₆alkyl, C₃₋₈cycloalkyl, phenyl, 4 to 7-membered heterocycloalkyl, 5 to 6-membered heteroaryl, halo, oxo, —CN, —N(R^{6a})₂, —OR^{6a}, —C(═O)R^{6a}, —C(═O)N(R^{6a})₂, —S(═O)₂R^{6a}, —S(═O)₂N(R^{6a})₂, —NR^{6a}C(═O)R^{6a}, —NR^{6a}C(═O)OR^{6a}, —NR^{6a}C(═O)N(R^{6a})₂, —NR^{6a}S(═O)₂R^{6a}, —C(═O)OR^{6a}, wherein the C₁₋₆alkyl, C₃₋₈cycloalkyl, 4 to 7-membered heterocycloalkyl, 5 to 6-membered heteroaryl and phenyl represented by R⁶ are each optionally substituted with 1 to 3 substituents independently selected from C₁₋₆alkyl, C₃₋₅cycloalkyl, 5 to 6-membered heterocycloalkyl optionally substituted with 1 to 2 oxo, phenyl, halo, —OR^{6a}, —N(R^{6a})₂, and —C(═O)N(R^{6a})₂, wherein the C₁₋₆alkyl is optionally substituted with 1 to 3 substitutents independently selected from halo and OH;
or two R⁶ together with the intervening atoms on ring A form a phenyl, 5 to 6-membered heteroaryl, or 4 to 7-membered heterocycloalkyl fused with ring A, each of which is optionally substituted with 1 to 3 substituents independently selected from C₁₋₆alkyl, halo, oxo, —OR^{6a}, —N(R^{6a})₂, and —C(═O)N(R^{6a})₂;
R^{6a}, for each occurrence, is independently selected from H, C₁₋₆alkyl, C₃₋₈cycloalkyl, 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆haloalkyl, C₁₋₆alkoxy, C₃₋₆cycloalkyl, 5 to 6-membered heterocycloalkyl, and phenyl; and
m is 1 or 2.

2. The compound of claim 1, wherein:
X is selected from O and CH₂;
R¹ is selected from H, C₁₋₆alkyl and —C(═O)—C₁₋₆alkyl;
Z is H and W is —OR¹, or Z is —OR¹ and W is selected from H, halo, —OR¹, C₁₋₆alkyl and —NH₂,
R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl, 5 to 6-membered heteroaryl, halo, —CN, —OR^{2a}, —N(R^{2a})₂, and —C(═O)N(R^{2a})₂, wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, —C₁₋₆alkyl-C₁₋₃alkoxy, C₁₋₆haloalkyl, C₃₋₈cycloalkyl, 4 to 7-membered heterocycloalkyl, phenyl, 5- to 6-membered heteroaryl, halo, —CN, —OR^{2a}, —C(═O)N(R^{2a})₂, and —N(R^{2a})₂;
R^{2a}, for each occurrence, is independently selected from H, C₁₋₆alkyl, C₃₋₈cycloalkyl, and 4 to 6-membered heterocycloalkyl;
R³, for each occurrence, is H or C₁₋₆alkyl optionally substituted with 1 to 3 substituents independently selected from C₃₋₆cycloalkyl, phenyl and halo;
R⁴ is H, C₁₋₆alkyl, C₃₋₈cycloalkyl, 4 to 6-membered heterocycloalkyl or 5 to 6-membered heteroaryl, wherein the C₁₋₆alkyl, C₃₋₈cycloalkyl, 4 to 6-membered heterocycloalkyl and 5 to 6-membered heteroaryl represented by R⁴ are each optionally substituted with 1 to 4 substituents independently selected from C₁₋₆alkyl, —CN, —N(R^{4a})₂, —OR^{4a}, and —C(O)OR^{4a};
R^{4a} is H or C₁₋₄alkyl optionally substituted with —OH or C₁₋₆alkoxy,
R⁵ is C₁₋₆alkyl, ring A, or —C₁₋₆alkylene-ring A, each of which is optionally substituted with 1 to 4 R⁶;
or R⁴ and R⁵ together with the N atom from which they are attached form a 4 to 10-membered heterocycloalkyl optionally containing an additional heteroatom selected from O, N and S, wherein the heterocycloalkyl is optionally substituted with 1 to 3 R⁶;
ring A is C₃₋₈cycloalkyl, phenyl, 4 to 6-membered heterocycloalkyl, 5 to 6-membered heteroaryl, or 8- to 10-membered bicyclic heteroaryl, each of which is optionally substituted with 1 to 4 R⁶;
R⁶, for each occurrence, is independently C₁₋₆alkyl, C₃₋₈cycloalkyl, phenyl, 4 to 7-membered heterocycloalkyl, 5 to 6-membered heteroaryl, halo, oxo, —CN, —N(R^{6a})₂, —OR^{6a}, —C(═O)R^{6a}, —C(═O)N(R^{6a})₂, —S(═O)₂R^{6a}, —S(═O)₂N(R^{6a})₂, —NR^{6a}C(═O)R^{6a}, —NR^{6a}C(═O)NR^{6a}, —NR^{6a}S(═O)₂R^{6a}, —C(═O)OR^{6a}, wherein the C₁₋₆alkyl, C₃₋₈cycloalkyl, 4 to 7-membered heterocycloalkyl, 5 to 6-membered heteroaryl and phenyl represented by R⁶ are each optionally substituted with 1 to 3 substituents independently selected from C₁₋₆alkyl, halo, —OR^{6a}, —N(R^{6a})₂, and —C(═O)N(R^{6a})₂, wherein the C₁₋₆alkyl is optionally substituted with 1 to 3 substitutents independently selected from halo and OH;
or two R⁶ together with the intervening atoms on ring A form a phenyl, 5 to 6-membered heteroaryl, or 4 to 6-membered heterocycloalkyl fused with ring A, each of which is optionally substituted with 1 to 3 substituents independently C₁₋₆alkyl, halo, —OR^{6a}, —N(R^{6a})₂, and —C(═O)N(R^{6a})₂;
R^{6a}, for each occurrence, is independently selected from H, C₁₋₆alkyl, C₃₋₈cycloalkyl, 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆haloalkyl, C₁₋₆alkoxy, C₃₋₆cycloalkyl; and
m is 1 or 2.

3. The compound of claim 1, wherein the compound is represented by formula (I): 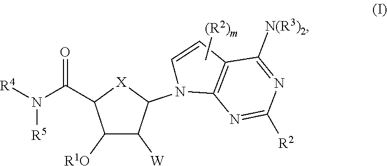
or a pharmaceutically acceptable salt thereof.

4. The compound of claim 3, wherein the compound is represented by formula (II): 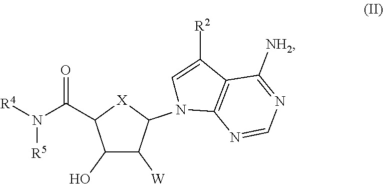
or a pharmaceutically acceptable salt thereof, wherein W is H, F or OH.

5. The compound of claim 4, wherein the compound is represented by the following formula: 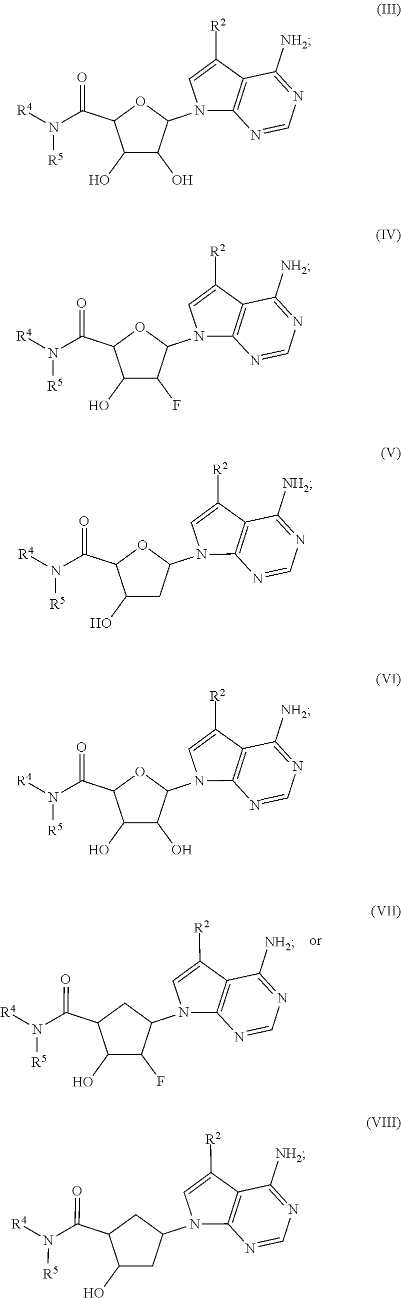
or a pharmaceutically acceptable salt thereof.

6. The compound of claim 5, wherein the compound is represented by the following formula: 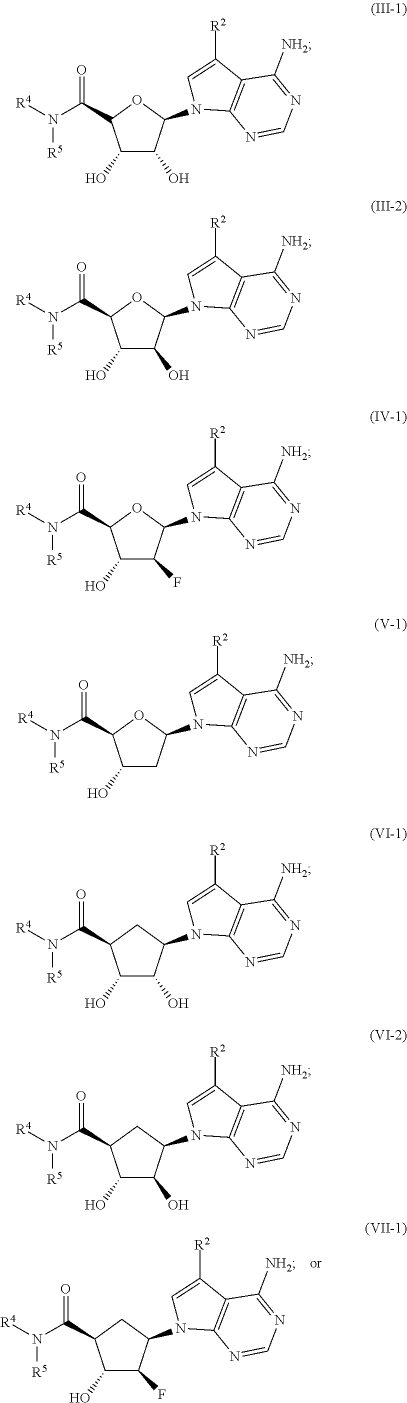 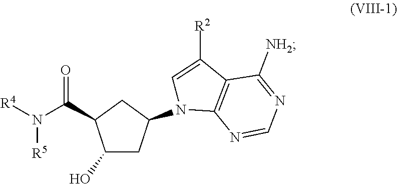
or a pharmaceutically acceptable salt thereof.

7. The compound of claim 5, wherein the compound is represented by the following formula: 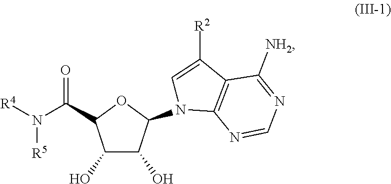
or a pharmaceutically acceptable salt thereof.

8. The compound of claim 1, or a pharmaceutically acceptable salt thereof, wherein (i) R² is H, halo, —CN, C₁₋₆alkyl, C₃₋₈cycloalkyl, 4 to 6-membered heterocycloalkyl, or 5 to 6-membered heteroaryl, wherein the C₁₋₆alkyl, C₃₋₈cycloalkyl, 4 to 6-membered heterocycloalkyl, and 5 to 6-membered heteroaryl are each optionally substituted with 1 to 3 substituents independently selected from halo, C₁₋₄alkyl, C₁₋₄haloalkyl and C₃₋₆cycloalkyl: (ii) R² is halo, —CN, cyclopentyl, pyrazolyl, or tetrahydrofuranyl; (iii) R² is H, —CN

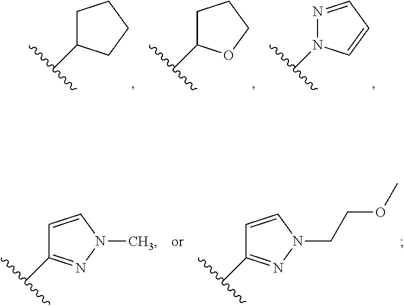

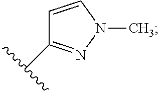 [SMILES: *c1ccn(C)n1]

9-15. (canceled)

16. The compound of claim 8, or a pharmaceutically acceptable salt thereof, wherein (i) R⁴ is H, C₁₋₄alkyl, or 5 to 6-membered heterocycloalkyl, wherein the C₁₋₄alkyl and 5 to 6-membered heterocycloalkyl are each optionally substituted with 1 or 2 substituents independently selected from C₁₋₃alkyl, —CN, N(R^{4a})₂, OR^{4a}, and C(O)OR^{4a}; and R^{4a} is H or C₁₋₃alkyl optionally substituted with —OH or C₁₋₃alkoxy; (ii) R⁴ is H, CH₂CH₃, 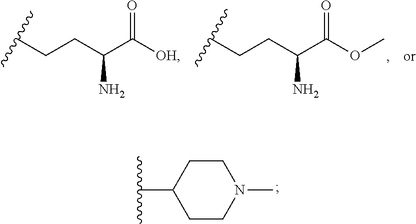
(iii) R⁴ is H, 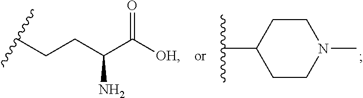
or (iv) R⁴ is H.

17-20. (canceled)

21. The compound of claim 16, or a pharmaceutically acceptable salt thereof, wherein Ring A is cyclopropyl, cyclobutyl, cyclopentyl, cyclohexanyl, azetidinyl, pyrrolidinyl, imidazolidinyl, tetrahydrofuranyl, tetrahydrothiophenyl, piperidinyl, piperazinyl, morpholinyl, tetrahydropyranyl, azaspiro[3.3]heptanyl, 2-azaspiro[3.3]heptanyl, 2-azaspiro[3.5]nonanyl, octahydroindolizinyl, (1R,5S)-8-methyl-8-azabicyclo[3.2.1]octanyl, phenyl, pyrazolyl, imidazolyl, tetrazolyl, isoxazolyl, thiazolyl, 1,3,4-thiadiazolyl, 1,2,4-thiadiazolyl, pyridinyl, pyrimidinyl, pyrazinyl, quinolinyl, isoquinolinyl, 1H-benzo[d]imidazolyl, benzo[d]oxazolyl, benzo[d]thiazolyl, or benzo[c][1,2,5]thiadiazolyl, each of which is optionally substituted with 1 to 3 R⁶.

22. The compound of claim 21, or a pharmaceutically acceptable salt thereof, wherein (i) R⁵ is H, C₁₋₄alkyl, —C₁₋₃alkylene-C₃₋₆cycloalkyl, —C₁₋₃alkylene-(4 to 6-membered heterocycloalkyl), —C₁₋₄alkylene-phenyl, —C₁₋₃-alkylene-(5 to 6-membered heteroaryl), C₃₋₆cycloalkyl, 4 to 6-membered heterocycloalkyl, 7 to 10-membered spiro or bridged bicyclic heterocycloalkyl, phenyl, 5 to 6-membered heteroaryl, or 8- to 10-membered bicyclic heteroaryl, each of which is optionally substituted with 1 to 3 R⁶; or R⁴ and R⁵ together with the N atom from which they are attached form a 5 to 7-membered heterocycloalkyl optionally containing an additional heteroatom selected from O and N, wherein the heterocycloalkyl is optionally substituted with 1 to 3 R⁶; or the heterocycloalkyl is optionally fused with a phenyl or a 5 to 6-membered heteroaryl;
(ii) R⁵ is H, —CH₃, —CH₂CH₃, —CH₂CH₂CH₃, —CH(CH₃)CH₃, —(CH₂)₃CH₃, —CH₂-cyclohexane, —CH₂CH₂-cyclohexane, —CH₂-azetidine, —CH₂-pyrrolidine, —(CH₂)₃-pyrrolidine, —CH₂CH₂-imidazolidine, —CH₂-tetrahydrofuran, —CH₂-piperidine, —CH(CH₃)-piperidine, —CH₂-(tetrahydropyran), —CH₂CH₂-(tetrahydropyran), —CH₂CH₂-morpholine, —CH₂-phenyl, —CH₂CH₂-phenyl, —CH(CH₃)-phenyl, —CH(CH₂CH₃)-phenyl, —CH₂CH(CH₃)-phenyl, —CH(CH₃)CH₂CH₂-phenyl, —CH₂—CH₂-imidazole, —(CH₂)₃-imidazole, —(CH₂)₃-tetrazole, —CH₂-isoxazole, —(CH₂)₃-isoxazole, —CH₂-pyridine, —CH₂—CH₂-pyridine, —CH₂-pyrazine, —CH₂—CH₂-pyrimidine, cyclopropyl, cyclobutyl, cyclopentyl, cyclohexanyl, azetidinyl, pyrrolidinyl, tetrahydrothiophenyl, piperidinyl, piperazinyl, tetrahydropyranyl, azaspiro[3.3]heptanyl, 2-azaspiro[3.3]heptanyl, 2-azaspiro[3.5]nonanyl, octahydroindolizinyl, (1R,5S)-8-methyl-8-azabicyclo[3.2.1]octanyl, phenyl, pyrazolyl, isoxazolyl, thiazolyl, 1,3,4-thiadiazolyl, 1,2,4-thiadiazolyl, pyridinyl, pyrimidinyl, pyrazinyl, quinolinyl, isoquinolinyl, 1H-benzo[d]imidazolyl, benzo[d]oxazolyl, benzo[d]thiazolyl, or benzo[c][1,2,5]thiadiazolyl, each of which is optionally substituted with 1 to 3 R⁶; or
R⁵ is —CH₂-naphthalene, —CH₂CH₂-naphthalene, naphthalenyl, 2,3-dihydro-1H-indenyl, 4,5,6,7-tetrahydro-1H-benzo[d]imidazolyl, 5,6,7,8-tetrahydroimidazo[1,2-a]pyridinyl, 4,5,6,7-tetrahydrobenzo[d]thiazolyl, 5,6,7,8-tetrahydroquinazolinyl, 1,2,3,4-tetrahydroquinolinyl, or 3,4-dihydro-2H-benzo[b][1,4]dioxepinyl, each of which is optionally substituted with 1 to 3 substituents independently selected from C₁₋₆alkyl, halo, oxo, —OR^{6a}, —N(R^{6a})₂, and —C(═O)N(R^{6a})₂;
(iii) R⁵ is 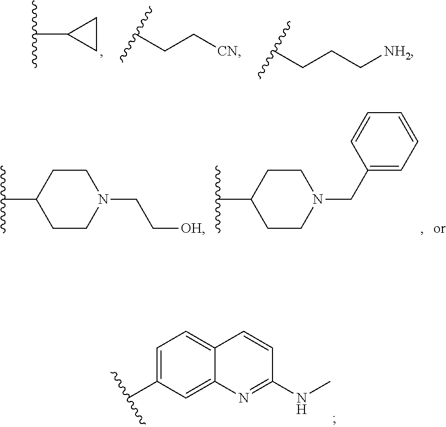
or
(iv) R⁵ is represented by the following formula: 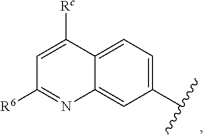
wherein R^{c} is selected from H, halo, C₁₋₄alkyl, —OR^{c1} and —N(R^{c1})₂, and R^{c1}, for each occurrence, is independently H or C₁₋₄alkyl optionally substituted with C₃₋₆cycloalkyl or phenyl.

23-27. (canceled)

28. The compound of claim 22, or a pharmaceutically acceptable salt thereof, wherein (i) R⁴ and R⁵ together with the N atom from which they are attached form pyrrolidine, piperidine or piperazine ring, each or which is optionally substituted with 1 to 3 R⁶, or each of which is optionally fused with a phenyl or a 5 to 6-membered heteroaryl; or (ii) R⁴ and R⁵ together with the N atom from which they are attached form one of the following cyclic rings:

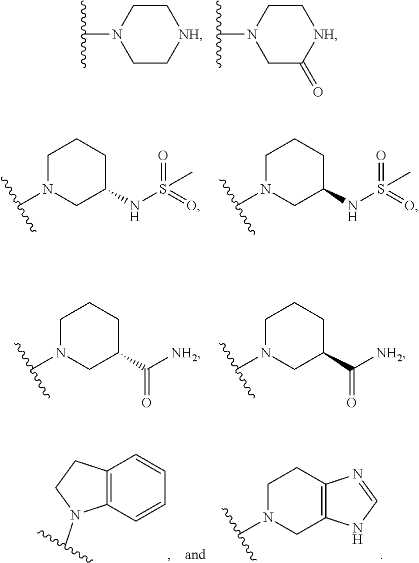

29. (canceled)

30. The compound of claim 28, or a pharmaceutically acceptable salt thereof, wherein:
(i) R⁶ is halo, oxo, C₁₋₄alkyl, —CN, —C(═O)R^{6a}, —C(═O)OR^{6a}, —C(═O)N(R^{6a})₂, —N(R^{6a})₂, —NR^{6a}C(═O)R^{6a}, —NR^{6a}C(═O)OR^{6a}, —NR^{6a}C(═O)NR^{6a}, —NR^{6a}S(═O)₂R^{6a}, —OR^{6a}, —S(═O)₂R^{6a}, —S(═O)₂N(R^{6a})₂, C₃₋₄cycloalkyl, 4 to 6-membered heterocycloalkyl, phenyl, or 5 to 6-membered heteroaryl, wherein the C₁₋₄alkyl, 4 to 6-membered heterocycloalkyl, and 5 to 6-membered heteroaryl, are each optionally substituted with 1 to 3 substituents independently selected from halo, C₁₋₃alkyl, C₃₋₄cycloalkyl, 5 to 6-membered heterocycloalkyl substituted with 2 oxo, phenyl, —OR^{6a}, and N(R^{6a})₂,
R^{6a} is H, C₁₋₃alkyl, C₃₋₆cycloalkyl, 4 to 6-membered heterocycloalkyl, phenyl, or 5 to 6-membered heteroaryl, wherein the C₁₋₃alkyl, C₃₋₆cycloalkyl, and 4 to 6-membered heterocycloalkyl are each optionally substituted with 1 to 3 substituents independently selected from halo, —OH, —CN, C₁₋₄alkyl, C₁₋₄haloalkyl, C₁₋₄alkoxy, C₃₋₆cycloalkyl, 5 to 6-membered heterocycloalkyl, and phenyl; or
(ii) R⁶ is Cl, F, Br, oxo, —CH₃, —CH₂CH₃, isopropyl, butyl, cyclobutyl, —CH₂(cyclobutane), —CF₃, —CH₂CHF₂, —CH₂CH₂OH, —CH₂CH₂OCH₃, —CN, —CH₂CH₂NH₂, —CH₂CH₂N(CH₃)₂, —(CH₂)₃N(CH₃)₂, 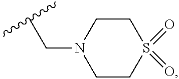 [SMILES: *CN1CCS(=O)(=O)CC1]
—C(═O)CH₃, —C(═O)OH, —C(═O)OCH₃, 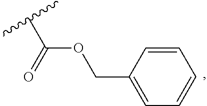 [SMILES: *C(=O)OCc1ccccc1]
—C(═O)NH₂, —C(═O)NHCH₃, 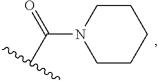 [SMILES: *C(=O)N1CCCCC1]
—NH₂, —NHCH₃, —N(CH₃)₂, —NHCH₂CH₃, —NH(CH₂-cyclopropane), —NH(cyclobutane), —NHCH₂CHF₂, —NHCH₂CH₂OCH₃, 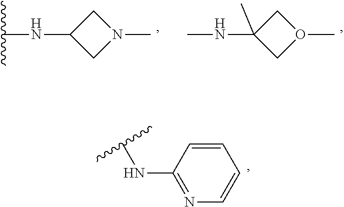
—NHC(═O)CH₃, —NHC(═O)NHCH₃, 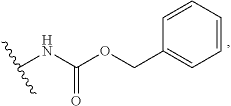 [SMILES: *NC(=O)OCc1ccccc1]
—NHS(═O)₂CH₃, azetidinyl, 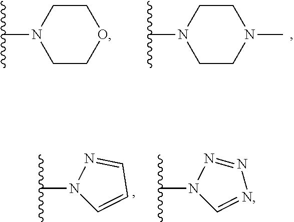
—OH, —OCH₃, —OCH(CH₃)₂, —OCHF₂, —OCF₃, —OCH₂CH₂OH, 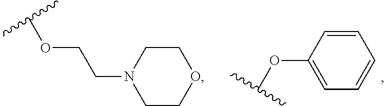 [SMILES: *OCCN1CCOCC1 ; *Oc1ccccc1]
—S(═O)₂CH₃, —S(═O)₂N(CH₃)₂, 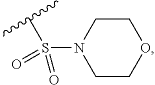 [SMILES: *S(=O)(=O)N1CCOCC1]
phenyl, benzyl, or pyridinyl.

31-33. (canceled)

34. The compound of claim 1, wherein the compound is represented by the following formula: 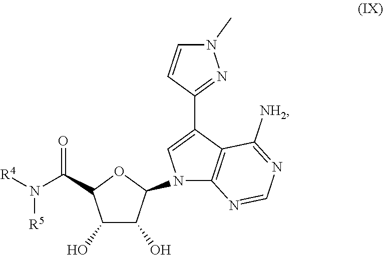
or a pharmaceutically acceptable salt thereof, wherein:
R⁴ is H, C₁₋₄alkyl, or 5 to 6-membered heterocycloalkyl, wherein the C₁₋₄alkyl and 5 to 6-membered heterocycloalkyl are each optionally substituted with 1 or 2 substituents independently selected from C₁₋₃alkyl, —CN, N(R^{4a})₂, OR^{4a}, and C(O)OR^{4a};
R^{4a} is H or C₁₋₃alkyl optionally substituted with —OH or C₁₋₃alkoxy;
R⁵ is C₁₋₄alkyl, 4 to 6-membered heterocycloalkyl, 8- to 10-membered bicyclic heteroaryl, or C₃₋₆cycloalkyl, each of which is optionally substituted with 1 or 2 R⁶;
R⁶ is —CN, —N(R^{6a})₂, or C₁₋₄alkyl optionally substituted with phenyl or —OR^{6a}; and
R^{6a} is H or C₁₋₃alkyl.

35. The compound of claim 34, or a pharmaceutically acceptable salt thereof, wherein:
R⁴ is H, 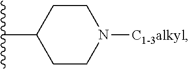
or C₁₋₄alkyl optionally substituted with 1 or 2 substituents independently selected from —N(R^{4a})₂ and C(O)OR^{4a};
R^{4a} is H or C₁₋₃alkyl;
R⁵ is cyclopropyl, 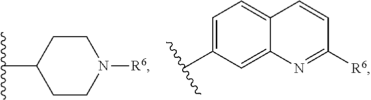
or C₁₋₄alkyl optionally substituted with —CN or —N(R^{6a})₂
R⁶ is —N(R^{6a})₂ or C₁₋₄alkyl optionally substituted with phenyl or —OR^{6a}; and
R^{6a} is H or C₁₋₃alkyl.

36. The compound of claim 35, or a pharmaceutically acceptable salt thereof, wherein:
(i) R⁴ is H or C₁₋₄alkyl optionally substituted with 1 or 2 substituents independently selected from —N(R^{4a})₂ and C(O)OR^{4a}; and
R⁵ is 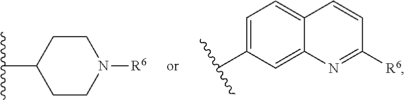
or
(ii) R⁴ is 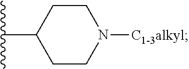
and
R⁵ is cyclopropyl, or C₁₋₄alkyl optionally substituted with —CN or —N(R^{6a})₂.

37. The compound of claim 36, wherein:
R^{4a} is H; and
R^{6a} is H or —CH₃.

38. The compound of claim 34, or a pharmaceutically acceptable salt thereof, wherein:
R⁴ is H, 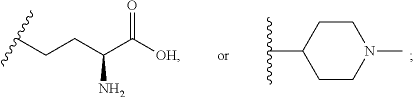
and
R⁵ is

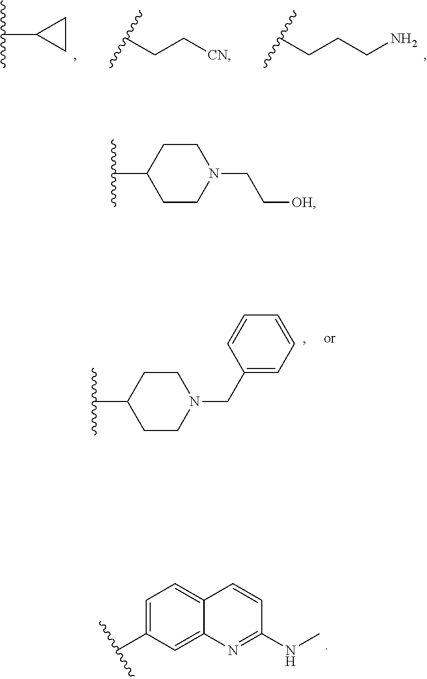

39. A pharmaceutical composition comprising a compound of claim 1, or a pharmaceutically acceptable salt thereof, and a pharmaceutically acceptable excipient.

40. A method of treating a disease or disorder responsive to inhibition of METTL3 activity in a subject comprising administering to the subject an effective amount of the compound according claim 1, or a pharmaceutically acceptable salt thereof, wherein the disease or disorder is an infection or cancer.

41-45. (canceled)
